SEQUENCE LISTING (43 sequences)

SEQ ID NO: 1 - protein, 179 aa, Homo sapiens
RFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 2 - protein, 201 aa, Homo sapiens
ARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 3 - protein, 117 aa, synthetic construct [sequence omitted]

SEQ ID NO: 4 - protein, 112 aa, synthetic construct
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKR

SEQ ID NO: 5 - protein, 10 aa, synthetic construct
GYTFSSYWIE

SEQ ID NO: 6 - protein, 18 aa, synthetic construct
GEILPGGGDTNYNEIFKG

SEQ ID NO: 7 - protein, 10 aa, synthetic construct
TRRVPIRLDY

SEQ ID NO: 8 - protein, 15 aa, synthetic construct
KASQSVDYEGDSFLN

SEQ ID NO: 9 - protein, 7 aa, synthetic construct
AASNLES

SEQ ID NO: 10 - protein, 9 aa, synthetic construct
QQSNEDPLT

SEQ ID NO: 11 - protein, 218 aa, synthetic construct
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 446 aa, synthetic construct
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 446 aa, synthetic construct
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 218 aa, synthetic construct
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 15 - protein, 447 aa, synthetic construct
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 6 aa, synthetic construct
GYAFSY

SEQ ID NO: 17 - protein, 8 aa, synthetic construct
FPGDGDTD

SEQ ID NO: 18 - protein, 10 aa, synthetic construct
NVFDGYWLVY

SEQ ID NO: 19 - protein, 16 aa, synthetic construct
RSSKSLLHSNGITYLY

SEQ ID NO: 20 - protein, 7 aa, synthetic construct
QMSNLVS

SEQ ID NO: 21 - protein, 9 aa, synthetic construct
AQNLELPYT

SEQ ID NO: 22 - protein, 119 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 23 - protein, 115 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTV

SEQ ID NO: 24 - protein, 448 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 219 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 5 aa, synthetic construct
SYNMH

SEQ ID NO: 27 - protein, 17 aa, synthetic construct
AIYPGNGDTSYNQKFKG

SEQ ID NO: 28 - protein, 12 aa, synthetic construct
STYYGGDWYFNV

SEQ ID NO: 29 - protein, 10 aa, synthetic construct
RASSSVSYIH

SEQ ID NO: 30 - protein, 7 aa, synthetic construct
ATSNLAS

SEQ ID NO: 31 - protein, 9 aa, synthetic construct
QQWTSNPPT

SEQ ID NO: 32 - protein, 23 aa, synthetic construct
QIVLSQSPAILSASPGEKVTMTC

SEQ ID NO: 33 - protein, 15 aa, synthetic construct
WFQQKPGSSPKPWIY

SEQ ID NO: 34 - protein, 32 aa, synthetic construct
GVPVRFSGSGSGTSYSLTISRVEAEDAATYYC

SEQ ID NO: 35 - protein, 10 aa, synthetic construct
FGGGTKLEIK

SEQ ID NO: 36 - protein, 30 aa, synthetic construct
QVQLQQPGAELVKPGASVKMSCKASGYTFT

SEQ ID NO: 37 - protein, 14 aa, synthetic construct
WVKQTPGRGLEWIG

SEQ ID NO: 38 - protein, 32 aa, synthetic construct
KATLTADKSSSTAYMQLSSLTSEDSAVYYCAR

SEQ ID NO: 39 - protein, 11 aa, synthetic construct
WGAGTTVTVSA

SEQ ID NO: 40 - protein, 121 aa, synthetic construct [sequence omitted]

SEQ ID NO: 41 - protein, 106 aa, synthetic construct [sequence omitted]

SEQ ID NO: 42 - protein, 5 aa, synthetic construct
YSWIN

SEQ ID NO: 43 - protein, 16 aa, synthetic construct
RIFPGDGDTDYNGKFK

(19) United States
(12) Patent Application Publication
(10) Pub. No.: US 2026/0137798 A1
(43) Pub. Date: May 21, 2026

(54) MACROPHAGE SIGNATURES FOR DIAGNOSTIC AND THERAPEUTIC METHODS FOR LYMPHOMA

(71) Applicant: Genentech, Inc., South San Francisco, CA (US)

(72) Inventors: Aikaterini CHATZI, Millbrae, CA (US); Carl Wilson Harris, III, Alameda, CA (US); Yanwen JIANG, Millbrae, CA (US)

(73) Assignee: Genentech, Inc., South San Francisco, CA (US)

(21) Appl. No.: 19/410,705

(22) Filed: Dec. 5, 2025

Related U.S. Application Data
(63) Continuation of application No. PCT/US2024/033016, filed on Jun. 7, 2024.
(60) Provisional application No. 63/507,091, filed on Jun. 8, 2023.

(51) Int. Cl. A61K 47/68; A61K 31/573; A61K 31/675; A61K 31/704; A61K 39/00; A61K 39/395; A61P 35/00; C12Q 1/6886

(52) U.S. Cl. CPC A61K 47/6849; A61K 31/573; A61K 31/675; A61K 31/704; A61K 39/3955; A61K 47/68031; A61P 35/00; C12Q 1/6886; A61K 2039/505; C12Q 2600/106; C12Q 2600/158

(57) ABSTRACT

The present invention provides diagnostic methods, therapeutic methods, and compositions for the treatment of lymphoma (e.g., a diffuse large B-cell lymphoma (e.g., a germinal-center B-cell-like or activated B-cell-like diffuse large B-cell lymphoma). The invention is based, at least in part, on the discovery that macrophage biomarkers are useful in methods of identifying, diagnosing, or predicting the therapeutic efficacy of treatment with an anti-CD79b immunoconjugate (e.g., polatuzumab vedotin) and an anti-CD20 antibody (e.g., obinutuzumab or rituximab).

### CROSS-REFERENCE TO RELATED APPLICATIONS

This application is a continuation of PCT Application No. PCT/US2024/033016, filed internationally on Jun. 7, 2024, which claims the benefit of U.S. Provisional Application No. 63/507,091, filed on Jun. 8, 2023, the content of each of which is hereby incorporated by reference in its entirety.

### REFERENCE TO AN ELECTRONIC SEQUENCE LISTING

The content of the electronic sequence listing (146392066901seglist.xml; Size: 41,725 bytes; and Date of Creation: Oct. 29, 2025) is herein incorporated by reference in its entirety.

### FIELD OF THE INVENTION

The present disclosure relates to methods of treating B-cell proliferative disorders, e.g., diffuse large B-cell lymphoma (DLBCL), by administering an immunoconjugate comprising an anti-CD79b immunoconjugate in combination with an anti-CD20 antibody, one or more chemotherapeutic agents, and a corticosteroid. Also provided are related assays and kits.

### BACKGROUND OF THE INVENTION

Non-Hodgkin lymphoma (NHL) is the most common hematologic malignancy in the world, and the thirteenth most common cancer overall (Bray et al., (2018) CA Cancer J Clin, 68:394-424). Diffuse large B-cell lymphoma (DLBCL) is an aggressive subtype of NHL, accounting for approximately 32.5% of all NHL cases. Patients with DLBCL present with rapidly enlarging masses, often with local and systemic symptoms of fever, recurrent night sweats, and/or weight loss. Approximately 45% to 60% of patients present with advanced-stage disease (Ann Arbor Stage III or IV). The incidence of DLBCL increases with age, with a median age of 64 years at presentation (Armitage and Weisenburger, J Clin Oncol (1998) 6:2780-95). If left untreated, patients with DLBCL have a median survival of approximately 6 months.

Rituximab plus cyclophosphamide, doxorubicin, vincristine, and prednisone (R-CHOP) was established as the standard of care (SoC) therapy for DLBCL over 20 years ago. Although first-line treatment for DLBCL is potentially curative, many patients do not respond or eventually relapse. Approaches to improve on the current SoC therapy for DLBCL have largely been unsuccessful. This includes attempts at maximizing dose density of R-CHOP (Cunningham et al., Lancet (2013) 381:1817-26; Delarue et al., Lancet Oncol (2013) 14:525-33), and experimental treatments such as those tested in large studies in DLBCL, including BO21005/GOYA (Vitolo et al., Blood (2016) 128:470), DA-EPOCH-R (Wilson et al., Blood (2016) 128:469), and REMARC (Thieblemont et al., Blood (2016) 128:471). In total, since the establishment of R-CHOP as the SoC therapy for DLBLC, 11 randomized Phase III studies have failed to show any benefit in first-line DLBCL compared to R-CHOP.

One recently developed alternative to R-CHOP is polatuzumab vedotin plus rituximab, cyclophosphamide, doxorubicin, and prednisone (Pola-R-CHP). Pola-R-CHP was approved by the FDA in April 2023 based on results of the POLARIX clinical trial (Clinical Trial ID No. NCT03274492). POLARIX was a randomized, double-blind, placebo-controlled trial comparing the treatment of previously untreated DLBCL patients with Pola-R-CHP or R-CHOP. In the trial, patients receiving Pola-R-CHP demonstrated significantly longer progression-free survival (PFS) and significant improvement in modified event-free survival. However, the Pola-R-CHP treatment arm did not demonstrate a significant difference in complete response rate or overall survival (FDA. “FDA approves polatuzumab vedotin-piiq for previously untreated diffuse large B-cell lymphoma, not otherwise specified, and high-grade B-cell lymphoma” (2023)).

With newly available Pola-R-CHP as an alternative to the SoC therapy, there is a need in the art for better tailoring of treatment of patients with DLBCL, such as previously untreated DLBCL.

All references cited herein, including patent applications and publications, are hereby incorporated by reference in their entirety.

### SUMMARY OF THE INVENTION

The present invention relates to using macrophage biomarkers in methods of identifying, diagnosing, or predicting the therapeutic efficacy of treating lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) with diagnostic methods, therapeutic methods, and compositions for the treatment of an anti-CD79b immunoconjugate (e.g., polatuzumab vedotin) and an anti-CD20 antibody (e.g., obinutuzumab or rituximab)

In some embodiments, the invention features a method of identifying, diagnosing, and/or predicting whether a patient having a diffuse large B-cell lymphoma (DLBCL) may benefit from a treatment comprising an immunoconjugate and an anti-CD20 antibody. The method can comprise measuring a macrophage biomarker in a sample from the patient, wherein an amount or level of the macrophage biomarker in the sample that is below a reference macrophage biomarker amount or level identifies, diagnoses, and/or predicts the patient as one who may benefit from the treatment comprising the immunoconjugate and the anti-CD20 antibody, wherein the immunoconjugate comprises the formula:

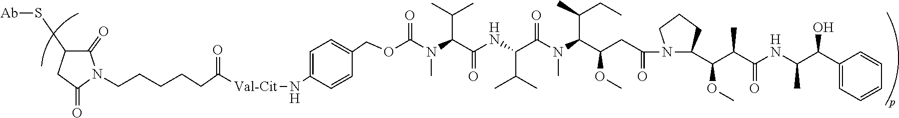

wherein Ab is an anti-CD79b antibody comprising: (i) an HVR-H1 that comprises the amino acid sequence of SEQ ID NO: 5; (ii) an HVR-H2 comprising the amino acid sequence of SEQ ID NO: 6; (iii) an HVR-H3 comprising the amino acid sequence of SEQ ID NO: 7; (iv) an HVR-L1 comprising the amino acid sequence of SEQ ID NO: 8; (v) an HVR-L2 comprising the amino acid sequence of SEQ ID NO: 9; and (vi) an HVR-L3 comprising the amino acid sequence of SEQ ID NO:10, and wherein p is between 1 and 8.

In some embodiments, the invention features a method of selecting a therapy for a patient having a DLBCL. The method can comprise measuring a macrophage biomarker in a sample from the patient, wherein an amount or level of the macrophage biomarker in the sample that is below a reference macrophage biomarker amount or level identifies the patient as one who may benefit from a treatment comprising an immunoconjugate and an anti-CD20 antibody, wherein the immunoconjugate comprises the formula:

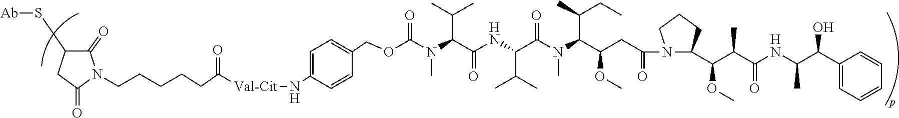

wherein Ab is an anti-CD79b antibody comprising: (i) an HVR-H1 that comprises the amino acid sequence of SEQ ID NO: 5; (ii) an HVR-H2 comprising the amino acid sequence of SEQ ID NO: 6; (iii) an HVR-H3 comprising the amino acid sequence of SEQ ID NO: 7; (iv) an HVR-L1 comprising the amino acid sequence of SEQ ID NO: 8; (v) an HVR-L2 comprising the amino acid sequence of SEQ ID NO: 9; and (vi) an HVR-L3 comprising the amino acid sequence of SEQ ID NO:10, and wherein p is between 1 and 8.

In some method embodiments, which may be combined with any of the preceding aspects or embodiments, the amount or level of the macrophage biomarker from the patient is below the reference macrophage biomarker amount or level, and the method further comprises administering to the patient an effective amount of the immunoconjugate and an effective amount of the anti-CD20 antibody.

In some embodiments, the invention features a method of treating a patient having a DLBCL. The method can comprise: (a) measuring a macrophage biomarker in a sample from the patient, wherein the amount or level of the macrophage biomarker in the sample is below a reference macrophage biomarker amount or level, and (b) administering an effective amount of an immunoconjugate and an effective amount of an anti-CD20 antibody to the patient based on the macrophage biomarker measured in step (a), and wherein the immunoconjugate comprises the formula:

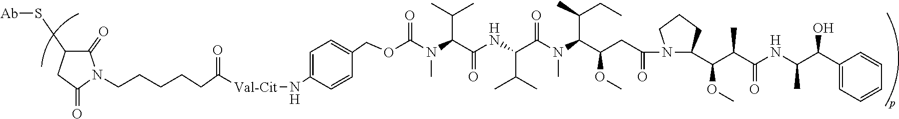

wherein Ab is an anti-CD79b antibody comprising: (i) an HVR-H1 that comprises the amino acid sequence of SEQ ID NO: 5; (ii) an HVR-H2 comprising the amino acid sequence of SEQ ID NO: 6; (iii) an HVR-H3 comprising the amino acid sequence of SEQ ID NO: 7; (iv) an HVR-L1 comprising the amino acid sequence of SEQ ID NO: 8; (v) an HVR-L2 comprising the amino acid sequence of SEQ ID NO: 9; and (vi) an HVR-L3 comprising the amino acid sequence of SEQ ID NO:10, and wherein p is between 1 and 8.

In some embodiments, the invention features a method of treating a patient having a DLBCL. The method can comprise administering to the patient an effective amount of an immunoconjugate and an effective amount of an anti-CD20 antibody, wherein prior to treatment the amount or level of a macrophage biomarker in a sample from the patient has been determined to be below a reference macrophage biomarker amount or level, and wherein the immunoconjugate comprises the formula:

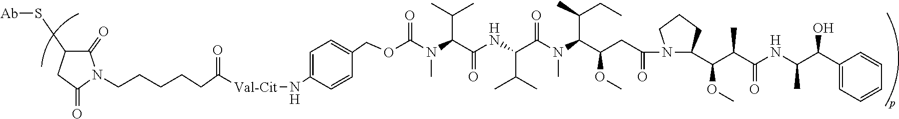

wherein Ab is an anti-CD79b antibody comprising: (i) an HVR-H1 that comprises the amino acid sequence of SEQ ID NO: 5; (ii) an HVR-H2 comprising the amino acid sequence of SEQ ID NO: 6; (iii) an HVR-H3 comprising the amino acid sequence of SEQ ID NO: 7; (iv) an HVR-L1 comprising the amino acid sequence of SEQ ID NO: 8; (v) an HVR-L2 comprising the amino acid sequence of SEQ ID NO: 9; and (vi) an HVR-L3 comprising the amino acid sequence of SEQ ID NO:10, and wherein p is between 1 and 8.

In some embodiments, the invention features a method of treating a patient having a DLBCL and having an amount or level of a macrophage biomarker in a sample from the patient that is below a reference macrophage biomarker amount or level comprising administering to the patient an effective amount of an immunoconjugate and an effective amount of an anti-CD20 antibody, wherein the immunoconjugate comprises the formula:

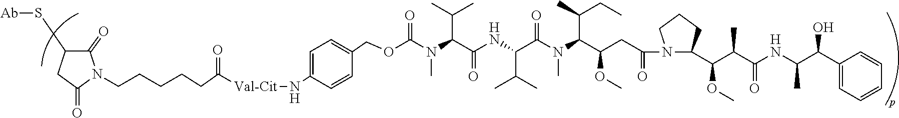

wherein Ab is an anti-CD79b antibody comprising: (i) an HVR-H1 that comprises the amino acid sequence of SEQ ID NO: 5; (ii) an HVR-H2 comprising the amino acid sequence of SEQ ID NO: 6; (iii) an HVR-H3 comprising the amino acid sequence of SEQ ID NO: 7; (iv) an HVR-L1 comprising the amino acid sequence of SEQ ID NO: 8; (v) an HVR-L2 comprising the amino acid sequence of SEQ ID NO: 9; and (vi) an HVR-L3 comprising the amino acid sequence of SEQ ID NO:12, and wherein p is between 1 and 8.

In some method embodiments, which may be combined with any of the preceding aspects or embodiments, the patient is a human patient.

In some method embodiments, which may be combined with any of the preceding aspects or embodiments, the reference macrophage biomarker amount or level is a pre-assigned macrophage biomarker amount or level. In some method embodiments, which may be combined with any of the preceding aspects or embodiments, the reference macrophage biomarker amount or level is an amount or level of a macrophage biomarker in a reference population. In some method embodiments, the amount or level of the macrophage biomarker in a reference population is a median amount or level of the macrophage biomarker of the reference population.

In some method embodiments, the reference macrophage biomarker amount or level is an amount or level of a macrophage biomarker that is at the 25th percentile of the reference population. In some method embodiments, the reference macrophage biomarker amount or level is an amount or level of a macrophage biomarker that is at the 50th percentile of the reference population. In some method embodiments, the reference macrophage biomarker amount or level is an amount or level of a macrophage biomarker that is at the 75th percentile of the reference population.

In some method embodiments, the reference population is a population of patients having the DLBCL. In some method embodiments, the population of patients having the DLBCL was previously treated with the immunoconjugate and the anti-CD20 antibody. In some method embodiments, the population of patients having the DLBCL was previously treated with the anti-CD20 antibody. In some method embodiments, the reference macrophage biomarker amount or level is the amount or level of macrophage biomarker of the reference population prior to initiating treatment with the immunoconjugate and the anti-CD20 antibody.

In some method embodiments, the reference macrophage biomarker amount or level is an amount of macrophages as measured by gene expression. In some method embodiments, the amount of macrophages is between about 0% to about 56.5%.

In some method embodiments, the benefit is an extension of progression-free survival (PFS). In some method embodiments, the benefit is an increase in overall survival (OS). In some method embodiments, the method can further comprise achieving an improvement of PFS or OS.

In some method embodiments, which may be combined with any of the preceding aspects or embodiments, the macrophage biomarker is an average of M1 macrophage gene signature set scores of one or more M1 macrophage gene signature sets. In some method embodiments, each M1 macrophage gene signature set score is an average of the expression level of one or more genes of an M1 macrophage gene signature set. In some method embodiments, each M1 macrophage gene signature set score is an average of the normalized expression level of one or more genes of an M1 macrophage gene signature set. In some method embodiments, the one or more M1 macrophage gene signature sets are: (a) ACP2, ABCD1, C1QA, FDX1, CCL22, CD163, SCAMP2, ADAMDEC1, ARL8B, and HAMP; (b) ACP2, ABCD1, FDX1, CCL8, CCL22, CD163, ADAMDEC1, TREM2, and HAMP; (c) ACP2, ADRA2B, ALCAM, ABCD1, ATOX1, ATP6VOC, ATP6V1E1, BLVRA, C1QA, CD48, CD63, CLCN7, TPP1, CLTC, CCR1, CMKLR1, SLC31A1, COX5B, FCER1G, FDX1, FOLR2, FPR3, FTL, HEXB, HK3, IL10, IL12B, ITGAE, LAIR1, CXCL9, MMP19, NARS, NDUFS2, P2RX7, PDCL, MAPK13, PTGIR, PTPRA, RELA, CCL7, CCL8, CCL19, CCL22, SRC, STX4, TCEB1, TFRC, AGPS, MARCO, SNX3, CD84, USP14, ITGB1BP1, ATP6V1F, TRIP4, CD163, CIAO1, WTAP, ARHGEF11, ABI1, SCAMP2, ACTR2, BCAP31, ZMPSTE24, BCKDK, EXOC5, STIP1, UQCR11, SDS, LILRB4, OGFR, TFEC, FKBP15, DNAJC13, TDRD7, STX12, IL17RA, ABTB2, FAM32A, SIGLEC7, SIGLEC9, ADAMDEC1, CECR5, SLC25A24, NRBP1, MS4A4A, TREM2, OTUD4, PQLC2, HAUS2, ARL8B, NECAP2, WDR11, ZC3H15, CCDC47, UTP3, MRS2, HAMP, MRPL40, VPS33A, CORO7, LIMD2, TMX1, DOT1L, ADO, and ADCK2; (d) ACP2, ADRA2B, ALCAM, TSPO, C3AR1, DAGLA, CALR, CHIT1, CYBB, CYC1, CYP19A1, DLAT, FCER1G, GP1BA, GPD1, IFNAR1, IL10, KCNJ5, KIFC3, MT2A, MYBPH, MYH11, MYO7A, P2RX7, PRDX1, RAB3IL1, RNH1, MRPL12, CCL1, CCL7, CCL8, CCL24, SRC, VIM, RRP1, MARCO, S1PR2, AP1M2, ACTR3, LILRB1, AFG3L2, SDS, LILRB4, EMILIN1, VSIG4, HSPB7, COQ2, ADAMDEC1, CECR5, WSB2, SLAMF8, DNASE2B, CLPB, MFSD7, and ADCK2; (e) ACP2, ADCY3, ADRA2B, ALCAM, TSPO, C1QA, C1QB, C3AR1, DAGLA, CD63, CHIT1, CMKLR1, SLC31A1, CSF1, CSF1R, CYBB, CYC1, CYP19A1, FANCE, FCER1G, FDX1, FPR3, FTL, GP1BA, GPD1, HEXB, IL10, KCNJ1, KCNJ5, KIFC3, LAMP1, MMP19, MSR1, MT2A, MYBPH, MYO7A, P2RX7, PRDX1, RAB3IL1, MRPL12, CCL1, CCL7, CCL8, CCL18, CCL19, CCL24, SLC6A12, SPR, SRC, RRP1, MARCO, PKD2L1, S1PR2, CD163, LONP1, AP1M2, IGSF6, LILRB1, SDS, LILRB4, EMILIN1, VSIG4, TFEC, PHLDB1, CYFIP1, FKBP15, NCAPH, MYOF, HSPB7, ADAMDEC1, GLRX2, NDUFAF1, SPG21, MS4A4A, ATP6V1D, ATP6V1H, TREM2, PQLC2, TMEM70, PLEKHB2, TMEM33, SLAMF8, HAMP, DNASE2B, MYOZ1, LONRF3, CLPB, MFSD7, and ADCK2; and/or (f) ACP2, ADCY3, ADRA2B, ALCAM, ABCD1, ANXA2, ATP6V1A, C1QA, C1QB, C3AR1, DAGLA, CD80, CD63, CHIT1, CMKLR1, SLC31A1, CSF1, CSF1R, CYBB, CYC1, CYP19A1, FANCE, FDX1, FPR2, FPR3, GPD1, HEXB, KCNJ1, KCNJ5, KIFC3, MMP19, MSR1, MT2A, MYBPH, P2RX7, MAPK13, S100A11, CCL1, CCL7, CCL8, CCL18, CCL19, CCL22, CCL24, SLC1A2, SLC6A12, SLC11A1, SIGLEC1, SRC, TIE1, MARCO, HYAL2, CD163, LONP1, IGSF6, LILRB1, CD300C, SDS, LILRB4, EMILIN1, VSIG4, PHLDB1, NCAPH, CLEC4E, MYOF, HSPB7, ADAMDEC1, GLRX2, MS4A4A, ATP6V1H, TREM2, TMEM70, TMEM33, KCNK13, SLAMF8, HAMP, DNASE2B, MYOZ1, MFSD7, ADO, ADCK2, and TBC1D16.

In some method embodiments, which may be combined with any of the preceding aspects or embodiments, the macrophage biomarker is an average of TAM gene signature set scores of one or more TAM gene signature sets. In some method embodiments, each TAM gene signature set score is an average of the expression level of one or more genes of a TAM gene signature set. In some method embodiments, each TAM gene signature set score is an average of the normalized expression level of one or more genes of a TAM gene signature set. In some method embodiments, the one or more TAM gene signature sets are: (a) MARCO, ACP5, VSIG4, MRC1, MSR1, MCEMP1, CYP27A1, OLR1, GRN, GLIPR2, ARRDC4, C1QC, APOE, FOLR2, CTSD and SPP1.

In some method embodiments, the macrophage biomarker is a gene expression value. In some method embodiments, the gene expression value is a median gene expression value. In some method embodiments, the gene expression value is measured using a gene signature matrix. In some method embodiments, the gene signature matrix comprises the following genes: (a) CD200, KLHL14, TCL1A, NRG1, EOMES, PPP2R2B, RNF165, WNT7A, CCR4, PDGFD, EBF1, FCGBP, PCDH9, MLC1, TSHZ2, S1PR5, NCALD, LAYN, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, ABCB4, CD5, HPGD, BLNK, PLCL1, HPSE, SLFN13, HOPX, CD1D, GNG7, TCF4, BANK1, FHIT, FCMR, GNG2, GFRA2, KBTBD11, RALGPS2, TSPOAP1, PLEKHF1, MEF2C, MAOA, TTYH2, HLA-DOB, DGAT2, FXYD6, TMCC3, MGAM, TTC38, LRRC32, ARHGAP24, STAT4, SLC7A8, CD72, FZD1, GK5, DYSF, PLTP, SMARCD3, FAM160B1, PDPN, AKAP2, ACVRL1, KCNJ15, ALDH1A2, ENPP2, COLEC12, PTGS1, TMEM170B, TREM2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, LILRA2, ACE, NUPR1, CISH, EREG, ADAMDEC1, RNASE6, CXCL3, VSIG4, CXCL2, CD86, LILRB4, SERPING1, SQOR, INHBA, and ICAM1; or (b) CD200, KLHL14, TCL1A, NRG1, CYP4F3, EOMES, PPP2R2B, RNF165, WNT7A, CCR4, PDGFD, EBF1, FCGBP, PCDH9, MLC1, TSHZ2, S1PR5, NCALD, LAYN, CD248, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, NINJ2, ABCB4, CD5, HAL, HPGD, BLNK, PLCL1, CEP19, HPSE, SLFN13, HOPX, CD1D, GNG7, TMEM154, TCF4, BANK1, FHIT, FCMR, GNG2, GFRA2, KBTBD11, TECPR2, RALGPS2, TSPOAP1, PLEKHF1, MEF2C, MAOA, TTYH2, HLA-DOB, NRGN, DGAT2, FXYD6, TMCC3, MGAM, TTC38, LRRC32, ARHGAP24, PPP1R3B, STAT4, SLC7A8, CD72, FZD1, GK5, DYSF, PLTP, SMARCD3, FAM160B1, PDPN, AKAP2, ACVRL1, KCNJ15, CD36, ALDH1A2, ENPP2, COLEC12, PTGS1, TMEM170B, DOCK5, TREM2, C5AR2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, TLR4, LILRA2, ACE, TLR1, LRRK2, LY96, NUPR1, CISH, CSTA, EREG, ADAMDEC1, RNASE6, CXCL3, VSIG4, CXCL2, CD86, LILRB4, SERPING1, SQOR, INHBA, and ICAM1.

In some method embodiments, the gene signature matrix consists of the following genes: CD200, KLHL14, TCL1A, NRG1, EOMES, PPP2R2B, RNF165, WNT7A, CCR4, PDGFD, EBF1, FCGBP, PCDH9, MLC1, TSHZ2, S1PR5, NCALD, LAYN, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, ABCB4, CD5, HPGD, BLNK, PLCL1, HPSE, SLFN13, HOPX, CD1D, GNG7, TCF4, BANK1, FHIT, FCMR, GNG2, GFRA2, KBTBD11, RALGPS2, TSPOAP1, PLEKHF1, MEF2C, MAOA, TTYH2, HLA-DOB, DGAT2, FXYD6, TMCC3, MGAM, TTC38, LRRC32, ARHGAP24, STAT4, SLC7A8, CD72, FZD1, GK5, DYSF, PLTP, SMARCD3, FAM160B1, PDPN, AKAP2, ACVRL1, KCNJ15, ALDH1A2, ENPP2, COLEC12, PTGS1, TMEM170B, TREM2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, LILRA2, ACE, NUPR1, CISH, EREG, ADAMDEC1, RNASE6, CXCL3, VSIG4, CXCL2, CD86, LILRB4, SERPING1, SQOR, INHBA, and ICAM1. In some method embodiments, the gene signature matrix is used to determine a number of M1 macrophages or tumor-associated macrophages.

In some method embodiments, which may be combined with any of the preceding aspects or embodiments, the macrophage biomarker is an amount of M1 macrophages or an amount of tumor-associated macrophages. In some method embodiments, the amount of M1 macrophages or tumor-associated macrophages is measured directly or indirectly. In some method embodiments, the amount of M1 macrophages or tumor-associated macrophages is measured directly using flow cytometry, spatial transcriptomics, spatial proteomics, or combination thereof. In some method embodiments, the amount of M1 macrophages or tumor-associated macrophages is measured indirectly using nucleic acid or protein. In some method embodiments, the nucleic acid is measured using RNA-seq, RT-qPCR, qPCR, multiplex qPCR or RT-qPCR, microarray analysis, SAGE, MassARRAY technique, ISH, or a combination thereof. In some method embodiments, the amount of M1 macrophages or tumor-associated macrophages is measured using a marker gene approach or a deconvolution approach. In some method embodiments, the marker gene approach uses xCell. In some method embodiments, the deconvolution approach uses quanTIseq.

In some method embodiments, the macrophage biomarker in the sample from the patient is measured using nucleic acid or protein. In some method embodiments, the macrophage biomarker in the sample from the patient is determined using a nucleic acid expression level. In some method embodiments, the nucleic acid expression level is determined by RNA-seq, RT-qPCR, qPCR, multiplex qPCR or RT-qPCR, microarray analysis, SAGE, MassARRAY technique, ISH, or a combination thereof. In some method embodiments, the nucleic acid expression level is an mRNA expression level. In some method embodiments, the mRNA expression level is determined by RNA-seq.

In some method embodiments, which may be combined with any of the preceding aspects or embodiments, the sample is a tissue sample, tumor sample, whole blood sample, a plasma sample, a serum sample, or a combination thereof. In some method embodiments, the sample is a tissue sample. In some method embodiments, the tissue sample is a tumor tissue sample. In some method embodiments, the tumor tissue sample contains tumor cells, tumor-infiltrating immune cells, stromal cells, normal adjacent tissue (NAT) cells, or a combination thereof. In some method embodiments, the tumor tissue sample is a biopsy. In some method embodiments, the sample is an archival sample, a fresh sample, or a frozen sample.

In some method embodiments, which may be combined with any of the preceding aspects or embodiments, the DLBCL is a germinal-center B-cell-like (GCB) or activated B-cell-like (ABC) cell-of-origin subgroup of DLBCL. In some method embodiments, which may be combined with any of the preceding aspects or embodiments, the DLBCL is a CD79b- and/or CD20-positive DLBCL. In some method embodiments, which may be combined with any of the preceding aspects or embodiments, the patient has not been previously treated for the DLBCL. In some method embodiments, which may be combined with any of the preceding aspects or embodiments, the patient has not been previously administered the immunoconjugate and the anti-CD20 antibody.

In some method embodiments, which may be combined with any of the preceding aspects or embodiments, the anti-CD79b antibody comprises a heavy chain variable domain (VH) comprising the amino acid sequence of SEQ ID NO: 3 and a light chain variable domain (VL) comprising the amino acid sequence of SEQ ID NO: 4. In some method embodiments, which may be combined with any of the preceding aspects or embodiments, the anti-CD79b antibody comprises: (a) a heavy chain comprising the amino acid sequence of SEQ ID NO: 13 and a light chain comprising the amino acid sequence of SEQ ID NO: 11; (b) a heavy chain comprising the amino acid sequence of SEQ ID NO: 12 and a light chain comprising the amino acid sequence of SEQ ID NO: 14; or (c) a heavy chain comprising the amino acid sequence of SEQ ID NO: 12 and a light chain comprising the amino acid sequence of SEQ ID NO: 11. In some method embodiments, which may be combined with any of the preceding aspects or embodiments, p is between 2 and 7, between 2 and 6, between 2 and 5, between 3 and 5, or between 3 and 4. In some method embodiments, p is 3.4. In some method embodiments, p is 3.5. In some method embodiments, which may be combined with any of the preceding aspects or embodiments, the immunoconjugate is polatuzumab vedotin.

In some method embodiments, which may be combined with any of the preceding aspects or embodiments, the anti-CD20 antibody is a type I anti-CD20 antibody or a type II anti-CD20 antibody. In some method embodiments, the anti-CD20 antibody is a type I anti-CD20 antibody. In some method embodiments, the type I anti-CD20 antibody comprises the following CDRs: (a) a CDR-H1 with an amino acid sequence of SEQ ID NO: 26; (b) a CDR-H2 with an amino acid sequence of SEQ ID NO: 27; (c) a CDR-H3 with an amino acid sequence of SEQ ID NO: 28; (d) a CDR-L1 with an amino acid sequence of SEQ ID NO: 29; (e) a CDR-L2 with an amino acid sequence of SEQ ID NO: 30; and (f) a CDR-L3 with an amino acid sequence of SEQ ID NO: 31. In some method embodiments, the type I anti-CD20 antibody comprises a VH domain comprising an amino acid sequence of SEQ ID NO: 40 and a VL domain comprising an amino acid sequence of SEQ ID NO: 41. In some method embodiments, the type I anti-CD20 antibody is rituximab.

In some method embodiments, polatuzumab vedotin is administered at a dose of about 1.0 mg/kg to about 1.8 mg/kg. In some method embodiments, polatuzumab vedotin is administered at a dose of about 1.8 mg/kg. In some method embodiments, rituximab is administered at a dose of about 375 mg/m². In some method embodiments, polatuzumab vedotin and/or rituximab is administered intravenously.

In some method embodiments, the method can further comprise administering to the patient an effective amount of an additional therapeutic agent. In some method embodiments, the additional therapeutic agent is one or more of a chemotherapeutic agent, a corticosteroid, an anti-neoplastic agent, a growth inhibitory agent, an anti-angiogenic agent, a radiation therapy, a cytotoxic agent, or a combination thereof. In some method embodiments, the additional therapeutic agent is a chemotherapeutic agent and a corticosteroid. In some method embodiments, the chemotherapeutic agent is cyclophosphamide and/or doxorubicin. In some method embodiments, the corticosteroid is prednisone, prednisolone, or methylprednisolone. In some method embodiments, cyclophosphamide is administered at a dose of about 375 mg/m² to about 750 mg/m². In some method embodiments, doxorubicin is administered at a dose of about 25 mg/m² to about 50 mg/m². In some embodiments: (a) prednisone is administered at a dose of about 100 mg; (b) prednisolone is administered at a dose of about 100 mg; or (c) methylprednisolone is administered at a dose of about 80 mg. In some method embodiments, cyclophosphamide and/or doxorubicin are administered intravenously.

In some method embodiments, prednisone, prednisolone, or methylprednisolone is administered orally. In some method embodiments, polatuzumab vedotin, rituximab, cyclophosphamide, doxorubicin, and/or prednisone, prednisolone, or methylprednisolone are administered in at least one 21-day cycle. In some embodiments: (a) the polatuzumab vedotin, rituximab, cyclophosphamide, and/or doxorubicin are administered on day 1 of each 21-day cycle; and/or (b) prednisone, prednisolone, or methylprednisolone is administered on days 1-5 of each 21-day cycle. In some method embodiments, polatuzumab vedotin, rituximab, cyclophosphamide, doxorubicin, and/or prednisone, prednisolone, or methylprednisolone are administered for one, two, three, four, five, or six 21-day cycles.

In some embodiments, the invention features a use of an immunoconjugate and an anti-CD20 antibody for treating a patient having an amount or level of a macrophage biomarker in a sample from the patient that is below a reference macrophage biomarker amount or level in the manufacture of a medicament for the treatment of a DLBCL, wherein the immunoconjugate comprises the formula:

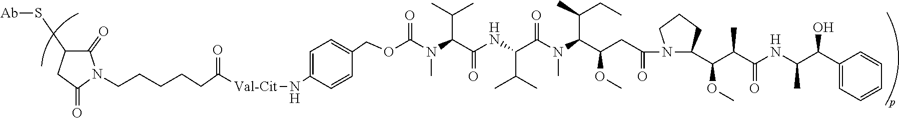

wherein Ab is an anti-CD79b antibody comprising: (i) an HVR-H1 that comprises the amino acid sequence of SEQ ID NO: 5; (ii) an HVR-H2 comprising the amino acid sequence of SEQ ID NO: 6; (iii) an HVR-H3 comprising the amino acid sequence of SEQ ID NO: 7; (iv) an HVR-L1 comprising the amino acid sequence of SEQ ID NO: 8; (v) an HVR-L2 comprising the amino acid sequence of SEQ ID NO: 9; and (vi) an HVR-L3 comprising the amino acid sequence of SEQ ID NO:10, and wherein p is between 1 and 8.

In some use embodiments, the patient is a human patient.

In some use embodiments, which may be combined with any of the preceding aspects or embodiments, the reference macrophage biomarker amount or level is a pre-assigned macrophage biomarker amount or level. In some use embodiments, which may be combined with any of the preceding aspects or embodiments, the reference macrophage biomarker amount or level is an amount or level of a macrophage biomarker in a reference population. In some use embodiments, the amount or level of the macrophage biomarker in a reference population is a median amount or level of the macrophage biomarker of the reference population.

In some use embodiments, the reference macrophage biomarker amount or level is an amount or level of a macrophage biomarker that is at the 25th percentile of the reference population. In some use embodiments, the reference macrophage biomarker amount or level is an amount or level of a macrophage biomarker that is at the 50th percentile of the reference population. In some use embodiments, the reference macrophage biomarker amount or level is an amount or level of a macrophage biomarker that is at the 75th percentile of the reference population.

In some use embodiments, the reference population is a population of patients having the DLBCL. In some use embodiments, the population of patients having the DLBCL was previously treated with the immunoconjugate and the anti-CD20 antibody. In some use embodiments, the population of patients having the DLBCL was previously treated with the anti-CD20 antibody. In some use embodiments, the reference macrophage biomarker amount or level is the amount or level of macrophage biomarker of the reference population prior to initiating treatment with the immunoconjugate and the anti-CD20 antibody.

In some use embodiments, which may be combined with any of the preceding aspects or embodiments, the reference macrophage biomarker amount or level is an amount of macrophages as measured by gene expression. In some use embodiments, the amount of macrophages is between about 0% to about 56.5%.

In some use embodiments, which may be combined with any of the preceding aspects or embodiments, the treatment achieves an improvement of PFS or OS.

In some use embodiments, which may be combined with any of the preceding aspects or embodiments, the macrophage biomarker is an average of M1 macrophage gene signature set scores of one or more M1 macrophage gene signature sets. In some use embodiments, each M1 macrophage gene signature set score is an average of the expression level of one or more genes of an M1 macrophage gene signature set. In some use embodiments, each M1 macrophage gene signature set score is an average of the normalized expression level of one or more genes of an M1 macrophage gene signature set. In some use embodiments, the one or more M1 macrophage gene signature sets are: (a) ACP2, ABCD1, C1QA, FDX1, CCL22, CD163, SCAMP2, ADAMDEC1, ARL8B, and HAMP; (b) ACP2, ABCD1, FDX1, CCL8, CCL22, CD163, ADAMDEC1, TREM2, and HAMP; (c) ACP2, ADRA2B, ALCAM, ABCD1, ATOX1, ATP6VOC, ATP6V1E1, BLVRA, C1QA, CD48, CD63, CLCN7, TPP1, CLTC, CCR1, CMKLR1, SLC31A1, COX5B, FCER1G, FDX1, FOLR2, FPR3, FTL, HEXB, HK3, IL10, IL12B, ITGAE, LAIR1, CXCL9, MMP19, NARS, NDUFS2, P2RX7, PDCL, MAPK13, PTGIR, PTPRA, RELA, CCL7, CCL8, CCL19, CCL22, SRC, STX4, TCEB1, TFRC, AGPS, MARCO, SNX3, CD84, USP14, ITGB1BP1, ATP6V1F, TRIP4, CD163, CIAO1, WTAP, ARHGEF11, ABI1, SCAMP2, ACTR2, BCAP31, ZMPSTE24, BCKDK, EXOC5, STIP1, UQCR11, SDS, LILRB4, OGFR, TFEC, FKBP15, DNAJC13, TDRD7, STX12, IL17RA, ABTB2, FAM32A, SIGLEC7, SIGLEC9, ADAMDEC1, CECR5, SLC25A24, NRBP1, MS4A4A, TREM2, OTUD4, PQLC2, HAUS2, ARL8B, NECAP2, WDR11, ZC3H15, CCDC47, UTP3, MRS2, HAMP, MRPL40, VPS33A, CORO7, LIMD2, TMX1, DOT1L, ADO, and ADCK2; (d) ACP2, ADRA2B, ALCAM, TSPO, C3AR1, DAGLA, CALR, CHIT1, CYBB, CYC1, CYP19A1, DLAT, FCER1G, GP1BA, GPD1, IFNAR1, IL10, KCNJ5, KIFC3, MT2A, MYBPH, MYH11, MYO7A, P2RX7, PRDX1, RAB3IL1, RNH1, MRPL12, CCL1, CCL7, CCL8, CCL24, SRC, VIM, RRP1, MARCO, S1PR2, AP1M2, ACTR3, LILRB1, AFG3L2, SDS, LILRB4, EMILIN1, VSIG4, HSPB7, COQ2, ADAMDEC1, CECR5, WSB2, SLAMF8, DNASE2B, CLPB, MFSD7, and ADCK2; (e) ACP2, ADCY3, ADRA2B, ALCAM, TSPO, C1QA, C1QB, C3AR1, DAGLA, CD63, CHIT1, CMKLR1, SLC31A1, CSF1, CSF1R, CYBB, CYC1, CYP19A1, FANCE, FCER1G, FDX1, FPR3, FTL, GP1BA, GPD1, HEXB, IL10, KCNJ1, KCNJ5, KIFC3, LAMP1, MMP19, MSR1, MT2A, MYBPH, MYO7A, P2RX7, PRDX1, RAB3IL1, MRPL12, CCL1, CCL7, CCL8, CCL18, CCL19, CCL24, SLC6A12, SPR, SRC, RRP1, MARCO, PKD2L1, S1PR2, CD163, LONP1, AP1M2, IGSF6, LILRB1, SDS, LILRB4, EMILIN1, VSIG4, TFEC, PHLDB1, CYFIP1, FKBP15, NCAPH, MYOF, HSPB7, ADAMDEC1, GLRX2, NDUFAF1, SPG21, MS4A4A, ATP6V1D, ATP6V1H, TREM2, PQLC2, TMEM70, PLEKHB2, TMEM33, SLAMF8, HAMP, DNASE2B, MYOZ1, LONRF3, CLPB, MFSD7, and ADCK2; and/or (f) ACP2, ADCY3, ADRA2B, ALCAM, ABCD1, ANXA2, ATP6V1A, C1QA, C1QB, C3AR1, DAGLA, CD80, CD63, CHIT1, CMKLR1, SLC31A1, CSF1, CSF1R, CYBB, CYC1, CYP19A1, FANCE, FDX1, FPR2, FPR3, GPD1, HEXB, KCNJ1, KCNJ5, KIFC3, MMP19, MSR1, MT2A, MYBPH, P2RX7, MAPK13, S100A11, CCL1, CCL7, CCL8, CCL18, CCL19, CCL22, CCL24, SLC1A2, SLC6A12, SLC11A1, SIGLEC1, SRC, TIE1, MARCO, HYAL2, CD163, LONP1, IGSF6, LILRB1, CD300C, SDS, LILRB4, EMILIN1, VSIG4, PHLDB1, NCAPH, CLEC4E, MYOF, HSPB7, ADAMDEC1, GLRX2, MS4A4A, ATP6V1H, TREM2, TMEM70, TMEM33, KCNK13, SLAMF8, HAMP, DNASE2B, MYOZ1, MFSD7, ADO, ADCK2, and TBC1D16.

In some use embodiments, which may be combined with any of the preceding aspects or embodiments, the macrophage biomarker is an average of tumor-associated macrophage (TAM) gene signature set scores of one or more TAM gene signature sets. In some use embodiments, each TAM gene signature set score is an average of the expression level of one or more genes of a TAM gene signature set. In some use embodiments, each TAM gene signature set score is an average of the normalized expression level of one or more genes of a TAM gene signature set. In some use embodiments, the one or more TAM gene signature sets are: MARCO, ACP5, VSIG4, MRC1, MSR1, MCEMP1, CYP27A1, OLR1, GRN, GLIPR2, ARRDC4, C1QC, APOE, FOLR2, CTSD and SPP1.

In some use embodiments, which may be combined with any of the preceding aspects or embodiments, the macrophage biomarker is a gene expression value. In some use embodiments, the gene expression value is a median gene expression value. In some use embodiments, the gene expression value is measured using a gene signature matrix. In some use embodiments, the gene signature matrix comprises the following genes: (a) CD200, KLHL14, TCL1A, NRG1, EOMES, PPP2R2B, RNF165, WNT7A, CCR4, PDGFD, EBF1, FCGBP, PCDH9, MLC1, TSHZ2, S1PR5, NCALD, LAYN, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, ABCB4, CD5, HPGD, BLNK, PLCL1, HPSE, SLFN13, HOPX, CD1D, GNG7, TCF4, BANK1, FHIT, FCMR, GNG2, GFRA2, KBTBD11, RALGPS2, TSPOAP1, PLEKHF1, MEF2C, MAOA, TTYH2, HLA-DOB, DGAT2, FXYD6, TMCC3, MGAM, TTC38, LRRC32, ARHGAP24, STAT4, SLC7A8, CD72, FZD1, GK5, DYSF, PLTP, SMARCD3, FAM160B1, PDPN, AKAP2, ACVRL1, KCNJ15, ALDH1A2, ENPP2, COLEC12, PTGS1, TMEM170B, TREM2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, LILRA2, ACE, NUPR1, CISH, EREG, ADAMDEC1, RNASE6, CXCL3, VSIG4, CXCL2, CD86, LILRB4, SERPING1, SQOR, INHBA, and ICAM1; or (b) CD200, KLHL14, TCL1A, NRG1, CYP4F3, EOMES, PPP2R2B, RNF165, WNT7A, CCR4, PDGFD, EBF1, FCGBP, PCDH9, MLC1, TSHZ2, S1PR5, NCALD, LAYN, CD248, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, NINJ2, ABCB4, CD5, HAL, HPGD, BLNK, PLCL1, CEP19, HPSE, SLFN13, HOPX, CD1D, GNG7, TMEM154, TCF4, BANK1, FHIT, FCMR, GNG2, GFRA2, KBTBD11, TECPR2, RALGPS2, TSPOAP1, PLEKHF1, MEF2C, MAOA, TTYH2, HLA-DOB, NRGN, DGAT2, FXYD6, TMCC3, MGAM, TTC38, LRRC32, ARHGAP24, PPP1R3B, STAT4, SLC7A8, CD72, FZD1, GK5, DYSF, PLTP, SMARCD3, FAM160B1, PDPN, AKAP2, ACVRL1, KCNJ15, CD36, ALDH1A2, ENPP2, COLEC12, PTGS1, TMEM170B, DOCK5, TREM2, C5AR2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, TLR4, LILRA2, ACE, TLR1, LRRK2, LY96, NUPR1, CISH, CSTA, EREG, ADAMDEC1, RNASE6, CXCL3, VSIG4, CXCL2, CD86, LILRB4, SERPING1, SQOR, INHBA, and ICAM1.

In some use embodiments, the gene signature matrix consists of the following genes: CD200, KLHL14, TCL1A, NRG1, EOMES, PPP2R2B, RNF165, WNT7A, CCR4, PDGFD, EBF1, FCGBP, PCDH9, MLC1, TSHZ2, S1PR5, NCALD, LAYN, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, ABCB4, CD5, HPGD, BLNK, PLCL1, HPSE, SLFN13, HOPX, CD1D, GNG7, TCF4, BANK1, FHIT, FCMR, GNG2, GFRA2, KBTBD11, RALGPS2, TSPOAP1, PLEKHF1, MEF2C, MAOA, TTYH2, HLA-DOB, DGAT2, FXYD6, TMCC3, MGAM, TTC38, LRRC32, ARHGAP24, STAT4, SLC7A8, CD72, FZD1, GK5, DYSF, PLTP, SMARCD3, FAM160B1, PDPN, AKAP2, ACVRL1, KCNJ15, ALDH1A2, ENPP2, COLEC12, PTGS1, TMEM170B, TREM2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, LILRA2, ACE, NUPR1, CISH, EREG, ADAMDEC1, RNASE6, CXCL3, VSIG4, CXCL2, CD86, LILRB4, SERPING1, SQOR, INHBA, and ICAM1. In some use embodiments, the gene signature matrix is used to determine a number of M1 macrophages or tumor-associated macrophages.

In some use embodiments, which may be combined with any of the preceding aspects or embodiments, the macrophage biomarker is an amount of M1 macrophages or an amount of tumor-associated macrophages. In some use embodiments, the amount of M1 macrophages or tumor-associated macrophages is measured directly or indirectly. In some use embodiments, the amount of M1 macrophages or tumor-associated macrophages is measured directly using flow cytometry, spatial transcriptomics, spatial proteomics, or combination thereof. In some use embodiments, the amount of M1 macrophages or tumor-associated macrophages is measured indirectly using nucleic acid or protein. In some use embodiments, the nucleic acid is measured using RNA-seq, RT-qPCR, qPCR, multiplex qPCR or RT-qPCR, microarray analysis, SAGE, MassARRAY technique, ISH, or a combination thereof. In some use embodiments, the amount of M1 macrophages or tumor-associated macrophages is measured using a marker gene approach or a deconvolution approach. In some use embodiments, the marker gene approach uses xCell. In some use embodiments, the deconvolution approach uses quanTIseq.

In some use embodiments, the macrophage biomarker in the sample from the patient is measured using nucleic acid or protein. In some use embodiments, the macrophage biomarker in the sample from the patient is determined using a nucleic acid expression level. In some use embodiments, the nucleic acid expression level is determined by RNA-seq, RT-qPCR, qPCR, multiplex qPCR or RT-qPCR, microarray analysis, SAGE, MassARRAY technique, ISH, or a combination thereof. In some use embodiments, the nucleic acid expression level is an mRNA expression level. In some use embodiments, the mRNA expression level is determined by RNA-seq.

In some use embodiments, which may be combined with any of the preceding aspects or embodiments, the sample is a tissue sample, tumor sample, whole blood sample, a plasma sample, a serum sample, or a combination thereof. In some use embodiments, the sample is a tissue sample. In some use embodiments, the tissue sample is a tumor tissue sample. In some use embodiments, the tumor tissue sample contains tumor cells, tumor-infiltrating immune cells, stromal cells, normal adjacent tissue (NAT) cells, or a combination thereof. In some use embodiments, the tumor tissue sample is a biopsy. In some use embodiments, the sample is an archival sample, a fresh sample, or a frozen sample.

In some use embodiments, which may be combined with any of the preceding aspects or embodiments, the DLBCL is a germinal-center B-cell-like (GCB) or activated B-cell-like (ABC) cell-of-origin subgroup of DLBCL. In some use embodiments, which may be combined with any of the preceding aspects or embodiments, the DLBCL is a CD79a- and/or CD20-positive DLBCL. In some use embodiments, which may be combined with any of the preceding aspects or embodiments, the patient has not been previously treated for the DLBCL. In some use embodiments, which may be combined with any of the preceding aspects or embodiments, the patient has not been previously administered the immunoconjugate and the anti-CD20 antibody.

In some use embodiments, which may be combined with any of the preceding aspects or embodiments, the anti-CD79b antibody comprises a heavy chain variable domain (VH) comprising the amino acid sequence of SEQ ID NO: 3 and a light chain variable domain (VL) comprising the amino acid sequence of SEQ ID NO: 4. In some use embodiments, the anti-CD79b antibody comprises: (a) a heavy chain comprising the amino acid sequence of SEQ ID NO: 13 and a light chain comprising the amino acid sequence of SEQ ID NO: 11; (b) a heavy chain comprising the amino acid sequence of SEQ ID NO: 12 and a light chain comprising the amino acid sequence of SEQ ID NO: 14; or (c) a heavy chain comprising the amino acid sequence of SEQ ID NO: 12 and a light chain comprising the amino acid sequence of SEQ ID NO: 11. In some use embodiments, which may be combined with any of the preceding aspects or embodiments, p is between 2 and 7, between 2 and 6, between 2 and 5, between 3 and 5, or between 3 and 4. In some use embodiments, p is 3.4. In some use embodiments, p is 3.5. In some use embodiments, which may be combined with any of the preceding aspects or embodiments, the immunoconjugate is polatuzumab vedotin.

In some use embodiments, which may be combined with any of the preceding aspects or embodiments, the anti-CD20 antibody is a type I anti-CD20 antibody or a type II anti-CD20 antibody. In some use embodiments, the anti-CD20 antibody is a type I anti-CD20 antibody. In some use embodiments, the type I anti-CD20 antibody comprises the following CDRs: (a) a CDR-H1 with an amino acid sequence of SEQ ID NO: 26; (b) a CDR-H2 with an amino acid sequence of SEQ ID NO: 27; (c) a CDR-H3 with an amino acid sequence of SEQ ID NO: 28; (d) a CDR-L1 with an amino acid sequence of SEQ ID NO: 29; (e) a CDR-L2 with an amino acid sequence of SEQ ID NO: 30; and (f) a CDR-L3 with an amino acid sequence of SEQ ID NO: 31. In some use embodiments, the type I anti-CD20 antibody comprises a VH domain comprising an amino acid sequence of SEQ ID NO: 40 and a VL domain comprising an amino acid sequence of SEQ ID NO: 41. In some use embodiments, the type I anti-CD20 antibody is rituximab.

In some use embodiments, polatuzumab vedotin is administered at a dose of about 1.0 mg/kg to about 1.8 mg/kg. In some use embodiments, polatuzumab vedotin is administered at a dose of about 1.8 mg/kg. In some use embodiments, rituximab is administered at a dose of about 375 mg/m². In some use embodiments, polatuzumab vedotin and/or rituximab is administered intravenously.

In some use embodiments, which may be combined with any of the preceding aspects or embodiments, the medicament is to be administered to the patient in combination with an effective amount of an additional therapeutic agent. In some use embodiments, the additional therapeutic agent is one or more of a chemotherapeutic agent, a corticosteroid, an anti-neoplastic agent, a growth inhibitory agent, an anti-angiogenic agent, a radiation therapy, a cytotoxic agent, or a combination thereof. In some use embodiments, the additional therapeutic agent is a chemotherapeutic agent and a corticosteroid. In some use embodiments, the chemotherapeutic agent is cyclophosphamide and/or doxorubicin. In some use embodiments, the corticosteroid is prednisone, prednisolone, or methylprednisolone. In some use embodiments, cyclophosphamide is administered at a dose of about 375 mg/m² to about 750 mg/m². In some use embodiments, doxorubicin is administered at a dose of about 25 mg/m² to about 50 mg/m². In some use embodiments: (a) prednisone is administered at a dose of about 100 mg; (b) prednisolone is administered at a dose of about 100 mg; or (c) methylprednisolone is administered at a dose of about 80 mg. In some use embodiments, cyclophosphamide and/or doxorubicin are administered intravenously. In some use embodiments, prednisone, prednisolone, or methylprednisolone is administered orally.

In some use embodiments, polatuzumab vedotin, rituximab, cyclophosphamide, doxorubicin, and/or prednisone, prednisolone, or methylprednisolone are administered in at least one 21-day cycle. In some use embodiments: (a) the polatuzumab vedotin, rituximab, cyclophosphamide, and/or doxorubicin are administered on day 1 of each 21-day cycle; and/or (b) prednisone, prednisolone, or methylprednisolone is administered on days 1-5 of each 21-day cycle. In some use embodiments, polatuzumab vedotin, rituximab, cyclophosphamide, doxorubicin, and/or prednisone, prednisolone, or methylprednisolone are administered for one, two, three, four, five, or six 21-day cycles.

In some embodiments, the invention features an immunoconjugate and an anti-CD20 antibody for use in the treatment of a patient having a DLBCL and having an amount or level of a macrophage biomarker in a sample from the patient that is below a reference macrophage biomarker amount or level, wherein the immunoconjugate comprises the formula:

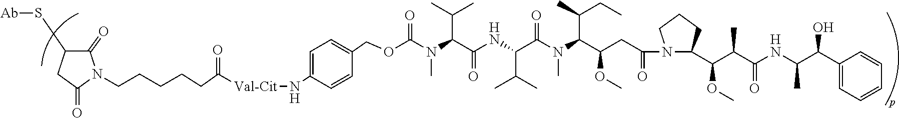

wherein Ab is an anti-CD79b antibody comprising: (i) an HVR-H1 that comprises the amino acid sequence of SEQ ID NO: 5; (ii) an HVR-H2 comprising the amino acid sequence of SEQ ID NO: 6; (iii) an HVR-H3 comprising the amino acid sequence of SEQ ID NO: 7; (iv) an HVR-L1 comprising the amino acid sequence of SEQ ID NO: 8; (v) an HVR-L2 comprising the amino acid sequence of SEQ ID NO: 9; and (vi) an HVR-L3 comprising the amino acid sequence of SEQ ID NO:10, and wherein p is between 1 and 8.

In some embodiments regarding the immunoconjugate and the anti-CD20 antibody for use, the patient is a human patient.

In some embodiments regarding the immunoconjugate and the anti-CD20 antibody for use, which may be combined with any of the preceding aspects or embodiments, the reference macrophage biomarker amount or level is a pre-assigned macrophage biomarker amount or level. In some embodiments regarding the immunoconjugate and the anti-CD20 antibody for use, which may be combined with any of the preceding aspects or embodiments, the reference macrophage biomarker amount or level is an amount or level of a macrophage biomarker in a reference population. In some embodiments regarding the immunoconjugate and the anti-CD20 antibody for use, the amount or level of the macrophage biomarker in a reference population is a median amount or level of the macrophage biomarker of the reference population.

In some embodiments regarding the immunoconjugate and the anti-CD20 antibody for use, the reference macrophage biomarker amount or level is an amount or level of a macrophage biomarker that is at the 25th percentile of the reference population. In some embodiments regarding the immunoconjugate and the anti-CD20 antibody for use, the reference macrophage biomarker amount or level is an amount or level of a macrophage biomarker that is at the 50th percentile of the reference population. In some embodiments regarding the immunoconjugate and the anti-CD20 antibody for use, the reference macrophage biomarker amount or level is an amount or level of a macrophage biomarker that is at the 75th percentile of the reference population.

In some embodiments regarding the immunoconjugate and the anti-CD20 antibody for use, the reference population is a population of patients having the DLBCL. In some embodiments regarding the immunoconjugate and the anti-CD20 antibody for use, the population of patients having the DLBCL was previously treated with the immunoconjugate and the anti-CD20 antibody. In some embodiments regarding the immunoconjugate and the anti-CD20 antibody for use, the population of patients having the DLBCL was previously treated with the anti-CD20 antibody. In some embodiments regarding the immunoconjugate and the anti-CD20 antibody for use, the reference macrophage biomarker amount or level is the amount or level of macrophage biomarker of the reference population prior to initiating treatment with the immunoconjugate and the anti-CD20 antibody.

In some embodiments regarding the immunoconjugate and the anti-CD20 antibody for use, which may be combined with any of the preceding aspects or embodiments, the reference macrophage biomarker amount or level is an amount of macrophages as measured by gene expression. In some embodiments regarding the immunoconjugate and the anti-CD20 antibody for use, the amount of macrophages is between about 0% to about 56.5%.

In some embodiments regarding the immunoconjugate and the anti-CD20 antibody for use, which may be combined with any of the preceding aspects or embodiments, the treatment achieves an improvement of PFS or OS.

In some embodiments regarding the immunoconjugate and the anti-CD20 antibody for use, which may be combined with any of the preceding aspects or embodiments, the macrophage biomarker is an average of M1 macrophage gene signature set scores of one or more M1 macrophage gene signature sets. In some embodiments regarding the immunoconjugate and the anti-CD20 antibody for use, each M1 macrophage gene signature set score is an average of the expression level of one or more genes of an M1 macrophage gene signature set. In some embodiments regarding the immunoconjugate and the anti-CD20 antibody for use, each M1 macrophage gene signature set score is an average of the normalized expression level of one or more genes of an M1 macrophage gene signature set. In some embodiments regarding the immunoconjugate and the anti-CD20 antibody for use, the one or more M1 macrophage gene signature sets are: (a) ACP2, ABCD1, C1QA, FDX1, CCL22, CD163, SCAMP2, ADAMDEC1, ARL8B, and HAMP; (b) ACP2, ABCD1, FDX1, CCL8, CCL22, CD163, ADAMDEC1, TREM2, and HAMP; (c) ACP2, ADRA2B, ALCAM, ABCD1, ATOX1, ATP6VOC, ATP6V1E1, BLVRA, C1QA, CD48, CD63, CLCN7, TPP1, CLTC, CCR1, CMKLR1, SLC31A1, COX5B, FCER1G, FDX1, FOLR2, FPR3, FTL, HEXB, HK3, IL10, IL12B, ITGAE, LAIR1, CXCL9, MMP19, NARS, NDUFS2, P2RX7, PDCL, MAPK13, PTGIR, PTPRA, RELA, CCL7, CCL8, CCL19, CCL22, SRC, STX4, TCEB1, TFRC, AGPS, MARCO, SNX3, CD84, USP14, ITGB1BP1, ATP6V1F, TRIP4, CD163, CIAO1, WTAP, ARHGEF11, ABI1, SCAMP2, ACTR2, BCAP31, ZMPSTE24, BCKDK, EXOC5, STIP1, UQCR11, SDS, LILRB4, OGFR, TFEC, FKBP15, DNAJC13, TDRD7, STX12, IL17RA, ABTB2, FAM32A, SIGLEC7, SIGLEC9, ADAMDEC1, CECR5, SLC25A24, NRBP1, MS4A4A, TREM2, OTUD4, PQLC2, HAUS2, ARL8B, NECAP2, WDR11, ZC3H15, CCDC47, UTP3, MRS2, HAMP, MRPL40, VPS33A, CORO7, LIMD2, TMX1, DOT1L, ADO, and ADCK2; (d) ACP2, ADRA2B, ALCAM, TSPO, C3AR1, DAGLA, CALR, CHIT1, CYBB, CYC1, CYP19A1, DLAT, FCER1G, GP1BA, GPD1, IFNAR1, IL10, KCNJ5, KIFC3, MT2A, MYBPH, MYH11, MYO7A, P2RX7, PRDX1, RAB3IL1, RNH1, MRPL12, CCL1, CCL7, CCL8, CCL24, SRC, VIM, RRP1, MARCO, S1PR2, AP1M2, ACTR3, LILRB1, AFG3L2, SDS, LILRB4, EMILIN1, VSIG4, HSPB7, COQ2, ADAMDEC1, CECR5, WSB2, SLAMF8, DNASE2B, CLPB, MFSD7, and ADCK2; (e) ACP2, ADCY3, ADRA2B, ALCAM, TSPO, C1QA, C1QB, C3AR1, DAGLA, CD63, CHIT1, CMKLR1, SLC31A1, CSF1, CSF1R, CYBB, CYC1, CYP19A1, FANCE, FCER1G, FDX1, FPR3, FTL, GP1BA, GPD1, HEXB, IL10, KCNJ1, KCNJ5, KIFC3, LAMP1, MMP19, MSR1, MT2A, MYBPH, MYO7A, P2RX7, PRDX1, RAB3IL1, MRPL12, CCL1, CCL7, CCL8, CCL18, CCL19, CCL24, SLC6A12, SPR, SRC, RRP1, MARCO, PKD2L1, S1PR2, CD163, LONP1, AP1M2, IGSF6, LILRB1, SDS, LILRB4, EMILIN1, VSIG4, TFEC, PHLDB1, CYFIP1, FKBP15, NCAPH, MYOF, HSPB7, ADAMDEC1, GLRX2, NDUFAF1, SPG21, MS4A4A, ATP6V1D, ATP6V1H, TREM2, PQLC2, TMEM70, PLEKHB2, TMEM33, SLAMF8, HAMP, DNASE2B, MYOZ1, LONRF3, CLPB, MFSD7, and ADCK2; and/or (f) ACP2, ADCY3, ADRA2B, ALCAM, ABCD1, ANXA2, ATP6V1A, C1QA, C1QB, C3AR1, DAGLA, CD80, CD63, CHIT1, CMKLR1, SLC31A1, CSF1, CSF1R, CYBB, CYC1, CYP19A1, FANCE, FDX1, FPR2, FPR3, GPD1, HEXB, KCNJ1, KCNJ5, KIFC3, MMP19, MSR1, MT2A, MYBPH, P2RX7, MAPK13, S100A11, CCL1, CCL7, CCL8, CCL18, CCL19, CCL22, CCL24, SLC1A2, SLC6A12, SLC11A1, SIGLEC1, SRC, TIE1, MARCO, HYAL2, CD163, LONP1, IGSF6, LILRB1, CD300C, SDS, LILRB4, EMILIN1, VSIG4, PHLDB1, NCAPH, CLEC4E, MYOF, HSPB7, ADAMDEC1, GLRX2, MS4A4A, ATP6V1H, TREM2, TMEM70, TMEM33, KCNK13, SLAMF8, HAMP, DNASE2B, MYOZ1, MFSD7, ADO, ADCK2, and TBC1D16.

In some embodiments regarding the immunoconjugate and the anti-CD20 antibody for use, which may be combined with any of the preceding aspects or embodiments, the macrophage biomarker is an average of tumor-associate macrophage gene signature set scores of one or more tumor-associated macrophage gene signature sets. In some embodiments regarding the immunoconjugate and the anti-CD20 antibody for use, each tumor-associated macrophage gene signature set score is an average of the expression level of one or more genes of a tumor-associated macrophage gene signature set. In some embodiments regarding the immunoconjugate and the anti-CD20 antibody for use, each tumor-associated macrophage gene signature set score is an average of the normalized expression level of one or more genes of a tumor-associated macrophage gene signature set. In some embodiments regarding the immunoconjugate and the anti-CD20 antibody for use, the one or more tumor-associated macrophage gene signature sets are: (a) MARCO, ACP5, VSIG4, MRC1, MSR1, MCEMP1, CYP27A1, OLR1, GRN, GLIPR2, ARRDC4, C1QC, APOE, FOLR2, CTSD and SPP1.

In some embodiments regarding the immunoconjugate and the anti-CD20 antibody for use, the macrophage biomarker is a gene expression value. In some embodiments regarding the immunoconjugate and the anti-CD20 antibody for use, the gene expression value is a median gene expression value. In some embodiments regarding the immunoconjugate and the anti-CD20 antibody for use, the gene expression value is measured using a gene signature matrix. In some embodiments regarding the immunoconjugate and the anti-CD20 antibody for use, the gene signature matrix comprises the following genes: (a) CD200, KLHL14, TCL1A, NRG1, EOMES, PPP2R2B, RNF165, WNT7A, CCR4, PDGFD, EBF1, FCGBP, PCDH9, MLC1, TSHZ2, S1PR5, NCALD, LAYN, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, ABCB4, CD5, HPGD, BLNK, PLCL1, HPSE, SLFN13, HOPX, CD1D, GNG7, TCF4, BANK1, FHIT, FCMR, GNG2, GFRA2, KBTBD11, RALGPS2, TSPOAP1, PLEKHF1, MEF2C, MAOA, TTYH2, HLA-DOB, DGAT2, FXYD6, TMCC3, MGAM, TTC38, LRRC32, ARHGAP24, STAT4, SLC7A8, CD72, FZD1, GK5, DYSF, PLTP, SMARCD3, FAM160B1, PDPN, AKAP2, ACVRL1, KCNJ15, ALDH1A2, ENPP2, COLEC12, PTGS1, TMEM170B, TREM2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, LILRA2, ACE, NUPR1, CISH, EREG, ADAMDEC1, RNASE6, CXCL3, VSIG4, CXCL2, CD86, LILRB4, SERPING1, SQOR, INHBA, and ICAM1; or (b) CD200, KLHL14, TCL1A, NRG1, CYP4F3, EOMES, PPP2R2B, RNF165, WNT7A, CCR4, PDGFD, EBF1, FCGBP, PCDH9, MLC1, TSHZ2, S1PR5, NCALD, LAYN, CD248, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, NINJ2, ABCB4, CD5, HAL, HPGD, BLNK, PLCL1, CEP19, HPSE, SLFN13, HOPX, CD1D, GNG7, TMEM154, TCF4, BANK1, FHIT, FCMR, GNG2, GFRA2, KBTBD11, TECPR2, RALGPS2, TSPOAP1, PLEKHF1, MEF2C, MAOA, TTYH2, HLA-DOB, NRGN, DGAT2, FXYD6, TMCC3, MGAM, TTC38, LRRC32, ARHGAP24, PPP1R3B, STAT4, SLC7A8, CD72, FZD1, GK5, DYSF, PLTP, SMARCD3, FAM160B1, PDPN, AKAP2, ACVRL1, KCNJ15, CD36, ALDH1A2, ENPP2, COLEC12, PTGS1, TMEM170B, DOCK5, TREM2, C5AR2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, TLR4, LILRA2, ACE, TLR1, LRRK2, LY96, NUPR1, CISH, CSTA, EREG, ADAMDEC1, RNASE6, CXCL3, VSIG4, CXCL2, CD86, LILRB4, SERPING1, SQOR, INHBA, and ICAM1.

In some embodiments regarding the immunoconjugate and the anti-CD20 antibody for use, the gene signature matrix consists of the following genes: CD200, KLHL14, TCL1A, NRG1, EOMES, PPP2R2B, RNF165, WNT7A, CCR4, PDGFD, EBF1, FCGBP, PCDH9, MLC1, TSHZ2, S1PR5, NCALD, LAYN, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, ABCB4, CD5, HPGD, BLNK, PLCL1, HPSE, SLFN13, HOPX, CD1D, GNG7, TCF4, BANK1, FHIT, FCMR, GNG2, GFRA2, KBTBD11, RALGPS2, TSPOAP1, PLEKHF1, MEF2C, MAOA, TTYH2, HLA-DOB, DGAT2, FXYD6, TMCC3, MGAM, TTC38, LRRC32, ARHGAP24, STAT4, SLC7A8, CD72, FZD1, GK5, DYSF, PLTP, SMARCD3, FAM160B1, PDPN, AKAP2, ACVRL1, KCNJ15, ALDH1A2, ENPP2, COLEC12, PTGS1, TMEM170B, TREM2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, LILRA2, ACE, NUPR1, CISH, EREG, ADAMDEC1, RNASE6, CXCL3, VSIG4, CXCL2, CD86, LILRB4, SERPING1, SQOR, INHBA, and ICAM1. In some embodiments regarding the immunoconjugate and the anti-CD20 antibody for use, the gene signature matrix is used to determine a number of M1 macrophages or tumor-associated macrophages.

In some embodiments regarding the immunoconjugate and the anti-CD20 antibody for use, which may be combined with any of the preceding aspects or embodiments, the macrophage biomarker is an amount of M1 macrophages or an amount of tumor-associated macrophages. In some embodiments regarding the immunoconjugate and the anti-CD20 antibody for use, the amount of M1 macrophages or tumor-associated macrophages is measured directly or indirectly. In some embodiments regarding the immunoconjugate and the anti-CD20 antibody for use, the amount of M1 macrophages or tumor-associated macrophages is measured directly using flow cytometry, spatial transcriptomics, spatial proteomics, or combination thereof. In some embodiments regarding the immunoconjugate and the anti-CD20 antibody for use, the amount of M1 macrophages or tumor-associated macrophages is measured indirectly using nucleic acid or protein. In some embodiments regarding the immunoconjugate and the anti-CD20 antibody for use, the nucleic acid is measured using RNA-seq, RT-qPCR, qPCR, multiplex qPCR or RT-qPCR, microarray analysis, SAGE, MassARRAY technique, ISH, or a combination thereof. In some embodiments regarding the immunoconjugate and the anti-CD20 antibody for use, the amount of M1 macrophages or tumor-associated macrophages is measured using a marker gene approach or a deconvolution approach. In some embodiments regarding the immunoconjugate and the anti-CD20 antibody for use, the marker gene approach uses xCell. In some embodiments regarding the immunoconjugate and the anti-CD20 antibody for use, the deconvolution approach uses quanTIseq.

In some embodiments regarding the immunoconjugate and the anti-CD20 antibody for use, the macrophage biomarker in the sample from the patient is measured using nucleic acid or protein. In some embodiments regarding the immunoconjugate and the anti-CD20 antibody for use, the macrophage biomarker in the sample from the patient is determined using a nucleic acid expression level. In some embodiments regarding the immunoconjugate and the anti-CD20 antibody for use, the nucleic acid expression level is determined by RNA-seq, RT-qPCR, qPCR, multiplex qPCR or RT-qPCR, microarray analysis, SAGE, MassARRAY technique, ISH, or a combination thereof. In some embodiments regarding the immunoconjugate and the anti-CD20 antibody for use, the nucleic acid expression level is an mRNA expression level. In some embodiments regarding the immunoconjugate and the anti-CD20 antibody for use, the mRNA expression level is determined by RNA-seq.

In some embodiments regarding the immunoconjugate and the anti-CD20 antibody for use, which may be combined with any of the preceding aspects or embodiments, the sample is a tissue sample, tumor sample, whole blood sample, a plasma sample, a serum sample, or a combination thereof. In some embodiments regarding the immunoconjugate and the anti-CD20 antibody for use, the sample is a tissue sample. In some embodiments regarding the immunoconjugate and the anti-CD20 antibody for use, the tissue sample is a tumor tissue sample. In some embodiments regarding the immunoconjugate and the anti-CD20 antibody for use, the tumor tissue sample contains tumor cells, tumor-infiltrating immune cells, stromal cells, normal adjacent tissue (NAT) cells, or a combination thereof. In some embodiments regarding the immunoconjugate and the anti-CD20 antibody for use, the tumor tissue sample is a biopsy. In some embodiments regarding the immunoconjugate and the anti-CD20 antibody for use, the sample is an archival sample, a fresh sample, or a frozen sample.

In some embodiments regarding the immunoconjugate and the anti-CD20 antibody for use, which may be combined with any of the preceding aspects or embodiments, the sample is a tissue sample, the DLBCL is a germinal-center B-cell-like (GCB) or activated B-cell-like (ABC) cell-of-origin subgroup of DLBCL. In some embodiments regarding the immunoconjugate and the anti-CD20 antibody for use, which may be combined with any of the preceding aspects or embodiments, the DLBCL is a CD79a- and/or CD20-positive DLBCL. In some embodiments regarding the immunoconjugate and the anti-CD20 antibody for use, which may be combined with any of the preceding aspects or embodiments, the patient has not been previously treated for the DLBCL. In some embodiments regarding the immunoconjugate and the anti-CD20 antibody for use, which may be combined with any of the preceding aspects or embodiments, the patient has not been previously administered the immunoconjugate and the anti-CD20 antibody.

In some embodiments regarding the immunoconjugate and the anti-CD20 antibody for use, which may be combined with any of the preceding aspects or embodiments, the sample is a tissue sample, the anti-CD79b antibody comprises a heavy chain variable domain (VH) comprising the amino acid sequence of SEQ ID NO: 3 and a light chain variable domain (VL) comprising the amino acid sequence of SEQ ID NO: 4. In some embodiments regarding the immunoconjugate and the anti-CD20 antibody for use, the anti-CD79b antibody comprises: (a) a heavy chain comprising the amino acid sequence of SEQ ID NO: 13 and a light chain comprising the amino acid sequence of SEQ ID NO: 11; (b) a heavy chain comprising the amino acid sequence of SEQ ID NO: 12 and a light chain comprising the amino acid sequence of SEQ ID NO: 14; or (c) a heavy chain comprising the amino acid sequence of SEQ ID NO: 12 and a light chain comprising the amino acid sequence of SEQ ID NO: 11. In some embodiments regarding the immunoconjugate and the anti-CD20 antibody for use, which may be combined with any of the preceding aspects or embodiments, p is between 2 and 7, between 2 and 6, between 2 and 5, between 3 and 5, or between 3 and 4. In some embodiments regarding the immunoconjugate and the anti-CD20 antibody for use, p is 3.4. In some embodiments regarding the immunoconjugate and the anti-CD20 antibody for use, p is 3.5. In some embodiments regarding the immunoconjugate and the anti-CD20 antibody for use, which may be combined with any of the preceding aspects or embodiments, the immunoconjugate is polatuzumab vedotin.

In some embodiments regarding the immunoconjugate and the anti-CD20 antibody for use, which may be combined with any of the preceding aspects or embodiments, the sample is a tissue sample, the anti-CD20 antibody is a type I anti-CD20 antibody or a type II anti-CD20 antibody. In some embodiments regarding the immunoconjugate and the anti-CD20 antibody for use, the anti-CD20 antibody is a type I anti-CD20 antibody. In some embodiments regarding the immunoconjugate and the anti-CD20 antibody for use, the type I anti-CD20 antibody comprises the following CDRs: (a) a CDR-H1 with an amino acid sequence of SEQ ID NO: 26; (b) a CDR-H2 with an amino acid sequence of SEQ ID NO: 27; (c) a CDR-H3 with an amino acid sequence of SEQ ID NO: 28; (d) a CDR-L1 with an amino acid sequence of SEQ ID NO: 29; (e) a CDR-L2 with an amino acid sequence of SEQ ID NO: 30; and (f) a CDR-L3 with an amino acid sequence of SEQ ID NO: 31. In some embodiments regarding the immunoconjugate and the anti-CD20 antibody for use, the type I anti-CD20 antibody comprises a VH domain comprising an amino acid sequence of SEQ ID NO: 40 and a VL domain comprising an amino acid sequence of SEQ ID NO: 41. In some embodiments regarding the immunoconjugate and the anti-CD20 antibody for use, the type I anti-CD20 antibody is rituximab.

In some embodiments regarding the immunoconjugate and the anti-CD20 antibody for use, polatuzumab vedotin is for use at a dose of about 1.0 mg/kg to about 1.8 mg/kg. In some embodiments regarding the immunoconjugate and the anti-CD20 antibody for use, polatuzumab vedotin is for use at a dose of about 1.8 mg/kg. In some embodiments regarding the immunoconjugate and the anti-CD20 antibody for use, rituximab is for use at a dose of about 375 mg/m². In some embodiments regarding the immunoconjugate and the anti-CD20 antibody for use, polatuzumab vedotin and/or rituximab is for intravenous use.

In some embodiments regarding the immunoconjugate and the anti-CD20 antibody for use, which may be combined with any of the preceding aspects or embodiments, the treatment further comprises use of an effective amount of an additional therapeutic agent. In some embodiments regarding the immunoconjugate and the anti-CD20 antibody for use, the additional therapeutic agent is one or more of a chemotherapeutic agent, a corticosteroid, an anti-neoplastic agent, a growth inhibitory agent, an anti-angiogenic agent, a radiation therapy, a cytotoxic agent, or a combination thereof. In some embodiments regarding the immunoconjugate and the anti-CD20 antibody for use, the additional therapeutic agent is a chemotherapeutic agent and a corticosteroid. In some embodiments regarding the immunoconjugate and the anti-CD20 antibody for use, the chemotherapeutic agent is cyclophosphamide and/or doxorubicin. In some embodiments regarding the immunoconjugate and the anti-CD20 antibody for use, the corticosteroid is prednisone, prednisolone, or methylprednisolone. In some embodiments regarding the immunoconjugate and the anti-CD20 antibody for use, cyclophosphamide is for use at a dose of about 375 mg/m² to about 750 mg/m². In some embodiments regarding the immunoconjugate and the anti-CD20 antibody for use, doxorubicin is for use at a dose of about 25 mg/m² to about 50 mg/m². In some embodiments regarding the immunoconjugate and the anti-CD20 antibody for use: (a) prednisone is for use at a dose of about 100 mg; (b) prednisolone is for use at a dose of about 100 mg; or (c) methylprednisolone for use at a dose of about 80 mg. In some embodiments regarding the immunoconjugate and the anti-CD20 antibody for use, cyclophosphamide and/or doxorubicin is for intravenous use. In some embodiments regarding the immunoconjugate and the anti-CD20 antibody for use, prednisone, prednisolone, or methylprednisolone is for oral use.

In some embodiments regarding the immunoconjugate and the anti-CD20 antibody for use, polatuzumab vedotin, rituximab, cyclophosphamide, doxorubicin and/or prednisone, prednisolone, or methylprednisolone are for use in at least one 21-day cycle. In some embodiments regarding the immunoconjugate and the anti-CD20 antibody for use: (a) the polatuzumab vedotin, rituximab, cyclophosphamide, and/or doxorubicin are for use on day 1 of each 21-day cycle; and/or (b) prednisone, prednisolone, or methylprednisolone is for use on days 1-5 of each 21-day cycle. In some embodiments regarding the immunoconjugate and the anti-CD20 antibody for use, polatuzumab vedotin, rituximab, cyclophosphamide, doxorubicin, and/or prednisone, prednisolone, or methylprednisolone are for use for one, two, three, four, five, or six 21-day cycles.

In some embodiments, the invention features a method of identifying, diagnosing, and/or predicting whether a patient having a diffuse large B-cell lymphoma (DLBCL) may benefit from a treatment comprising an immunoconjugate, an anti-CD20 antibody, a chemotherapeutic agent, and a corticosteroid, the method comprising measuring a macrophage biomarker in a sample from the patient, wherein an amount or level of the macrophage biomarker in the sample that is below a reference macrophage biomarker amount or level identifies, diagnoses, and/or predicts the patient as one who may benefit from the treatment comprising the immunoconjugate, the anti-CD20 antibody, the chemotherapeutic agent, and the corticosteroid, wherein the immunoconjugate comprises the formula:

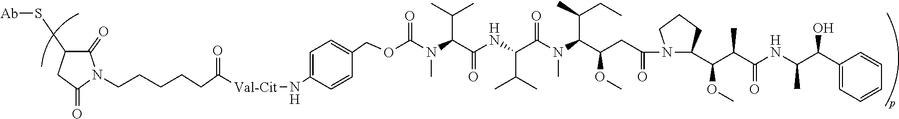

wherein Ab is an anti-CD79b antibody comprising a heavy chain comprising the amino acid sequence of SEQ ID NO: 12 and a light chain comprising the amino acid sequence of SEQ ID NO: 11 and wherein p is 3.5, wherein the anti-CD20 antibody is rituximab, wherein the chemotherapeutic agent comprises cyclophosphamide and doxorubicin, and wherein the corticosteroid comprises prednisone.

In some embodiments, the invention features a method of selecting a therapy for a patient having a DLBCL, the method comprising measuring a macrophage biomarker in a sample from the patient, wherein an amount or level of the macrophage biomarker in the sample that is below a reference macrophage biomarker amount or level identifies the patient as one who may benefit from a treatment comprising an immunoconjugate, an anti-CD20 antibody, a chemotherapeutic agent, and a corticosteroid, wherein the immunoconjugate comprises the formula:

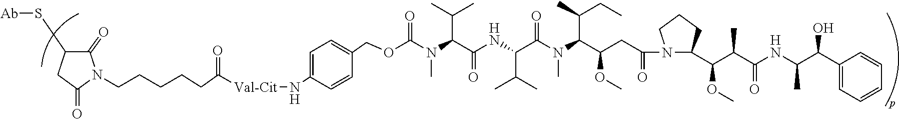

wherein Ab is an anti-CD79b antibody comprising a heavy chain comprising the amino acid sequence of SEQ ID NO: 12 and a light chain comprising the amino acid sequence of SEQ ID NO: 11 and wherein p is 3.5, wherein the anti-CD20 antibody is rituximab, wherein the chemotherapeutic agent comprises cyclophosphamide and doxorubicin, and wherein the corticosteroid comprises prednisone.

In some method embodiments, which may be combined with any of the preceding aspects or embodiments, the amount or level of the macrophage biomarker from the patient is below the reference macrophage biomarker amount or level, and the method further comprises administering to the patient an effective amount of the immunoconjugate, an effective amount of the anti-CD20 antibody, an effective amount of the chemotherapeutic agent, and an effective amount of the corticosteroid.

In some embodiments, the invention features a method of treating a patient having a DLBCL, the method comprising: (a) measuring a macrophage biomarker in a sample from the patient, wherein the amount or level of the macrophage biomarker in the sample is below a reference macrophage biomarker amount or level, and (b) administering an effective amount of an immunoconjugate, an effective amount of an anti-CD20 antibody, an effective amount of a chemotherapeutic agent, and an effective amount of a corticosteroid, to the patient based on the macrophage biomarker measured in step (a), and wherein the immunoconjugate comprises the formula:

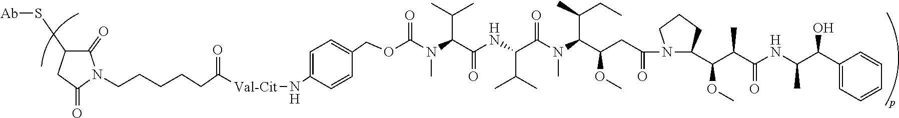

wherein Ab is an anti-CD79b antibody comprising a heavy chain comprising the amino acid sequence of SEQ ID NO: 12 and a light chain comprising the amino acid sequence of SEQ ID NO: 11 and wherein p is 3.5, wherein the anti-CD20 antibody is rituximab, wherein the chemotherapeutic agent comprises cyclophosphamide and doxorubicin, and wherein the corticosteroid comprises prednisone.

In some embodiments, the invention features a method of treating a patient having a DLBCL, the method comprising administering to the patient an effective amount of an immunoconjugate, an effective amount of an anti-CD20 antibody, an effective amount of a chemotherapeutic agent, and an effective amount of a corticosteroid, wherein prior to treatment the amount or level of a macrophage biomarker in a sample from the patient has been determined to be below a reference macrophage biomarker amount or level, and wherein the immunoconjugate comprises the formula:

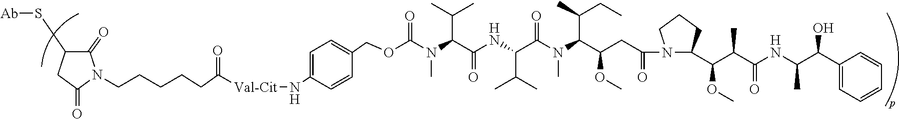

wherein Ab is an anti-CD79b antibody comprising a heavy chain comprising the amino acid sequence of SEQ ID NO: 12 and a light chain comprising the amino acid sequence of SEQ ID NO: 11 and wherein p is 3.5, wherein the anti-CD20 antibody is rituximab, wherein the chemotherapeutic agent comprises cyclophosphamide and doxorubicin, and wherein the corticosteroid comprises prednisone.

In some embodiments, the invention features a method of treating a patient having a DLBCL and having an amount or level of a macrophage biomarker in a sample from the patient that is below a reference macrophage biomarker amount or level comprising administering to the patient an effective amount of an immunoconjugate, an effective amount of an anti-CD20 antibody, an effective amount of a chemotherapeutic agent, and an effective amount of a corticosteroid, wherein the immunoconjugate comprises the formula:

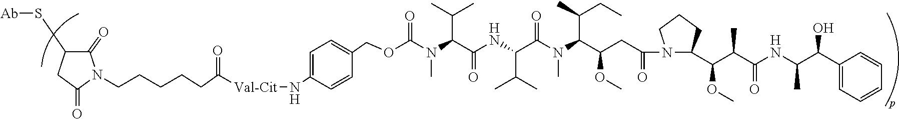

wherein Ab is an anti-CD79b antibody comprising a heavy chain comprising the amino acid sequence of SEQ ID NO: 12 and a light chain comprising the amino acid sequence of SEQ ID NO: 11 and wherein p is 3.5, wherein the anti-CD20 antibody is rituximab, wherein the chemotherapeutic agent comprises cyclophosphamide and doxorubicin, and wherein the corticosteroid comprises prednisone.

In some method embodiments, which may be combined with any of the preceding aspects or embodiments, the immunoconjugate is polatuzumab vedotin.

In some method embodiments, which may be combined with any of the preceding aspects or embodiments, the immunoconjugate is administered at a dose of about 1.8 mg/kg, the rituximab is administered at a dose of about 375 mg/m², the cyclophosphamide is administered at a dose of about 750 mg/m², the doxorubicin is administered at a dose of about 50 mg/m², and the prednisone is administered at a dose of about 100 mg.

### BRIEF DESCRIPTION OF DRAWINGS

FIG. 1 shows a schematic diagram of the POLARIX trial study design (Clinical Trial ID No. NCT03274492) which was a randomized double-blinded study in previously untreated diffuse large B-cell lymphoma (DLBCL) patients. Pola-R-CHP is a polatuzumab vedotin (1.8 mg/kg) plus rituximab (375 mg/m²), cyclophosphamide (750 mg/m²), doxorubicin (50 mg/m²), and prednisone (100 mg once daily on Days 1-5). *=IV on Day 1; †=R-CHOP: IV rituximab 375 mg/m², cyclophosphamide 750 mg/m², doxorubicin 50 mg/m², and vincristine 1.4 mg/m² (max. 2 mg) on Day 1, plus oral prednisone 100 mg once daily on Days 1-5; IPI=International prognostic index; ECOG PS=Eastern Cooperative Oncology Group performance status; and R=randomized.

FIG. 2 shows characteristics of RNA-seq-evaluable patients for the Pola-R-CHP and R-CHOP treatment arms. IPI=International prognostic index; n=number of patients; COO=cell of origin; ABC=activated B-cell-like; and GCB=germinal center B-cell-like.

FIGS. 3A & 3B show low M1 macrophage levels are associated with worse progression-free survival (PFS) after R-CHOP treatment but not after Pola-R-CHP treatment. Shown is PFS probability as a function of time (months) for high/low M1 macrophage signatures leveraging QuanTIseq in the R-CHOP (FIG. 3A) and Pola-R-CHP (FIG. 3B) treatment arms. In the inset of FIGS. 3A & 3B, the PFS hazard ratio (HR) for high versus low M1 macrophage levels for each treatment arm is shown. Also shown in the inset of FIGS. 3A & 3B are 3-year PFS estimates for high and low M1 macrophage levels. Reference group is below median. HRs were adjusted for IPI score (2 vs 3-5), age (<60 vs >60 years), COO (ABC, GCB, unclassified, unknown). IPI=International prognostic index; COO=cell of origin; ABC=activated B-cell-like; and GCB=germinal center B-cell-like.

FIG. 4 shows low M1 macrophage levels are associated with lower PFS at 24 months in the R-CHOP treatment arm but not the Pola-R-CHP treatment arm. Shown is the percentage of patients within a subgroup with progressive disease (PD) by 24 months. Patient subgroups shown are those having low M1 macrophage levels treated with R-CHOP (R-CHOP low), those having high M1 macrophage levels treated with R-CHOP (R-CHOP high), those having low M1 macrophage levels treated with Pola-R-CHP (Pola-R-CHP low), and those having high M1 macrophage levels treated with Pola-R-CHP (R-CHP high). QuanTIseq macrophage M1 levels are represented by median values (p-values by Fisher's exact test). Clopper-Pearson binomial 95% confidence intervals are displayed. M1 macrophage signature distribution was comparable between treatment arms.

FIGS. 5A & 5B show low M1 macrophage levels are associated with worse overall survival (OS) after R-CHOP treatment but not after Pola-R-CHP treatment. Shown is OS probability as a function of time (months) for high/low M1 macrophage signatures leveraging QuanTIseq in the R-CHOP (FIG. 5A) and Pola-R-CHP (FIG. 5B) treatment arms. In the inset of FIGS. 5A & 5B, the OS HR for high versus low M1 macrophage levels for each treatment arm is shown. Also shown in the inset of FIGS. 5A & 5B are 3-year OS estimates for high and low M1 macrophage levels. QuanTIseq macrophage M1 levels are represented by median values.

FIG. 6 shows PFS HRs for M1 macrophage levels derived from QuanTIseq and xCell for the Pola-R-CHP and R-CHOP treatment arms. Corresponding forest plots are shown. CI=confidence interval.

FIG. 7 shows baseline patient characteristics from the subset of patients (n=665) whose transcriptomic profiles were generated and analyzed via RNA-seq.

FIG. 8 shows PFS of patients with high vs low TAM signatures ranked by median in GOYA, MAIN or POLARIX clinical studies.

FIG. 9 shows correlation of TAM signature with spatially derived macrophage signatures from reactive lymph nodes and DLBCL tumors using scRNA macrophage data from a published pan-lymphoid tissue atlas and projection of TAM and MacroSig4 signatures onto corresponding UMAP.

FIG. 10 shows PFS of patients with high vs low TAM signatures in the POLARIX study treated with Pola-R-CHP.

### DETAILED DESCRIPTION OF THE INVENTION

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### II. Definitions

It is to be understood that aspects and embodiments of the invention described herein include “comprising,” “consisting,” and “consisting essentially of” aspects and embodiments. As used herein, the singular form “a,” “an,” and “the” includes plural references unless indicated otherwise.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X.” In some embodiments, “about” may refer to ±15%, ±10%, ±5%, or ±1% as understood by a person of skill in the art.

The “amount,” “level,” or “expression level,” used herein interchangeably, of a biomarker is a detectable level in a biological sample (e.g., a blood sample or a biopsy). “Expression” generally refers to the process by which information (e.g., gene-encoded and/or epigenetic) is converted into the structures present and operating in the cell. Therefore, as used herein, “expression” may refer to transcription into a polynucleotide, translation into a polypeptide, or even polynucleotide and/or polypeptide modifications (e.g., post-translational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs). Expression levels can be measured by methods known to one skilled in the art and also disclosed herein. The expression level or amount of a biomarker can be used to identify/characterize a subject having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) who may be likely to respond to, or benefit from, a particular therapy (e.g., a therapy comprising one or more dosing cycles of an anti-CD79b immunoconjugate (e.g., polatuzumab vedotin) and an anti-CD20 antibody (e.g., obinutuzumab or rituximab)).

The presence and/or expression level/amount of various biomarkers described herein in a sample (e.g., a blood sample or a biopsy) can be analyzed by a number of methodologies, many of which are known in the art and understood by the skilled artisan, including, but not limited to, immunohistochemistry (“IHC”), western blot analysis, immunoprecipitation, molecular binding assays, ELISA, ELIFA, flow cytometry, fluorescence activated cell sorting (“FACS”), spatial transcriptomics, spatial proteomics, MassARRAY, proteomics, quantitative blood based assays (e.g., Serum ELISA), biochemical enzymatic activity assays, in situ hybridization (ISH), fluorescence in situ hybridization (FISH), Southern analysis, Northern analysis, whole genome sequencing, massively parallel DNA sequencing (e.g., next-generation sequencing), NANOSTRING®, polymerase chain reaction (PCR) including quantitative real time PCR (qRT-PCR) and other amplification type detection methods, such as, for example, branched DNA, SISBA, TMA and the like, RNA-seq, microarray analysis, gene expression profiling, and/or serial analysis of gene expression (“SAGE”), as well as any one of the wide variety of assays that can be performed by protein, gene, and/or tissue array analysis. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis). Multiplexed immunoassays such as those available from Rules Based Medicine or Meso Scale Discovery (“MSD”) may also be used.

The term “antagonist” is used in the broadest sense, and includes any molecule that partially or fully blocks, inhibits, or neutralizes a biological activity of a native polypeptide disclosed herein. Suitable antagonist molecules specifically include antagonist antibodies or antibody fragments (e.g., antigen-binding fragments), fragments or amino acid sequence variants of native polypeptides, peptides, antisense oligonucleotides, small organic molecules, etc. Methods for identifying antagonists of a polypeptide may comprise contacting a polypeptide with a candidate antagonist molecule and measuring a detectable change in one or more biological activities normally associated with the polypeptide.

The term “anti-CD20 antibody” according to the invention refers to an antibody that is capable of binding CD20 with sufficient affinity such that the antibody is useful as a diagnostic and/or therapeutic agent in targeting CD20. Preferably, the extent of binding of an anti-CD20 antibody to an unrelated, non-CD20 protein is less than about 10% of the binding of the antibody to CD20 as measured, e.g., by a radioimmunoassay (RIA). In certain embodiments, an antibody that binds to CD20 has a dissociation constant (Kd) of <1 μM, <100 nM, <10 nM, <1 nM, or <0.1 nM. In certain embodiments, anti-CD20 antibody binds to an epitope of CD20 that is conserved among CD20 from different species.

The meaning of “Type I” and “Type II” anti-CD20 antibodies are well known in the art. In general, anti-CD20 monoclonal antibodies fall into two distinct categories based on their mechanism of action in eradicating lymphoma cells. “Type I” anti-CD20 antibodies primarily utilize complement to kill target cells, while “Type II” anti-CD20 antibodies operate by different mechanisms, primarily apoptosis. Rituximab (see, e.g., U.S. Pat. No. 5,736,137, which is incorporated herein by reference in its entirety) and 1F5 are examples of Type I anti-CD20 antibodies, whereas obinutuzumab (see, e.g., WO 2005/044859 and U.S. Patent Publication No. 2005/0123546, which are incorporated by reference herein in their entirety) and B1 are examples of a Type II antibody. See, e.g., Cragg (Blood 103(7), 2004, 2738-2743); Teeling (Blood 104(6), 2004, 1793-1800); EP2380910 and WO 2005/044859, the entire contents of which are hereby incorporated by reference.

“CD20” as used herein refers to the human B-lymphocyte antigen CD20 (also known as CD20, B-lymphocyte surface antigen B1, Leu-16, Bp35, BM5, and LF5; the sequence is characterized by the SwissProt database entry P11836) is a hydrophobic transmembrane protein with a molecular weight of approximately 35 kD located on pre-B and mature B lymphocytes. (Valentine, M. A., et al., J. Biol. Chem. 264(19) (1989 11282-11287; Tedder, T. F., et al, Proc. Natl. Acad. Sci. U.S.A. 85 (1988) 208-12; Stamenkovic, I., et al., J. Exp. Med. 167 (1988) 1975-80; Einfeld, D. A. et al., EMBO J. 7 (1988) 711-7; Tedder, T. F., et al., J. Immunol. 142 (1989) 2560-8). The corresponding human gene is Membrane-spanning 4-domains, subfamily A, member 1, also known as MS4A1. This gene encodes a member of the membrane-spanning 4A gene family. Members of this nascent protein family are characterized by common structural features and similar intron/exon splice boundaries and display unique expression patterns among hematopoietic cells and nonlymphoid tissues. This gene encodes the B-lymphocyte surface molecule which plays a role in the development and differentiation of B-cells into plasma cells. This family member is localized to 11q12, among a cluster of family members. Alternative splicing of this gene results in two transcript variants which encode the same protein.

The terms “CD20” and “CD20 antigen” are used interchangeably herein, and include any variants, isoforms and species homologs of human CD20 which are naturally expressed by cells or are expressed on cells transfected with the CD20 gene. Binding of an antibody of the invention to the CD20 antigen mediates the killing of cells expressing CD20 (e.g., a tumor cell) by inactivating CD20. The killing of the cells expressing CD20 may occur by one or more of the following mechanisms: Cell death/apoptosis induction, ADCC and CDC. Synonyms of CD20, as recognized in the art, include B-lymphocyte antigen CD20, B-lymphocyte surface antigen B1, Leu-16, Bp35, BM5, and LF5.

The term “expression of the CD20” antigen is intended to indicate a significant level of expression of the CD20 antigen in a cell, e.g., a T- or B-Cell. In one embodiment, patients to be treated according to the methods of this invention express significant levels of CD20 on a B-cell tumor or cancer. Patients having a “CD20 expressing cancer” can be determined by standard assays known in the art. E.g., CD20 antigen expression is measured using immunohistochemical (IHC) detection, FACS or via PCR-based detection of the corresponding mRNA.

As used herein, “administering” is meant as a method of giving a dosage of a compound (e.g., an anti-CD79b immunoconjugate (e.g., polatuzumab vedotin) and an anti-CD20 antibody (e.g., obinutuzumab or rituximab)), or a composition (e.g., a pharmaceutical composition, e.g., a pharmaceutical composition including an anti-CD79b immunoconjugate (e.g., polatuzumab vedotin) and an anti-CD20 antibody (e.g., obinutuzumab or rituximab)) to a subject. The compounds and/or compositions utilized in the methods described herein can be administered, for example, intravenously (e.g., by intravenous infusion), subcutaneously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. The method of administration can vary depending on various factors (e.g., the compound or composition being administered and the severity of the condition, disease, or disorder being treated).

A “fixed” or “flat” dose of a therapeutic agent (e.g., an anti-CD79b immunoconjugate (e.g., polatuzumab vedotin) and an anti-CD20 antibody (e.g., obinutuzumab or rituximab)) herein refers to a dose that is administered to a patient without regard for the weight or body surface area (BSA) of the patient. The fixed or flat dose is therefore not provided as a mg/kg dose or a mg/m² dose, but rather as an absolute amount of the therapeutic agent (e.g., mg).

As used herein, the term “treatment” or “treating” refers to clinical intervention designed to alter the natural course of the individual or cell being treated during the course of clinical pathology. Desirable effects of treatment include delaying or decreasing the rate of disease progression, ameliorating or palliating the disease state, and remission or improved prognosis. For example, an individual is successfully “treated” if one or more symptoms associated with cancer (e.g., lymphoma, e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, delaying the progression of the disease, and/or prolonging survival of individuals.

As used herein, “in combination with” or “in conjunction with” refers to administration of one treatment modality in addition to another treatment modality. As such, “in combination with” or “in conjunction with” refers to administration of one treatment modality before, during, or after administration of the other treatment modality to the individual. The co-administration can be simultaneous or sequential in either order, wherein preferably there is a time period while both or all active agents simultaneously exert their biological activities. Said antibody and said further agent(s) are co-administered either simultaneously or sequentially (e.g., intravenous (i.v.)), for example through a continuous infusion. When both therapeutic agents are co-administered sequentially the dose administered either on the same day in two separate administrations, or one of the agents may be administered on day 1 and the second may be co-administered on day 2 to day 7, preferably on day 2 to 4. Thus, in one embodiment, the term “sequentially” means within (about) 7 days after the dose of the first component, preferably within (about) 4 days after the dose of the first component; and the term “simultaneously” means at the same time. The term “coadministration” with respect to the maintenance doses of the antibody and/or further agent(s) means that the maintenance doses can be either co-administered simultaneously, if the treatment cycle is appropriate for both drugs, e.g., every week or the further agent is, e.g., administered, e.g., every first to third day and said antibody is administered every week. Or the maintenance doses are co-administered sequentially, either within one or within several days. In a preferred embodiment, the anti-CD79b immunoconjugates (e.g., polatuzumab vedotin or its functional equivalents) and anti-CD20 antibodies (e.g., obinutuzumab, rituximab, or their functional equivalents) described herein may be administered in combination with a chemotherapy, for example with a CHP chemotherapy or with variants of a CHP chemotherapy. Therefore, in a preferred embodiment, the additional chemotherapeutic agents to be co-administered are selected from the group consisting of cyclophosphamide, hydroxydaunorubicin, prednisone or prednisolone and, optionally, etoposide.

A “disorder” or “disease” is any condition that would benefit from treatment including, but not limited to, disorders that are associated with some degree of abnormal cell proliferation, e.g., cancer, e.g., lymphoma.

The terms “cancer” and “cancerous” refer to or describe the physiological condition in mammals that is typically characterized by unregulated cell growth. Examples of cancer include, but are not limited to, lymphoma, carcinoma, blastoma, sarcoma, and leukemia or lymphoid malignancies. More particular examples of such cancers include, but are not limited to, multiple myeloma and B-cell lymphoma (including low grade/follicular non-Hodgkin's lymphoma (NHL)); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); acute myelogenous leukemia (AML); hairy cell leukemia; chronic myeloblastic leukemia (CML); post-transplant lymphoproliferative disorder (PTLD); and myelodysplastic syndromes (MDS), and associated metastases. In some embodiments, the cancer is a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)). In some embodiments, the lymphoma is an indolent lymphoma. In some embodiments, the lymphoma is a B-cell lymphoma. In some embodiments, the B-cell lymphoma is a germinal center derived B-cell lymphoma. In some embodiments, the B-cell lymphoma is an NHL. In some embodiments, the lymphoma is a diffuse large B-cell lymphoma (DLBCL). In some embodiments, the lymphoma is a DLBCL. In some embodiments, the DLBCL is a germinal-center B-cell-like (GCB) or activated B-cell-like (ABC) cell-of-origin subgroup of DLBCL. In some embodiments, the lymphoma is a CD20-positive lymphoma.

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

As used herein, “metastasis” is meant the spread of cancer (e.g., lymphoma, e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

The term “anti-cancer therapy” refers to a therapy useful in treating cancer (e.g., lymphoma, e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)). Examples of anti-cancer therapeutic agents include, but are limited to, e.g., immunomodulatory agents, or an agent that increases or activates one or more immune co-stimulatory receptors, chemotherapeutic agents, growth inhibitory agents, cytotoxic agents, agents used in radiation therapy, anti-angiogenesis agents, apoptotic agents, anti-tubulin agents, and other agents to treat cancer. Combinations thereof are also included in the invention. In some embodiments, the anti-cancer therapy includes cyclophosphamide, doxorubicin, and prednisone (CHP) or variants thereof (e.g., a CHOEP chemotherapy, a CHOP-14 chemotherapy or an ACVBP chemotherapy (see, for example, the examples and also EP-B1 2380910, WO 2005/044859 and Scott, 2014 and 2015, loc. cit.)).

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anti-cancer agents disclosed below.

“Chemotherapeutic agent” includes chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors (e.g., an anaplastic lymphoma kinase (Alk) inhibitor, such as AF-802 (also known as CH-5424802 or alectinib)); (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds described include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG1 λ antibody genetically modified to recognize interleukin-12 p40 protein.

Chemotherapeutic agent also includes “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6.3 and E7.6.3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; inhibitors of insulin receptor tyrosine kinases, including anaplastic lymphoma kinase (Alk) inhibitors, such as AF-802 (also known as CH-5424802 or alectinib), ASP3026, X396, LDK378, AP26113, crizotinib (XALKORI®), and ceritinib (ZYKADIA®); small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g., those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTa1/β2 blockers such as Anti-lymphotoxin alpha (LTa); radioactive isotopes (e.g., At211, I131, I125, Y90, Re186, Re188, Sm153, Bi212, P32, Pb212 and radioactive isotopes of Lu); miscellaneous investigational agents such as thioplatin, PS-341, phenylbutyrate, ET-18-OCH3, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g., celecoxib or etoricoxib), proteosome inhibitor (e.g., PS341); CCI-779; tipifarnib (RI1577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above such as CHP, an abbreviation for a combined therapy of cyclophosphamide, doxorubicin, and prednisolone (prednisone); and FOLFOX, an abbreviation for a treatment regimen with oxaliplatin (ELOXATIN™) combined with 5-FU and leucovorin.

Chemotherapeutic agents also include non-steroidal anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

An “effective amount” of a compound, for example, an anti-CD79b immunoconjugate (e.g., polatuzumab vedotin) and an anti-CD20 antibody (e.g., obinutuzumab or rituximab)), or a composition (e.g., pharmaceutical composition) thereof, is at least the minimum amount required to achieve the desired therapeutic result, such as a measurable increase in overall survival or progression-free survival of a particular disease or disorder (e.g., lymphoma, e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)). An effective amount herein may vary according to factors such as the disease state, age, sex, and weight of the patient, and the ability of the antibody to elicit a desired response in the subject. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications, and intermediate pathological phenotypes presenting during development of the disease. For therapeutic use, beneficial or desired results include clinical results such as decreasing one or more symptoms resulting from the disease (e.g., reduction or delay in cancer-related pain, reduction in symptoms per the European Organization for Research and Treatment of Cancer Quality-of-Life Questionnaire (EORTC QLQ-C30, e.g., fatigue, nausea, vomiting, pain, dyspnea, insomnia, appetite loss, constipation, diarrhea, or general level of physical emotional, cognitive, or social functioning), increase from baseline in functional assessment of cancer therapy-lymphoma (FACT-Lym) subscale score, decreasing the dose of other medications required to treat the disease, enhancing effect of another medication such as via targeting, delaying the progression of the disease (e.g., progression-free survival, delay of unequivocal clinical progression (e.g., cancer-related pain progression, deterioration in Eastern Cooperative Group Oncology Group (ECOG) Performance Status (PS) (e.g., how the disease affects the daily living abilities of the patient), and/or initiation of next systemic anti-cancer therapy, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

“Immunogenicity” refers to the ability of a particular substance to provoke an immune response. Tumors are immunogenic and enhancing tumor immunogenicity aids in the clearance of the tumor cells by the immune response. Examples of enhancing tumor immunogenicity include but are not limited to treatment with an anti-CD79b immunoconjugate (e.g., polatuzumab vedotin) and an anti-CD20 antibody (e.g., obinutuzumab or rituximab).

“Individual response” or “response” can be assessed using any endpoint indicating a benefit to the subject, including, without limitation, (1) inhibition, to some extent, of disease progression (e.g., progression of cancer, e.g., lymphoma) including slowing down and complete arrest; (2) a reduction in tumor size; (3) inhibition (i.e., reduction, slowing down or complete stopping) of cancer cell infiltration into adjacent peripheral organs and/or tissues; (4) inhibition (i.e., reduction, slowing down or complete stopping) of metastasis; (5) relief, to some extent, of one or more symptoms associated with the disease or disorder (e.g., lymphoma, e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)); (6) increase or extend in the length of survival, including overall survival and progression-free survival; and/or (9) decreased mortality at a given point of time following treatment.

An “effective response” of a subject or a subject's “responsiveness” to treatment with a medicament and similar wording refers to the clinical or therapeutic benefit imparted to a subject as risk for, or suffering from, a disease or disorder, such as cancer. In one embodiment, such benefit includes any one or more of: extending survival (including overall survival and progression free survival); resulting in an objective response (including a complete response or a partial response); or improving signs or symptoms of cancer.

A subject who “does not have an effective response” to treatment refers to a subject who does not have any one of extending survival (including overall survival and progression free survival); resulting in an objective response (including a complete response or a partial response); or improving signs or symptoms of cancer.

As used herein, “survival” refers to the patient remaining alive, and includes overall survival as well as progression-free survival.

As used herein, “overall survival” (OS) refers to the time from entry into a study to death from any cause. As used herein, “overall survival rate” refers to the percentage of subjects in a group who are alive after a particular duration of time, e.g., six months, 1 year, or 5 years from the time of diagnosis or treatment.

As used herein, “complete response” or “CR” refers to disappearance of all evidence of disease.

As used herein, “partial response” or “PR” refers to a measurable alleviation of symptoms, diminishment of any direct or indirect pathological consequences of the lymphoma, decrease in the rate of disease progression, amelioration or palliation of the disease state, or prevention of metastasis that does not eliminate all evidence of disease.

As used herein, “progression-free survival” (PFS) refers to the length of time during and after treatment during which the disease being treated (e.g., cancer, e.g., e.g., lymphoma, e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) does not get worse (e.g., lymphoma progression or death as a result of any cause). PFS may include the amount of time patients have experienced a complete response or a partial response, as well as the amount of time patients have experienced stable disease.

As used herein “extending survival” refers to increasing overall survival or progression free survival in a treated patient relative to an untreated patient (e.g., relative to a patient not treated with the medicament), or relative to a patient who does not express a biomarker at the designated level, and/or relative to a patient treated with an approved anti-tumor agent. An objective response refers to a measurable response, including complete response (CR) or partial response (PR).

The skilled person is readily able to decide whether a given clinical outcome is improved in accordance with the invention (e.g., improved as compared to a treatment without an anti-CD79b immunoconjugate (e.g., polatuzumab vedotin) and an anti-CD20 antibody (e.g., obinutuzumab or rituximab)). For example, “improved” in this context means that the clinical outcome (resulting from the treatment with the anti-CD79b immunoconjugate (e.g., polatuzumab vedotin or its functional equivalents) and the anti-CD20 antibody (e.g., obinutuzumab/a functional equivalent of obinutuzumab or rituximab), particularly in combination with a chemotherapy, particularly in combination with a CHP chemotherapy) is at least 3% higher, at least 5% higher, at least 7% higher, at least 10% higher, at least 15% higher, at least 20% higher, at least 25% higher, at least 30% higher, at least 40% higher, at least 50% higher, at least 75% higher, at least 100% higher, or at least 120% higher, as compared to the clinical outcome resulting from a comparable treatment without an anti-CD79b immunoconjugate (e.g., polatuzumab vedotin) and an anti-CD20 antibody (e.g., obinutuzumab or rituximab), particularly in combination with a chemotherapy, particularly in combination with a CHP chemotherapy.

The time at which the clinical outcome/clinical endpoint is assessed can readily be determined by the skilled person. In principle, it is determined at a timepoint when the difference in the clinical outcome/clinical endpoint between the two treatments (e.g., polatuzumab vedotin and rituximab (or obinutuzumab) treatment vs. rituximab (or obinutuzumab) treatment) becomes evident. This time may, for example, be at least 1 month, at least 2 months, at least 3 months, at least 6 months, at least 12 months, at least 18 months, at least 24 months, at least 30 months, at least 36 months, at least 42 months, or at least 48 months, after the beginning of the treatment.

As used herein, “delaying progression” of a disorder or disease means to defer, hinder, slow, retard, stabilize, and/or postpone development of the disease or disorder (e.g., cancer, e.g., lymphoma, e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)). This delay can be of varying lengths of time, depending on the history of the disease and/or subject being treated. As is evident to one skilled in the art, a sufficient or significant delay can, in effect, encompass prevention, in that the subject does not develop the disease.

As used herein, the term “reducing or inhibiting cancer relapse” means to reduce or inhibit tumor or cancer relapse, or tumor or cancer progression.

By “reduce or inhibit” is meant the ability to cause an overall decrease of 20%, 30%, 40%, 50%, 60%, 70%, 75%, 80%, 85%, 90%, 95%, or greater. Reduce or inhibit can refer to the symptoms of the disorder being treated (e.g., lymphoma, e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)), the presence or size of metastases, or the size of the primary tumor.

As used herein, “subject” or “individual” is meant a mammal, including, but not limited to, a human or non-human mammal, such as a bovine, equine, canine, ovine, or feline. In some embodiments, the subject is a human. Patients are also humans herein.

The terms “detecting” and “detection” are used herein in the broadest sense to include both qualitative and quantitative measurements of a target molecule. Detecting includes identifying the mere presence of the target molecule in a sample as well as determining whether the target molecule is present in the sample at detectable levels. Detecting may be direct or indirect.

A “tumor-infiltrating immune cell,” as used herein, refers to any immune cell present in a tumor or a sample thereof. Tumor-infiltrating immune cells include, but are not limited to, intratumoral immune cells, peritumoral immune cells, other tumor stroma cells (e.g., fibroblasts), or any combination thereof. Such tumor-infiltrating immune cells can be, for example, macrophages (e.g., M1 macrophages, tumor-associated macrophages, or M2 macrophages), monocytes, T lymphocytes (such as CD8+ T lymphocytes and/or CD4+ T lymphocytes), B lymphocytes, or other bone marrow-lineage cells, including granulocytes (e.g., neutrophils, eosinophils, and basophils), dendritic cells (e.g., interdigitating dendritic cells), histiocytes, and natural killer cells.

The term “biomarker” as used herein refers to an indicator, e.g., predictive, diagnostic, and/or prognostic, which can be detected in a sample (e.g., a tumor tissue sample (e.g., a lymphoma tumor tissue sample, e.g., a B-cell lymphoma tumor tissue sample, e.g., a non-Hodgkin lymphoma tumor tissue sample, e.g., a diffuse large B-cell lymphoma (e.g., a germinal-center B-cell-like or activated B-cell-like diffuse large B-cell lymphoma) tumor tissue sample), a blood sample, or a biopsy). The biomarker may serve as an indicator of a particular subtype of a disease or disorder (e.g., a diffuse large B-cell lymphoma (e.g., a germinal-center B-cell-like or activated B-cell-like diffuse large B-cell lymphoma)) characterized by certain, molecular, pathological, histological, and/or clinical features. In some aspects, a biomarker is a gene (e.g., any of the genes described herein). Biomarkers include, but are not limited to, polypeptides, polynucleotides (e.g., DNA, and/or RNA), polynucleotide copy number alterations (e.g., DNA copy numbers), polypeptide and polynucleotide modifications (e.g., posttranslational modifications), carbohydrates, and/or glycolipid-based molecular markers. In some embodiments, the biomarker is a gene expression value. In some embodiments, the biomarker is a M1 macrophage gene signature set score. In some embodiments, the biomarker is a tumor-associated macrophage (TAM) gene signature set score. In some embodiments, the biomarker is a cell (e.g., an immune cell, e.g., a macrophage, e.g., an M1 macrophage or an M2 macrophage). In some embodiments, the biomarker is an amount of M1 macrophages. In some embodiments, the biomarker is an amount of tumor-associated macrophages.

The term “macrophage biomarker” as used herein refers to a biomarker that indicates an amount, level, characteristic, or phenotype of macrophages within a sample (e.g., a tumor tissue sample (e.g., a lymphoma tumor tissue sample, e.g., a B-cell lymphoma tumor tissue sample, e.g., a non-Hodgkin lymphoma tumor tissue sample, e.g., a diffuse large B-cell lymphoma (e.g., a germinal-center B-cell-like or activated B-cell-like diffuse large B-cell lymphoma) tumor tissue sample), a blood sample, or a biopsy). In some aspects, a macrophage biomarker is a gene (e.g., any of the genes described herein). In some aspects, a macrophage biomarker is a polypeptide, polynucleotide (e.g., DNA, and/or RNA), polynucleotide copy number alteration (e.g., DNA copy number), polypeptide and polynucleotide modification (e.g., posttranslational modification), carbohydrate, and/or glycolipid-based molecular marker. In some embodiments, the macrophage biomarker is a gene expression value, which can be reflective of one or more genes (e.g., one or more of the genes described herein). In some embodiments, the macrophage biomarker is a M1 macrophage gene signature set score. In some embodiments, the macrophage biomarker is a tumor-associated macrophage (TAM) gene signature set score. In some embodiments, the macrophage biomarker is a cell (e.g., an M1 macrophage, a tumor-associated macrophage). In some embodiments, the macrophage biomarker is an amount of M1 macrophages. In some embodiments, the macrophage biomarker is an amount of tumor-associated macrophages.

A biomarker is “predictive” in accordance with the invention if it can be used to identify a patient defined herein (optionally in combination with one or more other biomarkers), e.g., a patient that responds to treatment with an anti-CD79b immunoconjugate (e.g., polatuzumab vedotin) and anti-CD20 antibody (e.g., obinutuzumab or rituximab) (particularly in combination with a chemotherapy, particularly in combination with a CHP chemotherapy). In some embodiments, a biomarker is predictive if the treatment effect differs between the biomarker-defined subgroups of patients. It is preferred in this context, that the predictive biomarker(s) is (are) the biomarker(s) as defined herein elsewhere. Particular examples of predictive biomarkers to be assessed in the context of the invention are the macrophage biomarkers described herein.

The term “antibody” includes monoclonal antibodies (including full-length antibodies which have an immunoglobulin Fc region), antibody compositions with polyepitopic specificity, multispecific antibodies (e.g., bispecific antibodies), diabodies, and single-chain molecules, as well as antibody fragments, including antigen-binding fragments, such as Fab, F(ab′)₂, and Fv. The term “immunoglobulin” (Ig) is used interchangeably with “antibody” herein.

The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 Daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for p and E isotypes. Each L chain has at the N-terminus, a variable domain (V_{L}) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th Edition, Daniel P. Sties, Abba I. Terr and Tristram G. Parsolw (eds), Appleton & Lange, Norwalk, CT, 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated α, δ, ε, γ, and μ, respectively. The γ and α classes are further divided into subclasses on the basis of relatively minor differences in the CH sequence and function, e.g., humans express the following subclasses: IgG1, IgG2A, IgG2B, IgG3, IgG4, IgAQ1 and IgA2.

The term “hypervariable region” or “HVR” as used herein refers to each of the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, NJ, 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, MD. (1991)). Chothia refers instead to the location of the structural loops (Chothia and Lesk, J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below in Table 1.

TABLE 1
Residues of HVRs

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B (Kabat |
| | | | | numbering) |
| H1 | H31-H35 | H26-H35 | H26-H32 | H30-H35 (Chothia |
| | | | | numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g., residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, MD (1991)). The constant domains are not involved directly in the binding of antibody to an antigen, but exhibit various effector functions, such as participation of the antibody in antibody-dependent cellular toxicity.

The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full-length antibody,” “intact antibody,” and “whole antibody” are used interchangeably to refer to an antibody in its substantially intact form, as opposed to an antibody fragment. Specifically, whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, preferably the antigen-binding and/or the variable region of the intact antibody. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10): 1057-1062 (1995)); single-chain antibody molecules and multispecific antibodies formed from antibody fragments. Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}I domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

“Functional fragments” of the antibodies described comprise a portion of an intact antibody, generally including the antigen binding or variable region of the intact antibody or the Fc region of an antibody which retains or has modified FcR binding capability. Examples of antibody fragments include linear antibody, single-chain antibody molecules and multispecific antibodies formed from antibody fragments.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies described include human IgG1, IgG2 (IgG2A, IgG2B), IgG3 and IgG4. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see M. Daëron, Annu. Rev. Immunol. 15:203-234 (1997). FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med. 126: 330-41 (1995). Other FcRs, including those to be identified in the future, are encompassed by the term “FcR” herein.

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO 93/11161; Hollinger et al., Proc. Natl. Acad. Sci. USA 90: 6444-6448 (1993).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is used a subset of “chimeric antibodies.”

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

“Affinity” refers to the strength of the sum total of non-covalent interactions between a single binding site of a molecule (e.g., an antibody) and its binding partner (e.g., an antigen, e.g., CD20). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary embodiments for measuring binding affinity are described in the following.

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from an HVR (hereinafter defined) of the recipient are replaced by residues from an HVR of a non-human species (donor antibody) such as mouse, rat, rabbit or non-human primate having the desired specificity, affinity, and/or capacity. In some instances, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR are typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

The term “isolated antibody” when used to describe the various antibodies disclosed herein, means an antibody that has been identified and separated and/or recovered from a cell or cell culture from which it was expressed. Contaminant components of its natural environment are materials that would typically interfere with diagnostic or therapeutic uses for the polypeptide, and can include enzymes, hormones, and other proteinaceous or non-proteinaceous solutes. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For a review of methods for assessment of antibody purity, see, e.g., Flatman et al., J. Chromatogr. B 848:79-87 (2007). In preferred embodiments, the antibody will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated antibody includes antibodies in situ within recombinant cells, because at least one component of the polypeptide natural environment will not be present. Ordinarily, however, isolated polypeptide will be prepared by at least one purification step.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein., Nature, 256:495-97 (1975); Hongo et al., Hybridoma, 14 (3): 253-260 (1995), Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

As used herein, the term “binds,” “specifically binds to,” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, for example, by a radioimmunoassay (RIA). In certain embodiments, an antibody that specifically binds to a target has a dissociation constant (K_{D}) of ≤1 μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding. The term as used herein can be exhibited, for example, by a molecule having a K_{D} for the target of 10⁻⁴ M or lower, alternatively 10⁻⁵ M or lower, alternatively 10⁻⁶ M or lower, alternatively 10⁻⁷ M or lower, alternatively 10⁻⁸ M or lower, alternatively 10⁻⁹ M or lower, alternatively 10⁻⁰ M or lower, alternatively 10⁻¹¹ M or lower, alternatively 10⁻¹² M or lower or a K_{D} in the range of 10⁻⁴ M to 10⁻⁶ M or 10⁻⁶ M to 10⁻¹⁰ M or 10⁻⁷ M to 10⁻⁹ M. As will be appreciated by the skilled artisan, affinity and K_{D} values are inversely related. A high affinity for an antigen is measured by a low K_{D} value. In one embodiment, the term “specific binding” refers to binding where a molecule binds to a particular polypeptide or epitope on a particular polypeptide without substantially binding to any other polypeptide or polypeptide epitope.

The phrase “substantially reduced” or “substantially different,” as used herein, denotes a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., K_{D} values). The difference between said two values is, for example, greater than about 10%, greater than about 20%, greater than about 30%, greater than about 40%, and/or greater than about 50% as a function of the value for the reference/comparator molecule.

The term “substantially similar” or “substantially the same,” as used herein, denotes a sufficiently high degree of similarity between two numeric values (for example, one associated with an antibody of the invention and the other associated with a reference/comparator antibody), such that one of skill in the art would consider the difference between the two values to be of little or no biological and/or statistical significance within the context of the biological characteristic measured by said values (e.g., K_{D} values). The difference between said two values is, for example, less than about 50%, less than about 40%, less than about 30%, less than about 20%, and/or less than about 10% as a function of the reference/comparator value.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

$$100 times the fraction X / Y$$

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “sample,” as used herein, refers to a composition that is obtained or derived from a subject and/or individual of interest that contains a cellular and/or other molecular entity that is to be characterized and/or identified, for example based on physical, biochemical, chemical and/or physiological characteristics. For example, the phrase “tumor sample,” “disease sample,” and variations thereof refers to any sample (e.g., a biopsy or a blood sample) obtained from a subject of interest that would be expected or is known to contain the cellular and/or molecular entity that is to be characterized. In some embodiments, the sample is a tumor tissue sample (e.g., a lymphoma tumor tissue sample, e.g., a B-cell lymphoma tumor tissue sample, e.g., a non-Hodgkin lymphoma tumor tissue sample, e.g., a diffuse large B-cell lymphoma (e.g., a germinal-center B-cell-like or activated B-cell-like diffuse large B-cell lymphoma) tumor tissue sample). Other samples include, but are not limited to, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, stool, tumor lysates, and tissue culture medium, tissue extracts such as homogenized tissue, cellular extracts, and combinations thereof. Samples may be fresh or may be processed (e.g., frozen, fixed, or formalin-fixed, paraffin-embedded (FFPE)) for storage.

By “tissue sample” or “cell sample” is meant a collection of similar cells obtained from a tissue of a subject or individual. The source of the tissue or cell sample may be solid tissue as from a fresh, frozen, and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a diseased tissue/organ. The tissue sample may contain compounds which are not naturally intermixed with the tissue in nature such as preservatives, anticoagulants, buffers, fixatives, wax, nutrients, antibiotics, or the like.

A “reference sample,” “reference cell,” “reference tissue,” “control sample,” “control cell,” or “control tissue,” as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject. For example, healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject. In yet another embodiment, a reference sample, reference cell, reference tissue, control sample, control cell, or control tissue is obtained from a healthy and/or non-diseased part of the body (e.g., tissues or cells) of a subject who is not the subject. In even another embodiment, a reference sample, reference cell, reference tissue, control sample, control cell, or control tissue is obtained from an untreated tissue and/or cell of the body of an individual who is not the subject.

In general, as used in the context of the present invention, a non-limiting example of a “control” is preferably a “non-responder” control, for example a sample/cell/tissue obtained from one or more patients that do not suffer from the particular lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) as defined herein (non-“patient defined herein”) and that are known to be not advantageously responsive to an anti-CD79b immunoconjugate (e.g., polatuzumab vedotin) and an anti-CD20 antibody (e.g., obinutuzumab or rituximab) (in particular in combination with a chemotherapy, more particular in combination with a CHP chemotherapy) in accordance with the invention. Another example for a “non-responder” control is a cell line/sample/cell/tissue that shows no improved response to an anti-CD79b immunoconjugate (e.g., polatuzumab vedotin) and an anti-CD20 antibody (e.g., obinutuzumab or rituximab) (particularly in combination with a chemotherapy, particularly in combination with a CHP chemotherapy) in an ex-vivo test. Another non-limiting example of a “control” is an “internal standard”, for example purified or synthetically produced proteins, peptides, DNA and/or RNA, or a mixture thereof, where the amount of each protein/peptide/DNA/RNA is gauged by using the “non-responder” control described herein. In principle, the patient to be treated in the context of the invention is envisaged to be a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) patient. In other words, the patient is a patient with/suffering from lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)). Accordingly, it is particularly envisaged that also the patient defined with respect to any of the aspects/embodiments is a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) patient and a patient with/suffering from lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)), respectively. However, it is not necessarily required that a given patient is diagnosed as being a lymphoma patient, for example prior to (or after) the determination/identification/diagnosis of being a patient as defined herein, in particular as defined in one or more of the aspects/embodiments. It is, however, preferred that the patient to be treated in accordance with the invention is, in a first step, diagnosed as being a lymphoma patient and, in a second step, determined/identified/diagnosed as being a patient defined herein, in particular a patient as defined in one or more of the aspects/embodiments. In principle, in accordance with the invention, a given patient may, in a first step, also be determined/identified/diagnosed as being a patient defined herein, and, in a second step, diagnosed as being a lymphoma patient. However, the latter option is less preferred and, as mentioned, the (foregoing or subsequent) step of diagnosing whether the patient to be treated is a lymphoma (e.g., DLBCL) patient may also be omitted.

The term “protein,” as used herein, refers to any native protein from any vertebrate source, including mammals such as primates (e.g., humans) and rodents (e.g., mice and rats), unless otherwise indicated. The term encompasses “full-length,” unprocessed protein as well as any form of the protein that results from processing in the cell. The term also encompasses naturally occurring variants of the protein, e.g., splice variants or allelic variants.

“Polynucleotide” or “nucleic acid,” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The terms “polynucleotide” and “nucleic acid” specifically includes mRNA and cDNAs.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like), those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro-, or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The phrase “pharmaceutically acceptable” indicates that the substance or composition must be compatible chemically and/or toxicologically, with the other ingredients comprising a formulation, and/or the mammal being treated therewith.

The term “pharmaceutical formulation” refers to a preparation which is in such form as to permit the biological activity of an active ingredient contained therein to be effective, and which contains no additional components which are unacceptably toxic to a subject to which the formulation would be administered.

An “article of manufacture” is any manufacture (e.g., a package or container) or kit comprising at least one reagent, e.g., a medicament for treatment of a disease or disorder (e.g., lymphoma, e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)), and a package insert. In certain embodiments, the manufacture or kit is promoted, distributed, or sold as a unit for performing the methods described herein.

A “package insert” refers to instructions customarily included in commercial packages of medicaments that contain information about the indications customarily included in commercial packages of medicaments that contain information about the indications, usage, dosage, administration, contraindications, other medicaments to be combined with the packaged product, and/or warnings concerning the use of such medicaments.

The term “CD79b,” as used herein, refers to any native CD79b from any vertebrate source, including mammals such as primates (e.g., humans, cynomolgus monkey (“cyno”)) and rodents (e.g., mice and rats), unless otherwise indicated. Human CD79b is also referred to herein as “Igβ,” “B29,” “DNA225786” or “PR036249.” An exemplary CD79b sequence including the signal sequence is shown in SEQ ID NO: 1. An exemplary CD79b sequence without the signal sequence is shown in SEQ ID NO: 2. The term “CD79b” encompasses “full-length,” unprocessed CD79b as well as any form of CD79b that results from processing in the cell. The term also encompasses naturally occurring variants of CD79b, e.g., splice variants, allelic variants and isoforms. The CD79b polypeptides described herein may be isolated from a variety of sources, such as from human tissue types or from another source, or prepared by recombinant or synthetic methods. A “native sequence CD79b polypeptide” comprises a polypeptide having the same amino acid sequence as the corresponding CD79b polypeptide derived from nature. Such native sequence CD79b polypeptides can be isolated from nature or can be produced by recombinant or synthetic means. The term “native sequence CD79b polypeptide” specifically encompasses naturally occurring truncated or secreted forms of the specific CD79b polypeptide (e.g., an extracellular domain sequence), naturally occurring variant forms (e.g., alternatively spliced forms) and naturally occurring allelic variants of the polypeptide.

The term “anti-CD79b antibody” or “an antibody that binds to CD79b” refers to an antibody that is capable of binding CD79b with sufficient affinity such that the antibody is useful as a diagnostic and/or therapeutic agent in targeting CD79b. Preferably, the extent of binding of an anti-CD79b antibody to an unrelated, non-CD79b protein is less than about 10% of the binding of the antibody to CD79b as measured, e.g., by a radioimmunoassay (RIA). In certain embodiments, an antibody that binds to CD79b has a dissociation constant (Kd) of ≤1 μM, ≤100 nM, ≤10 nM, ≤1 nM, or ≤0.1 nM. In certain embodiments, an anti-CD79b antibody binds to an epitope of CD79b that is conserved among CD79b from different species.

The term “CD79b-positive cancer” refers to a cancer comprising cells that express CD79b on their surface. In some embodiments, expression of CD79b on the cell surface is determined, for example, using antibodies to CD79b in a method such as immunohistochemistry, FACS, etc. Alternatively, CD79b mRNA expression is considered to correlate to CD79b expression on the cell surface and can be determined by a method selected from in situ hybridization and RT-PCR (including quantitative RT-PCR).

“Alkyl” is C₁-C₁₈ hydrocarbon containing normal, secondary, tertiary or cyclic carbon atoms. Examples are methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃.

The term “C₁-C₈ alkyl,” as used herein refers to a straight chain or branched, saturated or unsaturated hydrocarbon having from 1 to 8 carbon atoms. Representative “C₁-C₈ alkyl” groups include, but are not limited to, -methyl, -ethyl, -n-propyl, -n-butyl, -n-pentyl, -n-hexyl, -n-heptyl, -n-octyl, -n-nonyl and -n-decyl; while branched C₁-C₈ alkyls include, but are not limited to, -isopropyl, -sec-butyl, -isobutyl, -tert-butyl, -isopentyl, 2-methylbutyl, unsaturated C₁-C₈ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, -3-methyl-1 butynyl. A C₁-C₈ alkyl group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —SO₃R′, —S(O)₂R′, —S(O)R′, —OH, -halogen, —N₃, —NH₂, —NH(R′), —N(R′)₂ and —CN; where each R′ is independently selected from H, —C₁-C₈ alkyl and aryl.

The term “C₁-C₁₂ alkyl,” as used herein refers to a straight chain or branched, saturated or unsaturated hydrocarbon having from 1 to 12 carbon atoms. A C₁-C₁₂ alkyl group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —SO₃R′, —S(O)₂R′, —S(O)R′, —OH, -halogen, —N₃, —NH₂, —NH(R′), —N(R′)₂ and —CN; where each R′ is independently selected from H, —C₁-C₈ alkyl and aryl.

The term “C₁-C₆ alkyl,” as used herein refers to a straight chain or branched, saturated or unsaturated hydrocarbon having from 1 to 6 carbon atoms. Representative “C₁-C₆ alkyl” groups include, but are not limited to, -methyl, -ethyl, -n-propyl, -n-butyl, -n-pentyl, -and n-hexyl; while branched C₁-C₆ alkyls include, but are not limited to, -isopropyl, -sec-butyl, -isobutyl, -tert-butyl, -isopentyl, and 2-methylbutyl; unsaturated C₁-C₆ alkyls include, but are not limited to, -vinyl, -allyl, -1-butenyl, -2-butenyl, and -isobutylenyl, -1-pentenyl, -2-pentenyl, -3-methyl-1-butenyl, -2-methyl-2-butenyl, -2,3-dimethyl-2-butenyl, 1-hexyl, 2-hexyl, and 3-hexyl. A C₁-C₆ alkyl group can be unsubstituted or substituted with one or more groups, as described above for C₁-C₈ alkyl group.

The term “C₁-C₄ alkyl,” as used herein refers to a straight chain or branched, saturated or unsaturated hydrocarbon having from 1 to 4 carbon atoms. Representative “C₁-C₄ alkyl” groups include, but are not limited to, -methyl, -ethyl, -n-propyl, -n-butyl; while branched C₁-C₄ alkyls include, but are not limited to, -isopropyl, -sec-butyl, -isobutyl, -tert-butyl; unsaturated C₁-C₄ alkyls include, but are not limited to, -vinyl, -allyl, -1-butenyl, -2-butenyl, and -isobutylenyl. A C₁-C₄ alkyl group can be unsubstituted or substituted with one or more groups, as described above for C₁-C₈ alkyl group.

“Alkoxy” is an alkyl group singly bonded to an oxygen. Exemplary alkoxy groups include, but are not limited to, methoxy (—OCH₃) and ethoxy (—OCH₂CH₃). A “C₁-C₅ alkoxy” is an alkoxy group with 1 to 5 carbon atoms. Alkoxy groups may can be unsubstituted or substituted with one or more groups, as described above for alkyl groups.

“Alkenyl” is C₂-C₁₈ hydrocarbon containing normal, secondary, tertiary or cyclic carbon atoms with at least one site of unsaturation, i.e. a carbon-carbon, sp² double bond. Examples include, but are not limited to: ethylene or vinyl (—CH═CH₂), allyl (—CH₂CH═CH₂), cyclopentenyl (—C₅H₇), and 5-hexenyl (—CH₂CH₂CH₂CH₂CH═CH₂). A “C₂-C₈ alkenyl” is a hydrocarbon containing 2 to 8 normal, secondary, tertiary or cyclic carbon atoms with at least one site of unsaturation, i.e. a carbon-carbon, sp² double bond.

“Alkynyl” is C₂-C₁₈ hydrocarbon containing normal, secondary, tertiary or cyclic carbon atoms with at least one site of unsaturation, i.e. a carbon-carbon, sp triple bond. Examples include, but are not limited to: acetylenic (—C≡CH) and propargyl (—CH₂C≡CH). A “C₂-C₈ alkynyl” is a hydrocarbon containing 2 to 8 normal, secondary, tertiary or cyclic carbon atoms with at least one site of unsaturation, i.e. a carbon-carbon, sp triple bond.

“Alkylene” refers to a saturated, branched or straight chain or cyclic hydrocarbon radical of 1-18 carbon atoms, and having two monovalent radical centers derived by the removal of two hydrogen atoms from the same or two different carbon atoms of a parent alkane. Typical alkylene radicals include, but are not limited to: methylene (—CH₂—) 1,2-ethyl (—CH₂CH₂—), 1,3-propyl (—CH₂CH₂CH₂—), 1,4-butyl (—CH₂CH₂CH₂CH₂—), and the like.

A “C₁-C₁₀ alkylene” is a straight chain, saturated hydrocarbon group of the formula —(CH₂)₁₋₁₀—. Examples of a C₁-C₁₀alkylene include methylene, ethylene, propylene, butylene, pentylene, hexylene, heptylene, ocytylene, nonylene and decalene.

“Alkenylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkene. Typical alkenylene radicals include, but are not limited to: 1,2-ethylene (—CH═CH—).

“Alkynylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkyne. Typical alkynylene radicals include, but are not limited to: acetylene (—C≡C—), propargyl (—CH₂C≡C—), and 4-pentynyl (—CH₂CH₂CH₂C≡C—).

“Aryl” refers to a carbocyclic aromatic group. Examples of aryl groups include, but are not limited to, phenyl, naphthyl and anthracenyl. A carbocyclic aromatic group or a heterocyclic aromatic group can be unsubstituted or substituted with one or more groups including, but not limited to, —C₁-C₅ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; wherein each R′ is independently selected from H, —C₁-C₅ alkyl and aryl.

A “C₅-C₂₀ aryl” is an aryl group with 5 to 20 carbon atoms in the carbocyclic aromatic rings. Examples of C₅-C₂₀ aryl groups include, but are not limited to, phenyl, naphthyl and anthracenyl. A C₅-C₂₀ aryl group can be substituted or unsubstituted as described above for aryl groups. A “C₅-C₁₄ aryl” is an aryl group with 5 to 14 carbon atoms in the carbocyclic aromatic rings. Examples of C₅-C₁₄ aryl groups include, but are not limited to, phenyl, naphthyl and anthracenyl. A C₅-C₁₄ aryl group can be substituted or unsubstituted as described above for aryl groups.

An “arylene” is an aryl group which has two covalent bonds and can be in the ortho, meta, or para configurations as shown in the following structures:

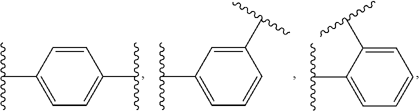

in which the phenyl group can be unsubstituted or substituted with up to four groups including, but not limited to, —C₁-C₅ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; wherein each R′ is independently selected from H, —C₁-C₅ alkyl and aryl.

“Arylalkyl” refers to an acyclic alkyl radical in which one of the hydrogen atoms bonded to a carbon atom, typically a terminal or sp³ carbon atom, is replaced with an aryl radical. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. The arylalkyl group comprises 6 to 20 carbon atoms, e.g., the alkyl moiety, including alkanyl, alkenyl or alkynyl groups, of the arylalkyl group is 1 to 6 carbon atoms and the aryl moiety is 5 to 14 carbon atoms.

“Heteroarylalkyl” refers to an acyclic alkyl radical in which one of the hydrogen atoms bonded to a carbon atom, typically a terminal or sp³ carbon atom, is replaced with a heteroaryl radical. Typical heteroarylalkyl groups include, but are not limited to, 2-benzimidazolylmethyl, 2-furylethyl, and the like. The heteroarylalkyl group comprises 6 to 20 carbon atoms, e.g., the alkyl moiety, including alkanyl, alkenyl or alkynyl groups, of the heteroarylalkyl group is 1 to 6 carbon atoms and the heteroaryl moiety is 5 to 14 carbon atoms and 1 to 3 heteroatoms selected from N, O, P, and S. The heteroaryl moiety of the heteroarylalkyl group may be a monocycle having 3 to 7 ring members (2 to 6 carbon atoms or a bicycle having 7 to 10 ring members (4 to 9 carbon atoms and 1 to 3 heteroatoms selected from N, O, P, and S), for example: a bicyclo[4,5], [5,5], [5,6], or [6,6] system.

“Substituted alkyl,” “substituted aryl,” and “substituted arylalkyl” mean alkyl, aryl, and arylalkyl respectively, in which one or more hydrogen atoms are each independently replaced with a substituent. Typical substituents include, but are not limited to, —X, —R, —O⁻, —OR, —SR, —S⁻, —NR₂, —NR₃, ═NR, —CX₃, —CN, —OCN, —SCN, —N═C═O, —NCS, —NO, —NO₂, ═N₂, —N₃, NC(═O)R, —C(═O)R, —C(═O)NR₂, —SO₃—, —SO₃H, —S(═O)₂R, —OS(═O)₂OR, —S(═O)₂NR, —S(═O)R, —OP(═O)(OR)₂, —P(═O)(OR)₂, —PO⁻₃, —PO₃H₂, —C(═O)R, —C(═O)X, —C(═S)R, —CO₂R, —CO₂⁻, —C(═S)OR, —C(═O)SR, —C(═S)SR, —C(═O)NR₂, —C(═S)NR₂, —C(═NR)NR₂, where each X is independently a halogen: F, Cl, Br, or I; and each R is independently —H, C₂-C₁₈ alkyl, C₆-C₂₀ aryl, C₃-C₁₄ heterocycle, protecting group or prodrug moiety. Alkylene, alkenylene, and alkynylene groups as described above may also be similarly substituted.

“Heteroaryl” and “heterocycle” refer to a ring system in which one or more ring atoms is a heteroatom, e.g., nitrogen, oxygen, and sulfur. The heterocycle radical comprises 3 to 20 carbon atoms and 1 to 3 heteroatoms selected from N, O, P, and S. A heterocycle may be a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S), for example: a bicyclo[4,5], [5,5], [5,6], or [6,6] system.

Exemplary heterocycles are described, e.g., in Paquette, Leo A., “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566.

Examples of heterocycles include by way of example and not limitation pyridyl, dihydroypyridyl, tetrahydropyridyl (piperidyl), thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, bis-tetrahydrofuranyl, tetrahydropyranyl, bis-tetrahydropyranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, and isatinoyl.

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl.

A “C₃-C₈ heterocycle” refers to an aromatic or non-aromatic C₃-C₈ carbocycle in which one to four of the ring carbon atoms are independently replaced with a heteroatom from the group consisting of O, S and N. Representative examples of a C₃-C₈ heterocycle include, but are not limited to, benzofuranyl, benzothiophene, indolyl, benzopyrazolyl, coumarinyl, isoquinolinyl, pyrrolyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl and tetrazolyl. A C₃-C₈ heterocycle can be unsubstituted or substituted with up to seven groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; wherein each R′ is independently selected from H, —C₁-C₈ alkyl and aryl.

“C₃-C₈ heterocyclo” refers to a C₃-C₈ heterocycle group defined above wherein one of the heterocycle group's hydrogen atoms is replaced with a bond. A C₃-C₈ heterocyclo can be unsubstituted or substituted with up to six groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; wherein each R′ is independently selected from H, —C₁-C₈ alkyl and aryl.

A “C₃-C_{2M} heterocycle” refers to an aromatic or non-aromatic C₃-C₈ carbocycle in which one to four of the ring carbon atoms are independently replaced with a heteroatom from the group consisting of O, S and N. A C₃-C₂₀ heterocycle can be unsubstituted or substituted with up to seven groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; wherein each R′ is independently selected from H, —C₁-C₈ alkyl and aryl.

“C₃-C₂₀ heterocyclo” refers to a C₃-C₂₀ heterocycle group defined above wherein one of the heterocycle group's hydrogen atoms is replaced with a bond.

“Carbocycle” means a saturated or unsaturated ring having 3 to 7 carbon atoms as a monocycle or 7 to 12 carbon atoms as a bicycle. Monocyclic carbocycles have 3 to 6 ring atoms, still more typically 5 or 6 ring atoms. Bicyclic carbocycles have 7 to 12 ring atoms, e.g., arranged as a bicyclo[4,5], [5,5], [5,6] or [6,6] system, or 9 or 10 ring atoms arranged as a bicyclo[5,6] or [6,6]system. Examples of monocyclic carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cycloheptyl, and cyclooctyl.

A “C₃-C₈ carbocycle” is a 3-, 4-, 5-, 6-, 7- or 8-membered saturated or unsaturated non-aromatic carbocyclic ring. Representative C₃-C₈ carbocycles include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. A C₃-C₈ carbocycle group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; where each R′ is independently selected from H, —C₁-C₈ alkyl and aryl.

A “C₃-C₈ carbocyclo” refers to a C₃-C₈ carbocycle group defined above wherein one of the carbocycle groups' hydrogen atoms is replaced with a bond.

“Linker” refers to a chemical moiety comprising a covalent bond or a chain of atoms that covalently attaches an antibody to a drug moiety. In various embodiments, linkers include a divalent radical such as an alkyldiyl, an aryldiyl, a heteroaryldiyl, moieties such as: —(CR₂)ₙO(CR₂)ₙ—, repeating units of alkyloxy (e.g., polyethylenoxy, PEG, polymethyleneoxy) and alkylamino (e.g., polyethyleneamino, Jeffamine™); and diacid ester and amides including succinate, succinamide, diglycolate, malonate, and caproamide. In various embodiments, linkers can comprise one or more amino acid residues, such as valine, phenylalanine, lysine, and homolysine.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

“Leaving group” refers to a functional group that can be substituted by another functional group. Certain leaving groups are well known in the art, and examples include, but are not limited to, a halide (e.g., chloride, bromide, iodide), methanesulfonyl (mesyl), p-toluenesulfonyl (tosyl), trifluoromethylsulfonyl (triflate), and trifluoromethylsulfonate.

The term “protecting group” refers to a substituent that is commonly employed to block or protect a particular functionality while reacting other functional groups on the compound. For example, an “amino-protecting group” is a substituent attached to an amino group that blocks or protects the amino functionality in the compound. Suitable amino-protecting groups include, but are not limited to, acetyl, trifluoroacetyl, t-butoxycarbonyl (BOC), benzyloxycarbonyl (CBZ) and 9-fluorenylmethylenoxycarbonyl (Fmoc). For a general description of protecting groups and their use, see T. W. Greene, Protective Groups in Organic Synthesis, John Wiley & Sons, New York, 1991, or a later edition.

### III. Diagnostic Methods and Assays

Provided herein are methods and assays for identifying, diagnosing, and/or predicting whether a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) may benefit from a treatment comprising an anti-CD79b immunoconjugate (e.g., polatuzumab vedotin) and an anti-CD20 antibody (e.g., obinutuzumab or rituximab). The methods and assays described herein are based on the finding that an amount or level of a macrophage biomarker (e.g., a gene expression value (e.g., a gene expression value derived from any of the gene signature sets described herein (e.g., any of the exemplified gene signature sets in Table 2 or Table 3)) or an amount of macrophages (e.g., M1 macrophages or tumor-associated macrophages)) in a sample (e.g., a tissue sample, e.g., a tumor tissue sample, such as a biopsy) from the patient may be used to identify, diagnose, and/or predict the patient as one who may benefit from the treatment comprising an anti-CD79b immunoconjugate (e.g., polatuzumab vedotin) and an anti-CD20 antibody (e.g., obinutuzumab or rituximab). Any of the methods provided herein may include administering to the patient an anti-CD79b immunoconjugate (e.g., polatuzumab vedotin) and an anti-CD20 antibody (e.g., obinutuzumab or rituximab).

### A. Macrophage Biomarkers

In particular instances, the methods and assays provided herein may be used to determine the amount or level of a macrophage biomarker. Various diagnostic methods based on a determination of the amount or level of the macrophage biomarker are further described below.

In one aspect, provided herein are methods for identifying, diagnosing, and/or predicting whether a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma) may benefit from a treatment comprising an anti-CD79b immunoconjugate and anti-CD20 antibody, the method including measuring a macrophage biomarker in a sample from the patient, wherein an amount or level of the macrophage biomarker in the sample that is below a reference macrophage biomarker amount or level identifies, diagnoses, and/or predicts the patient as one who may benefit from a treatment comprising an anti-CD79b immunoconjugate and an anti-CD20 antibody. In some instances, the methods comprise administering an anti-CD79b immunoconjugate and an anti-CD20 antibody.

In another aspect, provided herein are methods for selecting a therapy for a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma), the method including measuring a macrophage biomarker in a sample from the patient, wherein an amount or level of the macrophage biomarker in the sample that is below a reference macrophage biomarker amount or level identifies the patient as one who may benefit from a treatment comprising an anti-CD79b immunoconjugate and an anti-CD20 antibody. In some instances, the methods comprise administering an anti-CD79b immunoconjugate and an anti-CD20 antibody.

### (i) Decreased Macrophage Biomarker

An amount or level of the macrophage biomarker in a sample from a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) that is below a reference macrophage biomarker amount or level may identify, diagnose, and/or predict the patient as one who may benefit from a treatment comprising an anti-CD79b immunoconjugate (e.g., polatuzumab vedotin) and an anti-CD20 antibody (e.g., obinutuzumab or rituximab).

In some instances, an amount or level of the macrophage biomarker in the sample that is in about the bottom 99th percentile (equal to, or lower than, about the 99% prevalence level), about the bottom 95th percentile (equal to, or lower than, about the 95% prevalence level), about the bottom 90th percentile (equal to, or lower than, about the 90% prevalence level), about the bottom 85th percentile (equal to, or lower than, about the 85% prevalence level), about the bottom 80th percentile (equal to, or lower than, about the 80% prevalence level), about the bottom 75th percentile (equal to, or lower than, about the 75% prevalence level), about the bottom 70th percentile (equal to, or lower than, about the 70% prevalence level), about the bottom 65th percentile (equal to, or lower than, about the 65% prevalence level), about the bottom 60th percentile (equal to, or lower than, about the 60% prevalence level), about the bottom 55th percentile (equal to, or lower than, about the 55% prevalence level), about the bottom 50th percentile (equal to, or lower than, about the 50% prevalence level), about the bottom 45th percentile (equal to, or lower than, about the 45% prevalence level), about the bottom 40th percentile (equal to, or lower than, about the 40% prevalence level), about the bottom 35th percentile (equal to, or lower than, about the 35% prevalence level), about the bottom 30th percentile (equal to, or lower than, about the 30% prevalence level), about the bottom 25th percentile (equal to, or lower than, about the 25% prevalence level), about the bottom 20th percentile (equal to, or lower than, about the 20% prevalence level), about the bottom 15th percentile (equal to, or lower than, about the 15% prevalence level), about the bottom 10th percentile (equal to, or lower than, about the 10% prevalence level), about the bottom 5th percentile (equal to, or lower than, about the 5% prevalence level), or about the bottom 1st percentile (equal to, or lower than, about the 1% prevalence level) of the amount or level of the macrophage biomarker in the reference population identifies the individual as one who is likely to benefit from a treatment including an anti-CD79b immunoconjugate (e.g., polatuzumab vedotin) and an anti-CD20 antibody (e.g., obinutuzumab or rituximab).

In some instances, an amount or level of the macrophage biomarker in the sample that is in about the bottom 10th to about the bottom 90th percentile, about the bottom 20th to about the bottom 80th percentile, about the bottom 30th to about the bottom 70th percentile, about the bottom 40th to about the bottom 60th percentile, about the bottom 45th to about the bottom 55th percentile, about the bottom 48th to about the bottom 52th percentile, about the bottom 49.5th to about the bottom 50.5th percentile, about the bottom 49.9th to about the bottom 50.1th percentile, or about the bottom 50th percentile of the amount or level of the macrophage biomarker in the reference population identifies the individual as one who is likely to benefit from a treatment including an anti-CD79b immunoconjugate (e.g., polatuzumab vedotin) and an anti-CD20 antibody (e.g., obinutuzumab or rituximab). For example, in some instances, an amount or level of the macrophage biomarker in the sample that is between about 10% to about 90% prevalence, about 15 to about 85% prevalence, about 20% to about 80% prevalence, about 25% to about 75% prevalence, about 30% to about 70% prevalence, about 35% to about 65% prevalence, about 40% to about 60% prevalence, about 45% to about 55% prevalence, about 48% to about 52% prevalence, about 49.5% to about 50.5% prevalence, about 49.9% to about 50.1% prevalence, or about 50% prevalence in the reference population identifies the individual as one who is likely to benefit from a treatment including an anti-CD79b immunoconjugate (e.g., polatuzumab vedotin) and an anti-CD20 antibody (e.g., obinutuzumab or rituximab).

In some instances, an amount or level of the macrophage biomarker that is lower than a reference amount or level of the macrophage biomarker refers to a decrease of about 10%, 20%, 30%, 40%, 50%, 60%, 70%, 80%, 90%, 95%, 96%, 97%, 98%, or 99% or greater in the amount or level of the macrophage biomarker, detected by standard art-known methods such as those described herein, as compared to the amount or level of the macrophage biomarker in a reference sample, reference cell, reference tissue, control sample, control cell, or control tissue. In certain instances, an amount or level of the macrophage biomarker that is lower than a reference amount or level of the macrophage biomarker refers to a decrease in the amount or level of the macrophage biomarker in the sample, wherein the decrease is at least about 1.5×, 1.75×, 2×, 3×, 4×, 5×, 6×, 7×, 8×, 9×, 10×, 25×, 50×, 75×, or 100× the amount or level of the macrophage biomarker in a reference sample, reference cell, reference tissue, control sample, control cell, or control tissue. In some instances, an amount or level of the macrophage biomarker that is lower than a reference amount or level of the macrophage biomarker refers to a decrease in the amount or level of the macrophage biomarker that is greater than about 1.5-fold, about 1.75-fold, about 2-fold, about 2.25-fold, about 2.5-fold, about 2.75-fold, about 3.0-fold, or about 3.25-fold as compared to the amount or level of the macrophage biomarker in a reference sample, reference cell, reference tissue, control sample, control cell, or control tissue.

In some instances, an amount or level of the macrophage biomarker that is lower than a reference amount or level of the macrophage biomarker refers to an overall decrease of about 10%, 20%, 30%, 40%, 50%, 60%, 70%, 80%, 90%, 95%, 96%, 97%, 98%, or 99% or greater in the amount or level of the macrophage biomarker, detected by standard art-known methods such as those described herein, as compared to a pre-assigned amount or level of the macrophage biomarker. In certain instances, an amount or level of the macrophage biomarker that is lower than a reference amount or level of the macrophage biomarker refers to a decrease in the amount or level of the macrophage biomarker in the sample, wherein the decrease is at least about 1.5×, 1.75×, 2×, 3×, 4×, 5×, 6×, 7×, 8×, 9×, 10×, 25×, 50×, 75×, or 100× a pre-assigned amount or level of the macrophage biomarker. In some instances, an amount or level of the macrophage biomarker that is lower than a reference amount or level of the macrophage biomarker refers to an overall decrease in the amount or level of the macrophage biomarker that is greater than about 1.5-fold, about 1.75-fold, about 2-fold, about 2.25-fold, about 2.5-fold, about 2.75-fold, about 3.0-fold, or about 3.25-fold as compared to a pre-assigned amount or level of the macrophage biomarker.

### (ii) Increased Macrophage Biomarker

Previous studies, as described in WO2022/031749, determined that an amount or level of the macrophage biomarker in a sample from a patient that is above a reference macrophage biomarker amount or level identifies, diagnoses. and/or predicts the patient as one who may benefit from a treatment comprising an anti-CD20 antibody. Based on the studies described herein, an amount or level of the macrophage biomarker in a sample from a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) that is above a reference macrophage biomarker amount or level may identify, diagnose, and/or predict the patient as one who may benefit from a treatment comprising either an anti-CD79b immunoconjugate (e.g., polatuzumab vedotin) and an anti-CD20 antibody (e.g., obinutuzumab or rituximab) or an anti-CD20 antibody (e.g., obinutuzumab or rituximab). Selection of therapy comprising an anti-CD79b immunoconjugate (e.g., polatuzumab vedotin) and an anti-CD20 antibody (e.g., obinutuzumab or rituximab) or selection of a therapy comprising an anti-CD20 antibody (e.g., obinutuzumab or rituximab) where the patient sample has an amount or level of the macrophage biomarker that is above a reference macrophage biomarker amount or level may be based on additional factors in addition to the amount or level of the macrophage biomarker.

(iii) Reference Macrophage Biomarker

The reference macrophage biomarker amount or level can be a pre-assigned macrophage biomarker amount or level. In some instances, the amount or level of the macrophage biomarker in a reference population is a median amount or level of the macrophage biomarker of the reference population. In some instances, the amount or level of the macrophage biomarker in a reference population is a mean amount or level of the macrophage biomarker of the reference population.

In some instances, the pre-assigned macrophage biomarker amount or level is a percentage of cellular subtypes within a sample. In some instances, the percentage of cellular subtypes within a sample is between about 0% and 40% (e.g., 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40%). In some instances, the percentage of cellular subtypes within a sample is between about 0% and 10% (e.g., 0%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%). In some instances, the percentage of cellular subtypes within a sample is less than 10% (e.g., 0%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%). In some instances, the percentage of cellular subtypes within a sample is about 6%. In some instances, the percentage of cellular subtypes within a sample is about 5%. In some instances, the percentage of cellular subtypes within a sample is about 4.74%. In some instances, the percentage of cellular subtypes within a sample is about 4%. In some instances, the percentage of cellular subtypes within a sample is about 3.35%. In some instances, the percentage of cellular subtypes within a sample is about 3%. In some instances, the percentage of cellular subtypes within a sample is about 2.5%. In some instances, the percentage of cellular subtypes within a sample is about 2%. In some instances, the percentage of cellular subtypes within a sample is about 1.67%. In some instances, the percentage of cellular subtypes within a sample is about 1%. In some instances, the percentage of cellular subtypes within a sample is about 0%.

The reference amount or level of the macrophage biomarker described herein may be based on the amount or level of the macrophage biomarker in a reference population. In some instances, the reference macrophage biomarker described herein is an amount or level of the macrophage biomarker in a reference population that includes one or more (e.g., one or more, two or more, three or more, four or more, or five or more) subsets of patients.

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)).

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) who have been administered one or more doses (e.g., at least one, two, three, four, five, six, seven, eight, nine, or ten or more doses) of either an anti-CD79b immunoconjugate (e.g., polatuzumab vedotin) and an anti-CD20 antibody (e.g., obinutuzumab or rituximab) or an anti-CD20 antibody (e.g., obinutuzumab or rituximab).

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) who have received treatment with an anti-CD20 antibody (e.g., obinutuzumab or rituximab) as a monotherapy.

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) who have received treatment with an anti-CD79b immunoconjugate (e.g., polatuzumab vedotin) and an anti-CD20 antibody (e.g., obinutuzumab or rituximab) as a combination therapy (e.g., a combination therapy including an anti-CD79b immunoconjugate (e.g., polatuzumab vedotin) and an anti-CD20 antibody (e.g., obinutuzumab or rituximab) and an additional therapeutic agent (e.g., anti-cancer therapy (e.g., a cytotoxic agent, a growth-inhibitory agent, a radiation therapy, an anti-angiogenic agent, or a combination thereof), e.g., CHP).

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) who have received treatment with an anti-CD20 antibody (e.g., obinutuzumab or rituximab) as a combination therapy (e.g., a combination therapy including an anti-CD20 antibody (e.g., obinutuzumab or rituximab) and an additional therapeutic agent (e.g., anti-cancer therapy (e.g., a cytotoxic agent, a growth-inhibitory agent, a radiation therapy, an anti-angiogenic agent, or a combination thereof), e.g., CHOP).

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) who have received treatment with a therapy that does not include an anti-CD79b immunoconjugate (e.g., polatuzumab vedotin) and an anti-CD20 antibody (e.g., obinutuzumab or rituximab) and includes an anti-cancer therapy (e.g., a cytotoxic agent, a growth-inhibitory agent, a radiation therapy, an anti-angiogenic agent, or a combination thereof), e.g., CHP).

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) who have received treatment with a therapy that does not include an anti-CD20 antibody (e.g., obinutuzumab or rituximab) and includes an anti-cancer therapy (e.g., a cytotoxic agent, a growth-inhibitory agent, a radiation therapy, an anti-angiogenic agent, or a combination thereof), e.g., CHOP).

For example, in some instances, the reference population includes a first subset of patients who have been treated with an anti-CD79b immunoconjugate (e.g., polatuzumab vedotin) and an anti-CD20 antibody (e.g., obinutuzumab or rituximab) and a second subset of patients who have not been treated with an anti-CD79b immunoconjugate (e.g., polatuzumab vedotin) and an anti-CD20 antibody (e.g., obinutuzumab or rituximab).

For example, in some instances, the reference population includes a first subset of patients who have been treated with an anti-CD20 antibody (e.g., obinutuzumab or rituximab) and a second subset of patients who have not been treated an anti-CD20 antibody (e.g., obinutuzumab or rituximab).

For example, in some instances, the reference population includes a first subset of patients who have been treated with an anti-CD79b immunoconjugate (e.g., polatuzumab vedotin) and an anti-CD20 antibody (e.g., obinutuzumab or rituximab) and a second subset of patients who have been treated with an anti-CD20 antibody (e.g., obinutuzumab or rituximab).

In some instances, the reference amount or level of the macrophage biomarker significantly separates each of the first and second subsets of patients based on a significant difference between a patient's responsiveness (e.g., PFS or OS) to treatment with the anti-CD79b immunoconjugate (e.g., polatuzumab vedotin) and the anti-CD20 antibody (e.g., obinutuzumab or rituximab) and a patient's responsiveness to treatment with the anti-CD20 antibody (e.g., obinutuzumab or rituximab) below the reference macrophage biomarker, wherein the patient's responsiveness to treatment with the anti-CD79b immunoconjugate (e.g., polatuzumab vedotin) and the anti-CD20 antibody (e.g., obinutuzumab or rituximab) is significantly improved relative to the patient's responsiveness to treatment with the anti-CD20 antibody (e.g., obinutuzumab or rituximab). For example, in some instances, the reference amount or level of the macrophage biomarker optimally separates each of the first and second subsets of patients based on a maximum difference between a patient's responsiveness (e.g., PFS or OS) to treatment with the anti-CD79b immunoconjugate (e.g., polatuzumab vedotin) and the anti-CD20 antibody (e.g., obinutuzumab or rituximab) and a patient's responsiveness to treatment with the anti-CD20 antibody (e.g., obinutuzumab or rituximab) below the reference macrophage biomarker, wherein the patient's responsiveness to treatment with the anti-CD79b immunoconjugate (e.g., polatuzumab vedotin) and the anti-CD20 antibody (e.g., obinutuzumab or rituximab) is significantly improved relative to the patient's responsiveness to treatment with the anti-CD20 antibody (e.g., obinutuzumab or rituximab). In some instances, the aforementioned first subset of patients is patients treated with Pola-R-CHP (polatuzumab vedotin plus rituximab, cyclophosphamide, doxorubicin, and prednisone). In some instances, the aforementioned second subset of patients is patients treated with R-CHOP (Rituximab plus cyclophosphamide, doxorubicin, vincristine, and prednisone).

In some instances, the reference amount or level of the macrophage biomarker significantly separates each of the first and second subsets of patients based on a significant difference between a patient's responsiveness (e.g., PFS or OS) to treatment with the anti-CD79b immunoconjugate (e.g., polatuzumab vedotin) and the anti-CD20 antibody (e.g., obinutuzumab or rituximab) and a patient's responsiveness to treatment without the anti-CD79b immunoconjugate (e.g., polatuzumab vedotin) and the anti-CD20 antibody (e.g., obinutuzumab or rituximab) below the reference macrophage biomarker, wherein the patient's responsiveness to treatment with the anti-CD79b immunoconjugate (e.g., polatuzumab vedotin) and the anti-CD20 antibody (e.g., obinutuzumab or rituximab) is significantly improved relative to the patient's responsiveness to treatment without the anti-CD79b immunoconjugate (e.g., polatuzumab vedotin) and the anti-CD20 antibody (e.g., obinutuzumab or rituximab). For example, in some instances, the reference amount or level of the macrophage biomarker optimally separates each of the first and second subsets of patients based on a maximum difference between a patient's responsiveness (e.g., PFS or OS) to treatment with the anti-CD79b immunoconjugate (e.g., polatuzumab vedotin) and the anti-CD20 antibody (e.g., obinutuzumab or rituximab) and a patient's responsiveness to treatment without the anti-CD79b immunoconjugate (e.g., polatuzumab vedotin) and the anti-CD20 antibody (e.g., obinutuzumab or rituximab) below the reference macrophage biomarker, wherein the patient's responsiveness to treatment with the anti-CD79b immunoconjugate (e.g., polatuzumab vedotin) and the anti-CD20 antibody (e.g., obinutuzumab or rituximab) is significantly improved relative to the patient's responsiveness to treatment without the anti-CD79b immunoconjugate (e.g., polatuzumab vedotin) and the anti-CD20 antibody (e.g., obinutuzumab or rituximab). In some instances, the aforementioned first subset of patients is patients treated with Pola-R-CHP (polatuzumab vedotin plus rituximab, cyclophosphamide, doxorubicin, and prednisone).

In some instances, the reference amount or level of the macrophage biomarker significantly separates each of the first and second subsets of patients based on a significant difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) below the reference macrophage biomarker, wherein the patient's responsiveness to treatment without the anti-CD20 antibody (e.g., obinutuzumab or rituximab) is significantly improved relative to the patient's responsiveness to treatment with the anti-CD20 antibody (e.g., obinutuzumab or rituximab). For example, in some instances, the reference amount or level of the macrophage biomarker optimally separates each of the first and second subsets of patients based on a maximum difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) below the reference macrophage biomarker, wherein the patient's responsiveness to treatment without the anti-CD20 antibody (e.g., obinutuzumab or rituximab) is significantly improved relative to the patient's responsiveness to treatment with the anti-CD20 antibody (e.g., obinutuzumab or rituximab). In some instances, the aforementioned first subset of patients is patients treated with R-CHOP (Rituximab plus cyclophosphamide, doxorubicin, vincristine, and prednisone).

In some instances, an optimal separation or significant separation may be based on a hazard ratio (HR) determined from an analysis of the amount or level of the macrophage biomarker in the first and second subsets of patients, wherein the HR is less than 1, e.g., an HR of about 0.95, about 0.9, about 0.8, about 0.7, about 0.6, about 0.5, about 0.4, about 0.3, about 0.2, about 0.1 or lower. For example, in particular instances, an optimal separation or significant separation may be based on a hazard ratio (HR) determined from an analysis of the amount or level of the macrophage biomarker in the first and second subsets of patients, wherein the upper bound of the 95% confidence interval of the HR is less than 1, e.g., an upper bound of the 95% confidence interval of the HR of about 0.95, about 0.9, about 0.8, about 0.7, about 0.6, about 0.5, about 0.4, about 0.3, about 0.2, about 0.1 or lower.

Additionally, or alternatively, the reference macrophage biomarker may be an amount or level of the macrophage biomarker in a reference population, wherein the reference population includes at least one subset of patients who do not have a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) or have a lymphoma but are treatment naïve.

### (iv) Indications

The methods described herein are useful for predicting the therapeutic response of an individual having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) to treatment with an anti-CD79b immunoconjugate (e.g., polatuzumab vedotin) and an anti-CD20 antibody (e.g., obinutuzumab or rituximab).

In some instances, the lymphoma may be indolent lymphoma. In some instances, the lymphoma may be a B-cell lymphoma, e.g., a non-Hodgkin lymphoma, e.g., a diffuse large B-cell lymphoma (e.g., a germinal-center B-cell-like or activated B-cell-like diffuse large B-cell lymphoma). In some instances, the lymphoma may be a CD20-positive lymphoma.

In certain instances, the cancer may be a B-cell lymphoma. For example, the B-cell lymphoma may be a non-Hodgkin lymphoma, including but not limited to a diffuse large B-cell lymphoma (e.g., a germinal-center B-cell-like or activated B-cell-like diffuse large B-cell lymphoma)). For example, the methods described herein may be used for identifying, diagnosing, and/or predicting whether a patient having a B-cell lymphoma (e.g., non-Hodgkin lymphoma (e.g., a diffuse large B-cell lymphoma (e.g., a germinal-center B-cell-like or activated B-cell-like diffuse large B-cell lymphoma)) may benefit from a treatment comprising an anti-CD79b immunoconjugate (e.g., polatuzumab vedotin) and an anti-CD20 antibody (e.g., obinutuzumab or rituximab), the method including measuring a macrophage biomarker in a sample from the patient, wherein an amount or level of the macrophage biomarker in the sample that is below a reference macrophage biomarker amount or level identifies, diagnoses, and/or predicts the patient as one who may benefit from a treatment comprising an anti-CD79b immunoconjugate and an anti-CD20 antibody.

In some instances, the individual having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) has not been previously treated for the lymphoma (treatment naïve). For example, in some instances, the individual having a lymphoma has not previously received either an anti-CD79b immunoconjugate (e.g., polatuzumab vedotin) and an anti-CD20 antibody (e.g., obinutuzumab or rituximab) or an anti-CD20 antibody (e.g., obinutuzumab or rituximab).

In some instances, the individual having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) has previously received treatment for the lymphoma. In some instances, the individual having a lymphoma has previously received treatment including either an anti-CD79b immunoconjugate (e.g., polatuzumab vedotin) and an anti-CD20 antibody (e.g., obinutuzumab or rituximab) or an anti-CD20 antibody (e.g., obinutuzumab or rituximab).

### (v) Treatment Benefits

A patient who benefits from receiving treatment with an anti-CD79b immunoconjugate (e.g., polatuzumab vedotin) and an anti-CD20 antibody (e.g., obinutuzumab or rituximab) may experience, for example, a delay or prevention in the occurrence or recurrence of a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)), alleviation of symptoms, diminishment of any direct or indirect pathological consequences of the cancer, prevention of metastasis, decrease in the rate of disease progression, amelioration or palliation of the disease state, or remission or improved prognosis. In some instances, the treatments described herein are used to delay development of a cancer or to slow the progression of a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)). In some instances, the benefit may be an increase in overall survival (OS), progression-free survival (PFS), complete response (CR), partial response (PR), or a combination thereof.

In some instances, an amount or level of a macrophage biomarker that is below a reference macrophage biomarker amount or level (e.g., an amount or level of a macrophage biomarker in a reference population) identifies the individual as one who may benefit from a treatment including an anti-CD79b immunoconjugate (e.g., polatuzumab vedotin) and an anti-CD20 antibody (e.g., obinutuzumab or rituximab), wherein the benefit is an increase in OS (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) relative to a treatment that does not include an anti-CD79b immunoconjugate (e.g., polatuzumab vedotin) and an anti-CD20 antibody (e.g., obinutuzumab or rituximab).

In some instances, an amount or level of a macrophage biomarker that is below a reference macrophage biomarker amount or level (e.g., an amount or level of a macrophage biomarker in a reference population) identifies the individual as one who may benefit from a treatment including an anti-CD79b immunoconjugate (e.g., polatuzumab vedotin) and an anti-CD20 antibody (e.g., obinutuzumab or rituximab), wherein the benefit is an increase in PFS (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) relative to a treatment that does not include an anti-CD79b immunoconjugate (e.g., polatuzumab vedotin) and an anti-CD20 antibody (e.g., obinutuzumab or rituximab).

### B. Determination of Macrophage Biomarkers

### (i) Detection Methods

The macrophage biomarkers described herein may be based on an amount or expression level of a nucleic acid (e.g., an mRNA), a protein, or a cell (e.g., an M1 macrophage, or a tumor-associated macrophage). Presence and/or expression levels/amount of the genes described herein (see, e.g., Table 2 or Table 3) can be determined qualitatively and/or quantitatively based on any suitable criterion known in the art, including but not limited to DNA, mRNA, cDNA, proteins, protein fragments, and/or gene copy number. Presence and/or levels/amounts of the cells described herein can be determined qualitatively and/or quantitatively based on any suitable criterion known in the art, including but not limited to microscopy, cytometry, DNA, mRNA, cDNA, proteins, protein fragments, and/or gene copy number.

TABLE 2
Exemplary M1 macrophage gene signature sets

| Gene | |
|---|---|
| signature | |
| set | Genes |
| 1 | ACP2, ABCD1, C1QA, FDX1, CCL22, CD163, SCAMP2, ADAMDEC1, ARL8B, and HAMP |
| 2 | ACP2, ABCD1, FDX1, CCL8, CCL22, CD163, ADAMDEC1, TREM2, and HAMP |
| 3 | ACP2, ADRA2B, ALCAM, ABCD1, ATOX1, ATP6V0C, ATP6V1E1, BLVRA, C1QA, CD48, |
| | CD63, CLCN7, TPP1, CLTC, CCR1, CMKLR1, SLC31A1, COX5B, FCER1G, FDX1, FOLR2, |
| | FPR3, FTL, HEXB, HK3, IL10, IL12B, ITGAE, LAIR1, CXCL9, MMP19, NARS, NDUFS2, |
| | P2RX7, PDCL, MAPK13, PTGIR, PTPRA, RELA, CCL7, CCL8, CCL19, CCL22, SRC, STX4, |
| | TCEB1, TFRC, AGPS, MARCO, SNX3, CD84, USP14, ITGB1BP1, ATP6V1F, TRIP4, |
| | CD163, CIAO1, WTAP, ARHGEF11, ABI1, SCAMP2, ACTR2, BCAP31, ZMPSTE24, |
| | BCKDK, EXOC5, STIP1, UQCR11, SDS, LILRB4, OGFR, TFEC, FKBP15, DNAJC13, |
| | TDRD7, STX12, IL17RA, ABTB2, FAM32A, SIGLEC7, SIGLEC9, ADAMDEC1, CECR5, |
| | SLC25A24, NRBP1, MS4A4A, TREM2, OTUD4, PQLC2, HAUS2, ARL8B, NECAP2, |
| | WDR11, ZC3H15, CCDC47, UTP3, MRS2, HAMP, MRPL40, VPS33A, CORO7, LIMD2, |
| | TMX1, DOT1L, ADO, and ADCK2 |
| 4 | ACP2, ADRA2B, ALCAM, TSPO, C3AR1, DAGLA, CALR, CHIT1, CYBB, CYC1, |
| | CYP19A1, DLAT, FCER1G, GP1BA, GPD1, IFNAR1, IL10, KCNJ5, KIFC3, MT2A, |
| | MYBPH, MYH11, MYO7A, P2RX7, PRDX1, RAB3IL1, RNH1, MRPL12, CCL1, CCL7, |
| | CCL8, CCL24, SRC, VIM, RRP1, MARCO, S1PR2, AP1M2, ACTR3, LILRB1, AFG3L2, |
| | SDS, LILRB4, EMILIN1, VSIG4, HSPB7, COQ2, ADAMDEC1, CECR5, WSB2, SLAMF8, |
| | DNASE2B, CLPB, MFSD7, and ADCK2 |
| 5 | ACP2, ADCY3, ADRA2B, ALCAM, TSPO, C1QA, C1QB, C3AR1, DAGLA, CD63, CHIT1, |
| | CMKLR1, SLC31A1, CSF1, CSF1R, CYBB, CYC1, CYP19A1, FANCE, FCER1G, FDX1, |
| | FPR3, FTL, GP1BA, GPD1, HEXB, IL10, KCNJ1, KCNJ5, KIFC3, LAMP1, MMP19, MSR1, |
| | MT2A, MYBPH, MYO7A, P2RX7, PRDX1, RAB3IL1, MRPL12, CCL1, CCL7, CCL8, |
| | CCL18, CCL19, CCL24, SLC6A12, SPR, SRC, RRP1, MARCO, PKD2L1, S1PR2, CD163, |
| | LONP1, AP1M2, IGSF6, LILRB1, SDS, LILRB4, EMILIN1, VSIG4, TFEC, PHLDB1, |
| | CYFIP1, FKBP15, NCAPH, MYOF, HSPB7, ADAMDEC1, GLRX2, NDUFAF1, SPG21, |
| | MS4A4A, ATP6V1D, ATP6V1H, TREM2, PQLC2, TMEM70, PLEKHB2, TMEM33, |
| | SLAMF8, HAMP, DNASE2B, MYOZ1, LONRF3, CLPB, MFSD7, and ADCK2 |
| 6 | ACP2, ADCY3, ADRA2B, ALCAM, ABCD1, ANXA2, ATP6V1A, C1QA, C1QB, C3AR1, |
| | DAGLA, CD80, CD63, CHIT1, CMKLR1, SLC31A1, CSF1, CSF1R, CYBB, CYC1, |
| | CYP19A1, FANCE, FDX1, FPR2, FPR3, GPD1, HEXB, KCNJ1, KCNJ5, KIFC3, MMP19, |
| | MSR1, MT2A, MYBPH, P2RX7, MAPK13, S100A11, CCL1, CCL7, CCL8, CCL18, CCL19, |
| | CCL22, CCL24, SLC1A2, SLC6A12, SLC11A1, SIGLEC1, SRC, TIE1, MARCO, HYAL2, |
| | CD163, LONP1, IGSF6, LILRB1, CD300C, SDS, LILRB4, EMILIN1, VSIG4, PHLDB1, |
| | NCAPH, CLEC4E, MYOF, HSPB7, ADAMDEC1, GLRX2, MS4A4A, ATP6V1H, TREM2, |
| | TMEM70, TMEM33, KCNK13, SLAMF8, HAMP, DNASE2B, MYOZ1, MFSD7, ADO, |
| | ADCK2, and TBC1D16 |

TABLE 3
Exemplary tumor-associated macrophage (TAM) gene signature sets

| Gene | |
|---|---|
| signature | |
| set | Genes |
| 1 | MARCO, ACP5, VSIG4, MRC1, MSR1, MCEMP1, CYP27A1, OLR1, GRN, GLIPR2, |
| | ARRDC4, C1QC, APOE, FOLR2, CTSD and SPP1 |

In some instances, nucleic acid expression levels of the genes described herein may be measured by polymerase chain reaction (PCR)-based assays, e.g., quantitative PCR, real-time PCR, quantitative real-time PCR (qRT-PCR), reverse transcriptase PCR (RT-PCR), and reverse transcriptase quantitative PCR (RT-qPCR). Platforms for performing quantitative PCR assays include Fluidigm (e.g., BIOMARK™ HD System). Other amplification-based methods include, for example, transcript-mediated amplification (TMA), strand displacement amplification (SDA), nucleic acid sequence based amplification (NASBA), and signal amplification methods such as bDNA.

In some instances, nucleic acid expression levels of the genes described herein also may be measured by sequencing-based techniques, such as, for example, RNA-seq, serial analysis of gene expression (SAGE), high-throughput sequencing technologies (e.g., massively parallel sequencing), and Sequenom MassARRAY® technology. Nucleic acid expression levels also may be measured by, for example, NanoString nCounter, and high-coverage expression profiling (HiCEP). Additional protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis).

Other methods for detecting nucleic acid levels of the genes described herein include protocols which examine or detect mRNAs, such as target mRNAs, in a tissue or cell sample by microarray technologies. Using nucleic acid microarrays, test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. Hybridization of a labeled probe with a particular array member indicates that the sample from which the probe was derived expresses that gene.

Primers and probes may be labeled with a detectable marker, such as, for example, a radioisotope, fluorescent compound, bioluminescent compound, a chemiluminescent compound, metal chelator, or enzyme. Such probes and primers can be used to detect the presence of expressed genes (e.g., the genes described herein) in a sample. As will be understood by the skilled artisan, many different primers and probes may be prepared based on the sequences provided herein (or, in the case of genomic DNA, their adjacent sequences) and used effectively to amplify, clone, and/or determine the presence and/or expression levels of the genes described herein.

Other methods to detect nucleic acid expression levels of the genes described herein include electrophoresis, Northern and Southern blot analyses, in situ hybridization (e.g., single or multiplex nucleic acid in situ hybridization), RNAse protection assays, and microarrays (e.g., Illumina BEADARRAY™ technology; Beads Array for Detection of Gene Expression (BADGE)).

In some instances, the macrophage biomarker can be analyzed by a number of methodologies, including, but not limited to, RNA-seq, PCR, RT-qPCR, qPCR, multiplex qPCR, multiplex RT-qPCR, NANOSTRING® nCOUNTER® Gene Expression Assay, microarray analysis, serial analysis of gene expression (SAGE), Northern blot analysis, MassARRAY, ISH, whole genome sequencing, FACS, spatial transcriptomics, spatial proteomics, Western blot, ELISA, immunoprecipitation, immunohistochemistry, immunofluorescence, radioimmunoassay, dot blotting, immunodetection methods, surface plasmon resonance, optical spectroscopy, mass spectrometery, and HPLC, or combinations thereof.

### (ii) RT-qPCR

In some instances, nucleic acid expression levels of the genes described herein (e.g., genes in a M1 macrophage gene signature set, genes in a tumor-associated macrophage gene signature set, or genes in a gene signature matrix) can be detected using reverse transcription quantitative polymerase chain reaction (RT-qPCR). The technique of RT-qPCR is a form of PCR wherein the nucleic acid to be amplified is RNA that is first reverse transcribed into cDNA and the amount of PCR product is measured at each step in a PCR reaction. As RNA cannot serve as a template for PCR, the first step in gene expression profiling by PCR is the reverse transcription of the RNA template into cDNA, followed by its amplification in a PCR reaction. For example, reverse transcriptases may include avilo myeloblastosis virus reverse transcriptase (AMY-RT) or Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GENEAMP™ RNA PCR kit (Perkin Elmer, Calif, USA), following the manufacturer's instructions. The derived cDNA can then be used as a template in the subsequent PCR reaction.

A variation of the PCR technique is quantitative real time PCR (qRT-PCR), which measures PCR product accumulation through a dual-labeled fluorogenic probe (i.e., TAQMAN® probe). The technique of quantitative real time polymerase chain reaction refers to a form of PCR wherein the amount of PCR product is measured at each step in a PCR reaction. This technique has been described in various publications including Cronin et al., Am. J. Pathol. 164(1):35-42 (2004); and Ma et al., Cancer Cell 5:607-616 (2004). Real time PCR is compatible both with quantitative competitive PCR, where an internal competitor for each target sequence is used for normalization, and/or with quantitative comparative PCR using a normalization gene contained within the sample, or a housekeeping gene for PCR. For further details see, e.g., Held et al., Genome Research 6:986-994 (1996).

The steps of a representative protocol for profiling gene expression using fixed, paraffin-embedded tissues as the RNA source, including mRNA isolation, purification, primer extension and amplification are given in various published journal articles (for example: Godfrey et al., Malec. Diagnostics 2: 84-91 (2000); Specht et al., Am. J. Pathol. 158: 419-29 (2001)). Briefly, a representative process starts with cutting a section (e.g., a 10 microgram section) of a paraffin-embedded tumor tissue samples. The RNA is then extracted, and protein and DNA are removed. After analysis of the RNA concentration, RNA repair and/or amplification steps may be included, if necessary, and RNA is reverse transcribed using gene specific promoters followed by PCR.

The nucleic acid expression level determined by an amplification-based method (e.g., RT-qPCR) may be expressed as a cycle threshold value (Ct). From this value, a normalized expression level for each gene can be determined, e.g., using the delta Ct (dCt) method as follows: Ct(Control/Reference Gene)−Ct (Gene of Interest/Target Gene)=dCt (Gene of Interest/Target Gene). One of skill in the art will appreciate that the dCt value obtained may be a negative dCt value or a positive dCt value. As defined herein, a higher dCt value indicates a higher expression level of the gene of interest relative to the control gene. Conversely, a lower dCt value indicates a lower expression level of the gene of interest relative to the control gene. In cases where the expression levels of a plurality of genes has been determined, the expression level for each gene, e.g., expressed as a dCt value, may then be used to determine a single value that represents an aggregate or composite expression level for the plurality of genes (e.g., genes in a M1 macrophage gene signature set or genes in a TAM gene signature set). The aggregate or composite expression level may be the mean or median of dCt values determined for each target gene/gene of interest. As defined herein, a higher averaged dCt or median dCt value indicates a higher aggregative expression level of the plurality of target genes relative to the control gene (or plurality of control genes). A lower averaged dCt or median dCt value indicates a lower aggregative expression level of the plurality of target genes relative to the control gene (or plurality of control genes). Expression levels may be compared to a reference level.

In one particular instance, the nucleic acid expression levels described herein may be determined using a method including: (a) obtaining or providing a sample from the individual, wherein the sample includes a tumor tissue sample (e.g., a paraffin-embedded, formalin-fixed tumor tissue sample); (b) isolating mRNA from said sample; (c) performing reverse transcription of the mRNA into cDNA (e.g., for at least one of the genes described herein (e.g., genes in a M1 macrophage gene signature set, genes in a TAM gene signature set, or genes in a gene signature matrix)); (d) amplifying the cDNA (e.g., for at least one of the genes described herein (e.g., genes in a M1 macrophage gene signature set, genes in a TAM gene signature set, or genes in a gene signature matrix)) using PCR; and (e) quantifying the nucleic acid expression levels (e.g., for at least one of the genes described herein (e.g., genes in a M1 macrophage gene signature set, genes in a tumor-associated macrophage (TAM) gene signature set, or genes in a gene signature matrix)).

One or more genes (e.g., one, two, three, four, five, six, seven, eight, nine, ten or more genes (e.g., 55, 82, 89, 106, 153, or 170 genes)) may be detected in a single assay depending on the primers or probes used. Further, the assay may be performed across one or more tubes (e.g., one, two, three, four, five, six, seven, eight, nine, ten or more tubes (e.g., 55, 82, 89, 106, 153, or 170 tubes)).

In some instances, the method further comprises (f) normalizing the nucleic acid expression level of the gene(s) (e.g., at least one of the genes described herein (e.g., genes in a M1 macrophage gene signature set, genes in a tumor-associated macrophage (TAM) gene signature set, or genes in a gene signature matrix)) in said sample to the expression level of one or more reference genes (e.g., one, two, three, four, five, six, seven, eight, nine, or more reference genes, e.g., a housekeeping gene (e.g., β-actin)). For example, RT-qPCR may be used to analyze the expression level of the genes described herein ((e.g., at least one of the genes described herein (e.g., genes in a M1 macrophage gene signature set, genes in a tumor-associated macrophage gene signature set, or genes in a gene signature matrix)) to generate an expression level that reflects a normalized, averaged dCT value for the analyzed genes.

(iii) RNA-Seq and Microarray

In some instances, nucleic acid expression levels of the genes described herein (e.g., genes in a M1 macrophage gene signature set, genes in a TAM gene signature set, or genes in a gene signature matrix) can be detected using RNA-seq. RNA-seq, also called Whole Transcriptome Shotgun Sequencing (WTSS), refers to the use of high-throughput sequencing technologies to sequence and/or quantify cDNA in order to obtain information about a sample's RNA content. Publications describing RNA-Seq include: Wang et al. “RNA-Seq: a revolutionary tool for transcriptomics” Nature Reviews Genetics 10 (1): 57-63 (January 2009); Ryan et al. BioTechniques 45 (1): 81-94 (2008); and Maher et al. “Transcriptome sequencing to detect gene fusions in cancer”. Nature 458 (7234): 97-101 (January 2009). In some instances, sequencing quality control is performed. In some instances, counts are normalized to transcripts per million (TPM).

### (a) Marker Gene Approaches

Marker gene approaches use the expression of one or more genes within a gene signature set (see, e.g., Table 2 and Table 3) to determine a macrophage biomarker (e.g., a number of M1 macrophages or tumor-associated macrophages in a sample). In some instances, the marker gene approach uses xCell (see, e.g., Aran et al. Genome Biol. 18(1):220 (2017)).

The gene signature sets exemplified in Table 2 and Table 3 may be modified to remove, substitute, or add genes. In some instances, the number of genes in any of the exemplified gene signature sets can be increased or reduced by one or more genes (e.g., one, two, three, four, five, six, seven, eight, nine, ten, or more genes). In some instances, the number of genes in any of the exemplified gene signature sets can be increased or reduced by between about 5% and about 20% (e.g., 5%, 6%, 7%, 8%, 9%, 10%, 11%, 12%, 13%, 14%, 15%, 16%, 17%, 18%, 19%, or 20%). In some instances, genes from any of the exemplified gene signature sets can be substituted with genes in the same signaling pathway. In some instances, genes from any of the exemplified gene signature sets can be added to a different exemplified gene signature set (e.g., a gene from gene signature set 1 (e.g., C1QA) can be added to gene signature set 2) to generate a gene signature set. In some instances, a gene signature set can comprise genes that are present in all exemplified gene signature sets (i.e., ACP2 and ADAMDEC1) or most exemplified gene signature sets (e.g., FDX1, CD163, HAMP, ABCD1, C1QA, CCL22, and TREM2). In some instances, a gene signature set comprises ACP2 and ADAMDEC1. In some instances, a gene signature set comprises ACP2, ADAMDEC1, and FDX1. In some instances, a gene signature set comprises ACP2, ADAMDEC1, and CD163. In some instances, a gene signature set comprises ACP2, ADAMDEC1, and HAMP. In some instances, a gene signature set comprises ACP2, ADAMDEC1, FDX1, and CD163. In some instances, a gene signature set comprises ACP2, ADAMDEC1, FDX1, and HAMP. In some instances, a gene signature set comprises ACP2, ADAMDEC1, CD163, and HAMP. In some instances, a gene signature set comprises ACP2, ADAMDEC1, FDX1, CD163, and HAMP. In some instances, a gene signature set comprises ACP2, ADAMDEC1, FDX1, CD163, HAMP, and ABCD1. In some instances, a gene signature set comprises ACP2, ADAMDEC1, FDX1, CD163, HAMP, and CCL22. In some instances, a gene signature set comprises ACP2, ADAMDEC1, FDX1, CD163, HAMP, and C1QA. In some instances, a gene signature set comprises ACP2, ADAMDEC1, FDX1, CD163, HAMP, and TREM2. In some instances, a gene signature set comprises ACP2, ADAMDEC1, FDX1, CD163, HAMP, ABCD1, and CCL22.

The steps of a representative protocol for determining a number of macrophages (e.g., M1 macrophages or tumor-associated macrophages) using an RNA-seq marker gene approach can be found in Aran et al. Genome Biol. 18(1):220 (2017). Briefly, sequence scores (e.g., pre-processed raw sequence reads) obtained from a sample are transformed based on a power function derived from synthetic mixtures of a cell type (e.g., M1 macrophages or tumor-associated macrophages) and a control cell type (e.g., multipotent progenitor cells or endothelial cells) within a range (e.g., 0.8% to 25.6%) based on expected abundance of the cell type present in the sample. The transformed scores are further adjusted using a spillover compensation matrix (limited to 0.5 off the diagonal) derived from synthetic mixtures of 25% of a cell type (e.g., M1 macrophages or tumor-associated macrophages) and 75% of a control cell type (e.g., multipotent progenitor cells or endothelial cells). The final adjusted score represents the fraction of the cell type present in the sample.

### (b) Deconvolution Approaches

Deconvolution approaches use the expression of one or more genes within a gene signature matrix to determine a macrophage biomarker (e.g., a number of M1 macrophages or tumor-associated macrophages in a sample). In some instances, the deconvolution approach uses quanTIseq (see, e.g., Finotello et al. Genome Med. 11(1):34 (2019)).

The steps of a representative protocol for determining a number of M1 macrophages or tumor-associated macrophages using an RNA-seq deconvolution approach can be found in Finotello et al. Genome Med. 11(1):34 (2019). Briefly, sequence scores (e.g., pre-processed raw sequence reads) obtained from a sample are normalized and deconvoluted using a gene signature matrix to calculate proportions of cell types (e.g., M1 macrophages, M2 macrophages, tumor-associated macrophages, B cells, monocytes, neutrophils, NK cells, non-regulatory CD4⁺ T cells, CD8⁺ T cells, regulatory T cells, dendritic cells, or other cell types) present in the sample using constrained least squares regression.

### (iv) Immunohistochemistry

In some instances, macrophages (e.g., M1 macrophages or tumor-associated macrophages) can be detected using immunohistochemistry (IHC). In some instances, any of the genes described herein (e.g., CD68, genes in a M1 macrophage gene signature set, genes in a tumor-associated macrophage gene set, or genes in a gene signature matrix) may be used to identify macrophages (e.g., M1 macrophages or tumor-associated macrophages) or distinguish macrophages from other cell types. In some instances, an antibody specific for any of the genes described herein (e.g., CD68, genes in a M1 macrophage gene signature set, genes in a tumor-associated macrophage gene signature set, or genes in a gene signature matrix) is used as a primary antibody in the IHC assay. In some instances, a horseradish peroxidase (HRP)-conjugated secondary antibody is used in the IHC assay. In some instances, a signal from the IHC assay is compared to an IHC assay performed with a negative control antibody. In some instances, macrophages (e.g., M1 macrophages or tumor-associated macrophages) can be detected using an antibody that binds CD68.

### (v) Flow Cytometry

In some instances, macrophages (e.g., M1 macrophages or tumor-associated macrophages) can be detected using flow cytometry. In some instances, any of the genes described herein (e.g., genes in a M1 macrophage gene signature set, genes in a tumor-associated macrophage gene set, or genes in a gene signature matrix) may be used to identify macrophages (e.g., M1 macrophages or tumor-associated macrophages) or distinguish macrophages from other cell types. In some instances, an antibody specific for any of the genes described herein (e.g., genes in a M1 macrophage gene signature set, genes in a tumor-associated macrophage gene signature set, or genes in a gene signature matrix) is used to label macrophages (e.g., M1 macrophages or tumor-associated macrophages).

### (vi) Samples

The sample may be taken from an individual who is suspected of having, or is diagnosed as having, a lymphoma, and hence is likely in need of treatment, or from a healthy individual who is not suspected of having a lymphoma or who does not have lymphoma but has a family history of a lymphoma. For assessment of gene expression, samples, such as those containing cells, or proteins or nucleic acids produced by these cells, may be used in the methods of the present invention. The expression level of a gene can be determined by assessing the amount (e.g., the absolute amount or concentration) of the markers in a sample (e.g., a tissue sample, e.g., a tumor tissue sample, such as a biopsy). In addition, the level of a gene can be assessed in bodily fluids or excretions containing detectable levels of genes. Bodily fluids or secretions useful as samples in the present invention include, e.g., blood, urine, saliva, stool, pleural fluid, lymphatic fluid, sputum, ascites, prostatic fluid, cerebrospinal fluid (CSF), or any other bodily secretion or derivative thereof. The word blood is meant to include whole blood, plasma, serum, or any derivative of blood. Assessment of a gene in such bodily fluids or excretions can sometimes be preferred in circumstances where an invasive sampling method is inappropriate or inconvenient. In other embodiments, a tumor tissue sample is preferred.

The sample may be frozen, fresh, fixed (e.g., formalin fixed), centrifuged, and/or embedded (e.g., paraffin embedded), etc. The cell sample can be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the amount of the marker in the sample. Likewise, biopsies may also be subjected to post-collection preparative and storage techniques, e.g., fixation, such as formalin fixation.

In one particular instance, the sample is a clinical sample. In another instance, the sample is used in a diagnostic assay, such as a diagnostic assay or diagnostic method of the invention. In some instances, the sample is obtained from a primary or metastatic tumor. Tissue biopsy is often used to obtain a representative piece of tumor tissue. Alternatively, tumor cells can be obtained indirectly in the form of tissues or fluids that are known or thought to contain the tumor cells of interest. For example, samples of lymphoma lesions may be obtained by resection, fine needle aspiration, pleural fluid, or blood. Genes or gene products can be detected from cancer or tumor tissue or from other body samples such as urine, sputum, serum or plasma. The same techniques discussed above for detection of target genes or gene products in cancerous samples can be applied to other body samples. Cancer cells may be sloughed off from cancer lesions and appear in such body samples. By screening such body samples, a simple early diagnosis can be achieved for these cancers. In addition, the progress of therapy can be monitored more easily by testing such body samples for target genes or gene products.

In some instances, the sample from the individual is a tissue sample, a whole blood sample, a plasma sample, a serum sample, or a combination thereof. In some instances, the sample is a tissue sample. In some instances, the sample is a tumor tissue sample. In some instances, the sample is obtained prior to treatment. In some instances, the tissue sample is formalin-fixed and paraffin-embedded (FFPE) sample, an archival sample, a fresh sample, or a frozen sample. In some instances, the sample from the individual is a tissue sample. In some instances, the tissue sample is a tumor tissue sample (e.g., biopsy tissue). In some instances, the tumor tissue sample includes tumor cells, tumor infiltrating immune cells, stromal cells, normal adjacent tissue (NAT) cells, or a combination thereof. In some instances, the tissue sample is a biopsy. In some instances, the tissue sample is blood cells, lymph nodes, or bone/bone marrow.

In some instances, the tumor tissue sample is extracted from a malignant cancerous tumor (i.e., cancer). In some instances, the cancer is a solid tumor, or a non-solid or soft tissue tumor. In some instances, the tumor tissue sample is a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) sample.

(vii) RNA Extraction

Prior to detecting the level of a nucleic acid, mRNA may be isolated from a target sample. In some instances, the mRNA is total RNA isolated from tumors or tumor cell lines or, alternatively, normal tissues or cell lines. If the source of mRNA is a primary tumor, mRNA can be extracted, for example, from frozen or archived paraffin-embedded and fixed (e.g., formalin-fixed) tissue samples. General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andres et al., Bio Techniques 18:42044 (1995). In particular, RNA isolation can be performed using a purification kit, buffer set, and protease from commercial manufacturers, such as Qiagen, according to the manufacturer's instructions. For example, total RNA from cells in culture can be isolated using Qiagen RNeasy mini-columns. Other commercially available RNA isolation kits include MASTERPURE® Complete DNA and RNA Purification Kit (EPICENTRE®, Madison, Wis.), and Paraffin Block RNA Isolation Kit (Ambion, Inc.). Total RNA from tissue samples can be isolated, for example, by using RNA Stat-60 (TelTest). RNA prepared from tumor tissue samples can also be isolated, for example, by cesium chloride density gradient centrifugation.

(viii) Expression Level

The expression level may reflect the expression levels of one or more genes described herein (e.g., one or more genes in a M1 macrophage gene signature set, one or more genes in a tumor-associated macrophage gene signature set, or one or more genes in a gene signature matrix). In certain instances, the detected expression level of each gene is normalized using any one of the standard normalization methods known in the art. One of skill in the art will appreciate that the normalization method used may depend on the gene expression methodology used (e.g., one or more housekeeping genes may be used for normalization in the context of an RT-qPCR methodology, but a whole genome or substantially whole genome may be used as a normalization baseline in the context of an RNA-seq methodology). For example, the detected expression level of each gene assayed can be normalized for both differences in the amount of the gene(s) assayed, variability in the quality of the samples used, and/or variability between assay runs.

In some instances, normalization may be accomplished by detecting expression of certain one or more normalizing gene(s), including reference gene(s) (e.g., a housekeeping gene (e.g., β-actin)). For example, in some instances, the nucleic acid expression levels detected using the methods described herein (e.g., for at least one of the genes described herein (e.g., genes in a M1 macrophage gene signature set, genes in a tumor-associated macrophage gene signature set, or genes in a gene signature matrix)) may be normalized to the expression level of one or more reference genes (e.g., one, two, three, four, five, six, seven, eight, nine, or more reference genes, e.g., a housekeeping gene (e.g., β-actin)). Alternatively, normalization can be based on the average signal or median signal of all of the assayed genes. On a gene-by-gene basis, a measured normalized amount of an mRNA can be compared to the amount found in a reference expression level. The presence and/or expression level/amount measured in a particular subject sample to be analyzed will fall at some percentile within this range, which can be determined by methods well known in the art.

In other instances, to determine an expression level, the detected expression level of each assayed gene is not normalized.

The expression level may reflect the aggregate or composite expression level of a single gene or a plurality of genes described herein (e.g., for at least one of the genes described herein (e.g., genes in a M1 macrophage gene signature set, genes in a tumor-associated macrophage gene signature set, or genes in a gene signature matrix)). Any statistical approaches known in the art may be used to determine the expression level.

For example, the expression level may reflect the median expression level, mean expression level, or a numerical value that reflects the aggregated Z-score expression level for the combination of genes assayed (e.g., for at least one of the genes described herein (e.g., genes in a M1 macrophage gene signature set, genes in a tumor-associated macrophage gene signature set, or genes in a gene signature matrix)).

In some instances, the expression level reflects the median normalized expression level, mean normalized expression level, or a numerical value that reflects the aggregated Z-score normalized expression level for the combinations of genes assayed (e.g., for at least one of the genes described herein (e.g., genes in a M1 macrophage gene signature set, genes in a tumor-associated macrophage gene signature set, or genes in a gene signature matrix)).

### IV. Therapeutic Methods, Compositions, and Uses

Provided herein are methods, compositions, and uses thereof, for treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)), the method including administering to the patient an effective amount of an anti-CD79b immunoconjugate (e.g., polatuzumab vedotin) and an anti-CD20 antibody (e.g., obinutuzumab or rituximab) based on a macrophage biomarker (e.g., a gene expression value (e.g., a gene expression value derived from any of the gene signature sets described herein (e.g., any of the exemplified gene signature sets in Table 2 or Table 3)) or an amount of macrophages (e.g., M1 macrophages or tumor-associated macrophages)) that has been determined in a sample (e.g., a tissue sample, e.g., a tumor tissue sample, such as a biopsy) from the patient.

In some instances, the anti-CD79b immunoconjugate (e.g., polatuzumab vedotin) and the anti-CD20 antibody (e.g., obinutuzumab or rituximab) may be administered as a first-line therapy. Alternatively, the anti-CD79b immunoconjugate (e.g., polatuzumab vedotin) and the anti-CD20 antibody (e.g., obinutuzumab or rituximab) may be administered as a second-line therapy.

In any of the below sections, the lymphoma can be a B-cell lymphoma. In some instances, the B-cell lymphoma is a non-Hodgkin lymphoma. In preferred embodiments, the non-Hodgkin lymphoma is a DLBCL. In some instances, the DLBCL is a germinal-center B-cell-like or activated B-cell-like diffuse large B-cell lymphoma. In some instances, the lymphoma is an indolent lymphoma. In some instances, the lymphoma is a CD20-positive lymphoma.

In any of the below sections, the patient is a human. In some instances, the patient has had no prior treatment. In some instances, the patient has had prior treatment. In some instances, the patient was previously treated with either an anti-CD79b immunoconjugate (e.g., polatuzumab vedotin) and an anti-CD20 antibody (e.g., obinutuzumab or rituximab) or an anti-CD20 antibody. In some instances, the patient has not been previously treated with either an anti-CD79b immunoconjugate (e.g., polatuzumab vedotin) and an anti-CD20 antibody (e.g., obinutuzumab or rituximab) or an anti-CD20 antibody.

In any of the below sections, the sample can be a tissue sample, a tumor sample, a whole blood sample, a plasma sample, or a serum sample. In some instances, the tissue sample is a tumor tissue sample. In some instances, the tumor tissue sample contains tumor cells, tumor-infiltrating immune cells, stromal cells, normal adjacent tissue (NAT) cells, or a combination thereof. In some instances, the tumor tissue sample is a biopsy. In some instances, the sample is an archival sample, a fresh sample, or a frozen sample.

In any of the below sections, the macrophage biomarker can be directly or indirectly measured. In some instances, the macrophage biomarker is a cell, nucleic acid, protein, lipid, or carbohydrate. In some instances, the macrophage biomarker is a gene expression value. In some instances, the macrophage biomarker is an amount of macrophages (e.g., M1 macrophages or tumor-associated macrophages).

### A. Macrophage Biomarkers for Use in Therapeutic Methods

In particular instances, the methods for treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) with an effective amount of an anti-CD79b immunoconjugate (e.g., polatuzumab vedotin) and an anti-CD20 antibody (e.g., obinutuzumab or rituximab) are based on a macrophage biomarker (e.g., a gene expression value (e.g., a gene expression value derived from any of the gene signature sets described herein (e.g., any of the exemplified gene signature sets in Table 2 or Table 3)) or an amount of macrophages (e.g., M1 macrophages or tumor-associated macrophages)) that has been determined in a sample (e.g., a tissue sample, e.g., a tumor tissue sample, such as a biopsy) from the patient.

In one aspect, provided herein are methods for treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)), the method including: (a) measuring a macrophage biomarker (e.g., a gene expression value (e.g., a gene expression value derived from any of the gene signature sets described herein (e.g., any of the exemplified gene signature sets in Table 2 or Table 3)) or an amount of macrophages (e.g., M1 macrophages or tumor-associated macrophages)) in a sample (e.g., a tissue sample, e.g., a tumor tissue sample, such as a biopsy) from the patient, wherein the amount or level of the macrophage biomarker in the sample is below a reference macrophage biomarker amount or level, and (b) administering an effective amount of an anti-CD79b immunoconjugate and an anti-CD20 antibody to the patient based on the macrophage biomarker measured in step (a).

In another aspect, provided herein are methods for treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)), the method including administering to the patient an effective amount of an anti-CD79b immunoconjugate and an anti-CD20 antibody, wherein prior to treatment the amount or level of a macrophage biomarker macrophage biomarker (e.g., a gene expression value (e.g., a gene expression value derived from any of the gene signature sets described herein (e.g., any of the exemplified gene signature sets in Table 2 or Table 3)) or an amount of macrophages (e.g., M1 macrophages or tumor-associated macrophages)) in a sample (e.g., a tissue sample, e.g., a tumor tissue sample, such as a biopsy) from the patient has been determined to be below a reference macrophage biomarker amount or level.

In another aspect, provided herein are methods for treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) and having an amount or level of a macrophage biomarker (e.g., a gene expression value (e.g., a gene expression value derived from any of the gene signature sets described herein (e.g., any of the exemplified gene signature sets in Table 2 or Table 3)) or an amount of macrophages (e.g., M1 macrophages or tumor-associated macrophages)) in a sample (e.g., a tissue sample, e.g., a tumor tissue sample, such as a biopsy) from the patient that is below a reference macrophage biomarker amount or level including administering to the patient an effective amount of an anti-CD79b immunoconjugate and an anti-CD20 antibody.

The amount or level of the macrophage biomarker that determines the various methods described herein are further described below.

### (i) Decreased Macrophage Biomarker

An amount or level of the macrophage biomarker in a sample from a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) that is below a reference macrophage biomarker amount or level may determine that a patient is to be administered an anti-CD79b immunoconjugate (e.g., polatuzumab vedotin) and an anti-CD20 antibody (e.g., obinutuzumab or rituximab).

In some instances, a method of treating a patient with an amount or level of a macrophage biomarker in a sample that is in about the bottom 99th percentile (equal to, or lower than, about the 99% prevalence level), about the bottom 95th percentile (equal to, or lower than, about the 95% prevalence level), about the bottom 90th percentile (equal to, or lower than, about the 90% prevalence level), about the bottom 85th percentile (equal to, or lower than, about the 85% prevalence level), about the bottom 80th percentile (equal to, or lower than, about the 80% prevalence level), about the bottom 75th percentile (equal to, or lower than, about the 75% prevalence level), about the bottom 70th percentile (equal to, or lower than, about the 70% prevalence level), about the bottom 65th percentile (equal to, or lower than, about the 65% prevalence level), about the bottom 60th percentile (equal to, or lower than, about the 60% prevalence level), about the bottom 55th percentile (equal to, or lower than, about the 55% prevalence level), about the bottom 50th percentile (equal to, or lower than, about the 50% prevalence level), about the bottom 45th percentile (equal to, or lower than, about the 45% prevalence level), about the bottom 40th percentile (equal to, or lower than, about the 40% prevalence level), about the bottom 35th percentile (equal to, or lower than, about the 35% prevalence level), about the bottom 30th percentile (equal to, or lower than, about the 30% prevalence level), about the bottom 25th percentile (equal to, or lower than, about the 25% prevalence level), about the bottom 20th percentile (equal to, or lower than, about the 20% prevalence level), about the bottom 15th percentile (equal to, or lower than, about the 15% prevalence level), about the bottom 10th percentile (equal to, or lower than, about the 10% prevalence level), about the bottom 5th percentile (equal to, or lower than, about the 5% prevalence level), or about the bottom 1st percentile (equal to, or lower than, about the 1% prevalence level) of the amount or level of the macrophage biomarker in the reference population comprises administering a therapy that includes an anti-CD79b immunoconjugate (e.g., polatuzumab vedotin) and an anti-CD20 antibody (e.g., obinutuzumab or rituximab).

In some instances, a method of treating a patient with an amount or level of a macrophage biomarker in a sample that is in about the bottom 10th to about the bottom 90th percentile, about the bottom 20th to about the bottom 80th percentile, about the bottom 30th to about the bottom 70th percentile, about the bottom 40th to about the bottom 60th percentile, about the bottom 45th to about the bottom 55th percentile, about the bottom 48th to about the bottom 52th percentile, about the bottom 49.5th to about the bottom 50.5th percentile, about the bottom 49.9th to about the bottom 50.1th percentile, or about the bottom 50th percentile of the amount or level of the macrophage biomarker in the reference population comprises administering a therapy that includes an anti-CD79b immunoconjugate (e.g., polatuzumab vedotin) and an anti-CD20 antibody (e.g., obinutuzumab or rituximab). For example, in some instances, a method of treating a patient with an amount or level of a macrophage biomarker in a sample that is between about 10% to about 90% prevalence, about 15 to about 85% prevalence, about 20% to about 80% prevalence, about 25% to about 75% prevalence, about 30% to about 70% prevalence, about 35% to about 65% prevalence, about 40% to about 60% prevalence, about 45% to about 55% prevalence, about 48% to about 52% prevalence, about 49.5% to about 50.5% prevalence, about 49.9% to about 50.1% prevalence, or about 50% prevalence in the reference population comprises administering a therapy that includes an anti-CD79b immunoconjugate (e.g., polatuzumab vedotin) and an anti-CD20 antibody (e.g., obinutuzumab or rituximab).

In some instances, an amount or level of the macrophage biomarker that is lower than a reference amount or level of the macrophage biomarker refers to a decrease of about 10%, 20%, 30%, 40%, 50%, 60%, 70%, 80%, 90%, 95%, 96%, 97%, 98%, or 99% or greater in the amount or level of the macrophage biomarker, detected by standard art-known methods such as those described herein, as compared to the amount or level of the macrophage biomarker in a reference sample, reference cell, reference tissue, control sample, control cell, or control tissue. In certain instances, an amount or level of the macrophage biomarker that is lower than a reference amount or level of the macrophage biomarker refers to a decrease in the amount or level of the macrophage biomarker in the sample, wherein the decrease is at least about 1.5×, 1.75×, 2×, 3×, 4×, 5×, 6×, 7×, 8×, 9×, 10×, 25×, 50×, 75×, or 100× the amount or level of the macrophage biomarker in a reference sample, reference cell, reference tissue, control sample, control cell, or control tissue. In some instances, an amount or level of the macrophage biomarker that is lower than a reference amount or level of the macrophage biomarker refers to a decrease in the amount or level of the macrophage biomarker that is greater than about 1.5-fold, about 1.75-fold, about 2-fold, about 2.25-fold, about 2.5-fold, about 2.75-fold, about 3.0-fold, or about 3.25-fold as compared to the amount or level of the macrophage biomarker in a reference sample, reference cell, reference tissue, control sample, control cell, or control tissue.

In some instances, an amount or level of the macrophage biomarker that is lower than a reference amount or level of the macrophage biomarker refers to an overall decrease of about 10%, 20%, 30%, 40%, 50%, 60%, 70%, 80%, 90%, 95%, 96%, 97%, 98%, or 99% or greater in the amount or level of the macrophage biomarker, detected by standard art-known methods such as those described herein, as compared to a pre-assigned amount or level of the macrophage biomarker. In certain instances, an amount or level of the macrophage biomarker that is lower than a reference amount or level of the macrophage biomarker refers to a decrease in the amount or level of the macrophage biomarker in the sample, wherein the decrease is at least about 1.5×, 1.75×, 2×, 3×, 4×, 5×, 6×, 7×, 8×, 9×, 10×, 25×, 50×, 75×, or 100× a pre-assigned amount or level of the macrophage biomarker. In some instances, an amount or level of the macrophage biomarker that is lower than a reference amount or level of the macrophage biomarker refers to an overall decrease in the amount or level of the macrophage biomarker that is greater than about 1.5-fold, about 1.75-fold, about 2-fold, about 2.25-fold, about 2.5-fold, about 2.75-fold, about 3.0-fold, or about 3.25-fold as compared to a pre-assigned amount or level of the macrophage biomarker.

### (ii) Increased Macrophage Biomarker

Previous studies, as described in WO2022/031749, determined that an amount or level of the macrophage biomarker in a sample from a patient that is above a reference macrophage biomarker amount or level identifies, diagnoses, and/or predicts the patient as one who may benefit from a treatment comprising an anti-CD20 antibody. Based on the studies described herein, an amount or level of the macrophage biomarker in a sample from a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) that is above a reference macrophage biomarker amount or level may identify, diagnose, and/or predict the patient as one who may benefit from a treatment comprising either an anti-CD79b immunoconjugate (e.g., polatuzumab vedotin) and an anti-CD20 antibody (e.g., obinutuzumab or rituximab) or an anti-CD20 antibody (e.g., obinutuzumab or rituximab). Selection of therapy comprising an anti-CD79b immunoconjugate (e.g., polatuzumab vedotin) and an anti-CD20 antibody (e.g., obinutuzumab or rituximab) or selection of a therapy comprising an anti-CD20 antibody (e.g., obinutuzumab or rituximab) where the patient sample has an amount or level of the macrophage biomarker that is above a reference macrophage biomarker amount or level may be based on additional factors in addition to the amount or level of the macrophage biomarker.

(iii) Reference Macrophage Biomarker

The reference macrophage biomarker amount or level can be a pre-assigned macrophage biomarker amount or level. In some instances, the amount or level of the macrophage biomarker in a reference population is a median amount or level of the macrophage biomarker of the reference population. In some instances, the amount or level of the macrophage biomarker in a reference population is a mean amount or level of the macrophage biomarker of the reference population.

In some instances, the pre-assigned macrophage biomarker amount or level is a percentage of cellular subtypes within a sample. In some instances, the percentage of cellular subtypes within a sample is between about 0% and 40% (e.g., 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40%). In some instances, the percentage of cellular subtypes within a sample is between about 0% and 10% (e.g., 0%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%). In some instances, the percentage of cellular subtypes within a sample is less than 10% (e.g., 0%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%). In some instances, the percentage of cellular subtypes within a sample is about 6%. In some instances, the percentage of cellular subtypes within a sample is about 5%. In some instances, the percentage of cellular subtypes within a sample is about 4.74%. In some instances, the percentage of cellular subtypes within a sample is about 4%. In some instances, the percentage of cellular subtypes within a sample is about 3.35%. In some instances, the percentage of cellular subtypes within a sample is about 3%. In some instances, the percentage of cellular subtypes within a sample is about 2.5%. In some instances, the percentage of cellular subtypes within a sample is about 2%. In some instances, the percentage of cellular subtypes within a sample is about 1.67%. In some instances, the percentage of cellular subtypes within a sample is about 1%. In some instances, the percentage of cellular subtypes within a sample is about 0%.

The reference amount or level of the macrophage biomarker described herein may be based on the amount or level of the macrophage biomarker in a reference population. In some instances, the reference macrophage biomarker described herein is an amount or level of the macrophage biomarker in a reference population that includes one or more (e.g., two or more, three or more, four or more, or five or more) subsets of patients.

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)).

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) who have been administered one or more doses (e.g., at least one, two, three, four, five, six, seven, eight, nine, or ten or more doses) of either an anti-CD79b immunoconjugate (e.g., polatuzumab vedotin) and an anti-CD20 antibody (e.g., obinutuzumab or rituximab) or an anti-CD20 antibody (e.g., obinutuzumab or rituximab).

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) who have received treatment with an anti-CD20 antibody (e.g., obinutuzumab or rituximab) as a monotherapy.

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) who have received treatment with an anti-CD79b immunoconjugate (e.g., polatuzumab vedotin) and an anti-CD20 antibody (e.g., obinutuzumab or rituximab) as a combination therapy (e.g., a combination therapy including an anti-CD79b immunoconjugate (e.g., polatuzumab vedotin) and an anti-CD20 antibody (e.g., obinutuzumab or rituximab) and an additional therapeutic agent (e.g., anti-cancer therapy (e.g., a cytotoxic agent, a growth-inhibitory agent, a radiation therapy, an anti-angiogenic agent, or a combination thereof), e.g., CHP).

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) who have received treatment with an anti-CD20 antibody (e.g., obinutuzumab or rituximab) as a combination therapy (e.g., a combination therapy including an anti-CD20 antibody (e.g., obinutuzumab or rituximab) and an additional therapeutic agent (e.g., anti-cancer therapy (e.g., a cytotoxic agent, a growth-inhibitory agent, a radiation therapy, an anti-angiogenic agent, or a combination thereof), e.g., CHOP).

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) who have received treatment with a therapy that does not include an anti-CD79b immunoconjugate (e.g., polatuzumab vedotin) and an anti-CD20 antibody (e.g., obinutuzumab or rituximab) and includes an anti-cancer therapy (e.g., a cytotoxic agent, a growth-inhibitory agent, a radiation therapy, an anti-angiogenic agent, or a combination thereof), e.g., CHP).

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) who have received treatment with a therapy that does not include an anti-CD20 antibody (e.g., obinutuzumab or rituximab) and includes an anti-cancer therapy (e.g., a cytotoxic agent, a growth-inhibitory agent, a radiation therapy, an anti-angiogenic agent, or a combination thereof), e.g., CHOP).

For example, in some instances, the reference population includes a first subset of patients who have been treated with an anti-CD79b immunoconjugate (e.g., polatuzumab vedotin) and an anti-CD20 antibody (e.g., obinutuzumab or rituximab) and a second subset of patients who have not been treated with an anti-CD79b immunoconjugate (e.g., polatuzumab vedotin) and an anti-CD20 antibody (e.g., obinutuzumab or rituximab).

For example, in some instances, the reference population includes a first subset of patients who have been treated with an anti-CD20 antibody (e.g., obinutuzumab or rituximab) and a second subset of patients who have not been treated an anti-CD20 antibody (e.g., obinutuzumab or rituximab).

For example, in some instances, the reference population includes a first subset of patients who have been treated with an anti-CD79b immunoconjugate (e.g., polatuzumab vedotin) and an anti-CD20 antibody (e.g., obinutuzumab or rituximab) and a second subset of patients who have been treated with an anti-CD20 antibody (e.g., obinutuzumab or rituximab).

In some instances, the reference amount or level of the macrophage biomarker significantly separates each of the first and second subsets of patients based on a significant difference between a patient's responsiveness (e.g., PFS or OS) to treatment with the anti-CD79b immunoconjugate (e.g., polatuzumab vedotin) and the anti-CD20 antibody (e.g., obinutuzumab or rituximab) and a patient's responsiveness to treatment with the anti-CD20 antibody (e.g., obinutuzumab or rituximab) below the reference macrophage biomarker, wherein the patient's responsiveness to treatment with the anti-CD79b immunoconjugate (e.g., polatuzumab vedotin) and the anti-CD20 antibody (e.g., obinutuzumab or rituximab) is significantly improved relative to the patient's responsiveness to treatment with the anti-CD20 antibody (e.g., obinutuzumab or rituximab). For example, in some instances, the reference amount or level of the macrophage biomarker optimally separates each of the first and second subsets of patients based on a maximum difference between a patient's responsiveness (e.g., PFS or OS) to treatment with the anti-CD79b immunoconjugate (e.g., polatuzumab vedotin) and the anti-CD20 antibody (e.g., obinutuzumab or rituximab) and a patient's responsiveness to treatment with the anti-CD20 antibody (e.g., obinutuzumab or rituximab) below the reference macrophage biomarker, wherein the patient's responsiveness to treatment with the anti-CD79b immunoconjugate (e.g., polatuzumab vedotin) and the anti-CD20 antibody (e.g., obinutuzumab or rituximab) is significantly improved relative to the patient's responsiveness to treatment with the anti-CD20 antibody (e.g., obinutuzumab or rituximab). In some instances, the aforementioned first subset of patients is patients treated with Pola-R-CHP (polatuzumab vedotin plus rituximab, cyclophosphamide, doxorubicin, and prednisone). In some instances, the aforementioned second subset of patients is patients treated with R-CHOP (Rituximab plus cyclophosphamide, doxorubicin, vincristine, and prednisone).

In some instances, the reference amount or level of the macrophage biomarker significantly separates each of the first and second subsets of patients based on a significant difference between a patient's responsiveness (e.g., PFS or OS) to treatment with the anti-CD79b immunoconjugate (e.g., polatuzumab vedotin) and the anti-CD20 antibody (e.g., obinutuzumab or rituximab) and a patient's responsiveness to treatment without the anti-CD79b immunoconjugate (e.g., polatuzumab vedotin) and the anti-CD20 antibody (e.g., obinutuzumab or rituximab) below the reference macrophage biomarker, wherein the patient's responsiveness to treatment with the anti-CD79b immunoconjugate (e.g., polatuzumab vedotin) and the anti-CD20 antibody (e.g., obinutuzumab or rituximab) is significantly improved relative to the patient's responsiveness to treatment without the anti-CD79b immunoconjugate (e.g., polatuzumab vedotin) and the anti-CD20 antibody (e.g., obinutuzumab or rituximab). For example, in some instances, the reference amount or level of the macrophage biomarker optimally separates each of the first and second subsets of patients based on a maximum difference between a patient's responsiveness (e.g., PFS or OS) to treatment with the anti-CD79b immunoconjugate (e.g., polatuzumab vedotin) and the anti-CD20 antibody (e.g., obinutuzumab or rituximab) and a patient's responsiveness to treatment without the anti-CD79b immunoconjugate (e.g., polatuzumab vedotin) and the anti-CD20 antibody (e.g., obinutuzumab or rituximab) below the reference macrophage biomarker, wherein the patient's responsiveness to treatment with the anti-CD79b immunoconjugate (e.g., polatuzumab vedotin) and the anti-CD20 antibody (e.g., obinutuzumab or rituximab) is significantly improved relative to the patient's responsiveness to treatment without the anti-CD79b immunoconjugate (e.g., polatuzumab vedotin) and the anti-CD20 antibody (e.g., obinutuzumab or rituximab). In some instances, the aforementioned first subset of patients is patients treated with Pola-R-CHP (polatuzumab vedotin plus rituximab, cyclophosphamide, doxorubicin, and prednisone).

In some instances, the reference amount or level of the macrophage biomarker significantly separates each of the first and second subsets of patients based on a significant difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) below the reference macrophage biomarker, wherein the patient's responsiveness to treatment without the anti-CD20 antibody (e.g., obinutuzumab or rituximab) is significantly improved relative to the patient's responsiveness to treatment with the anti-CD20 antibody (e.g., obinutuzumab or rituximab). For example, in some instances, the reference amount or level of the macrophage biomarker optimally separates each of the first and second subsets of patients based on a maximum difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) below the reference macrophage biomarker, wherein the patient's responsiveness to treatment without the anti-CD20 antibody (e.g., obinutuzumab or rituximab) is significantly improved relative to the patient's responsiveness to treatment with the anti-CD20 antibody (e.g., obinutuzumab or rituximab). In some instances, the aforementioned first subset of patients is patients treated with R-CHOP (Rituximab plus cyclophosphamide, doxorubicin, vincristine, and prednisone).

In some instances, an optimal separation or significant separation may be based on a hazard ratio (HR) determined from an analysis of the amount or level of the macrophage biomarker in the first and second subsets of patients, wherein the HR is less than 1, e.g., an HR of about 0.95, about 0.9, about 0.8, about 0.7, about 0.6, about 0.5, about 0.4, about 0.3, about 0.2, about 0.1 or lower. For example, in particular instances, an optimal separation or significant separation may be based on a hazard ratio (HR) determined from an analysis of the amount or level of the macrophage biomarker in the first and second subsets of patients, wherein the upper bound of the 95% confidence interval of the HR is less than 1, e.g., an upper bound of the 95% confidence interval of the HR of about 0.95, about 0.9, about 0.8, about 0.7, about 0.6, about 0.5, about 0.4, about 0.3, about 0.2, about 0.1 or lower.

Additionally, or alternatively, the reference macrophage biomarker may be an amount or level of the macrophage biomarker in a reference population, wherein the reference population includes at least one subset of patients who do not have a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) or have a lymphoma but are treatment naïve.

### (iv) Indications

The methods described herein are useful for treating patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) with an anti-CD79b immunoconjugate (e.g., polatuzumab vedotin) and an anti-CD20 antibody (e.g., obinutuzumab or rituximab).

In some instances, the lymphoma may be indolent lymphoma. In some instances, the lymphoma may be a B-cell lymphoma, e.g., a non-Hodgkin lymphoma, e.g., a diffuse large B-cell lymphoma (e.g., a germinal-center B-cell-like or activated B-cell-like diffuse large B-cell lymphoma). In some instances, the lymphoma may be a CD20-positive lymphoma.

In certain instances, the cancer may be a B-cell lymphoma. For example, the B-cell lymphoma may be a non-Hodgkin lymphoma, including but not limited to a diffuse large B-cell lymphoma (e.g., a germinal-center B-cell-like or activated B-cell-like diffuse large B-cell lymphoma)). For example, the methods described herein may be used for treating a patient having a diffuse large B-cell lymphoma) with an anti-CD79b immunoconjugate and an anti-CD20 antibody (e.g., obinutuzumab or rituximab), the method including determining that a macrophage biomarker in a sample from the patient is an amount or level that is below a reference macrophage biomarker amount or level.

In some instances, the individual having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) has not been previously treated for the lymphoma (treatment naïve). For example, in some instances, the individual having a lymphoma has not previously received either an anti-CD79b immunoconjugate (e.g., polatuzumab vedotin) and an anti-CD20 antibody (e.g., obinutuzumab or rituximab) or an anti-CD20 antibody (e.g., obinutuzumab or rituximab).

In some instances, the individual having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) has previously received treatment for the lymphoma. In some instances, the individual having a lymphoma has previously received treatment including either an anti-CD79b immunoconjugate (e.g., polatuzumab vedotin) and an anti-CD20 antibody (e.g., obinutuzumab or rituximab) or an anti-CD20 antibody (e.g., obinutuzumab or rituximab)).

### (v) Treatment Benefits

A patient who benefits from receiving treatment with an anti-CD79b immunoconjugate (e.g., polatuzumab vedotin) and an anti-CD20 antibody (e.g., obinutuzumab or rituximab) may experience, for example, a delay or prevention in the occurrence or recurrence of a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)), alleviation of symptoms, diminishment of any direct or indirect pathological consequences of the cancer, prevention of metastasis, decrease in the rate of disease progression, amelioration or palliation of the disease state, or remission or improved prognosis. In some instances, the treatments described herein are used to delay development of a cancer or to slow the progression of a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)). In some instances, the benefit may be an increase in OS, PFS, CR, PR, or a combination thereof.

In some instances, a method of treating a patient having a diffuse large B-cell lymphoma with an amount or level of a macrophage biomarker in a sample that is below a reference macrophage biomarker amount or level (e.g., an amount or level of a macrophage biomarker in a reference population) comprises administering an anti-CD79b immunoconjugate (e.g., polatuzumab vedotin) and an anti-CD20 antibody (e.g., obinutuzumab or rituximab) achieves an improvement of OS (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) relative to a treatment that does not include an anti-CD79b immunoconjugate (e.g., polatuzumab vedotin) and an anti-CD20 antibody (e.g., obinutuzumab or rituximab).

In some instances, a method of treating a patient having a diffuse large B-cell lymphoma with an amount or level of a macrophage biomarker in a sample that is below a reference macrophage biomarker amount or level (e.g., an amount or level of a macrophage biomarker in a reference population) comprises administering an anti-CD79b immunoconjugate (e.g., polatuzumab vedotin) and an anti-CD20 antibody (e.g., obinutuzumab or rituximab) achieves an improvement of PFS (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) relative to a treatment that does not include an anti-CD79b immunoconjugate (e.g., polatuzumab vedotin) and an anti-CD20 antibody (e.g., obinutuzumab or rituximab).

### B. Uses of an Anti-CD79b Immunoconjugate and an Anti-CD20 Antibody in the Manufacture of Medicaments

In a further aspect, the invention provides for the use of an anti-CD79b immunoconjugate (e.g., polatuzumab vedotin) and an anti-CD20 antibody (e.g., obinutuzumab or rituximab) in the manufacture or preparation of a medicament for treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)). In some instances, the medicament is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) with an amount or level of a macrophage biomarker in a sample from the patient that is below a reference macrophage biomarker amount or level.

The amount or level of the macrophage biomarker that determines the various uses described herein are further described below.

### (i) Decreased Macrophage Biomarker

In some instances, a medicament is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) with an amount or level of the macrophage biomarker in the sample that is in about the bottom 99th percentile (equal to, or lower than, about the 99% prevalence level), about the bottom 95th percentile (equal to, or lower than, about the 95% prevalence level), about the bottom 90th percentile (equal to, or lower than, about the 90% prevalence level), about the bottom 85th percentile (equal to, or lower than, about the 85% prevalence level), about the bottom 80th percentile (equal to, or lower than, about the 80% prevalence level), about the bottom 75th percentile (equal to, or lower than, about the 75% prevalence level), about the bottom 70th percentile (equal to, or lower than, about the 70% prevalence level), about the bottom 65th percentile (equal to, or lower than, about the 65% prevalence level), about the bottom 60th percentile (equal to, or lower than, about the 60% prevalence level), about the bottom 55th percentile (equal to, or lower than, about the 55% prevalence level), about the bottom 50th percentile (equal to, or lower than, about the 50% prevalence level), about the bottom 45th percentile (equal to, or lower than, about the 45% prevalence level), about the bottom 40th percentile (equal to, or lower than, about the 40% prevalence level), about the bottom 35th percentile (equal to, or lower than, about the 35% prevalence level), about the bottom 30th percentile (equal to, or lower than, about the 30% prevalence level), about the bottom 25th percentile (equal to, or lower than, about the 25% prevalence level), about the bottom 20th percentile (equal to, or lower than, about the 20% prevalence level), about the bottom 15th percentile (equal to, or lower than, about the 15% prevalence level), about the bottom 10th percentile (equal to, or lower than, about the 10% prevalence level), about the bottom 5th percentile (equal to, or lower than, about the 5% prevalence level), or about the bottom 1st percentile (equal to, or lower than, about the 1% prevalence level) of the amount or level of the macrophage biomarker in the reference population.

In some instances, a medicament is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) with an amount or level of the macrophage biomarker in the sample that is in about the bottom 10th to about the bottom 90th percentile, about the bottom 20th to about the bottom 80th percentile, about the bottom 30th to about the bottom 70th percentile, about the bottom 40th to about the bottom 60th percentile, about the bottom 45th to about the bottom 55th percentile, about the bottom 48th to about the bottom 52th percentile, about the bottom 49.5th to about the bottom 50.5th percentile, about the bottom 49.9th to about the bottom 50.1th percentile, or about the bottom 50th percentile of the amount or level of the macrophage biomarker in the reference population. For example, in some instances, a medicament is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) with an amount or level of the macrophage biomarker in the sample that is between about 10% to about 90% prevalence, about 15 to about 85% prevalence, about 20% to about 80% prevalence, about 25% to about 75% prevalence, about 30% to about 70% prevalence, about 35% to about 65% prevalence, about 40% to about 60% prevalence, about 45% to about 55% prevalence, about 48% to about 52% prevalence, about 49.5% to about 50.5% prevalence, about 49.9% to about 50.1% prevalence, or about 50% prevalence in the reference population.

In some instances, an amount or level of the macrophage biomarker that is lower than a reference amount or level of the macrophage biomarker refers to a decrease of about 10%, 20%, 30%, 40%, 50%, 60%, 70%, 80%, 90%, 95%, 96%, 97%, 98%, or 99% or greater in the amount or level of the macrophage biomarker, detected by standard art-known methods such as those described herein, as compared to the amount or level of the macrophage biomarker in a reference sample, reference cell, reference tissue, control sample, control cell, or control tissue. In certain instances, an amount or level of the macrophage biomarker that is lower than a reference amount or level of the macrophage biomarker refers to a decrease in the amount or level of the macrophage biomarker in the sample, wherein the decrease is at least about 1.5×, 1.75×, 2×, 3×, 4×, 5×, 6×, 7×, 8×, 9×, 10×, 25×, 50×, 75×, or 100× the amount or level of the macrophage biomarker in a reference sample, reference cell, reference tissue, control sample, control cell, or control tissue. In some instances, an amount or level of the macrophage biomarker that is lower than a reference amount or level of the macrophage biomarker refers to a decrease in the amount or level of the macrophage biomarker that is greater than about 1.5-fold, about 1.75-fold, about 2-fold, about 2.25-fold, about 2.5-fold, about 2.75-fold, about 3.0-fold, or about 3.25-fold as compared to the amount or level of the macrophage biomarker in a reference sample, reference cell, reference tissue, control sample, control cell, or control tissue.

In some instances, an amount or level of the macrophage biomarker that is lower than a reference amount or level of the macrophage biomarker refers to an overall decrease of about 10%, 20%, 30%, 40%, 50%, 60%, 70%, 80%, 90%, 95%, 96%, 97%, 98%, or 99% or greater in the amount or level of the macrophage biomarker, detected by standard art-known methods such as those described herein, as compared to a pre-assigned amount or level of the macrophage biomarker. In certain instances, an amount or level of the macrophage biomarker that is lower than a reference amount or level of the macrophage biomarker refers to a decrease in the amount or level of the macrophage biomarker in the sample, wherein the decrease is at least about 1.5×, 1.75×, 2×, 3×, 4×, 5×, 6×, 7×, 8×, 9×, 10×, 25×, 50×, 75×, or 100× a pre-assigned amount or level of the macrophage biomarker. In some instances, an amount or level of the macrophage biomarker that is lower than a reference amount or level of the macrophage biomarker refers to an overall decrease in the amount or level of the macrophage biomarker that is greater than about 1.5-fold, about 1.75-fold, about 2-fold, about 2.25-fold, about 2.5-fold, about 2.75-fold, about 3.0-fold, or about 3.25-fold as compared to a pre-assigned amount or level of the macrophage biomarker.

### (ii) Reference Macrophage Biomarker

The reference macrophage biomarker amount or level can be a pre-assigned macrophage biomarker amount or level. In some instances, the amount or level of the macrophage biomarker in a reference population is a median amount or level of the macrophage biomarker of the reference population. In some instances, the amount or level of the macrophage biomarker in a reference population is a mean amount or level of the macrophage biomarker of the reference population.

In some instances, the pre-assigned macrophage biomarker amount or level is a percentage of cellular subtypes within a sample. In some instances, the percentage of cellular subtypes within a sample is between about 0% and 40% (e.g., 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40%). In some instances, the percentage of cellular subtypes within a sample is between about 0% and 10% (e.g., 0%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%). In some instances, the percentage of cellular subtypes within a sample is less than 10% (e.g., 0%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%). In some instances, the percentage of cellular subtypes within a sample is about 6%. In some instances, the percentage of cellular subtypes within a sample is about 5%. In some instances, the percentage of cellular subtypes within a sample is about 4.74%. In some instances, the percentage of cellular subtypes within a sample is about 4%. In some instances, the percentage of cellular subtypes within a sample is about 3.35%. In some instances, the percentage of cellular subtypes within a sample is about 3%. In some instances, the percentage of cellular subtypes within a sample is about 2.5%. In some instances, the percentage of cellular subtypes within a sample is about 2%. In some instances, the percentage of cellular subtypes within a sample is about 1.67%. In some instances, the percentage of cellular subtypes within a sample is about 1%. In some instances, the percentage of cellular subtypes within a sample is about 0%.

The reference amount or level of the macrophage biomarker described herein may be based on the amount or level of the macrophage biomarker in a reference population. In some instances, the reference macrophage biomarker described herein is an amount or level of the macrophage biomarker in a reference population that includes one or more (e.g., one or more, two or more, three or more, four or more, or five or more) subsets of patients.

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)).

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) who have been administered one or more doses (e.g., at least one, two, three, four, five, six, seven, eight, nine, or ten or more doses) of either an anti-CD79b immunoconjugate (e.g., polatuzumab vedotin) and an anti-CD20 antibody (e.g., obinutuzumab or rituximab) or an anti-CD20 antibody (e.g., obinutuzumab or rituximab).

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) who have received treatment with an anti-CD20 antibody (e.g., obinutuzumab or rituximab) as a monotherapy.

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) who have received treatment with an anti-CD79b immunoconjugate (e.g., polatuzumab vedotin) and an anti-CD20 antibody (e.g., obinutuzumab or rituximab) as a combination therapy (e.g., a combination therapy including an anti-CD79b immunoconjugate (e.g., polatuzumab vedotin) and an anti-CD20 antibody (e.g., obinutuzumab or rituximab) and an additional therapeutic agent (e.g., anti-cancer therapy (e.g., a cytotoxic agent, a growth-inhibitory agent, a radiation therapy, an anti-angiogenic agent, or a combination thereof), e.g., CHP).

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) who have received treatment with an anti-CD20 antibody (e.g., obinutuzumab or rituximab) as a combination therapy (e.g., a combination therapy including an anti-CD20 antibody (e.g., obinutuzumab or rituximab) and an additional therapeutic agent (e.g., anti-cancer therapy (e.g., a cytotoxic agent, a growth-inhibitory agent, a radiation therapy, an anti-angiogenic agent, or a combination thereof), e.g., CHOP).

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) who have received treatment with a therapy that does not include an anti-CD79b immunoconjugate (e.g., polatuzumab vedotin) and an anti-CD20 antibody (e.g., obinutuzumab or rituximab) and includes an anti-cancer therapy (e.g., a cytotoxic agent, a growth-inhibitory agent, a radiation therapy, an anti-angiogenic agent, or a combination thereof), e.g., CHP).

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) who have received treatment with a therapy that does not include an anti-CD20 antibody (e.g., obinutuzumab or rituximab) and includes an anti-cancer therapy (e.g., a cytotoxic agent, a growth-inhibitory agent, a radiation therapy, an anti-angiogenic agent, or a combination thereof), e.g., CHOP).

For example, in some instances, the reference population includes a first subset of patients who have been treated with an anti-CD79b immunoconjugate (e.g., polatuzumab vedotin) and an anti-CD20 antibody (e.g., obinutuzumab or rituximab) and a second subset of patients who have not been treated with an anti-CD79b immunoconjugate (e.g., polatuzumab vedotin) and an anti-CD20 antibody (e.g., obinutuzumab or rituximab).

For example, in some instances, the reference population includes a first subset of patients who have been treated with an anti-CD20 antibody (e.g., obinutuzumab or rituximab) and a second subset of patients who have not been treated an anti-CD20 antibody (e.g., obinutuzumab or rituximab).

For example, in some instances, the reference population includes a first subset of patients who have been treated with an anti-CD79b immunoconjugate (e.g., polatuzumab vedotin) and an anti-CD20 antibody (e.g., obinutuzumab or rituximab) and a second subset of patients who have been treated with an anti-CD20 antibody (e.g., obinutuzumab or rituximab).

In some instances, the reference amount or level of the macrophage biomarker significantly separates each of the first and second subsets of patients based on a significant difference between a patient's responsiveness (e.g., PFS or OS) to treatment with the anti-CD79b immunoconjugate (e.g., polatuzumab vedotin) and the anti-CD20 antibody (e.g., obinutuzumab or rituximab) and a patient's responsiveness to treatment with the anti-CD20 antibody (e.g., obinutuzumab or rituximab) below the reference macrophage biomarker, wherein the patient's responsiveness to treatment with the anti-CD79b immunoconjugate (e.g., polatuzumab vedotin) and the anti-CD20 antibody (e.g., obinutuzumab or rituximab) is significantly improved relative to the patient's responsiveness to treatment with the anti-CD20 antibody (e.g., obinutuzumab or rituximab). For example, in some instances, the reference amount or level of the macrophage biomarker optimally separates each of the first and second subsets of patients based on a maximum difference between a patient's responsiveness (e.g., PFS or OS) to treatment with the anti-CD79b immunoconjugate (e.g., polatuzumab vedotin) and the anti-CD20 antibody (e.g., obinutuzumab or rituximab) and a patient's responsiveness to treatment with the anti-CD20 antibody (e.g., obinutuzumab or rituximab) below the reference macrophage biomarker, wherein the patient's responsiveness to treatment with the anti-CD79b immunoconjugate (e.g., polatuzumab vedotin) and the anti-CD20 antibody (e.g., obinutuzumab or rituximab) is significantly improved relative to the patient's responsiveness to treatment with the anti-CD20 antibody (e.g., obinutuzumab or rituximab). In some instances, the aforementioned first subset of patients is patients treated with Pola-R-CHP (polatuzumab vedotin plus rituximab, cyclophosphamide, doxorubicin, and prednisone). In some instances, the aforementioned second subset of patients is patients treated with R-CHOP (Rituximab plus cyclophosphamide, doxorubicin, vincristine, and prednisone).

In some instances, the reference amount or level of the macrophage biomarker significantly separates each of the first and second subsets of patients based on a significant difference between a patient's responsiveness (e.g., PFS or OS) to treatment with the anti-CD79b immunoconjugate (e.g., polatuzumab vedotin) and the anti-CD20 antibody (e.g., obinutuzumab or rituximab) and a patient's responsiveness to treatment without the anti-CD79b immunoconjugate (e.g., polatuzumab vedotin) and the anti-CD20 antibody (e.g., obinutuzumab or rituximab) below the reference macrophage biomarker, wherein the patient's responsiveness to treatment with the anti-CD79b immunoconjugate (e.g., polatuzumab vedotin) and the anti-CD20 antibody (e.g., obinutuzumab or rituximab) is significantly improved relative to the patient's responsiveness to treatment without the anti-CD79b immunoconjugate (e.g., polatuzumab vedotin) and the anti-CD20 antibody (e.g., obinutuzumab or rituximab). For example, in some instances, the reference amount or level of the macrophage biomarker optimally separates each of the first and second subsets of patients based on a maximum difference between a patient's responsiveness (e.g., PFS or OS) to treatment with the anti-CD79b immunoconjugate (e.g., polatuzumab vedotin) and the anti-CD20 antibody (e.g., obinutuzumab or rituximab) and a patient's responsiveness to treatment without the anti-CD79b immunoconjugate (e.g., polatuzumab vedotin) and the anti-CD20 antibody (e.g., obinutuzumab or rituximab) below the reference macrophage biomarker, wherein the patient's responsiveness to treatment with the anti-CD79b immunoconjugate (e.g., polatuzumab vedotin) and the anti-CD20 antibody (e.g., obinutuzumab or rituximab) is significantly improved relative to the patient's responsiveness to treatment without the anti-CD79b immunoconjugate (e.g., polatuzumab vedotin) and the anti-CD20 antibody (e.g., obinutuzumab or rituximab). In some instances, the aforementioned first subset of patients is patients treated with Pola-R-CHP (polatuzumab vedotin plus rituximab, cyclophosphamide, doxorubicin, and prednisone).

In some instances, the reference amount or level of the macrophage biomarker significantly separates each of the first and second subsets of patients based on a significant difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) below the reference macrophage biomarker, wherein the patient's responsiveness to treatment without the anti-CD20 antibody (e.g., obinutuzumab or rituximab) is significantly improved relative to the patient's responsiveness to treatment with the anti-CD20 antibody (e.g., obinutuzumab or rituximab). For example, in some instances, the reference amount or level of the macrophage biomarker optimally separates each of the first and second subsets of patients based on a maximum difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) below the reference macrophage biomarker, wherein the patient's responsiveness to treatment without the anti-CD20 antibody (e.g., obinutuzumab or rituximab) is significantly improved relative to the patient's responsiveness to treatment with the anti-CD20 antibody (e.g., obinutuzumab or rituximab). In some instances, the aforementioned first subset of patients is patients treated with R-CHOP (Rituximab plus cyclophosphamide, doxorubicin, vincristine, and prednisone).

In some instances, an optimal separation or significant separation may be based on a hazard ratio (HR) determined from an analysis of the amount or level of the macrophage biomarker in the first and second subsets of patients, wherein the HR is less than 1, e.g., an HR of about 0.95, about 0.9, about 0.8, about 0.7, about 0.6, about 0.5, about 0.4, about 0.3, about 0.2, about 0.1 or lower. For example, in particular instances, an optimal separation or significant separation may be based on a hazard ratio (HR) determined from an analysis of the amount or level of the macrophage biomarker in the first and second subsets of patients, wherein the upper bound of the 95% confidence interval of the HR is less than 1, e.g., an upper bound of the 95% confidence interval of the HR of about 0.95, about 0.9, about 0.8, about 0.7, about 0.6, about 0.5, about 0.4, about 0.3, about 0.2, about 0.1 or lower.

Additionally, or alternatively, the reference macrophage biomarker may be an amount or level of the macrophage biomarker in a reference population, wherein the reference population includes at least one subset of patients who do not have a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) or have a lymphoma but are treatment naïve.

(iii) Indications

The medicaments described herein are useful for treating patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)).

In some instances, the lymphoma may be indolent lymphoma. In some instances, the lymphoma may be a B-cell lymphoma, e.g., a non-Hodgkin lymphoma, e.g., a diffuse large B-cell lymphoma (e.g., a germinal-center B-cell-like or activated B-cell-like diffuse large B-cell lymphoma). In some instances, the lymphoma may be a CD20-positive lymphoma.

In certain instances, the cancer may be a B-cell lymphoma. For example, the B-cell lymphoma may be a non-Hodgkin lymphoma, including but not limited to a diffuse large B-cell lymphoma (e.g., a germinal-center B-cell-like or activated B-cell-like diffuse large B-cell lymphoma)). In preferred embodiments, the cancer is diffuse large B-cell lymphoma. For example, the methods described herein may be used for treating a patient having a diffuse large B-cell lymphoma)) with an anti-CD79b immunoconjugate (e.g., polatuzumab vedotin) and an anti-CD20 antibody (e.g., obinutuzumab or rituximab), the method including determining that a macrophage biomarker in a sample from the patient is an amount or level that is below a reference macrophage biomarker amount or level.

In some instances, the individual having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) has not been previously treated for the lymphoma (treatment naïve). For example, in some instances, the individual having a lymphoma has not previously received either an anti-CD79b immunoconjugate (e.g., polatuzumab vedotin) and an anti-CD20 antibody (e.g., obinutuzumab or rituximab) or an anti-CD20 antibody (e.g., obinutuzumab or rituximab).

In some instances, the individual having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) has previously received treatment for the lymphoma. In some instances, the individual having a lymphoma has previously received treatment including either an anti-CD79b immunoconjugate (e.g., polatuzumab vedotin) and an anti-CD20 antibody (e.g., obinutuzumab or rituximab) or an anti-CD20 antibody (e.g., obinutuzumab or rituximab)).

### (iv) Treatment Benefits

A patient who benefits from receiving treatment with a medicament comprising either an anti-CD79b immunoconjugate (e.g., polatuzumab vedotin) and an anti-CD20 antibody (e.g., obinutuzumab or rituximab) may experience, for example, a delay or prevention in the occurrence or recurrence of a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)), alleviation of symptoms, diminishment of any direct or indirect pathological consequences of the cancer, prevention of metastasis, decrease in the rate of disease progression, amelioration or palliation of the disease state, or remission or improved prognosis. In some instances, the medicaments described herein are used to delay development of a cancer or to slow the progression of a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)). In some instances, the benefit may be an increase in OS, PFS, CR, PR, or a combination thereof.

In some instances, a medicament comprising an anti-CD79b immunoconjugate (e.g., polatuzumab vedotin) and an anti-CD20 antibody (e.g., obinutuzumab or rituximab) is for use in a method of treating a patient having a diffuse large B-cell lymphoma) with an amount or level of a macrophage biomarker in a sample that is below a reference macrophage biomarker amount or level (e.g., an amount or level of a macrophage biomarker in a reference population) achieves an improvement of OS (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) relative to a treatment that does not include an anti-CD79b immunoconjugate (e.g., polatuzumab vedotin) and an anti-CD20 antibody (e.g., obinutuzumab or rituximab).

In some instances, a medicament comprising an anti-CD79b immunoconjugate (e.g., polatuzumab vedotin) and an anti-CD20 antibody (e.g., obinutuzumab or rituximab) is for use in a method of treating a patient having a diffuse large B-cell lymphoma ( ) with an amount or level of a macrophage biomarker in a sample that is below a reference macrophage biomarker amount or level (e.g., an amount or level of a macrophage biomarker in a reference population) achieves an improvement of PFS (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) relative to a treatment that does not include an anti-CD79b immunoconjugate (e.g., polatuzumab vedotin) and an anti-CD20 antibody (e.g., obinutuzumab or rituximab).

### C. Therapeutic Uses of an Anti-CD79b Immunoconjugate (e.g., Polatuzumab Vedotin) and an Anti-CD20 Antibody

In a further aspect, the invention provides for the use of an anti-CD79b immunoconjugate (e.g., polatuzumab vedotin) and an anti-CD20 antibody (e.g., obinutuzumab or rituximab) in treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)).

In some instances, the anti-CD79b immunoconjugate (e.g., polatuzumab vedotin) and the anti-CD20 antibody (e.g., obinutuzumab or rituximab) is for use in the treatment of a patient having a lymphoma and having an amount or level of a macrophage biomarker in a sample from the patient that is below a reference macrophage biomarker amount or level.

### (i) Decreased Macrophage Biomarker

In some instances, the anti-CD79b immunoconjugate (e.g., polatuzumab vedotin) and the anti-CD20 antibody (e.g., obinutuzumab or rituximab) is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) and having an amount or level of the macrophage biomarker in the sample that is in about the bottom 99th percentile (equal to, or lower than, about the 99% prevalence level), about the bottom 95th percentile (equal to, or lower than, about the 95% prevalence level), about the bottom 90th percentile (equal to, or lower than, about the 90% prevalence level), about the bottom 85th percentile (equal to, or lower than, about the 85% prevalence level), about the bottom 80th percentile (equal to, or lower than, about the 80% prevalence level), about the bottom 75th percentile (equal to, or lower than, about the 75% prevalence level), about the bottom 70th percentile (equal to, or lower than, about the 70% prevalence level), about the bottom 65th percentile (equal to, or lower than, about the 65% prevalence level), about the bottom 60th percentile (equal to, or lower than, about the 60% prevalence level), about the bottom 55th percentile (equal to, or lower than, about the 55% prevalence level), about the bottom 50th percentile (equal to, or lower than, about the 50% prevalence level), about the bottom 45th percentile (equal to, or lower than, about the 45% prevalence level), about the bottom 40th percentile (equal to, or lower than, about the 40% prevalence level), about the bottom 35th percentile (equal to, or lower than, about the 35% prevalence level), about the bottom 30th percentile (equal to, or lower than, about the 30% prevalence level), about the bottom 25th percentile (equal to, or lower than, about the 25% prevalence level), about the bottom 20th percentile (equal to, or lower than, about the 20% prevalence level), about the bottom 15th percentile (equal to, or lower than, about the 15% prevalence level), about the bottom 10th percentile (equal to, or lower than, about the 10% prevalence level), about the bottom 5th percentile (equal to, or lower than, about the 5% prevalence level), or about the bottom 1st percentile (equal to, or lower than, about the 1% prevalence level) of the amount or level of the macrophage biomarker in the reference population.

In some instances, the anti-CD79b immunoconjugate (e.g., polatuzumab vedotin) and the anti-CD20 antibody (e.g., obinutuzumab or rituximab) is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) and having an amount or level of the macrophage biomarker in the sample that is in about the bottom 10th to about the bottom 90th percentile, about the bottom 20th to about the bottom 80th percentile, about the bottom 30th to about the bottom 70th percentile, about the bottom 40th to about the bottom 60th percentile, about the bottom 45th to about the bottom 55th percentile, about the bottom 48th to about the bottom 52th percentile, about the bottom 49.5th to about the bottom 50.5th percentile, about the bottom 49.9th to about the bottom 50.1th percentile, or about the bottom 50th percentile of the amount or level of the macrophage biomarker in the reference population. For example, in some instances, the anti-CD79b immunoconjugate (e.g., polatuzumab vedotin) and the anti-CD20 antibody (e.g., obinutuzumab or rituximab) is for use in a method of treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) and having an amount or level of the macrophage biomarker in the sample that is between about 10% to about 90% prevalence, about 15 to about 85% prevalence, about 20% to about 80% prevalence, about 25% to about 75% prevalence, about 30% to about 70% prevalence, about 35% to about 65% prevalence, about 40% to about 60% prevalence, about 45% to about 55% prevalence, about 48% to about 52% prevalence, about 49.5% to about 50.5% prevalence, about 49.9% to about 50.1% prevalence, or about 50% prevalence in the reference population.

In some instances, an amount or level of the macrophage biomarker that is lower than a reference amount or level of the macrophage biomarker refers to a decrease of about 10%, 20%, 30%, 40%, 50%, 60%, 70%, 80%, 90%, 95%, 96%, 97%, 98%, or 99% or greater in the amount or level of the macrophage biomarker, detected by standard art-known methods such as those described herein, as compared to the amount or level of the macrophage biomarker in a reference sample, reference cell, reference tissue, control sample, control cell, or control tissue. In certain instances, an amount or level of the macrophage biomarker that is lower than a reference amount or level of the macrophage biomarker refers to a decrease in the amount or level of the macrophage biomarker in the sample, wherein the decrease is at least about 1.5×, 1.75×, 2×, 3×, 4×, 5×, 6×, 7×, 8×, 9×, 10×, 25×, 50×, 75×, or 100× the amount or level of the macrophage biomarker in a reference sample, reference cell, reference tissue, control sample, control cell, or control tissue. In some instances, an amount or level of the macrophage biomarker that is lower than a reference amount or level of the macrophage biomarker refers to a decrease in the amount or level of the macrophage biomarker that is greater than about 1.5-fold, about 1.75-fold, about 2-fold, about 2.25-fold, about 2.5-fold, about 2.75-fold, about 3.0-fold, or about 3.25-fold as compared to the amount or level of the macrophage biomarker in a reference sample, reference cell, reference tissue, control sample, control cell, or control tissue.

In some instances, an amount or level of the macrophage biomarker that is lower than a reference amount or level of the macrophage biomarker refers to an overall decrease of about 10%, 20%, 30%, 40%, 50%, 60%, 70%, 80%, 90%, 95%, 96%, 97%, 98%, or 99% or greater in the amount or level of the macrophage biomarker, detected by standard art-known methods such as those described herein, as compared to a pre-assigned amount or level of the macrophage biomarker. In certain instances, an amount or level of the macrophage biomarker that is lower than a reference amount or level of the macrophage biomarker refers to a decrease in the amount or level of the macrophage biomarker in the sample, wherein the decrease is at least about 1.5×, 1.75×, 2×, 3×, 4×, 5×, 6×, 7×, 8×, 9×, 10×, 25×, 50×, 75×, or 100× a pre-assigned amount or level of the macrophage biomarker. In some instances, an amount or level of the macrophage biomarker that is lower than a reference amount or level of the macrophage biomarker refers to an overall decrease in the amount or level of the macrophage biomarker that is greater than about 1.5-fold, about 1.75-fold, about 2-fold, about 2.25-fold, about 2.5-fold, about 2.75-fold, about 3.0-fold, or about 3.25-fold as compared to a pre-assigned amount or level of the macrophage biomarker.

### (ii) Reference Macrophage Biomarker

The reference macrophage biomarker amount or level can be a pre-assigned macrophage biomarker amount or level. In some instances, the amount or level of the macrophage biomarker in a reference population is a median amount or level of the macrophage biomarker of the reference population. In some instances, the amount or level of the macrophage biomarker in a reference population is a mean amount or level of the macrophage biomarker of the reference population.

In some instances, the pre-assigned macrophage biomarker amount or level is a percentage of cellular subtypes within a sample. In some instances, the percentage of cellular subtypes within a sample is between about 0% and 40% (e.g., 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40%). In some instances, the percentage of cellular subtypes within a sample is between about 0% and 10% (e.g., 0%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%). In some instances, the percentage of cellular subtypes within a sample is less than 10% (e.g., 0%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%). In some instances, the percentage of cellular subtypes within a sample is about 6%. In some instances, the percentage of cellular subtypes within a sample is about 5%. In some instances, the percentage of cellular subtypes within a sample is about 4.74%. In some instances, the percentage of cellular subtypes within a sample is about 4%. In some instances, the percentage of cellular subtypes within a sample is about 3.35%. In some instances, the percentage of cellular subtypes within a sample is about 3%. In some instances, the percentage of cellular subtypes within a sample is about 2.5%. In some instances, the percentage of cellular subtypes within a sample is about 2%. In some instances, the percentage of cellular subtypes within a sample is about 1.67%. In some instances, the percentage of cellular subtypes within a sample is about 1%. In some instances, the percentage of cellular subtypes within a sample is about 0%.

The reference amount or level of the macrophage biomarker described herein may be based on the amount or level of the macrophage biomarker in a reference population. In some instances, the reference macrophage biomarker described herein is an amount or level of the macrophage biomarker in a reference population that includes one or more (e.g., one or more, two or more, three or more, four or more, or five or more) subsets of patients.

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)).

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) who have been administered one or more doses (e.g., at least one, two, three, four, five, six, seven, eight, nine, or ten or more doses) of either an anti-CD79b immunoconjugate (e.g., polatuzumab vedotin) and an anti-CD20 antibody (e.g., obinutuzumab or rituximab) or an anti-CD20 antibody (e.g., obinutuzumab or rituximab).

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) who have received treatment with an anti-CD20 antibody (e.g., obinutuzumab or rituximab) as a monotherapy.

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) who have received treatment with an anti-CD79b immunoconjugate (e.g., polatuzumab vedotin) and an anti-CD20 antibody (e.g., obinutuzumab or rituximab) as a combination therapy (e.g., a combination therapy including an anti-CD79b immunoconjugate (e.g., polatuzumab vedotin) and an anti-CD20 antibody (e.g., obinutuzumab or rituximab) and an additional therapeutic agent (e.g., anti-cancer therapy (e.g., a cytotoxic agent, a growth-inhibitory agent, a radiation therapy, an anti-angiogenic agent, or a combination thereof), e.g., CHP).

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) who have received treatment with an anti-CD20 antibody (e.g., obinutuzumab or rituximab) as a combination therapy (e.g., a combination therapy including an anti-CD20 antibody (e.g., obinutuzumab or rituximab) and an additional therapeutic agent (e.g., anti-cancer therapy (e.g., a cytotoxic agent, a growth-inhibitory agent, a radiation therapy, an anti-angiogenic agent, or a combination thereof), e.g., CHOP).

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) who have received treatment with a therapy that does not include an anti-CD79b immunoconjugate (e.g., polatuzumab vedotin) and an anti-CD20 antibody (e.g., obinutuzumab or rituximab) and includes an anti-cancer therapy (e.g., a cytotoxic agent, a growth-inhibitory agent, a radiation therapy, an anti-angiogenic agent, or a combination thereof), e.g., CHP).

In some instances, the reference macrophage biomarker is an amount or level of the macrophage biomarker in a reference population, wherein the reference population includes at least one subset of patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) who have received treatment with a therapy that does not include an anti-CD20 antibody (e.g., obinutuzumab or rituximab) and includes an anti-cancer therapy (e.g., a cytotoxic agent, a growth-inhibitory agent, a radiation therapy, an anti-angiogenic agent, or a combination thereof), e.g., CHOP).

For example, in some instances, the reference population includes a first subset of patients who have been treated with an anti-CD79b immunoconjugate (e.g., polatuzumab vedotin) and an anti-CD20 antibody (e.g., obinutuzumab or rituximab) and a second subset of patients who have not been treated with an anti-CD79b immunoconjugate (e.g., polatuzumab vedotin) and an anti-CD20 antibody (e.g., obinutuzumab or rituximab).

For example, in some instances, the reference population includes a first subset of patients who have been treated with an anti-CD20 antibody (e.g., obinutuzumab or rituximab) and a second subset of patients who have not been treated an anti-CD20 antibody (e.g., obinutuzumab or rituximab).

For example, in some instances, the reference population includes a first subset of patients who have been treated with an anti-CD79b immunoconjugate (e.g., polatuzumab vedotin) and an anti-CD20 antibody (e.g., obinutuzumab or rituximab) and a second subset of patients who have been treated with an anti-CD20 antibody (e.g., obinutuzumab or rituximab).

In some instances, the reference amount or level of the macrophage biomarker significantly separates each of the first and second subsets of patients based on a significant difference between a patient's responsiveness (e.g., PFS or OS) to treatment with the anti-CD79b immunoconjugate (e.g., polatuzumab vedotin) and the anti-CD20 antibody (e.g., obinutuzumab or rituximab) and a patient's responsiveness to treatment with the anti-CD20 antibody (e.g., obinutuzumab or rituximab) below the reference macrophage biomarker, wherein the patient's responsiveness to treatment with the anti-CD79b immunoconjugate (e.g., polatuzumab vedotin) and the anti-CD20 antibody (e.g., obinutuzumab or rituximab) is significantly improved relative to the patient's responsiveness to treatment with the anti-CD20 antibody (e.g., obinutuzumab or rituximab). For example, in some instances, the reference amount or level of the macrophage biomarker optimally separates each of the first and second subsets of patients based on a maximum difference between a patient's responsiveness (e.g., PFS or OS) to treatment with the anti-CD79b immunoconjugate (e.g., polatuzumab vedotin) and the anti-CD20 antibody (e.g., obinutuzumab or rituximab) and a patient's responsiveness to treatment with the anti-CD20 antibody (e.g., obinutuzumab or rituximab) below the reference macrophage biomarker, wherein the patient's responsiveness to treatment with the anti-CD79b immunoconjugate (e.g., polatuzumab vedotin) and the anti-CD20 antibody (e.g., obinutuzumab or rituximab) is significantly improved relative to the patient's responsiveness to treatment with the anti-CD20 antibody (e.g., obinutuzumab or rituximab). In some instances, the aforementioned first subset of patients is patients treated with Pola-R-CHP (polatuzumab vedotin plus rituximab, cyclophosphamide, doxorubicin, and prednisone). In some instances, the aforementioned second subset of patients is patients treated with R-CHOP (Rituximab plus cyclophosphamide, doxorubicin, vincristine, and prednisone).

In some instances, the reference amount or level of the macrophage biomarker significantly separates each of the first and second subsets of patients based on a significant difference between a patient's responsiveness (e.g., PFS or OS) to treatment with the anti-CD79b immunoconjugate (e.g., polatuzumab vedotin) and the anti-CD20 antibody (e.g., obinutuzumab or rituximab) and a patient's responsiveness to treatment without the anti-CD79b immunoconjugate (e.g., polatuzumab vedotin) and the anti-CD20 antibody (e.g., obinutuzumab or rituximab) below the reference macrophage biomarker, wherein the patient's responsiveness to treatment with the anti-CD79b immunoconjugate (e.g., polatuzumab vedotin) and the anti-CD20 antibody (e.g., obinutuzumab or rituximab) is significantly improved relative to the patient's responsiveness to treatment without the anti-CD79b immunoconjugate (e.g., polatuzumab vedotin) and the anti-CD20 antibody (e.g., obinutuzumab or rituximab). For example, in some instances, the reference amount or level of the macrophage biomarker optimally separates each of the first and second subsets of patients based on a maximum difference between a patient's responsiveness (e.g., PFS or OS) to treatment with the anti-CD79b immunoconjugate (e.g., polatuzumab vedotin) and the anti-CD20 antibody (e.g., obinutuzumab or rituximab) and a patient's responsiveness to treatment without the anti-CD79b immunoconjugate (e.g., polatuzumab vedotin) and the anti-CD20 antibody (e.g., obinutuzumab or rituximab) below the reference macrophage biomarker, wherein the patient's responsiveness to treatment with the anti-CD79b immunoconjugate (e.g., polatuzumab vedotin) and the anti-CD20 antibody (e.g., obinutuzumab or rituximab) is significantly improved relative to the patient's responsiveness to treatment without the anti-CD79b immunoconjugate (e.g., polatuzumab vedotin) and the anti-CD20 antibody (e.g., obinutuzumab or rituximab). In some instances, the aforementioned first subset of patients is patients treated with Pola-R-CHP (polatuzumab vedotin plus rituximab, cyclophosphamide, doxorubicin, and prednisone).

In some instances, the reference amount or level of the macrophage biomarker significantly separates each of the first and second subsets of patients based on a significant difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) below the reference macrophage biomarker, wherein the patient's responsiveness to treatment without the anti-CD20 antibody (e.g., obinutuzumab or rituximab) is significantly improved relative to the patient's responsiveness to treatment with the anti-CD20 antibody (e.g., obinutuzumab or rituximab). For example, in some instances, the reference amount or level of the macrophage biomarker optimally separates each of the first and second subsets of patients based on a maximum difference between a patient's responsiveness (e.g., PFS or OS) to treatment with the anti-CD20 antibody (e.g., obinutuzumab or rituximab) and a patient's responsiveness to treatment without the anti-CD20 antibody (e.g., obinutuzumab or rituximab) below the reference macrophage biomarker, wherein the patient's responsiveness to treatment without the anti-CD20 antibody (e.g., obinutuzumab or rituximab) is significantly improved relative to the patient's responsiveness to treatment with the anti-CD20 antibody (e.g., obinutuzumab or rituximab). In some instances, the aforementioned first subset of patients is patients treated with R-CHOP (Rituximab plus cyclophosphamide, doxorubicin, vincristine, and prednisone).

In some instances, an optimal separation or significant separation may be based on an HR determined from an analysis of the amount or level of the macrophage biomarker in the first and second subsets of patients, wherein the HR is less than 1, e.g., an HR of about 0.95, about 0.9, about 0.8, about 0.7, about 0.6, about 0.5, about 0.4, about 0.3, about 0.2, about 0.1 or lower. For example, in particular instances, an optimal separation or significant separation may be based on an HR determined from an analysis of the amount or level of the macrophage biomarker in the first and second subsets of patients, wherein the upper bound of the 95% confidence interval of the HR is less than 1, e.g., an upper bound of the 95% confidence interval of the HR of about 0.95, about 0.9, about 0.8, about 0.7, about 0.6, about 0.5, about 0.4, about 0.3, about 0.2, about 0.1 or lower.

Additionally, or alternatively, the reference macrophage biomarker may be an amount or level of the macrophage biomarker in a reference population, wherein the reference population includes at least one subset of patients who do not have a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) or have a lymphoma but are treatment naïve.

(iii) Indications

The anti-CD20 antibodies (e.g., obinutuzumab or rituximab) and the anti-CD20 antibodies (e.g., obinutuzumab and rituximab) described herein are useful for methods of treating patients having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)).

In some instances, the lymphoma may be indolent lymphoma. In some instances, the lymphoma may be a B-cell lymphoma, e.g., a non-Hodgkin lymphoma, e.g., a diffuse large B-cell lymphoma (e.g., a germinal-center B-cell-like or activated B-cell-like diffuse large B-cell lymphoma). In some instances, the lymphoma may be a CD20-positive lymphoma.

In certain instances, the cancer may be a B-cell lymphoma. For example, the B-cell lymphoma may be a non-Hodgkin lymphoma, including but not limited to a diffuse large B-cell lymphoma (e.g., a germinal-center B-cell-like or activated B-cell-like diffuse large B-cell lymphoma)). In preferred embodiments, the cancer is diffuse large B-cell lymphoma. For example, the methods described herein may be used for treating a patient having a diffuse large B-cell)) with an anti-CD20 antibody (e.g., obinutuzumab or rituximab) and an anti-CD20 antibody (e.g., obinutuzumab or rituximab), the method including determining that a macrophage biomarker in a sample from the patient is an amount or level that is below a reference macrophage biomarker amount or level.

In some instances, the individual having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) has not been previously treated for the lymphoma (treatment naïve). For example, in some instances, the individual having a lymphoma has not previously received either an anti-CD79b immunoconjugate (e.g., polatuzumab vedotin) and an anti-CD20 antibody (e.g., obinutuzumab or rituximab) or an anti-CD20 antibody (e.g., obinutuzumab or rituximab).

In some instances, the individual having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) has previously received treatment for the lymphoma. In some instances, the individual having a lymphoma has previously received treatment including either an anti-CD79b immunoconjugate (e.g., polatuzumab vedotin) and an anti-CD20 antibody (e.g., obinutuzumab or rituximab) or an anti-CD20 antibody (e.g., obinutuzumab or rituximab)).

### (iv) Treatment Benefits

A patient who benefits from receiving treatment with an anti-CD79b immunoconjugate (e.g., polatuzumab vedotin) and an anti-CD20 antibody (e.g., obinutuzumab or rituximab) may experience, for example, a delay or prevention in the occurrence or recurrence of a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)), alleviation of symptoms, diminishment of any direct or indirect pathological consequences of the cancer, prevention of metastasis, decrease in the rate of disease progression, amelioration or palliation of the disease state, or remission or improved prognosis. In some instances, either the anti-CD79b immunoconjugate (e.g., polatuzumab vedotin) and the anti-CD20 antibody (e.g., obinutuzumab or rituximab) or the anti-CD20 antibodies (e.g., obinutuzumab and rituximab) described herein are used to delay development of a cancer or to slow the progression of a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)). In some instances, the benefit may be an increase in OS, PFS, CR, PR, or a combination thereof.

In some instances, the anti-CD79b immunoconjugate (e.g., polatuzumab vedotin) and the anti-CD20 antibody (e.g., obinutuzumab or rituximab) is for use in a method of treating a patient having a a diffuse large B-cell lymphoma) and having an amount or level of a macrophage biomarker in a sample that is below a reference macrophage biomarker amount or level (e.g., an amount or level of a macrophage biomarker in a reference population) achieves an improvement of OS (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) relative to a treatment that does not include an anti-CD79b immunoconjugate (e.g., polatuzumab vedotin) and an anti-CD20 antibody (e.g., obinutuzumab or rituximab).

In some instances, the anti-CD79b immunoconjugate (e.g., polatuzumab vedotin) and the anti-CD20 antibody (e.g., obinutuzumab or rituximab) is for use in a method of treating a patient having a a diffuse large B-cell lymphoma) and having an amount or level of a macrophage biomarker in a sample that is below a reference macrophage biomarker amount or level (e.g., an amount or level of a macrophage biomarker in a reference population) achieves an improvement of PFS (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) relative to a treatment that does not include an anti-CD79b immunoconjugate (e.g., polatuzumab vedotin) and an anti-CD20 antibody (e.g., obinutuzumab or rituximab).

D. Exemplary anti-CD79b immunoconjugates

In some embodiments, the anti-CD79b immunoconjugate comprises an anti-CD79b antibody (Ab) which targets a cancer cell (such as a diffuse large B-cell lymphoma (DLBCL) cell), a drug moiety (D), and a linker moiety (L) that attaches Ab to D. In some embodiments, the anti-CD79b antibody is attached to the linker moiety (L) through one or more amino acid residues, such as lysine and/or cysteine. In some embodiments, the immunoconjugate comprises formula Ab-(L-D)p, wherein: (a) Ab is the anti-CD79b antibody which binds CD79b on the surface of a cancer cell (e.g., a DLBCL cell); (b) L is a linker; (c) D is a cytotoxic agent; and (d) p ranges from 1-8.

An exemplary anti-CD79b immunoconjugate comprises Formula I:

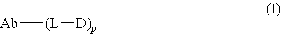

wherein p is 1 to about 20 (e.g., 1 to 15, 1 to 10, 1 to 8, 2 to 5, or 3 to 4). In some embodiments, the number of drug moieties that can be conjugated to the anti-CD79b antibody is limited by the number of free cysteine residues. In some embodiments, free cysteine residues are introduced into the antibody amino acid sequence by the methods described elsewhere herein. Exemplary anti-CD79b immunoconjugates of Formula I comprise, but are not limited to, anti-CD79b antibodies that comprise 1, 2, 3, or 4 engineered cysteine amino acids (Lyon, R. et al (2012) Methods in Enzym. 502:123-138). In some embodiments, one or more free cysteine residues are already present in the anti-CD79b antibody, without the use of engineering, in which case the existing free cysteine residues may be used to conjugate the anti-CD79b antibody to the drug/cytotoxic agent. In some embodiments, the anti-CD79b antibody is exposed to reducing conditions prior to conjugation of the antibody to the drug/cytotoxic agent in order to generate one or more free cysteine residues.

### (i) Exemplary Linkers

A “linker” (L) is a bifunctional or multifunctional moiety that can be used to link one or more drug moieties (D) to the anti-CD79b antibody (Ab) to form an anti-CD79b immunoconjugate of Formula I. In some embodiments, anti-CD79b immunoconjugates can be prepared using a linker having reactive functionalities for covalently attaching to the drug and to the anti-CD79b antibody. For example, in some embodiments, a cysteine thiol of the anti-CD79b antibody (Ab) can form a bond with a reactive functional group of a linker or a drug-linker intermediate to make the anti-CD79b immunoconjugate.

In one aspect, a linker has a functionality that is capable of reacting with a free cysteine present on the anti-CD79b antibody to form a covalent bond. Exemplary reactive functionalities include, without limitation, e.g., maleimide, haloacetamides, α-haloacetyl, activated esters such as succinimide esters, 4 nitrophenyl esters, pentafluorophenyl esters, tetrafluorophenyl esters, anhydrides, acid chlorides, sulfonyl chlorides, isocyanates, and isothiocyanates. See, e.g., the conjugation method at page 766 of Klussman, et al (2004), Bioconjugate Chemistry 15(4):765-773, and the Examples herein.

In some embodiments, a linker has a functionality that is capable of reacting with an electrophilic group present on the anti-CD79b antibody. Exemplary electrophilic groups include, without limitation, e.g., aldehyde and ketone carbonyl groups. In some embodiments, a heteroatom of the reactive functionality of the linker can react with an electrophilic group on an antibody and form a covalent bond to an antibody unit. Exemplary reactive functionalities include, but are not limited to, e.g., hydrazide, oxime, amino, hydrazine, thiosemicarbazone, hydrazine carboxylate, and arylhydrazide.

In some embodiments, the linker comprises one or more linker components. Exemplary linker components include, e.g., 6-maleimidocaproyl (“MC”), maleimidopropanoyl (“MP”), valine-citrulline (“val-cit” or “vc”), alanine-phenylalanine (“ala-phe”), p-aminobenzyloxycarbonyl (a “PAB”), N-Succinimidyl 4-(2-pyridylthio) pentanoate (“SPP”), and 4-(N-maleimidomethyl) cyclohexane-1 carboxylate (“MCC”). Various linker components are known in the art, some of which are described below.

In some embodiments, the linker is a “cleavable linker,” facilitating release of a drug. Non-limiting exemplary cleavable linkers include acid-labile linkers (e.g., comprising hydrazone), protease-sensitive (e.g., peptidase-sensitive) linkers, photolabile linkers, or disulfide-containing linkers (Chari et al., Cancer Research 52:127-131 (1992); U.S. Pat. No. 5,208,020).

In certain embodiments, a linker (L) has the following Formula II:

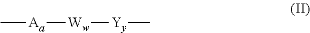

wherein A is a “stretcher unit,” and a is an integer from 0 to 1; W is an “amino acid unit,” and w is an integer from 0 to 12; Y is a “spacer unit,” and y is 0, 1, or 2; and Ab, D, and p are defined as above for Formula I. Exemplary embodiments of such linkers are described in U.S. Pat. No. 7,498,298, which is expressly incorporated herein by reference.

In some embodiments, a linker component comprises a “stretcher unit” that links an antibody to another linker component or to a drug moiety. Nonlimiting exemplary stretcher units are shown below (wherein the wavy line indicates sites of covalent attachment to an antibody, drug, or additional linker components):

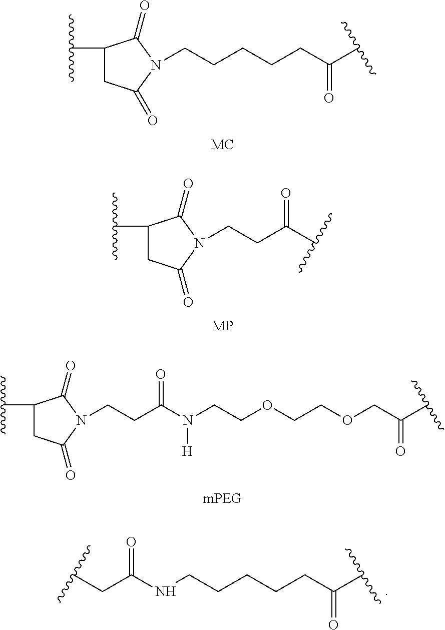 [SMILES: *C(=O)CCCCCN1C(=O)CC(*)C1=O ; *C(=O)CCN1C(=O)CC(*)C1=O ; *C(=O)COCCOCCNC(=O)CCN1C(=O)CC(*)C1=O ; *CC(=O)NCCCCCC(*)=O]

In some embodiments, a linker component comprises an “amino acid unit.” In some such embodiments, the amino acid unit allows for cleavage of the linker by a protease, thereby facilitating release of the drug/cytotoxic agent from the anti-CD79b immunoconjugate upon exposure to intracellular proteases, such as lysosomal enzymes (Doronina et al. (2003) Nat. Biotechnol. 21:778-784). Exemplary amino acid units include, but are not limited to, dipeptides, tripeptides, tetrapeptides, and pentapeptides. Exemplary dipeptides include, but are not limited to, valine-citrulline (vc or val-cit), alanine-phenylalanine (af or ala-phe); phenylalanine-lysine (fk or phe-lys); phenylalanine-homolysine (phe-homolys); and N-methyl-valine-citrulline (Me-val-cit). Exemplary tripeptides include, but are not limited to, glycine-valine-citrulline (gly-val-cit) and glycine-glycine-glycine (gly-gly-gly). An amino acid unit may comprise amino acid residues that occur naturally and/or minor amino acids and/or non-naturally occurring amino acid analogs, such as citrulline. Amino acid units can be designed and optimized for enzymatic cleavage by a particular enzyme, for example, a tumor-associated protease, cathepsin B, C and D, or a plasmin protease.

In some embodiments, a linker component comprises a “spacer” unit that links the antibody to a drug moiety, either directly or through a stretcher unit and/or an amino acid unit. A spacer unit may be “self-immolative” or a “non-self-immolative.” A “non-self-immolative” spacer unit is one in which part or all of the spacer unit remains bound to the drug moiety upon cleavage of the ADC. Examples of non-self-immolative spacer units include, but are not limited to, a glycine spacer unit and a glycine-glycine spacer unit. In some embodiments, enzymatic cleavage of an ADC containing a glycine-glycine spacer unit by a tumor-cell associated protease results in release of a glycine-glycine-drug moiety from the remainder of the ADC. In some such embodiments, the glycine-glycine-drug moiety is subjected to a hydrolysis step in the tumor cell, thus cleaving the glycine-glycine spacer unit from the drug moiety.

A “self-immolative” spacer unit allows for release of the drug moiety. In certain embodiments, a spacer unit of a linker comprises a p-aminobenzyl unit. In some such embodiments, a p-aminobenzyl alcohol is attached to an amino acid unit via an amide bond, and a carbamate, methylcarbamate, or carbonate is made between the benzyl alcohol and the drug (Hamann et al. (2005) Expert Opin. Ther. Patents (2005) 15:1087-1103). In some embodiments, the spacer unit is p-aminobenzyloxycarbonyl (PAB). In some embodiments, an anti-CD79b immunoconjugate comprises a self-immolative linker that comprises the structure:

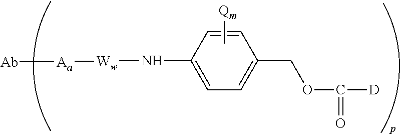

wherein Q is —C₁-C₈ alkyl, —O—(C₁-C₈ alkyl), -halogen, -nitro, or -cyno; m is an integer ranging from 0 to 4; and p ranges from 1 to about 20. In some embodiments, p ranges from 1 to 10, 1 to 7, 1 to 5, or 1 to 4.

Other examples of self-immolative spacers include, but are not limited to, aromatic compounds that are electronically similar to the PAB group, such as 2-aminoimidazol-5-methanol derivatives (U.S. Pat. No. 7,375,078; Hay et al. (1999) Bioorg. Med. Chem. Lett. 9:2237) and ortho- or para-aminobenzylacetals. In some embodiments, spacers can be used that undergo cyclization upon amide bond hydrolysis, such as substituted and unsubstituted 4-aminobutyric acid amides (Rodrigues et al (1995) Chemistry Biology 2:223), appropriately substituted bicyclo[2.2.1] and bicyclo[2.2.2] ring systems (Storm et al (1972) J. Amer. Chem. Soc. 94:5815) and 2-aminophenylpropionic acid amides (Amsberry, et al (1990) J. Org. Chem. 55:5867). Linkage of a drug to the α-carbon of a glycine residue is another example of a self-immolative spacer that may be useful in ADC (Kingsbury et al (1984) J. Med. Chem. 27:1447).

In some embodiments, linker L may be a dendritic type linker for covalent attachment of more than one drug moiety to an antibody through a branching, multifunctional linker moiety (Sun et al (2002) Bioorganic & Medicinal Chemistry Letters 12:2213-2215; Sun et al (2003) Bioorganic & Medicinal Chemistry 11:1761-1768). Dendritic linkers can increase the molar ratio of drug to antibody, i.e. loading, which is related to the potency of the ADC. Thus, where an antibody bears only one reactive cysteine thiol group, a multitude of drug moieties may be attached through a dendritic linker.

Nonlimiting exemplary linkers are shown below in the context of an anti-CD79b immunoconjugates of Formulas III, IV, V:

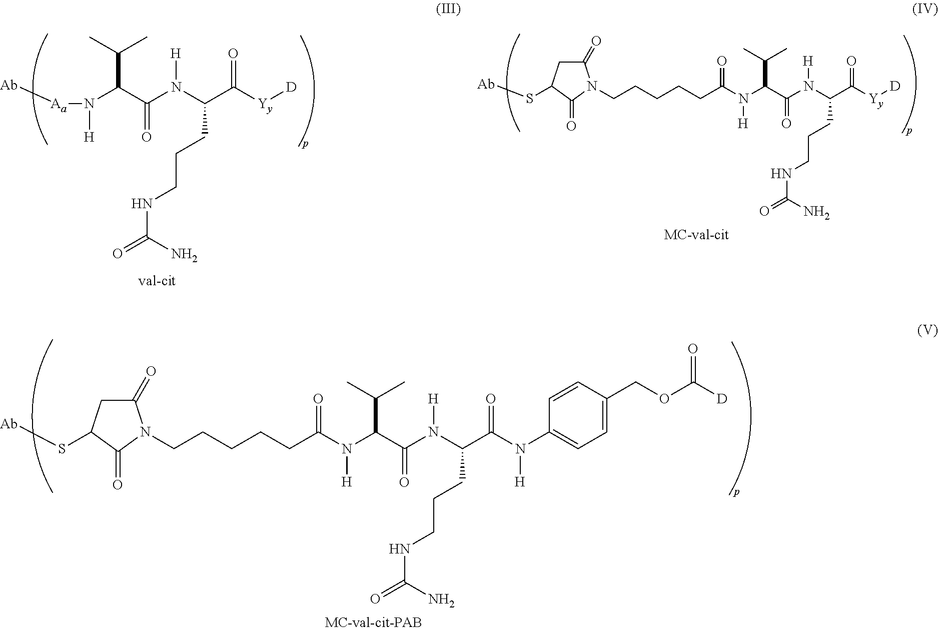

wherein (Ab) is an anti-CD79b antibody, (D) is a drug/cytotoxic agent, “Val-Cit” is a valine-citrulline dipeptide, MC is 6-maleimidocaproyl, PAB is p-aminobenzyloxycarbonyl, and p is 1 to about 20 (e.g., 1 to 15,1 to 10, 1 to 8, 2 to 5, or 3 to 4).

In some embodiments, the anti-CD79b immunoconjugate comprises a structure of any one of formulas VI-V below:

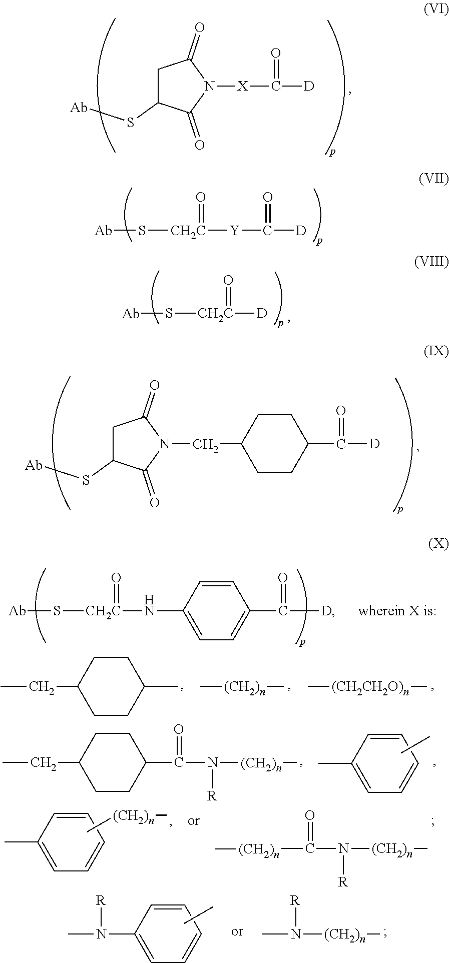

each R is independently H or C₁-C₆ alkyl; and n is 1 to 12.

Typically, peptide-type linkers can be prepared by forming a peptide bond between two or more amino acids and/or peptide fragments. Such peptide bonds can be prepared, for example, according to a liquid phase synthesis method (e.g., E. Schröder and K. Libke (1965) “The Peptides”, volume 1, pp 76-136, Academic Press).

In some embodiments, a linker is substituted with groups that modulate solubility and/or reactivity. As a non-limiting example, a charged substituent such as sulfonate (—SO₃) or ammonium may increase water solubility of the linker reagent and facilitate the coupling reaction of the linker reagent with the antibody and/or the drug moiety, or facilitate the coupling reaction of Ab-L (anti-CD79b antibody-linker intermediate) with D, or D-L (drug/cytotoxic agent-linker intermediate) with Ab, depending on the synthetic route employed to prepare the anti-CD79b immunoconjugate. In some embodiments, a portion of the linker is coupled to the antibody and a portion of the linker is coupled to the drug, and then the anti-CD79 Ab-(linker portion)^{a} is coupled to drug/cytotoxic agent-(linker portion)^{b} to form the anti-CD79b immunoconjugate of Formula I. In some such embodiments, the anti-CD79b antibody comprises more than one (linker portion)^{a} substituents, such that more than one drug/cytotoxic agent is coupled to the anti-CD79b antibody in the anti-CD79b immunoconjugate of Formula I.

The anti-CD79b immunoconjugates provided herein expressly contemplate, but are not limited to, anti-CD79b immunoconjugates prepared with the following linker reagents: bis-maleimido-trioxyethylene glycol (BMPEO), N-(β-maleimidopropyloxy)-N-hydroxy succinimide ester (BMPS), N-(ε-maleimidocaproyloxy) succinimide ester (EMCS), N-[γ-maleimidobutyryloxy]succinimide ester (GMBS), 1,6-hexane-bis-vinylsulfone (HBVS), succinimidyl 4-(N-maleimidomethyl)cyclohexane-1-carboxy-(6-amidocaproate) (LC-SMCC), m-maleimidobenzoyl-N-hydroxysuccinimide ester (MBS), 4-(4-N-Maleimidophenyl)butyric acid hydrazide (MPBH), succinimidyl 3-(bromoacetamido)propionate (SBAP), succinimidyl iodoacetate (SIA), succinimidyl (4-iodoacetyl)aminobenzoate (SIAB), N-succinimidyl-3-(2-pyridyldithio) propionate (SPDP), N-succinimidyl-4-(2-pyridylthio)pentanoate (SPP), succinimidyl 4-(N-maleimidomethyl)cyclohexane-1-carboxylate (SMCC), succinimidyl 4-(p-maleimidophenyl)butyrate (SMPB), succinimidyl 6-[(beta-maleimidopropionamido)hexanoate](SMPH), iminothiolane (IT), sulfo-EMCS, sulfo-GMBS, sulfo-KMUS, sulfo-MBS, sulfo-SIAB, sulfo-SMCC, and sulfo-SMPB, and succinimidyl-(4-vinylsulfone)benzoate (SVSB), and including bis-maleimide reagents: dithiobismaleimidoethane (DTME), 1,4-Bismaleimidobutane (BMB), 1,4 Bismaleimidyl-2,3-dihydroxybutane (BMDB), bismaleimidohexane (BMH), bismaleimidoethane (BMOE), BM(PEG)2 (shown below), and BM(PEG)3 (shown below); bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). In some embodiments, bis-maleimide reagents allow the attachment of the thiol group of a cysteine in the antibody to a thiol-containing drug moiety, linker, or linker-drug intermediate. Other functional groups that are reactive with thiol groups include, but are not limited to, iodoacetamide, bromoacetamide, vinyl pyridine, disulfide, pyridyl disulfide, isocyanate, and isothiocyanate.

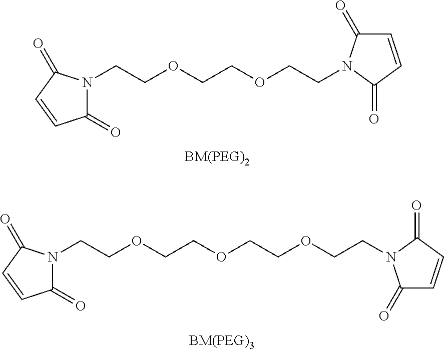 [SMILES: O=C1C=CC(=O)N1CCOCCOCCN1C(=O)C=CC1=O ; O=C1C=CC(=O)N1CCOCCOCCOCCN1C(=O)C=CC1=O]

Certain useful linker reagents can be obtained from various commercial sources, such as Pierce Biotechnology, Inc. (Rockford, IL), Molecular Biosciences Inc. (Boulder, CO), or synthesized in accordance with procedures described in the art; for example, in Toki et al (2002) J. Org. Chem. 67:1866-1872; Dubowchik, et al. (1997) Tetrahedron Letters, 38:5257-60; Walker, M. A. (1995) J. Org. Chem. 60:5352-5355; Frisch et al (1996) Bioconjugate Chem. 7:180-186; U.S. Pat. No. 6,214,345; WO 02/088172; US 2003130189; US2003096743; WO 03/026577; WO 03/043583; and WO 04/032828.

Carbon-14-labeled 1-isothiocyanatobenzyl-3-methyldiethylene triaminepentaacetic acid (MX-DTPA) is an exemplary chelating agent for conjugation of radionucleotide to the antibody. See, e.g., WO94/11026.

### (ii) Anti-CD79b Antibodies

In some embodiments, the immunoconjugate (e.g., anti-CD79b immunoconjugate) comprises an anti-CD79b antibody that comprises at least one, two, three, four, five, or six HVRs selected from (a) HVR-H1 comprising the amino acid sequence of SEQ ID NO: 5; (b) HVR-H2 comprising the amino acid sequence of SEQ ID NO: 6; (c) HVR-H3 comprising the amino acid sequence of SEQ ID NO: 7; (d) HVR-L1 comprising an amino acid sequence of SEQ ID NO: 8; (e) HVR-L2 comprising the amino acid sequence of SEQ ID NO: 9; and (f) HVR-L3 comprising the amino acid sequence of SEQ ID NO: 10. In some such embodiments, the immunoconjugate comprises an anti-CD79b antibody comprising at least one of: (i) HVR-H3 comprising the amino acid sequence of SEQ ID NO: 7, and/or (ii) HVR-L1 comprising an amino acid sequence of SEQ ID NO: 8. In some embodiments, the immunoconjugate comprises an anti-CD79b antibody comprising at least one of: (i) HVR-H3 comprising the amino acid sequence of SEQ ID NO: 7, and/or (ii) HVR-L1 comprising the amino acid sequence of SEQ ID NO: 8. In some embodiments, the immunoconjugate comprises an anti-CD79b antibody comprising at least one, at least two, or all three VH HVR sequences selected from (a) HVR-H1 comprising the amino acid sequence of SEQ ID NO: 5; (b) HVR-H2 comprising the amino acid sequence of SEQ ID NO: 6; and (c) HVR-H3 comprising the amino acid sequence of SEQ ID NO: 7. In some embodiments, the immunoconjugate comprises an anti-CD79b antibody that comprises an HVR-H3 comprising the amino acid sequence of SEQ ID NO: 7. In some embodiments, the immunoconjugate comprises an anti-CD79b antibody that comprises an HVR-H3 comprising the amino acid sequence of SEQ ID NO: 7 and an HVR-L3 comprising the amino acid sequence of SEQ ID NO: 10. In some embodiments, the immunoconjugate comprises an anti-CD79b antibody that comprises an HVR-H3 comprising the amino acid sequence of SEQ ID NO: 7, an HVR-L3 comprising the amino acid sequence of SEQ ID NO: 10, and an HVR-H2 comprising the amino acid sequence of SEQ ID NO: 6. In some embodiments, the immunoconjugate comprises an anti-CD79b antibody that comprises (a) HVR-H1 comprising the amino acid sequence of SEQ ID NO: 5; (b) HVR-H2 comprising the amino acid sequence of SEQ ID NO: 6; and (c) HVR-H3 comprising the amino acid sequence of SEQ ID NO: 7.

In some embodiments, the immunoconjugate comprises an anti-CD79b antibody comprising at least one, at least two, or all three VL HVR sequences selected from (a) HVR-L1 comprising an amino acid sequence of SEQ ID NO: 8; (b) HVR-L2 comprising the amino acid sequence of SEQ ID NO: 9; and (c) HVR-L3 comprising the amino acid sequence of SEQ ID NO: 10. In some embodiments, the immunoconjugate comprises an anti-CD79b antibody that comprises at least one, at least two, or all three VL HVR sequences selected from (a) HVR-L1 comprising the amino acid sequence of SEQ ID NO: 8; (b) HVR-L2 comprising the amino acid sequence of SEQ ID NO: 9; and (c) HVR-L3 comprising the amino acid sequence of SEQ ID NO: 10. In some embodiments, the immunoconjugate comprises (a) HVR-L1 comprising an amino acid sequence of SEQ ID NO: 8; (b) HVR-L2 comprising the amino acid sequence of SEQ ID NO: 9; and (c) HVR-L3 comprising the amino acid sequence of SEQ ID NO: 10. In some embodiments, the immunoconjugate comprises an anti-CD79b antibody that comprises an HVR-L1 comprising the amino acid sequence of SEQ ID NO: 8 In some embodiments, the immunoconjugate comprises an anti-CD79b antibody that comprises (a) HVR-L1 comprising the amino acid sequence of SEQ ID NO: 8; (b) HVR-L2 comprising the amino acid sequence of SEQ ID NO: 9; and (c) HVR-L3 comprising the amino acid sequence of SEQ ID NO: 10.

In some embodiments, the immunoconjugate comprises an anti-CD79b antibody comprising (a) a VH domain comprising at least one, at least two, or all three VH HVR sequences selected from (i) HVR-H1 comprising the amino acid sequence of SEQ ID NO: 5, (ii) HVR-H2 comprising the amino acid sequence of SEQ ID NO: 6, and (iii) HVR-H3 comprising the amino acid sequence of SEQ ID NO:7; and (b) a VL domain comprising at least one, at least two, or all three VL HVR sequences selected from (i) HVR-L1 comprising an amino acid sequence of SEQ ID NO: 8, (ii) HVR-L2 comprising the amino acid sequence of SEQ ID NO: 9, and (iii) HVR-L3 comprising the amino acid sequence of SEQ ID NO: 10. In some embodiments, the immunoconjugate comprises an anti-CD79b antibody that comprises at least one of: (i) HVR-H3 comprising the amino acid sequence of SEQ ID NO: 7, and/or (ii) HVR-L1 comprising the amino acid sequence of SEQ ID NO: 8.

In some embodiments, the immunoconjugate comprises an anti-CD79b antibody that comprises (a) HVR-H1 comprising the amino acid sequence of SEQ ID NO: 5; (b) HVR-H2 comprising the amino acid sequence of SEQ ID NO: 6; (c) HVR-H3 comprising the amino acid sequence of SEQ ID NO: 7; (d) HVR-L1 comprising an amino acid sequence of SEQ ID NO: 8; (e) HVR-L2 comprising the amino acid sequence of SEQ ID NO: 9; and (f) HVR-L3 comprising the amino acid sequence of SEQ ID NO: 10. In some embodiments, the immunoconjugate comprises at least one of: HVR-H3 comprising the amino acid sequence of SEQ ID NO: 7 and/or HVR-L1 comprising an amino acid sequence of SEQ ID NO: 8. In some embodiments, the immunoconjugate comprises an anti-CD79b antibody that comprises (a) HVR-H1 comprising the amino acid sequence of SEQ ID NO: 5; (b) HVR-H2 comprising the amino acid sequence of SEQ ID NO: 6; (c) HVR-H3 comprising the amino acid sequence of SEQ ID NO: 7; (d) HVR-L1 comprising the amino acid sequence of SEQ ID NO: 8; (e) HVR-L2 comprising the amino acid sequence of SEQ ID NO: 9; and (f) HVR-L3 comprising the amino acid sequence of SEQ ID NO: 10.

In some embodiments, the anti-CD79b immunoconjugates comprise a humanized anti-CD79b antibody. In some embodiments, an anti-CD79b antibody comprises HVRs as in any of the embodiments provided herein, and further comprises a human acceptor framework, e.g., a human immunoglobulin framework or a human consensus framework. In some embodiments, the human acceptor framework is the human VL kappa 1 (VLKI) framework and/or the VH framework VHIII. In some embodiments, a humanized anti-CD79b antibody comprises (a) HVR-H1 comprising the amino acid sequence of SEQ ID NO: 5; (b) HVR-H2 comprising the amino acid sequence of SEQ ID NO: 6; (c) HVR-H3 comprising the amino acid sequence of SEQ ID NO: 7; (d) HVR-L1 comprising an amino acid sequence of SEQ ID NO: 8; (e) HVR-L2 comprising the amino acid sequence of SEQ ID NO: 9; and (f) HVR-L3 comprising the amino acid sequence of SEQ ID NO: 10. In some embodiments, a humanized anti-CD79b antibody comprises (a) HVR-H1 comprising the amino acid sequence of SEQ ID NO: 5; (b) HVR-H2 comprising the amino acid sequence of SEQ ID NO: 6; (c) HVR-H3 comprising the amino acid sequence of SEQ ID NO: 7; (d) HVR-L1 comprising the amino acid sequence of SEQ ID NO: 8; (e) HVR-L2 comprising the amino acid sequence of SEQ ID NO: 9; and (f) HVR-L3 comprising the amino acid sequence of SEQ ID NO: 10.

In some embodiments, the immunoconjugate (e.g., the anti-CD79b immunoconjugate) comprises an anti-CD79b antibody comprising a heavy chain variable domain (VH) sequence having at least 90%, 91%, 92%, 93%, 94%, 95%, 96%, 97%, 98%, 99%, or 100% sequence identity to the amino acid sequence of SEQ ID NO: 3. In some embodiments, a VH sequence having at least 90%, 91%, 92%, 93%, 94%, 95%, 96%, 97%, 98%, or 99% identity to the amino acid sequence of SEQ ID NO: 3 contains substitutions (e.g., conservative substitutions), insertions, or deletions relative to the reference sequence, but an anti-CD79b immunoconjugate comprising that sequence retains the ability to bind to CD79b. In some embodiments, a total of 1 to 10 amino acids have been substituted, inserted and/or deleted in SEQ ID NO: 3. In some embodiments, a total of 1 to 5 amino acids have been substituted, inserted and/or deleted in SEQ ID NO: 3. In some embodiments, substitutions, insertions, or deletions occur in regions outside the HVRs (i.e., in the FRs). In some embodiments, the immunoconjugate (e.g., the anti-CD79b immunoconjugate) comprises the VH sequence of SEQ ID NO: 3, including post-translational modifications of that sequence. In some embodiments, the VH comprises one, two or three HVRs selected from: (a) HVR-H1 comprising the amino acid sequence of SEQ ID NO: 5, (b) HVR-H2 comprising the amino acid sequence of SEQ ID NO: 6, and (c) HVR-H3 comprising the amino acid sequence of SEQ ID NO: 7.

In some embodiments, the immunoconjugate (e.g., the anti-CD79b immunoconjugate) comprises an anti-CD79b antibody that comprises a light chain variable domain (VL) having at least 90%, 91%, 92%, 93%, 94%, 95%, 96%, 97%, 98%, 99%, or 100% sequence identity to the amino acid sequence of SEQ ID NO: 4. In certain embodiments, a VL sequence having at least 90%, 91%, 92%, 93%, 94%, 95%, 96%, 97%, 98%, or 99% identity to the amino acid sequence of SEQ ID NO: 4 contains substitutions (e.g., conservative substitutions), insertions, or deletions relative to the reference sequence, but an anti-CD79b immunoconjugate comprising that sequence retains the ability to bind to CD79b. In certain embodiments, a total of 1 to 10 amino acids have been substituted, inserted and/or deleted in SEQ ID NO: 4. In certain embodiments, a total of 1 to 5 amino acids have been substituted, inserted and/or deleted in SEQ ID NO: 4. In certain embodiments, the substitutions, insertions, or deletions occur in regions outside the HVRs (i.e., in the FRs). In some embodiments, the anti-CD79b immunoconjugate comprises an anti-CD79b antibody that comprises the VL sequence of SEQ ID NO: 4, including post-translational modifications of that sequence. In some embodiments, the VL comprises one, two or three HVRs selected from (a) HVR-L1 comprising an amino acid sequence of SEQ ID NO: 8; (b) HVR-L2 comprising the amino acid sequence of SEQ ID NO: 9; and (c) HVR-L3 comprising the amino acid sequence of SEQ ID NO: 10. In some embodiments, the VL comprises one, two or three HVRs selected from (a) HVR-L1 comprising the amino acid sequence of SEQ ID NO: 8; (b) HVR-L2 comprising the amino acid sequence of SEQ ID NO: 9; and (c) HVR-L3 comprising the amino acid sequence of SEQ ID NO: 10.

In some embodiments, the immunoconjugate (e.g., the anti-CD79b immunoconjugate) comprises an anti-CD79b antibody that comprises VH as in any of the embodiments provided herein, and a VL as in any of the embodiments provided herein. In some embodiments, the immunoconjugate comprises an anti-CD79b antibody that comprises the VH and VL sequences in SEQ ID NO: 3 and SEQ ID NO: 4, respectively, including post-translational modifications of those sequences.

In some embodiments, the immunoconjugate (e.g., anti-CD79b immunoconjugate) comprises an anti-CD79b antibody that binds to the same epitope as an anti-CD79b antibody described herein. For example, in some embodiments, the immunoconjugate (e.g., anti-CD79b immunoconjugate) comprises an anti-CD79b antibody that binds to the same epitope as an anti-CD79b antibody comprising a VH sequence of SEQ ID NO: 3 and a VL sequence of SEQ ID NO: 4.

In some embodiments, the immunoconjugate comprises an anti-CD79b antibody that is a monoclonal antibody, a chimeric antibody, humanized antibody, or human antibody. In some embodiments, immunoconjugate comprises an antigen-binding fragment of an anti-CD79b antibody described herein, e.g., a Fv, Fab, Fab′, scFv, diabody, or F(ab′)2 fragment. In some embodiments, the immunoconjugate comprises a substantially full length anti-CD79b antibody, e.g., an IgG1 antibody or other antibody class or isotype as described elsewhere herein.

In some embodiments, the immunoconjugate comprises an anti-CD79b antibody comprising a heavy chain comprising the amino acid sequence of SEQ ID NO: 12, and wherein the light chain comprises the amino acid sequence of SEQ ID NO: 11. In some embodiments, the immunoconjugate comprises an anti-CD79b antibody that comprises a heavy chain comprising the amino acid sequence of SEQ ID NO: 13 and a light chain comprising the amino acid sequence of SEQ ID NO: 11. In some embodiments, the immunoconjugate comprises an anti-CD79b antibody that comprises a heavy chain comprising the amino acid sequence of SEQ ID NO: 12 and a light chain comprising the amino acid sequence of SEQ ID NO: 14.

In some embodiments, the immunoconjugate is polatuzumab vedotin, as described in WHO Drug Information, Vol. 26, No. 4, 2012 (Proposed INN: List 108), which is expressly incorporated by reference herein in its entirety. As shown in WHO Drug Information, Vol. 26, No. 4, 2012, polatuzumab vedotin has the following structure: immunoglobulin G1-kappa auristatin E conjugate, anti-[Homo sapiens CD79B (immunoglobulin-associated CD79 beta)], humanized monoclonal antibody conjugated to auristatin E; gamma1 heavy chain (1-447) [humanized VH (Homo sapiens IGHV3-66*01 (79.60%)-(IGHD)-IGHJ4*01) [8.8.13](1-120)-Homo sapiens IGHG1*03 (CH1 R120>K (214) (121-218), hinge (219-233), CH2 (234-343), CH3 (344-448), CHS (449-450)) (121-450)], (220-218′)-disulfide (if not conjugated) with kappa light chain (1′-218′)[humanized V-KAPPA (Homo sapiens IGKV1-39*01 (80.00%)-IGKJ1*01) [11.3.9](1-112′)-Homo sapiens IGKC*01 (113′-218′)]; dimer (226-226″:229-229″)-bisdisulfide; conjugated, on an average of 3 to 4 cysteinyl, to monomethylauristatin E (MMAE), via a cleavable maleimidecaproyl-valyl-citrullinyl-p-aminobenzylcarbamate (mc-val-cit-PABC) linker; the heavy chain of polatuzumab vedotin has the following sequence:

(SEQ ID NO: 15)
EVQLVESGGG LVQPGGSLRL SCAASGYTFS SYWIEWVRQA PGKGLEWIGE 50
ILPGGGDTNY NEIFKGRATF SADTSKNTAY LQMNSLRAED TAVYYCTRRV 100
PIRLDYWGQG TLVTVSSAST KGPSVFPLAP SSKSTSGGTA ALGCLVKDYF 150
PEPVTVSWNS GALTSGVHTF PAVLQSSGLY SLSSVVTVPS SSLGTQTYIC 200
NVNHKPSNTK VDKKVEPKSC DKTHTCPPCP APELLGGPSV FLFPPKPKDT 250
LMISRTPEVT CVVVDVSHED PEVKFNWYVD GVEVHNAKTK PREEQYNSTY 300
RVVSVLTVLH QDWLNGKEYK CKVSNKALPA PIEKTISKAK GQPREPQVYT 350
LPPSREEMTK NQVSLTCLVK GFYPSDIAVE WESNGQPENN YKTTPPVLDS 400
DGSFFLYSKL TVDKSRWQQG NVFSCSVMHE ALHNHYTQKS LSLSPGK 447;

the light chain of polatuzumab vedotin has the following sequence:

(SEQ ID NO: 11)
DIQLTQSPSS LSASVGDRVT ITCKASQSVD YEGDSFLNWY QQKPGKAPKL 50
LIYAASNLES GVPSRFSGSG SGTDFTLTIS SLQPEDFATY YCQQSNEDPL 100
TFGQGTKVEI KRTVAAPSVF IFPPSDEQLK SGTASVVCLL NNFYPREAKV 150
QWKVDNALQS GNSQESVTEQ DSKDSTYSLS STLTLSKADY EKHKVYACEV 200
THQGLSSPVT KSFNRGEC 218;

the disulfide bridge locations are:
Intra-H 22-96 144-200 261-321 367-425
   22″-96″ 147-203″ 261″-321″ 367-425″
Intra-L 23′-92′ 138′-198′
   23′″-92′″ 138′″-198′″
Inter-H-L*220-218′ 220″-218′″
Inter-H-H*226-226″ 229-229″
*Two or three of the inter-chain disulfide bridges are not present, the antibody being conjugated to an average of 3 to 4 drug linkers each via a thioether bond; the N-glycosylation sites are H CH2 N84.4: 297, 297″ but lacking carbohydrate;
and other post-translational modifications are: lacking H chain C-terminal lysine. Thus, in some embodiments, the heavy chain of polatuzumab vedotin has the sequence of SEQ ID NO: 12.
   (iii) Drugs/Cytotoxic Agents

Anti-CD79b immunoconjugates comprise an anti-CD79b antibody (e.g., an anti-CD79b antibody described herein) conjugated to one or more drugs/cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes (i.e., a radioconjugate). Such immunoconjugates are targeted chemotherapeutic molecules which combine properties of both antibodies and cytotoxic drugs by targeting potent cytotoxic drugs to antigen-expressing cancer cells (such as tumor cells) (Teicher, B. A. (2009) Current Cancer Drug Targets 9:982-1004), thereby enhancing the therapeutic index by maximizing efficacy and minimizing off-target toxicity (Carter, P. J. and Senter P. D. (2008) The Cancer Jour. 14(3):154-169; Chari, R. V. (2008) Acc. Chem. Res. 41:98-107. That is, the anti-CD79b immunoconjugates selectively deliver an effective dose of a drug to cancerous cells/tissues whereby greater selectivity, i.e. a lower efficacious dose, may be achieved while increasing the therapeutic index (“therapeutic window”) (Polakis P. (2005) Current Opinion in Pharmacology 5:382-387).

Anti-CD79b immunoconjugates used in the methods provided herein include those with anticancer activity. In some embodiments, the anti-CD79b immunoconjugate comprises an anti-CD79b antibody conjugated, i.e. covalently attached, to the drug moiety. In some embodiments, the anti-CD79b antibody is covalently attached to the drug moiety through a linker. The drug moiety (D) of the anti-CD79b immunoconjugate may include any compound, moiety or group that has a cytotoxic or cytostatic effect. Drug moieties may impart their cytotoxic and cytostatic effects by mechanisms including but not limited to tubulin binding, DNA binding or intercalation, and inhibition of RNA polymerase, protein synthesis, and/or topoisomerase. Exemplary drug moieties include, but are not limited to, a maytansinoid, dolastatin, auristatin, calicheamicin, anthracycline, duocarmycin, vinca alkaloid, taxane, trichothecene, CC1065, camptothecin, elinafide, and stereoisomers, isosteres, analogs, and derivatives thereof that have cytotoxic activity.

### (1) Maytansine and Maytansinoids

In some embodiments, an anti-CD79b immunoconjugate comprises an anti-CD79b antibody conjugated to one or more maytansinoid molecules. Maytansinoids are derivatives of maytansine, and are mitototic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinoids are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533.

Maytansinoid drug moieties are attractive drug moieties in antibody-drug conjugates because they are: (i) relatively accessible to prepare by fermentation or chemical modification or derivatization of fermentation products, (ii) amenable to derivatization with functional groups suitable for conjugation through non-disulfide linkers to antibodies, (iii) stable in plasma, and (iv) effective against a variety of tumor cell lines.

Certain maytansinoids suitable for use as maytansinoid drug moieties are known in the art and can be isolated from natural sources according to known methods or produced using genetic engineering techniques (see, e.g., Yu et al (2002) PNAS 99:7968-7973). Maytansinoids may also be prepared synthetically according to known methods.

Exemplary maytansinoid drug moieties include, but are not limited to, those having a modified aromatic ring, such as: C-19-dechloro (U.S. Pat. No. 4,256,746) (prepared, for example, by lithium aluminum hydride reduction of ansamytocin P2); C-20-hydroxy (or C-20-demethyl)+/−C-19-dechloro (U.S. Pat. Nos. 4,361,650 and 4,307,016) (prepared, for example, by demethylation using Streptomyces or Actinomyces or dechlorination using LAH); and C-20-demethoxy, C-20-acyloxy (—OCOR), +/−dechloro (U.S. Pat. No. 4,294,757) (prepared, for example, by acylation using acyl chlorides), and those having modifications at other positions of the aromatic ring.

Exemplary maytansinoid drug moieties also include those having modifications such as: C-9-SH (U.S. Pat. No. 4,424,219) (prepared, for example, by the reaction of maytansinol with H₂S or P₂S₅); C-14-alkoxymethyl(demethoxy/CH₂OR)(U.S. Pat. No. 4,331,598); C-14-hydroxymethyl or acyloxymethyl (CH₂OH or Ch₂OAc) (U.S. Pat. No. 4,450,254) (prepared, for example, from Nocardia); C-15-hydroxy/acyloxy (U.S. Pat. No. 4,364,866) (prepared, for example, by the conversion of maytansinol by Streptomyces); C-15-methoxy (U.S. Pat. Nos. 4,313,946 and 4,315,929) (for example, isolated from Trewia nudlflora); C-18-N-demethyl (U.S. Pat. Nos. 4,362,663 and 4,322,348) (prepared, for example, by the demethylation of maytansinol by Streptomyces); and 4,5-deoxy (U.S. Pat. No. 4,371,533) (prepared, for example, by the titanium trichloride/LAH reduction of maytansinol).

Many positions on maytansinoid compounds are useful as the linkage position. For example, an ester linkage may be formed by reaction with a hydroxyl group using conventional coupling techniques. In some embodiments, the reaction may occur at the C-3 position having a hydroxyl group, the C-14 position modified with hydroxymethyl, the C-15 position modified with a hydroxyl group, and the C-20 position having a hydroxyl group. In some embodiments, the linkage is formed at the C-3 position of maytansinol or a maytansinol analogue.

Maytansinoid drug moieties include those having the structure:

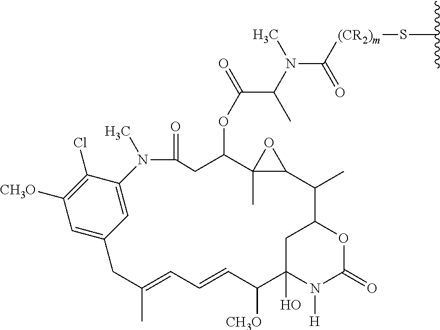

wherein the wavy line indicates the covalent attachment of the sulfur atom of the maytansinoid drug moiety to a linker of an anti-CD79b immunoconjugate. Each R may independently be H or a C₁-C₆ alkyl. The alkylene chain attaching the amide group to the sulfur atom may be methanyl, ethanyl, or propyl, i.e., m is 1, 2, or 3 (U.S. Pat. No. 633,410; U.S. Pat. No. 5,208,020; Chari et al (1992) Cancer Res. 52:127-131; Liu et al (1996) Proc. Natl. Acad. Sci USA 93:8618-8623).

All stereoisomers of the maytansinoid drug moiety are contemplated for the anti-CD79b immunoconjugate used in a method provided herein, i.e. any combination of R and S configurations at the chiral carbons (U.S. Pat. Nos. 7,276,497; 6,913,748; 6,441,163; 633,410 (RE39151); U.S. Pat. No. 5,208,020; Widdison et al (2006) J. Med. Chem. 49:4392-4408, which are incorporated by reference in their entirety). In some embodiments, the maytansinoid drug moiety has the following stereochemistry:

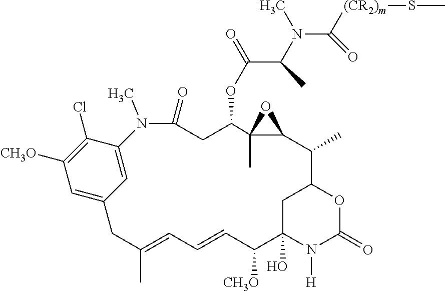

Exemplary embodiments of maytansinoid drug moieties include, but are not limited to, DM1; DM3; and DM4, having the structures:

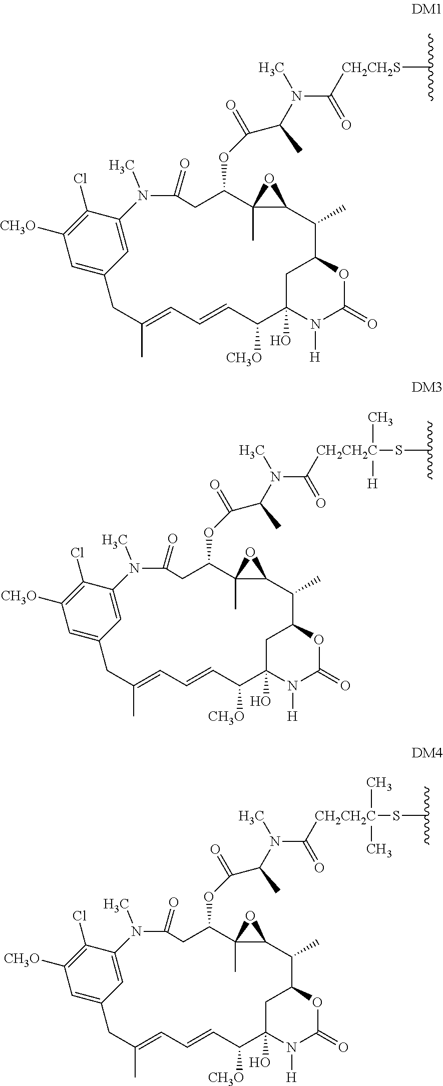

wherein the wavy line indicates the covalent attachment of the sulfur atom of the drug to a linker (L) of an anti-CD79b immunoconjugate.

Other exemplary maytansinoid anti-CD79b immunoconjugates have the following structures and abbreviations (wherein Ab is an anti-CD79b antibody and p is 1 to about 20. In some embodiments, p is 1 to 10, p is 1 to 7, p is 1 to 5, or p is 1 to 4):

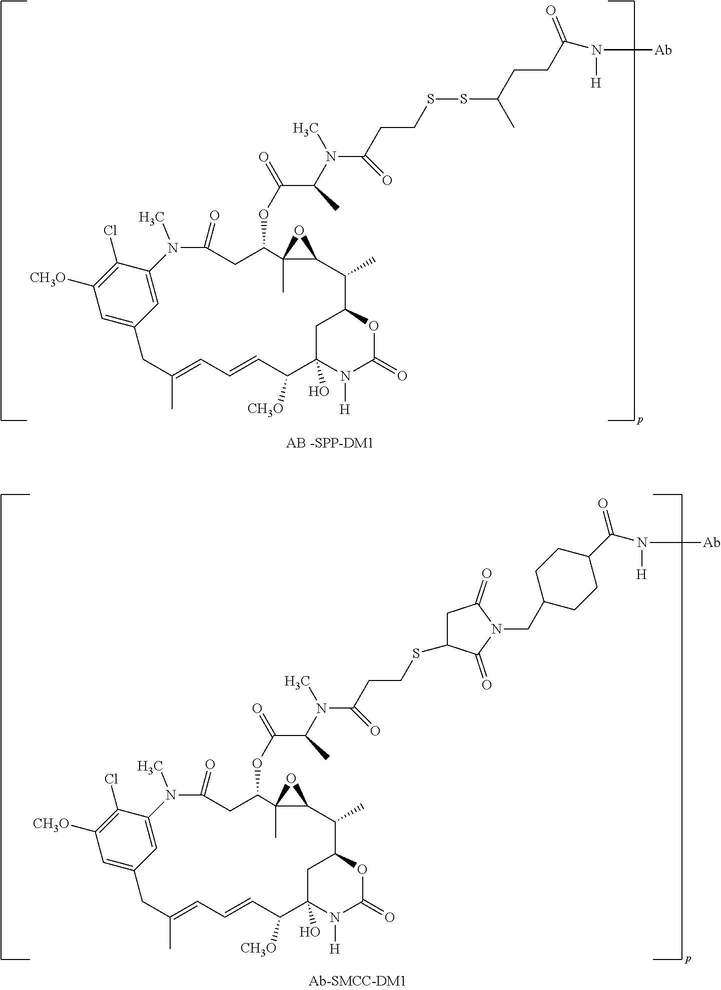

Exemplary antibody-drug conjugates wherein DM1 is linked through a BMPEO linker to a thiol group of the antibody have the structure and abbreviation:

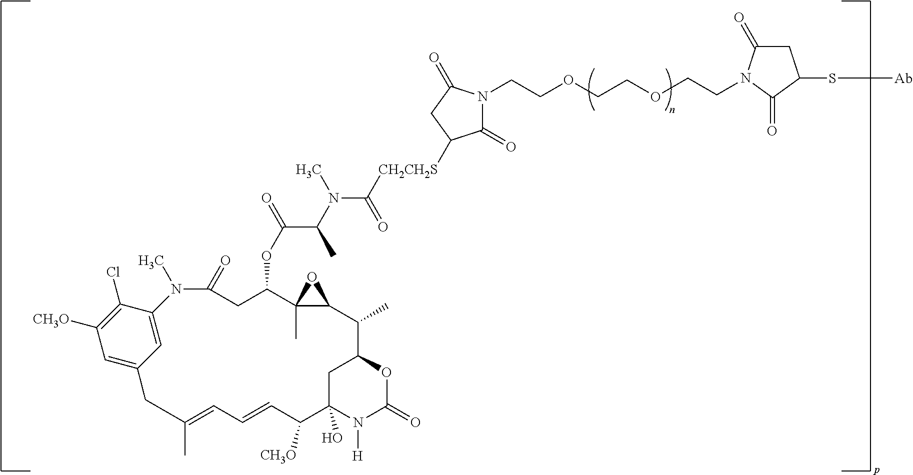

wherein Ab is an anti-CD79b antibody; n is 0, 1, or 2; and p is 1 to about 20. In some embodiments, p is 1 to 10, p is 1 to 7, p is 1 to 5, or p is 1 to 4.

Immunoconjugates containing maytansinoids, methods of making the same, and their therapeutic use are disclosed, for example, in U.S. Pat. Nos. 5,208,020 and 5,416,064; US 2005/0276812 A1; and European Patent EP 0 425 235 B1, the disclosures of which are hereby expressly incorporated by reference. See also Liu et al. Proc. Natl. Acad. Sci. USA 93:8618-8623 (1996); and Chari et al. Cancer Research 52:127-131 (1992).

In some embodiments, anti-CD79b antibody-maytansinoid conjugates may be prepared by chemically linking an anti-CD79b antibody to a maytansinoid molecule without significantly diminishing the biological activity of either the antibody or the maytansinoid molecule. See, e.g., U.S. Pat. No. 5,208,020 (the disclosure of which is hereby expressly incorporated by reference). In some embodiments, an anti-CD79b immunoconjugate with an average of 3-4 maytansinoid molecules conjugated per antibody molecule has shown efficacy in enhancing cytotoxicity of target cells without negatively affecting the function or solubility of the antibody. In some instances, even one molecule of toxin/antibody is expected to enhance cytotoxicity over the use of naked anti-CD79b antibody.

Exemplary linking groups for making antibody-maytansinoid conjugates include, for example, those described herein and those disclosed in U.S. Pat. No. 5,208,020; EP Patent 0 425 235 B1; Chari et al. Cancer Research 52:127-131 (1992); US 2005/0276812 A1; and US 2005/016993 A1, the disclosures of which are hereby expressly incorporated by reference.

### (2) Auristatins and Dolastatins

Drug moieties include dolastatins, auristatins, and analogs and derivatives thereof (U.S. Pat. Nos. 5,635,483; 5,780,588; 5,767,237; 6,124,431). Auristatins are derivatives of the marine mollusk compound dolastatin-10. While not intending to be bound by any particular theory, dolastatins and auristatins have been shown to interfere with microtubule dynamics, GTP hydrolysis, and nuclear and cellular division (Woyke et al (2001) Antimicrob. Agents and Chemother. 45(12):3580-3584) and have anticancer (U.S. Pat. No. 5,663,149) and antifungal activity (Pettit et al (1998) Antimicrob. Agents Chemother. 42:2961-2965). The dolastatin/auristatin drug moiety may be attached to the antibody through the N (amino) terminus or the C (carboxyl) terminus of the peptidic drug moiety (WO 02/088172; Doronina et al (2003) Nature Biotechnology 21(7):778-784; Francisco et al (2003) Blood 102(4):1458-1465).

Exemplary auristatin embodiments include the N-terminus linked monomethylauristatin drug moieties D_{E} and D_{F}, disclosed in U.S. Pat. Nos. 7,498,298 and 7,659,241, the disclosures of which are expressly incorporated by reference in their entirety:

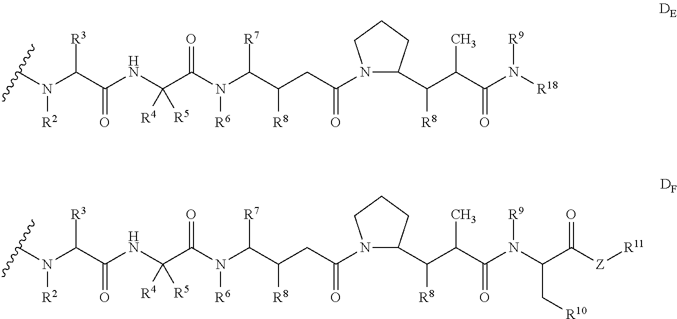

wherein the wavy line of D_{E} and D_{F} indicates the covalent attachment site to an antibody or antibody-linker component, and independently at each location:
   R² is selected from H and C₁-C₈ alkyl;
   R³ is selected from H, C₁-C₈ alkyl, C₃-C₈ carbocycle, aryl, C₁-C₈ alkyl-aryl, C₁-C₈ alkyl-(C₃-C₈ carbocycle), C₃-C₈ heterocycle and C₁-C₈ alkyl-(C₃-C₈ heterocycle);
   R⁴ is selected from H, C₁-C₈ alkyl, C₃-C₈ carbocycle, aryl, C₁-C₈ alkyl-aryl, C₁-C₈ alkyl-(C₃-C₈ carbocycle), C₃-C₈ heterocycle and C₁-C₈ alkyl-(C₃-C₈ heterocycle);
   R⁵ is selected from H and methyl;
   or R⁴ and R⁵ jointly form a carbocyclic ring and have the formula —(CR^{a}R^{b})ₙ wherein Ra and R are independently selected from H, C₁-C₈ alkyl and C₃-C₈ carbocycle and n is selected from 2, 3, 4, 5 and 6;
   R⁶ is selected from H and C₁-C₈ alkyl;
   R⁷ is selected from H, C₁-C₈ alkyl, C₃-C₈ carbocycle, aryl, C₁-C₈ alkyl-aryl, C₁-C₈ alkyl-(C₃-C₈ carbocycle), C₃-C₈ heterocycle and C₁-C₈ alkyl-(C₃-C₈ heterocycle);
   each R⁸ is independently selected from H, OH, C₁-C₈ alkyl, C₃-C₈ carbocycle and O—(C₁-C₈ alkyl);
   R⁹ is selected from H and C₁-C₈ alkyl;
   R¹⁰ is selected from aryl or C₃-C₈ heterocycle;
   Z is O, S, NH, or NR¹², wherein R¹² is C₁-C₈ alkyl;
   R¹¹ is selected from H, C₁-C₂₀ alkyl, aryl, C₃-C₈ heterocycle, —(R¹³O)ₘ—R¹⁴, or —(R¹³O)ₘ—CH(R)₂;
   m is an integer ranging from 1-1000;
   R¹³ is C₂-C₈ alkyl;
   R¹⁴ is H or C₁-C₈ alkyl;
   each occurrence of R¹⁵ is independently H, COOH, —(CH₂)ₙ—N(R¹⁶)₂, —(CH₂)ₙ—SO₃H, or —(CH₂)ₙ—SO₃—C₁-C₈ alkyl;
   each occurrence of R¹⁶ is independently H, C₁-C₈ alkyl, or —(CH₂)ₙ—COOH;
   R¹⁸ is selected from —C(R⁸)₂—C(R⁸)₂-aryl, —C(R⁸)₂—C(R⁸)₂—(C₃-C₈ heterocycle), and —C(R⁸)₂—C(R⁸)₂—(C₃-C₈ carbocycle); and
   n is an integer ranging from 0 to 6.

In one embodiment, R³, R⁴ and R⁷ are independently isopropyl or sec-butyl and R⁵ is —H or methyl. In an exemplary embodiment, R³ and R⁴ are each isopropyl, R⁵ is —H, and R⁷ is sec-butyl.

In yet another embodiment, R² and R⁶ are each methyl, and R⁹ is —H.

In still another embodiment, each occurrence of R⁸ is —OCH₃.

In an exemplary embodiment, R³ and R⁴ are each isopropyl, R² and R⁶ are each methyl, R⁵ is —H, R⁷ is sec-butyl, each occurrence of R⁸ is —OCH₃, and R⁹ is —H.

In one embodiment, Z is —O— or —NH—.

In one embodiment, R¹⁰ is aryl.

In an exemplary embodiment, R¹⁰ is -phenyl.

In an exemplary embodiment, when Z is —O—, R¹¹ is —H, methyl or t-butyl.

In one embodiment, when Z is —NH, R¹¹ is —CH(R¹⁵)₂, wherein R¹⁵ is —(CH₂)ₙ—N(R¹⁶)₂, and R¹⁶ is —C₁-C₈ alkyl or —(CH₂)ₙ—COOH.

In another embodiment, when Z is —NH, R¹¹ is —CH(R¹⁵)₂, wherein R¹⁵ is —(CH₂)ₙ—SO₃H.

An exemplary auristatin embodiment of formula D_{E} is MMAE, wherein the wavy line indicates the covalent attachment to a linker (L) of an anti-CD79b immunoconjugate:

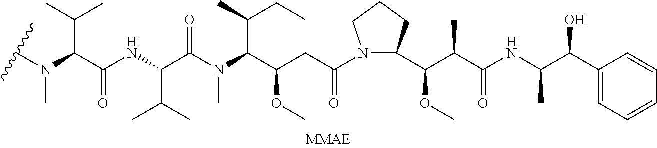 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

An exemplary auristatin embodiment of formula D_{F} is MMAF, wherein the wavy line indicates the covalent attachment to a linker (L) of an anti-CD79b immunoconjugate:

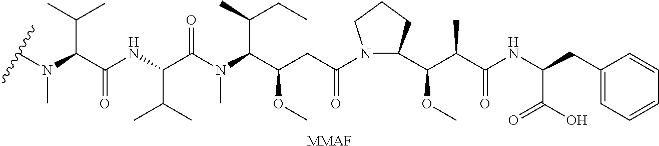

Other exemplary embodiments include monomethylvaline compounds having phenylalanine carboxy modifications at the C-terminus of the pentapeptide auristatin drug moiety (WO 2007/008848) and monomethylvaline compounds having phenylalanine sidechain modifications at the C-terminus of the pentapeptide auristatin drug moiety (WO 2007/008603).

Nonlimiting exemplary embodiments of an anti-CD79b immunoconjugate of Formula I comprising MMAE or MMAF and various linker components have the following structures and abbreviations (wherein “Ab” is an anti-CD79b antibody; p is 1 to about 8, “Val-Cit” is a valine-citrulline dipeptide; and “S” is a sulfur atom:

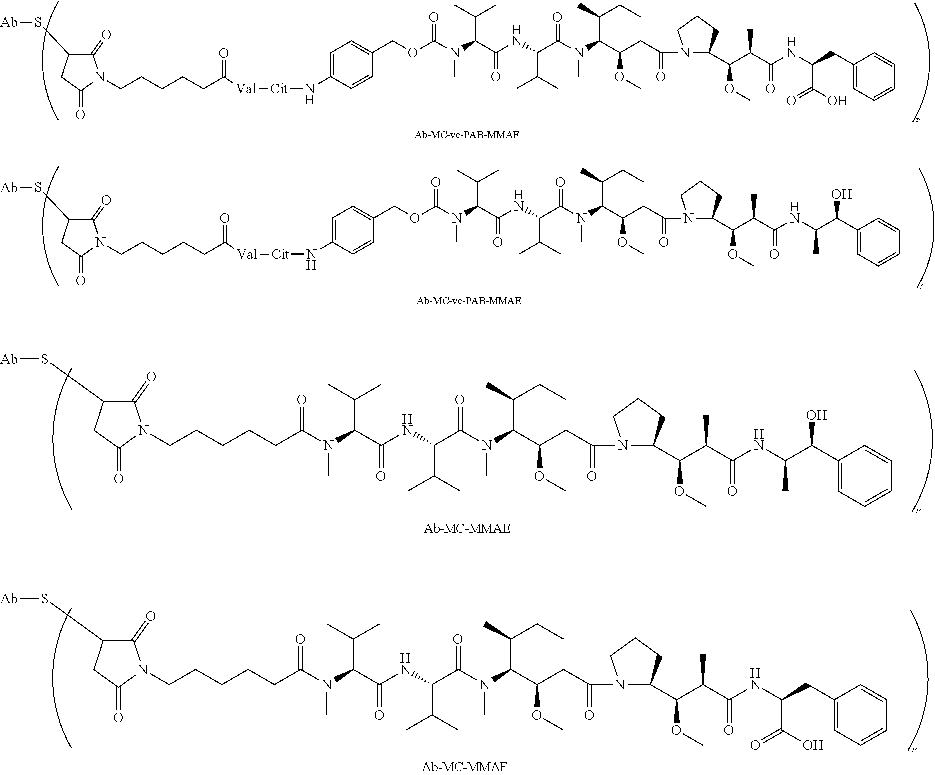

In certain embodiments, the anti-CD79b immunoconjugate comprises the structure of Ab-MC-vc-PAB-MMAE, wherein p is, e.g., about 1 to about 8; about 2 to about 7; about 3 to about 5; about 3 to about 4; or about 3.5. In some embodiments, the anti-CD79b immunoconjugate is huMA79bv28-MC-vc-PAB-MMAE, e.g., an anti-CD79b immunoconjugate comprising the structure of MC-vc-PAB-MMAE, wherein p is, e.g., about 1 to about 8; about 2 to about 7; about 3 to about 5; about 3 to about 4; or about 3.5, wherein the anti-CD79b antibody comprises a heavy chain comprising the amino acid sequence of SEQ ID NO: 12, and wherein the light chain comprises the amino acid sequence of SEQ ID NO: 11. In some embodiments, the anti-CD79b immunoconjugate is polatuzumab vedotin (CAS Number 1313206-42-6). Polatuzumab vedotin has the IUPHAR/BPS Number 8404, the KEGG Number D10761, the INN number 9714, and can also be referred to as “DCDS4501A,” or “RG7596.”

Nonlimiting exemplary embodiments of anti-CD79b immunoconjugates of Formula I comprising MMAF and various linker components further include Ab-MC-PAB-MMAF and Ab-PAB-MMAF. Immunoconjugates comprising MMAF attached to an antibody by a linker that is not proteolytically cleavable have been shown to possess activity comparable to immunoconjugates comprising MMAF attached to an antibody by a proteolytically cleavable linker (Doronina et al. (2006) Bioconjugate Chem. 17:114-124). In some such embodiments, drug release is believed to be effected by antibody degradation in the cell.

Typically, peptide-based drug moieties can be prepared by forming a peptide bond between two or more amino acids and/or peptide fragments. Such peptide bonds can be prepared, for example, according to a liquid phase synthesis method (see, e.g., E. Schröder and K. Lübke, “The Peptides”, volume 1, pp 76-136, 1965, Academic Press). Auristatin/dolastatin drug moieties may, in some embodiments, be prepared according to the methods of: U.S. Pat. Nos. 7,498,298; 5,635,483; 5,780,588; Pettit et al (1989) J. Am. Chem. Soc. 111:5463-5465; Pettit et al (1998) Anti-Cancer Drug Design 13:243-277; Pettit, G. R., et al. Synthesis, 1996, 719-725; Pettit et al (1996) J. Chem. Soc. Perkin Trans. 1 5:859-863; and Doronina (2003) Nat. Biotechnol. 21(7):778-784.

In some embodiments, auristatin/dolastatin drug moieties of formulas D_{E} such as MMAE, and DF, such as MMAF, and drug-linker intermediates and derivatives thereof, such as MC-MMAF, MC-MMAE, MC-vc-PAB-MMAF, and MC-vc-PAB-MMAE, may be prepared using methods described in U.S. Pat. No. 7,498,298; Doronina et al. (2006) Bioconjugate Chem. 17:114-124; and Doronina et al. (2003) Nat. Biotech. 21:778-784 and then conjugated to an antibody of interest.

### (3) Calicheamicin

In some embodiments, the anti-CD79b immunoconjugate comprises an anti-CD79b antibody conjugated to one or more calicheamicin molecules. The calicheamicin family of antibiotics, and analogues thereof, are capable of producing double-stranded DNA breaks at sub-picomolar concentrations (Hinman et al., (1993) Cancer Research 53:3336-3342; Lode et al., (1998) Cancer Research 58:2925-2928). Calicheamicin has intracellular sites of action but, in certain instances, does not readily cross the plasma membrane. Therefore, cellular uptake of these agents through antibody-mediated internalization may, in some embodiments, greatly enhance their cytotoxic effects. Nonlimiting exemplary methods of preparing anti-CD79b antibody immunoconjugates with a calicheamicin drug moiety are described, for example, in U.S. Pat. Nos. 5,712,374; 5,714,586; 5,739,116; and 5,767,285.

### (4) Other Drug Moieties

In some embodiments, an anti-CD79b immunoconjugate comprises geldanamycin (Mandler et al (2000) J. Nat. Cancer Inst. 92(19):1573-1581; Mandler et al (2000) Bioorganic & Med. Chem. Letters 10:1025-1028; Mandler et al (2002) Bioconjugate Chem. 13:786-791); and/or enzymatically active toxins and fragments thereof, including, but not limited to, diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes. See, e.g., WO 93/21232.

Drug moieties also include compounds with nucleolytic activity (e.g., a ribonuclease or a DNA endonuclease).

In certain embodiments, an anti-CD79b immunoconjugate comprises a highly radioactive atom. A variety of radioactive isotopes are available for the production of radioconjugated antibodies. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. In some embodiments, when an anti-CD79b immunoconjugate is used for detection, it may comprise a radioactive atom for scintigraphic studies, for example Tc⁹⁹ or I¹²³, or a spin label for nuclear magnetic resonance (NMR) imaging (also known as magnetic resonance imaging, MRI), such as zirconium-89, iodine-123, iodine-131, indium-111, fluorine-19, carbon-13, nitrogen-15, oxygen-17, gadolinium, manganese or iron. Zirconium-89 may be complexed to various metal chelating agents and conjugated to antibodies, e.g., for PET imaging (WO 2011/056983).

The radio- or other labels may be incorporated in the anti-CD79b immunoconjugate in known ways. For example, a peptide may be biosynthesized or chemically synthesized using suitable amino acid precursors comprising, for example, one or more fluorine-19 atoms in place of one or more hydrogens. In some embodiments, labels such as Tc⁹⁹, I¹²³, Re¹⁸⁶, Re¹⁸⁸ and In¹¹¹ can be attached via a cysteine residue in the anti-CD79b antibody. In some embodiments, yttrium-90 can be attached via a lysine residue of the anti-CD79b antibody. In some embodiments, the IODOGEN method (Fraker et al (1978) Biochem. Biophys. Res. Commun. 80: 49-57 can be used to incorporate iodine-123. “Monoclonal Antibodies in Immunoscintigraphy” (Chatal, CRC Press 1989) describes certain other methods.

In certain embodiments, an anti-CD79b immunoconjugate may comprise an anti-CD79b antibody conjugated to a prodrug-activating enzyme. In some such embodiments, a prodrug-activating enzyme converts a prodrug (e.g., a peptidyl chemotherapeutic agent, see WO 81/01145) to an active drug, such as an anti-cancer drug. Such immunoconjugates are useful, in some embodiments, in antibody-dependent enzyme-mediated prodrug therapy (“ADEPT”). Enzymes that may be conjugated to an anti-CD79b antibody include, but are not limited to, alkaline phosphatases, which are useful for converting phosphate-containing prodrugs into free drugs; arylsulfatases, which are useful for converting sulfate-containing prodrugs into free drugs; cytosine deaminase, which is useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), which are useful for converting peptide-containing prodrugs into free drugs; D-alanylcarboxypeptidases, which are useful for converting prodrugs that contain D-amino acid substituents; carbohydrate-cleaving enzymes such as β-galactosidase and neuraminidase, which are useful for converting glycosylated prodrugs into free drugs; β-lactamase, which is useful for converting drugs derivatized with β-lactams into free drugs; and penicillin amidases, such as penicillin V amidase and penicillin G amidase, which are useful for converting drugs derivatized at their amine nitrogens with phenoxyacetyl or phenylacetyl groups, respectively, into free drugs. In some embodiments, enzymes may be covalently bound to antibodies by recombinant DNA techniques well known in the art. See, e.g., Neuberger et al., Nature 312:604-608 (1984).

### (iv) Drug Loading

Drug loading is represented by p, the average number of drug moieties per anti-CD79b antibody in a molecule of Formula I. Drug loading may range from 1 to 20 drug moieties (D) per antibody. Anti-CD79b immunoconjugates of Formula I include collections of anti-CD79b antibodies conjugated with a range of drug moieties, from 1 to 20. The average number of drug moieties per anti-CD79b antibody in preparations of anti-CD79b immunoconjugates from conjugation reactions may be characterized by conventional means such as mass spectroscopy, ELISA assay, and HPLC. The quantitative distribution of anti-CD79b immunoconjugates in terms of p may also be determined. In some instances, separation, purification, and characterization of homogeneous anti-CD79b immunoconjugates where p is a certain value from anti-CD79b immunoconjugates with other drug loadings may be achieved by means such as reverse phase HPLC or electrophoresis.

For some anti-CD79b immunoconjugates, p may be limited by the number of attachment sites on the anti-CD79b antibody. For example, where the attachment is a cysteine thiol, as in certain exemplary embodiments above, an anti-CD79b antibody may have only one or several cysteine thiol groups, or may have only one or several sufficiently reactive thiol groups through which a linker may be attached. In certain embodiments, higher drug loading, e.g., p>5, may cause aggregation, insolubility, toxicity, or loss of cellular permeability of certain anti-CD79b immunoconjugates. In certain embodiments, the average drug loading for an anti-CD79b immunoconjugates ranges from 1 to about 8; from about 2 to about 6; from about 3 to about 5; or from about 3 to about 4. Indeed, it has been shown that for certain antibody-drug conjugates, the optimal ratio of drug moieties per antibody may be less than 8, and may be about 2 to about 5 (U.S. Pat. No. 7,498,298). In certain embodiments, the optimal ratio of drug moieties per antibody is about 3 to about 4. In certain embodiments, the optimal ratio of drug moieties per antibody is about 3.5.

In certain embodiments, fewer than the theoretical maximum of drug moieties are conjugated to the anti-CD79b antibody during a conjugation reaction. An antibody may contain, for example, lysine residues that do not react with the drug-linker intermediate or linker reagent, as discussed below. Generally, antibodies do not contain many free and reactive cysteine thiol groups which may be linked to a drug moiety; indeed most cysteine thiol residues in antibodies exist as disulfide bridges. In certain embodiments, an anti-CD79b antibody may be reduced with a reducing agent such as dithiothreitol (DTT) or tricarbonylethylphosphine (TCEP), under partial or total reducing conditions, to generate reactive cysteine thiol groups. In certain embodiments, an anti-CD79b antibody is subjected to denaturing conditions to reveal reactive nucleophilic groups such as lysine or cysteine.

The loading (drug/antibody ratio) of an anti-CD79b immunoconjugate may be controlled in different ways, and for example, by: (i) limiting the molar excess of drug-linker intermediate or linker reagent relative to antibody, (ii) limiting the conjugation reaction time or temperature, and (iii) partial or limiting reductive conditions for cysteine thiol modification.

It is to be understood that where more than one nucleophilic group reacts with a drug-linker intermediate or linker reagent, then the resulting product is a mixture of anti-CD79b immunoconjugate compounds with a distribution of one or more drug moieties attached to an anti-CD79b antibody. The average number of drugs per antibody may be calculated from the mixture by a dual ELISA antibody assay, which is specific for antibody and specific for the drug. Individual anti-CD79b immunoconjugate molecules may be identified in the mixture by mass spectroscopy and separated by HPLC, e.g., hydrophobic interaction chromatography (see, e.g., McDonagh et al (2006) Prot. Engr. Design & Selection 19(7):299-307; Hamblett et al (2004) Clin. Cancer Res. 10:7063-7070; Hamblett, K. J., et al. “Effect of drug loading on the pharmacology, pharmacokinetics, and toxicity of an anti-CD30 antibody-drug conjugate,” Abstract No. 624, American Association for Cancer Research, 2004 Annual Meeting, Mar. 27-31, 2004, Proceedings of the AACR, Volume 45, March 2004; Alley, S. C., et al. “Controlling the location of drug attachment in antibody-drug conjugates,” Abstract No. 627, American Association for Cancer Research, 2004 Annual Meeting, March 27-31, 2004, Proceedings of the AACR, Volume 45, March 2004). In certain embodiments, a homogeneous anti-CD79b immunoconjugate with a single loading value may be isolated from the conjugation mixture by electrophoresis or chromatography.

### (v) Methods of Preparing Anti-CD79b Immunoconjugates

An anti-CD79b immunoconjugate of Formula I may be prepared by several routes employing organic chemistry reactions, conditions, and reagents known to those skilled in the art, including, but not limited to, e.g., (1) reaction of a nucleophilic group of an anti-CD79b antibody with a bivalent linker reagent to form Ab-L via a covalent bond, followed by reaction with a drug moiety D; and (2) reaction of a nucleophilic group of a drug moiety with a bivalent linker reagent, to form D-L, via a covalent bond, followed by reaction with a nucleophilic group of an anti-CD79b antibody. Exemplary methods for preparing an anti-CD79b immunoconjugate of Formula I via the latter route are described in U.S. Pat. No. 7,498,298, which is expressly incorporated herein by reference.

Nucleophilic groups on antibodies include, but are not limited to: (i) N-terminal amine groups, (ii) side chain amine groups, e.g., lysine, (iii) side chain thiol groups, e.g., cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; and (iii) aldehydes, ketones, carboxyl, and maleimide groups. Certain antibodies have reducible interchain disulfides, i.e. cysteine bridges. Anti-CD79b antibodies may be made reactive for conjugation with linker reagents by treatment with a reducing agent such as DTT (dithiothreitol) or tricarbonylethylphosphine (TCEP), such that the anti-CD79b antibody is fully or partially reduced. Each cysteine bridge will thus form, theoretically, two reactive thiol nucleophiles. Additional nucleophilic groups can be introduced into anti-CD79b antibodies through modification of lysine residues, e.g., by reacting lysine residues with 2-iminothiolane (Traut's reagent), resulting in conversion of an amine into a thiol. Reactive thiol groups may also be introduced into an anti-CD79b antibody by introducing one, two, three, four, or more cysteine residues (e.g., by preparing variant antibodies comprising one or more non-native cysteine amino acid residues).

Anti-CD79b immunoconjugates described herein may also be produced by reaction between an electrophilic group on an anti-CD79b antibody, such as an aldehyde or ketone carbonyl group, with a nucleophilic group on a linker reagent or drug. Useful nucleophilic groups on a linker reagent include, but are not limited to, hydrazide, oxime, amino, hydrazine, thiosemicarbazone, hydrazine carboxylate, and arylhydrazide. In one embodiment, an anti-CD79b antibody is modified to introduce electrophilic moieties that are capable of reacting with nucleophilic substituents on the linker reagent or drug. In another embodiment, the sugars of glycosylated anti-CD79b antibodies may be oxidized, e.g., with periodate oxidizing reagents, to form aldehyde or ketone groups which may react with the amine group of linker reagents or drug moieties. The resulting imine Schiff base groups may form a stable linkage, or may be reduced, e.g., by borohydride reagents to form stable amine linkages. In one embodiment, reaction of the carbohydrate portion of a glycosylated anti-CD79b antibody with either galactose oxidase or sodium meta-periodate may yield carbonyl (aldehyde and ketone) groups in the anti-CD79b antibody that can react with appropriate groups on the drug (Hermanson, Bioconjugate Techniques). In another embodiment, anti-CD79b antibodies containing N-terminal serine or threonine residues can react with sodium meta-periodate, resulting in production of an aldehyde in place of the first amino acid (Geoghegan & Stroh, (1992) Bioconjugate Chem. 3:138-146; U.S. Pat. No. 5,362,852). Such an aldehyde can be reacted with a drug moiety or linker nucleophile.

Exemplary nucleophilic groups on a drug moiety include, but are not limited to: amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups.

Nonlimiting exemplary cross-linker reagents that may be used to prepare anti-CD79b immunoconjugates are described herein in the section titled “Exemplary Linkers.” Methods of using such cross-linker reagents to link two moieties, including a proteinaceous moiety and a chemical moiety, are known in the art. In some embodiments, a fusion protein comprising an anti-CD79b antibody and a cytotoxic agent may be made, e.g., by recombinant techniques or peptide synthesis. A recombinant DNA molecule may comprise regions encoding the antibody and cytotoxic portions of the conjugate either adjacent to one another or separated by a region encoding a linker peptide which does not destroy the desired properties of the conjugate. In yet another embodiment, an anti-CD79b antibody may be conjugated to a “receptor” (such as streptavidin) for utilization in tumor pre-targeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a drug or radionucleotide). Additional details regarding anti-CD79b immunoconjugates are provided in U.S. Pat. No. 8,545,850 and WO/2016/049214, the contents of which are expressly incorporated by reference herein in their entirety.

In some aspects, provided herein are immunoconjugates comprising the formula:

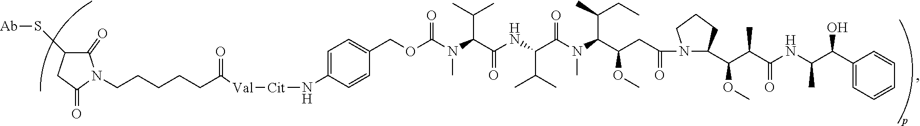

wherein Ab is an anti-CD79b antibody comprising (i) a hypervariable region-H1 (HVR-H1) that comprises the amino acid sequence of SEQ ID NO: 5; (ii) an HVR-H2 comprising the amino acid sequence of SEQ ID NO: 6; (iii) an HVR-H3 comprising the amino acid sequence of SEQ ID NO: 7; (iv) an HVR-L1 comprising the amino acid sequence of SEQ ID NO: 8; (v) an HVR-L2 comprising the amino acid sequence of SEQ ID NO: 9; and (vi) an HVR-L3 comprising the amino acid sequence of SEQ ID NO:10, and wherein p is between 1 and 8, for use in combination with an anti-CD20 antibody (e.g., rituximab or obinutuzumab), one or more chemotherapeutic agents (e.g., cyclophosphamide and/or doxorubicin), and a corticosteroid (e.g., prednisone, prednisolone, or methylprednisolone) for treating an individual, e.g., a human patient, in need thereof having diffuse large B-cell lymphoma (DLBCL; e.g., previously untreated DLBCL). In some embodiments, p is between 3 and 4. In some embodiments, the anti-CD79b antibody comprises (i) a heavy chain variable domain (VH) comprising the amino acid sequence of SEQ ID NO: 3 and (ii) a light chain variable domain (VL) comprising the amino acid sequence of SEQ ID NO: 4. In some embodiments, the antibody comprises (i) a heavy chain comprising the amino acid sequence of SEQ ID NO: 12 and (ii) a light chain comprising the amino acid sequence of SEQ ID NO: 11. In some embodiments, p is between 2 and 5. In some embodiments, p is 3.4 or 3.5. In some embodiments, the immunoconjugate is for use in a method described herein.

In some aspects, provided herein is a use of an immunoconjugate comprising the formula:

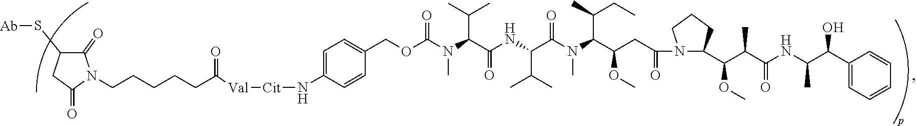

wherein Ab is an anti-CD79b antibody comprising (i) a hypervariable region-H1 (HVR-H1) that comprises the amino acid sequence of SEQ ID NO: 5; (ii) an HVR-H2 comprising the amino acid sequence of SEQ ID NO: 6; (iii) an HVR-H3 comprising the amino acid sequence of SEQ ID NO: 7; (iv) an HVR-L1 comprising the amino acid sequence of SEQ ID NO: 8; (v) an HVR-L2 comprising the amino acid sequence of SEQ ID NO: 9; and (vi) an HVR-L3 comprising the amino acid sequence of SEQ ID NO:10, and wherein p is between 1 and 8, for the manufacture of a medicament for treating an individual, e.g., a human patient, in need thereof having diffuse large B-cell lymphoma (DLBCL; e.g., previously untreated DLBCL), wherein the medicament is for (e.g., is formulated for) administration in combination with an anti-CD20 antibody (e.g., rituximab or obinutuzumab), one or more chemotherapeutic agents (e.g., cyclophosphamide and/or doxorubicin), and a corticosteroid (e.g., prednisone, prednisolone, or methylprednisolone). In some embodiments, p is between 2 and 5. In some embodiments, p is between 3 and 4. In some embodiments, the anti-CD79b antibody comprises (i) a heavy chain variable domain (VH) comprising the amino acid sequence of SEQ ID NO: 3 and (ii) a light chain variable domain (VL) comprising the amino acid sequence of SEQ ID NO: 4. In some embodiments, p is 3.4 or 3.5. In some embodiments, the antibody comprises (i) a heavy chain comprising the amino acid sequence of SEQ ID NO: 12 and (ii) a light chain comprising the amino acid sequence of SEQ ID NO: 11. In some embodiments, the immunoconjugate is polatuzumab vedotin. In some embodiments, the medicament (i.e., the medicament comprising the immunoconjugate) is for use in a method described herein.

In some aspects, provided herein are immunoconjugates comprising the formula:

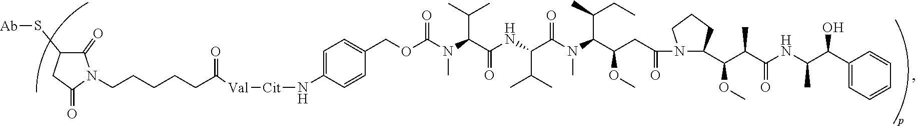

wherein Ab is an anti-CD79b antibody comprising (i) a heavy chain variable domain (VH) comprising the amino acid sequence of SEQ ID NO: 3 and (ii) a light chain variable domain (VL) comprising the amino acid sequence of SEQ ID NO: 4, and wherein p is between 2 and 5, for use in combination with an anti-CD20 antibody (e.g., rituximab or obinutuzumab), one or more chemotherapeutic agents (e.g., cyclophosphamide and/or doxorubicin), and a corticosteroid (e.g., prednisone, prednisolone, or methylprednisolone) for treating an individual, e.g., a human patient, in need thereof having diffuse large B-cell lymphoma (DLBCL; e.g., previously untreated DLBCL). In some embodiments, p is between 3 and 4. In some embodiments, p is 3.4 or 3.5. In some embodiments, the antibody comprises (i) a heavy chain comprising the amino acid sequence of SEQ ID NO: 12 and (ii) a light chain comprising the amino acid sequence of SEQ ID NO: 11. In some embodiments, the immunoconjugate is polatuzumab vedotin. In some embodiments, the immunoconjugate is for use in a method described herein.

In some aspects, provided herein is a use of an immunoconjugate comprising the formula:

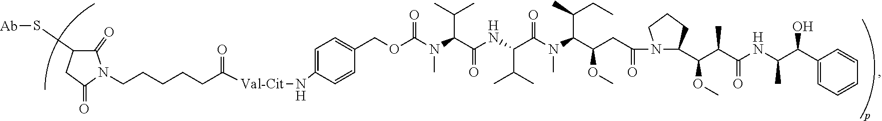

wherein Ab is an anti-CD79b antibody comprising (i) a heavy chain variable domain (VH) comprising the amino acid sequence of SEQ ID NO: 3 and (ii) a light chain variable domain (VL) comprising the amino acid sequence of SEQ ID NO: 4, and wherein p is between 2 and 5, for the manufacture of a medicament for treating an individual, e.g., a human patient, in need thereof having diffuse large B-cell lymphoma (DLBCL; e.g., previously untreated DLBCL), wherein the medicament is for (e.g., is formulated for) administration in combination with an anti-CD20 antibody (e.g., rituximab or obinutuzumab), one or more chemotherapeutic agents (e.g., cyclophosphamide and/or doxorubicin), and a corticosteroid (e.g., prednisone, prednisolone, or methylprednisolone). In some embodiments, p is between 3 and 4. In some embodiments, p is 3.4 or 3.5. In some embodiments, the antibody comprises (i) a heavy chain comprising the amino acid sequence of SEQ ID NO: 12 and (ii) a light chain comprising the amino acid sequence of SEQ ID NO: 11. In some embodiments, the immunoconjugate is polatuzumab vedotin. In some embodiments, the medicament (i.e., the medicament comprising the immunoconjugate) is for use in a method described herein.

In some aspects, provided herein is polatuzumab vedotin for use in combination with an anti-CD20 antibody (e.g., rituximab or obinutuzumab), one or more chemotherapeutic agents (e.g., cyclophosphamide and/or doxorubicin), and a corticosteroid (e.g., prednisone, prednisolone, or methylprednisolone) for treating an individual, e.g., a human patient, in need thereof having diffuse large B-cell lymphoma (DLBCL; e.g., previously untreated DLBCL). In some embodiments, the polatuzumab vedotin is for use in a method described herein.

In some aspects, provided herein is a use of polatuzumab vedotin for the manufacture of a medicament for treating an individual, e.g., a human patient, in need thereof having diffuse large B-cell lymphoma (DLBCL; e.g., previously untreated DLBCL), wherein the medicament is for (e.g., is formulated for) administration in combination with an anti-CD20 antibody (e.g., rituximab or obinutuzumab), one or more chemotherapeutic agents (e.g., cyclophosphamide and/or doxorubicin), and a corticosteroid (e.g., prednisone, prednisolone, or methylprednisolone). In some embodiments, the medicament (i.e., the medicament comprising the polatuzumab vedotin) is for use in a method described herein

### E. Exemplary Anti-CD20 Antibodies

### Type H Anti-CD20 Antibodies

In some instances, the above methods, compositions, and uses involve administration or use of type II anti-CD20 antibodies. In some instances, type II anti-CD20 antibodies mediate the death of cells that express CD20. In some instances, the type II anti-CD20 antibody is a monoclonal antibody. In some instances, the type II anti-CD20 antibody is an antibody fragment selected from the group consisting of Fab, Fab′-SH, Fv, scFv, and (Fab′)₂ fragments. In some instances, the type II anti-CD20 antibody is a humanized antibody. In some instances, the type II anti-CD20 antibody is a human antibody. In some instances, the type II anti-CD20 antibody described herein binds to human CD20. In some particular instances, the type II anti-CD20 antibody is obinutuzumab (see, e.g., WO 2005/044859 and U.S. Patent Publication No. 2005/0123546, which are incorporated by reference herein in entirety), which is incorporated by reference herein in its entirety). Obinutuzumab (Genentech) is also known as GAZYVA™/GAZYVARO™ and GA101.

In some instances, the type II anti-CD20 antibody comprises a heavy chain variable region (HVR-H) comprising an HVR-H1, HVR-H2, and HVR-H3 sequence, wherein: (a) the HVR-H1 sequence is GYAFSY (SEQ ID NO: 16); (b) the HVR-H2 sequence is FPGDGDTD (SEQ ID NO: 17); and (c) the HVR-H3 sequence is NVFDGYWLVY (SEQ ID NO: 18).

In some instances, the type II anti-CD20 antibody comprises a heavy chain variable region (HVR-H) comprising an HVR-H1, HVR-H2, and HVR-H3 sequence, wherein: (a) the HVR-H1 sequence is YSWIN (SEQ ID NO: 42); (b) the HVR-H2 sequence is RIFPGDGDTDYNGKFK (SEQ ID NO: 43); and (c) the HVR-H3 sequence is NVFDGYWLVY (SEQ ID NO: 18).

In some instances, the type II anti-CD20 antibody further comprises a light chain variable region (HVR-L) comprising an HVR-L1, HVR-L2, and HVR-L3 sequence, wherein: (a) the HVR-L1 sequence is RSSKSLLHSNGITYLY (SEQ ID NO: 19); (b) the HVR-L2 sequence is QMSNLVS (SEQ ID NO: 20); and (c) the HVR-L3 sequence is AQNLELPYT (SEQ ID NO: 21).

In some instances, the type II anti-CD20 antibody comprises a heavy chain and a light chain sequence, wherein: (a) the heavy chain variable (VH) region sequence comprises the amino acid sequence: QVQLVQSGAEVKKPGSSVKVSCKASGYAFSYSWINWVRQAPGQGLEWMGRIFPGDGDTDY NGKFKGRVTITADKSTSTAYMELSSLRSEDTAVYYCARNVFDGYWLVYWGQGTLVTVSS (SEQ ID NO: 22); and (b) the light chain variable (VL) region sequence comprises the amino acid sequence: DIVMTQTPLSLPVTPGEPASISCRSSKSLLHSNGITYLYWYLQKPGQSPQLLIYQMSNLVSGVP DRFSGSGSGTDFTLKISRVEAEDVGVYYCAQNLELPYTFGGGTKVEIKRTV (SEQ ID NO: 23).

In some instances, the type II anti-CD20 antibody comprises a heavy chain and a light chain sequence, wherein: (a) the heavy chain comprises the amino acid sequence: QVQLVQSGAEVKKPGSSVKVSCKASGYAFSYSWINWVRQAPGQGLEWMGRIFPGDGDTDY NGKFKGRVTITADKSTSTAYMELSSLRSEDTAVYYCARNVFDGYWLVYWGQGTLVTVSSAS TKGPSVFPLAPSSKSTSGGTAALGCLVKDYFPEPVTVSWNSGALTSGVHTFPAVLQSSGLYSL SSVVTVPSSSLGTQTYICNVNHKPSNTKVDKKVEPKSCDKTHTCPPCPAPELLGGPSVFLFPPK PKDTLMISRTPEVTCVVVDVSHEDPEVKFNWYVDGVEVHNAKTKPREEQYNSTYRVVSVLT VLHQDWLNGKEYKCKVSNKALPAPIEKTISKAKGQPREPQVYTLPPSRDELTKNQVSLTCLV KGFYPSDIAVEWESNGQPENNYKTTPPVLDSDGSFFLYSKLTVDKSRWQQGNVFSCSVMHE ALHNHYTQKSLSLSPG (SEQ ID NO: 24); and (b) the light chain comprises the amino acid sequence:

(SEQ ID NO: 25)
DIVMTQTPLSLPVTPGEPASISCRSSKSLLHSNGITYLYWYLQKPGQSPQ
LLIYQMSNLVSGVPDRFSGSGSGTDFTLKISRVEAEDVGVYYCAQNLELP
YTFGGGTKVEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

In some instances, the type II anti-CD20 antibody comprises (a) a VH domain comprising an amino acid sequence comprising having at least 95% sequence identity (e.g., at least 95%, 96%, 97%, 98%, or 99% sequence identity) to, or the sequence of (SEQ ID NO: 22); (b) a VL domain comprising an amino acid sequence comprising having at least 95% sequence identity (e.g., at least 95%, 96%, 97%, 98%, or 99% sequence identity) to, or the sequence of (SEQ ID NO: 23); or (c) a VH domain as in (a) and a VL domain as in (b). In another instances, a type II anti-CD20 antibody is provided, wherein the antibody comprises a VH as in any of the instances provided above, and a VL as in any of the instances provided above, wherein one or both of the variable domain sequences include post-translational modifications.

In certain instances, a type II anti-CD20 antibody may bind to CD20 on the surface of a lymphoma cell and induce apoptosis. In certain instances, a type II anti-CD20 antibody that binds to CD20 has a dissociation constant (K_{D}) of ≤1 M, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹¹ M, e.g., from 10⁻⁹ M to 10⁻¹¹ M). In certain instances, a type II anti-CD20 antibody that binds to CD20 has a K_{D} of <50 nM. In certain instances, a type II anti-CD20 antibody that binds to CD20 has a K_{D} of <20 nM. In certain instances, a type II anti-CD20 antibody that binds to CD20 has a K_{D} of <10 nM. In certain instances, the binding is at a K_{D} of <7.5 nM, <5 nM, between 1-5 nM, or <1 nM. In certain instances, the type II anti-CD20 antibody may bind to both human CD20 and cyno CD20.

In some instances, the methods or uses described herein may include using or administering an isolated anti-CD20 antibody that competes for binding to CD20 with any of the type II anti-CD20 antibodies described above. For example, the method may include administering an isolated anti-CD20 antibody that competes for binding to CD20 with a type II anti-CD20 antibody having the following six HVRs: (a) an HVR-H1 comprising the amino acid sequence of GYAFSY (SEQ ID NO: 16); (b) an HVR-H2 comprising the amino acid sequence of FPGDGDTD (SEQ ID NO: 17); (c) an HVR-H3 comprising the amino acid sequence of NVFDGYWLVY (SEQ ID NO: 18); (d) an HVR-L1 comprising the amino acid sequence of RSSKSLLHSNGITYLY (SEQ ID NO: 19), (e) an HVR-L2 comprising the amino acid sequence of QMSNLVS (SEQ ID NO: 20); and (f) an HVR-L3 comprising the amino acid sequence of AQNLELPYT (SEQ ID NO: 21). In another example, the method may include administering an isolated anti-CD20 antibody that competes for binding to CD20 with a type II anti-CD20 antibody having the following six HVRs: (a) an HVR-H1 comprising the amino acid sequence of YSWIN (SEQ ID NO: 42); (b) an HVR-H2 comprising the amino acid sequence of RIFPGDGDTDYNGKFK (SEQ ID NO: 43); (c) an HVR-H3 comprising the amino acid sequence of NVFDGYWLVY (SEQ ID NO: 18); (d) an HVR-L1 comprising the amino acid sequence of RSSKSLLHSNGITYLY (SEQ ID NO: 19), (e) an HVR-L2 comprising the amino acid sequence of QMSNLVS (SEQ ID NO: 20); and (f) an HVR-L3 comprising the amino acid sequence of AQNLELPYT (SEQ ID NO: 21). The methods described herein may also include administering an isolated anti-CD20 antibody that binds to the same epitope as a type II anti-CD20 antibody described above.

### Type I Anti-CD20 Antibodies

In some instances, the above methods, compositions, and uses involve administration or use of type I anti-CD20 antibodies. In some instances, type I anti-CD20 antibodies mediate the death of cells that express CD20. In some instances, the type I anti-CD20 antibody is a monoclonal antibody. In some instances, the type I anti-CD20 antibody is an antibody fragment selected from the group consisting of Fab, Fab′-SH, Fv, scFv, and (Fab′)₂ fragments. In some instances, the type I anti-CD20 antibody is a humanized antibody. In some instances, the type I anti-CD20 antibody is a human antibody. In some instances, the type I anti-CD20 antibody described herein binds to human CD20. In some particular instances, the type I anti-CD20 antibody is rituximab (RITUXAN®).

The terms “rituximab” or “RITUXAN®” herein refer to the genetically engineered chimeric murine/human monoclonal antibody directed against the CD20 antigen and designated “C2B8” in U.S. Pat. No. 5,736,137, which is incorporated herein by reference in its entirety. The antibody is an IgG1 kappa immunoglobulin containing murine light and heavy chain variable region sequences and human constant region sequences. Rituximab has a binding affinity for the CD20 antigen of approximately 8.0 nM.

In certain instances, the type I anti-CD20 antibodies includes at least one, two, three, four, five, or six HVRs selected from: (a) an HVR-H1 comprising the amino acid sequence of SYNMH (SEQ ID NO: 26); (b) an HVR-H2 comprising the amino acid sequence of AIYPGNGDTSYNQKFKG (SEQ ID NO: 27); (c) an HVR-H3 comprising the amino acid sequence of STYYGGDWYFNV (SEQ ID NO: 28); (d) an HVR-L1 comprising the amino acid sequence of RASSSVSYIH (SEQ ID NO: 29), (e) an HVR-L2 comprising the amino acid sequence of ATSNLAS (SEQ ID NO: 30); and/or (f) an HVR-L3 comprising the amino acid sequence of QQWTSNPPT (SEQ ID NO: 31), or a combination of one or more of the above HVRs and one or more variants thereof having at least about 90% sequence identity (e.g., 90%, 91%, 92%, 93%, 94%, 95%, 96%, 97%, 98%, or 99% identity) to any one of SEQ ID NOs: 26-31.

In some instances, any of the above type I anti-CD20 antibodies includes (a) an HVR-H1 comprising the amino acid sequence of SYNMH (SEQ ID NO: 26); (b) an HVR-H2 comprising the amino acid sequence of AIYPGNGDTSYNQKFKG (SEQ ID NO: 27); (c) an HVR-H3 comprising the amino acid sequence of STYYGGDWYFNV (SEQ ID NO: 28); (d) an HVR-L1 comprising the amino acid sequence of RASSSVSYIH (SEQ ID NO: 29); (e) an HVR-L2 comprising the amino acid sequence of ATSNLAS (SEQ ID NO: 30); and (f) an HVR-L3 comprising the amino acid sequence of QQWTSNPPT (SEQ ID NO: 31).

In some instances, the type I anti-CD20 antibody further comprises at least one, two, three, or four of the following light chain variable region framework regions (FRs): an FR-L1 comprising the amino acid sequence of QIVLSQSPAILSASPGEKVTMTC (SEQ ID NO: 32); an FR-L2 comprising the amino acid sequence of WFQQKPGSSPKPWIY (SEQ ID NO: 33); an FR-L3 comprising the amino acid sequence of GVPVRFSGSGSGTSYSLTISRVEAEDAATYYC (SEQ ID NO: 34); and/or an FR-L4 comprising the amino acid sequence of FGGGTKLEIK (SEQ ID NO: 35), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 32-35. In some instances, for example, the antibody further comprises an FR-L1 comprising the amino acid sequence of QIVLSQSPAILSASPGEKVTMTC (SEQ ID NO: 32); an FR-L2 comprising the amino acid sequence of WFQQKPGSSPKPWIY (SEQ ID NO: 33); an FR-L3 comprising the amino acid sequence of GVPVRFSGSGSGTSYSLTISRVEAEDAATYYC (SEQ ID NO: 34); and an FR-L4 comprising the amino acid sequence of FGGGTKLEIK (SEQ ID NO: 35).

In some instances, the type I anti-CD20 antibody further comprises at least one, two, three, or four of the following heavy chain variable region FRs: an FR-H1 comprising the amino acid sequence of QVQLQQPGAELVKPGASVKMSCKASGYTFT (SEQ ID NO: 36); an FR-H2 comprising the amino acid sequence of WVKQTPGRGLEWIG (SEQ ID NO: 37); an FR-H3 comprising the amino acid sequence of KATLTADKSSSTAYMQLSSLTSEDSAVYYCAR (SEQ ID NO: 38); and/or an FR-H4 comprising the amino acid sequence of WGAGTTVTVSA (SEQ ID NO: 39), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 36-39. In some instances, the type I anti-CD20 antibody includes an FR-H1 comprising the amino acid sequence of QVQLQQPGAELVKPGASVKMSCKASGYTFT (SEQ ID NO: 36); an FR-H2 comprising the amino acid sequence of WVKQTPGRGLEWIG (SEQ ID NO: 37); an FR-H3 comprising the amino acid sequence of KATLTADKSSSTAYMQLSSLTSEDSAVYYCAR (SEQ ID NO: 38); and an FR-H4 comprising the amino acid sequence of WGAGTTVTVSA (SEQ ID NO: 39).

In some instances, the type I anti-CD20 antibody has a VH domain comprising an amino acid sequence having at least at least 90% sequence identity (e.g., at least 91%, 92%, 93%, 94%, 95%, 96%, 97%, 98%, or 99% sequence identity) to, or the sequence of QVQLQQPGAELVKPGASVKMSCKASGYTFTSYNMHWVKQTPGRGLEWIGAIYPGNGDTSY NQKFKGKATLTADKSSSTAYMQLSSLTSEDSAVYYCARSTYYGGDWYFNVWGAGTTVTVS A (SEQ ID NO: 40) and/or a VL domain comprising an amino acid sequence having at least 90% sequence identity (e.g., at least 91%, 92%, 93%, 94%, 95%, 96%, 97%, 98%, or 99% sequence identity) to, or the sequence of

(SEQ ID NO: 41)
QIVLSQSPAILSASPGEKVTMTCRASSSVSYIHWFQQKPGSSPKPWIYAT
SNLASGVPVRESGSGSGTSYSLTISRVEAEDAATYYCQQWTSNPPTFGGG
TKLEIK.

In another instances, a type I anti-CD20 antibody is provided, wherein the antibody comprises a VH as in any of the instances provided above, and a VL as in any of the instances provided above, wherein one or both of the variable domain sequences include post-translational modifications.

In certain instances, a type I anti-CD20 antibody may bind to CD20 on the surface of a lymphoma cell and mediate cell lysis through the activation of complement-dependent lysis, antibody-dependent cellular cytotoxicity (ADCC), and apoptosis mediated by Fc cross-linking. In certain instances, a type I anti-CD20 antibody that binds to CD20 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸M or less, e.g., from 10⁻⁸ M to 10⁻¹³ M, e.g., from 10⁻⁹ M to 10⁻¹³ M). In certain instances, a type I anti-CD20 antibody that binds to CD20 has a K_{D} of <10 nM. In certain instances, the binding is at a K_{D} of <7.5 nM, <5 nM, between 1-5 nM, or <1 nM. In certain instances, the type I anti-CD20 antibody may bind to both human CD20 and cyno CD20.

In some instances, the methods or uses described herein may include using or administering an isolated anti-CD20 antibody that competes for binding to CD20 with any of the type I anti-CD20 antibodies described above. For example, the method may include administering an isolated anti-CD20 antibody that competes for binding to CD20 with a type I anti-CD20 antibody having the following six HVRs: (a) an HVR-H1 comprising the amino acid sequence of SYNMH (SEQ ID NO: 26); (b) an HVR-H2 comprising the amino acid sequence of AIYPGNGDTSYNQKFKG (SEQ ID NO: 27); (c) an HVR-H3 comprising the amino acid sequence of STYYGGDWYFNV (SEQ ID NO: 28); (d) an HVR-L1 comprising the amino acid sequence of RASSSVSYIH (SEQ ID NO: 29), (e) an HVR-L2 comprising the amino acid sequence of ATSNLAS (SEQ ID NO: 30); and (f) an HVR-L3 comprising the amino acid sequence of QQWTSNPPT (SEQ ID NO: 31). The methods described herein may also include administering an isolated anti-CD20 antibody that binds to the same epitope as a type I anti-CD20 antibody described above.

In a further instance, an anti-CD20 antibody according to any of the above instances (e.g., a type II or a type I anti-CD20 antibody) may incorporate any of the features, singly or in combination, as described in Sections (i)-(v) below.

### F. Antibodies

In some embodiments, an anti-CD20 antibody or an anti-CD79b antibody used in a method of treatment provided herein has a dissociation constant (Kd) for binding CD20 (e.g., human CD20) or CD79b (e.g., human CD79b), respectively, of ≤1 M, ≤100 nM, ≤50 nM, ≤10 nM, ≤5 nM, ≤1 nM, ≤0.1 nM, ≤0.01 nM, or ≤0.001 nM, and optionally is ≥10⁻¹³ M (e.g., 10⁻⁸ M or less, e.g., from 10⁻⁸ M to 10⁻¹³ M, e.g., from 10⁻⁹ M to 10⁻³ M). In some embodiments, the anti-CD20 antibody or anti-CD79b antibody is an antibody fragment. In some embodiments, the anti-CD20 antibody or anti-CD79b antibody is a chimeric or a humanized antibody. In some embodiments, the anti-CD20 antibody or anti-CD79b antibody is a human antibody. In some embodiments, the anti-CD20 antibody or anti-CD79b antibody is a multispecific antibody, e.g., a bispecific antibody.

In certain embodiments, amino acid sequence variants of an anti-CD79b antibody or an anti-CD20 antibody used in a method of treatment provided herein are contemplated. For example, it may be desirable to improve the binding affinity and/or other biological properties of the anti-CD79b antibody or anti-CD20 antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### (i) Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table under the heading of “preferred substitutions.” More substantial changes are provided in Table under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 4

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g., binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex is used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

### (ii) Glycosylation Variants

In certain embodiments, an antibody (e.g., an anti-CD79b antibody or an anti-CD20 antibody) used in a method of treatment provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

(iii) Fc Variants

In certain embodiments, one or more amino acid modifications may be introduced into the Fc region of an antibody (e.g., an anti-CD79b antibody or an anti-CD20 antibody) used in a method of treatment provided herein, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions.

In certain embodiments, the invention contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96© non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

### (iv) Cysteine Engineered Antibody Variants

In certain embodiments, it may be desirable to create cysteine engineered antibodies, e.g., “thioMAbs,” in which one or more residues of an anti-CD79b antibody or an anti-CD20 antibody used in a method of treatment provided herein are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

### (v) Antibody Derivatives

In certain embodiments, an antibody (e.g., an anti-CD79b antibody or an anti-CD20 antibody) used in a method of treatment provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### G. Administration of an Anti-CD79b Immunoconjugate and an Anti-CD20 Antibody

Anti-CD79b immunoconjugates and additional therapeutic agents (e.g., an anti-CD20 antibody, one or more chemotherapeutic agents, and a corticosteroid) provided herein for use in any of the therapeutic methods described herein would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The immunoconjugate need not be, but is optionally formulated with one or more agents currently used to prevent or treat the disorder in question. The amount of the anti-CD79b immunoconjugate and the additional therapeutic agents (e.g., an anti-CD20 antibody, one or more chemotherapeutic agents, and a corticosteroid), and the timing of co-administration will depend on the type (species, gender, age, weight, etc.) and condition of the patient being treated and the severity of the disease or condition being treated. The anti-CD79b immunoconjugate and the additional therapeutic agents (e.g., an anti-CD20 antibody, one or more chemotherapeutic agents, and a corticosteroid) are suitably co-administered to the patient at one time or over a series of treatments, e.g., according to any of the treatment regimens described below.

In some embodiments, the dosage of the anti-CD79b immunoconjugate (such as huMA79bv28-MC-vc-PAB-MMAE or polatuzumab vedotin) is between 1.0-1.8 mg/kg. In some embodiments of any of the methods, the dosage of anti-CD79b immunoconjugate is about any of 1.0, 1.4, 1.5, 1.6, 1.7, or 1.8 mg/kg. In some embodiments, the dosage of anti-CD79b immunoconjugate is about 1.0 mg/kg. In some embodiments, the dosage of anti-CD79b immunoconjugate is about 1.4 mg/kg. In some embodiments, the dosage of anti-CD79b immunoconjugate is about 1.8 mg/kg. In some embodiments of any of the methods, the anti-CD79b immunoconjugate is administered q3w (i.e., once every 3 weeks). In some embodiments of any of the methods, the anti-CD79b immunoconjugate is administered once every 21 days. In some embodiments, the anti-CD79b immunoconjugate is administered via intravenous infusion. In some embodiments, the dosage administered via infusion is in the range of about 1 mg to about 1,500 mg per dose. Alternatively, the dosage range is of about 1 mg to about 1,500 mg, about 1 mg to about 1,000 mg, about 400 mg to about 1200 mg, about 600 mg to about 1000 mg, about 10 mg to about 500 mg, about 10 mg to about 300 mg, about 10 mg to about 200 mg, and about 1 mg to about 200 mg. In some embodiments, the dosage administered via infusion is in the range of about 1 μg/m² to about 10,000 μg/m² per dose. Alternatively, the dosage range is of about 1 μg/m² to about 1000 μg/m2, about 1 μg/m² to about 800 μg/m², about 1 μg/m² to about 600 μg/m2, about 1 μg/m² to about 400 μg/m², about 10 μg/m² to about 500 μg/m², about 10 μg/m² to about 300 μg/m², about 10 μg/m² to about 200 μg/m², and about 1 μg/m² to about 200 μg/m². The dose may be administered once per day, once per week, multiple times per week, but less than once per day, multiple times per month but less than once per day, multiple times per month but less than once per week, once per month, once every 21 days, or intermittently to relieve or alleviate symptoms of the disease. In some embodiments, the dosage of the immunoconjugate is 1.8 mg/kg, administered in 21-day cycles. In some embodiments, the dosage of the immunoconjugate is 1.8 mg/kg, administered on day 1 of each 21-day cycle. Administration may continue at any of the disclosed intervals until remission of the tumor or symptoms of the B-cell proliferative disorder being treated. Administration may continue after remission or relief of symptoms is achieved where such remission or relief is prolonged by such continued administration.

In some embodiments, the immunoconjugate is polatuzumab vedotin. In some embodiments, the polatuzumab vedotin is administered at a dose of about 1.0-1.8 mg/kg (e.g., 1.0 mg/kg, 1.4 mg/kg, or 1.8 mg/kg). In some embodiments, the polatuzumab vedotin is administered at a dose of about 1.0 mg/kg. In some embodiments, the polatuzumab vedotin is administered at a dose of about 1.4 mg/kg. In some embodiments, the polatuzumab vedotin is administered at a dose of about 1.8 mg/kg. In some embodiments, the polatuzumab vedotin is administered intravenously. In some embodiments, the polatuzumab vedotin is administered in 21-day cycles. In some embodiments, the polatuzumab vedotin is administered on day 1 of each 21-day cycle. In some embodiments, the polatuzumab vedotin is administered for between one and six 21-day cycles, e.g., any of 1, 2, 3, 4, 5, or 6 21-day cycles. In some embodiments, the polatuzumab vedotin is administered for at least six 21-day cycles. In some embodiments, the polatuzumab vedotin is administered for six 21-day cycles. In some embodiments, the polatuzumab vedotin is administered at a dose of about 1.0-1.8 mg/kg (e.g., 1.0 mg/kg, 1.4 mg/kg, or 1.8 mg/kg) on day 1 of each 21-day cycle for at least six cycles. In some embodiments, the polatuzumab vedotin is administered at a dose of about 1.0-1.8 mg/kg (e.g., 1.0 mg/kg, 1.4 mg/kg, or 1.8 mg/kg) on day 1 of each 21-day cycle for six cycles.

In some embodiments, the dosage of the anti-CD20 agent (e.g., an anti-CD20 antibody, such as rituximab or obinutuzumab) is between about 300-1600 mg/m² and/or 300-2000 mg. In some embodiments, the dosage of the anti-CD20 antibody is about any of 300, 375, 600, 1000, or 1250 mg/m² and/or 300, 1000, or 2000 mg. In some embodiments, the anti-CD20 antibody is rituximab and the dosage administered is 375 mg/m². In some embodiments, the anti-CD20 antibody is obinutuzumab and the dosage administered is 1000 mg. In some embodiments, the anti-CD20 antibody is administered q3w (i.e., every 3 weeks). In some embodiments, the anti-CD20 antibody is administered once every 21 days. In some embodiments, the dosage of an afucosylated anti-CD20 antibody (preferably the afucosylated humanized B-Ly1 antibody) may be 800 to 1600 mg (in one embodiment 800 to 1200 mg, such as 1000 mg). In some embodiments, the dose is a flat 1000 mg dose. In some embodiments, the dosage of rituximab is 375 mg/m², administered on day 1 of each 21-day cycle. In some embodiments, the anti-CD20 antibody is administered via intravenous infusion.

In some embodiments, the anti-CD20 antibody is rituximab. In some embodiments, the rituximab is administered at a dose of about 375 mg/m². In some embodiments, the rituximab is administered intravenously. In some embodiments, the rituximab is administered in 21-day cycles. In some embodiments, the rituximab is administered on day 1 of each 21-day cycle. In some embodiments, the rituximab is administered for between one and eight 21-day cycles, e.g., any of 1, 2, 3, 4, 5, 6, 7, or 8 21-day cycles. In some embodiments, the rituximab is administered for between six and eight 21-day cycles, e.g., any of 6, 7, or 8 21-day cycles. In some embodiments, the rituximab is administered for at least six 21-day cycles. In some embodiments, the rituximab is administered for six 21-day cycles. In some embodiments, the rituximab is administered for seven 21-day cycles. In some embodiments, the rituximab is administered for eight 21-day cycles. In some embodiments, the rituximab is administered for up to eight 21-day cycles. In some embodiments, the rituximab is administered at a dose of about 375 mg/m² on day 1 of each 21-day cycle for at least six cycles. In some embodiments, the rituximab is administered at a dose of about 375 mg/m² on day 1 of each 21-day cycle for six cycles, seven cycles, or eight cycles.

In some embodiments, the one or more chemotherapeutic agents comprise cyclophosphamide. In some embodiments, the dosage of cyclophosphamide is between about 375 mg/m² and about 750 mg/m², between about 375 mg/m² and about 563 mg/m², or between about 563 mg/m² and about 750 mg/m². In some embodiments, the dosage of cyclophosphamide is about 375 mg/m². In some embodiments, the dosage of cyclophosphamide is about 563 mg/m². In some embodiments, the dosage of cyclophosphamide is about 750 mg/m². In some embodiments, the cyclophosphamide is administered q3w (i.e., every 3 weeks). In some embodiments, the cyclophosphamide is administered once every 21 days. In some embodiments, the cyclophosphamide is administered on day 1 of each 21-day cycle. In some embodiments, cyclophosphamide is administered via intravenous infusion. In some embodiments, the cyclophosphamide is administered for between one and eight 21-day cycles, e.g., any of 1, 2, 3, 4, 5, 6, 7, or 8 21-day cycles. In some embodiments, the cyclophosphamide is administered for between six and eight 21-day cycles, e.g., any of 6, 7, or 8 21-day cycles. In some embodiments, the cyclophosphamide is administered for at least six 21-day cycles. In some embodiments, the cyclophosphamide is administered for six 21-day cycles. In some embodiments, the cyclophosphamide is administered for seven 21-day cycles. In some embodiments, the cyclophosphamide is administered for eight 21-day cycles. In some embodiments, the cyclophosphamide is administered for up to eight 21-day cycles. In some embodiments, the cyclophosphamide is administered at a dose of between about 375 mg/m² and about 750 mg/m² (e.g., 375 mg/m², 562.5 mg/m², or 750 mg/m²) on day 1 of each 21-day cycle for at least six cycles. In some embodiments, the cyclophosphamide is administered at a dose of between about 375 mg/m² and about 750 mg/m² (e.g., 375 mg/m², 562.5 mg/m², or 750 mg/m²) on day 1 of each 21-day cycle for six cycles, seven cycles, or eight cycles.

In some embodiments, the one or more chemotherapeutic agents comprise doxorubicin. In some embodiments, the dosage of the doxorubicin is between about 25 mg/m² and about 50 mg/m², between about 25 mg/m² and about 38 mg/m², or between about 38 mg/m² and about 50 mg/m². In some embodiments, the dosage of the doxorubicin is about 25 mg/m². In some embodiments, the dosage of the doxorubicin is about 38 mg/m². In some embodiments, the dosage of the doxorubicin is about 50 mg/m². In some embodiments, the doxorubicin is administered q3w (i.e., every 3 weeks). In some embodiments, doxorubicin is administered once every 21 days. In some embodiments, the doxorubicin is administered on day 1 of each 21-day cycle. In some embodiments, doxorubicin is administered via intravenous infusion. In some embodiments, the doxorubicin is administered for between one and eight 21-day cycles, e.g., any of 1, 2, 3, 4, 5, 6, 7, or 8 21-day cycles. In some embodiments, the doxorubicin is administered for between six and eight 21-day cycles, e.g., any of 6, 7, or 8 21-day cycles. In some embodiments, the doxorubicin is administered for at least six 21-day cycles. In some embodiments, the doxorubicin is administered for six 21-day cycles. In some embodiments, the doxorubicin is administered for seven 21-day cycles. In some embodiments, the doxorubicin is administered for eight 21-day cycles. In some embodiments, the doxorubicin is administered for up to eight 21-day cycles. In some embodiments, the doxorubicin is administered at a dose of between about 25 mg/m² and about 50 mg/m² (e.g., 25 mg/m², 37.5 mg/m², or 50 mg/m²) on day 1 of each 21-day cycle for at least six cycles. In some embodiments, the doxorubicin is administered at a dose of between about 25 mg/m² and about 50 mg/m² (e.g., 25 mg/m², 37.5 mg/m², or 50 mg/m²) on day 1 of each 21-day cycle for six cycles, seven cycles, or eight cycles.

In some embodiments, the dosage of the corticosteroid is between about 50 mg and about 100 mg, between about 50 mg and about 80 mg, or between about 80 mg and about 100 mg. In some embodiments, the dosage of the corticosteroid is about 50 mg. In some embodiments, the dosage of the corticosteroid is about 80 mg. In some embodiments, the dosage of the corticosteroid is about 100 mg. In some embodiments, the corticosteroid is administered in 21-day cycles. In some embodiments, the corticosteroid is administered on days 1, 2, 3, 4, and 5 of each 21-day cycle. In some embodiments, the corticosteroid is administered via intravenous infusion or orally. In some embodiments, the corticosteroid is prednisone. In some embodiments, the dosage of prednisone is about 100 mg. In some embodiments, the prednisone is administered on days 1, 2, 3, 4, and 5 of each 21-day cycle at a dose of about 100 mg per day. In some embodiments, the prednisone is administered orally. In some embodiments, the corticosteroid is prednisolone. In some embodiments, the dosage of prednisolone is about 100 mg. In some embodiments, the prednisolone is administered on days 1, 2, 3, 4, and 5 of each 21-day cycle at a dose of about 100 mg per day. In some embodiments, the prednisolone is administered orally. In some embodiments, the corticosteroid is methylprednisolone. In some embodiments, the dosage of methylprednisolone is about 80 mg. In some embodiments, the methylprednisolone is administered on days 1, 2, 3, 4, and 5 of each 21-day cycle at a dose of about 80 mg per day. In some embodiments, the methylprednisolone is administered intravenously. In some embodiments, the corticosteroid (e.g., prednisone, prednisolone, or methylprednisolone) is administered for between one and eight 21-day cycles, e.g., any of 1, 2, 3, 4, 5, 6, 7, or 8 21-day cycles. In some embodiments, the corticosteroid (e.g., prednisone, prednisolone, or methylprednisolone) is administered for between six and eight 21-day cycles, e.g., any of 6, 7, or 8 21-day cycles. In some embodiments, the corticosteroid (e.g., prednisone, prednisolone, or methylprednisolone) is administered for at least six 21-day cycles. In some embodiments, the corticosteroid (e.g., prednisone, prednisolone, or methylprednisolone) is administered for six 21-day cycles. In some embodiments, the corticosteroid (e.g., prednisone, prednisolone, or methylprednisolone) is administered for seven 21-day cycles. In some embodiments, the corticosteroid (e.g., prednisone, prednisolone, or methylprednisolone) is administered for eight 21-day cycles. In some embodiments, the prednisone is administered on days 1, 2, 3, 4, and 5 of each 21-day cycle at a dose of about 100 mg per day for at least six cycles. In some embodiments, the prednisone is administered on days 1, 2, 3, 4, and 5 of each 21-day cycle at a dose of about 100 mg per day for six cycles, seven cycles, or eight cycles. In some embodiments, the prednisolone is administered on days 1, 2, 3, 4, and 5 of each 21-day cycle at a dose of about 100 mg per day for at least six cycles. In some embodiments, the prednisolone is administered on days 1, 2, 3, 4, and 5 of each 21-day cycle at a dose of about 100 mg per day for six cycles, seven cycles, or eight cycles. In some embodiments, the methylprednisolone is administered on days 1, 2, 3, 4, and 5 of each 21-day cycle at a dose of about 80 mg per day for at least six cycles. In some embodiments, the methylprednisolone is administered on days 1, 2, 3, 4, and 5 of each 21-day cycle at a dose of about 80 mg per day for six cycles, seven cycles, or eight cycles.

An exemplary dosing regimen for the combination therapy of anti-CD79b immunoconjugates (such as huMA79bv28-MC-vc-PAB-MMAE or polatuzumab vedotin) and one or more additional therapeutic agents includes the anti-CD79b immunoconjugate (such as huMA79bv28-MC-vc-PAB-MMAE or polatuzumab vedotin) administered at a dose of about 1.0-1.8 mg/kg (e.g., 1.0 mg/kg, 1.4 mg/kg, or 1.8 mg/kg) once every 21 days, e.g., on day 1 of each 21-day cycle; rituximab at a dose of about 375 mg/m² once every 21 days, e.g., on day 1 of each 21-day cycle; cyclophosphamide at a dose of between about 375 mg/m² and about 750 mg/m² (e.g., 375 mg/m², 562.5 mg/m², or 750 mg/m²) once every 21 days, e.g., on day 1 of each 21-day cycle; doxorubicin at a dose of between about 25 mg/m² and about 50 mg/m² (e.g., 25 mg/m², 37.5 mg/m², or 50 mg/m²) once every 21 days, e.g., on day 1 of each 21-day cycle; and a corticosteroid (e.g., prednisone at a dose of about 100 mg, prednisolone at a dose of about 100 mg, or methylprednisolone at a dose of about 80 mg) on days 1-5 of each 21-day cycle. In some embodiments, the anti-CD79b immunoconjugate is administered at a dose of about any of 1.0 mg/kg, 1.4 mg/kg, or 1.8 mg/kg. In some embodiments, the anti-CD79b immunoconjugate is administered at a dose of about 1.0 mg/kg. In some embodiments, the anti-CD79b immunoconjugate is administered at a dose of about 1.4 mg/kg. In some embodiments, the anti-CD79b immunoconjugate is administered at a dose of about 1.8 mg/kg. In some embodiments, the cyclophosphamide is administered at a dose of about 750 mg/m². In some embodiments, the doxorubicin is administered at a dose of about 50 mg/m². In some embodiments, the corticosteroid is prednisone administered at a dose of about 100 mg. In some embodiments, the corticosteroid is prednisolone administered at a dose of about 100 mg. In some embodiments, the corticosteroid is methylprednisolone administered at a dose of about 80 mg.

Another exemplary dosing regimen for the combination therapy of anti-CD79b immunoconjugates (such as huMA79bv28-MC-vc-PAB-MMAE or polatuzumab vedotin) and one or more additional therapeutic agents includes the anti-CD79b immunoconjugate (such as huMA79bv28-MC-vc-PAB-MMAE or polatuzumab vedotin) administered at a dose of about 1.0-1.8 mg/kg (e.g., 1.0 mg/kg, 1.4 mg/kg, or 1.8 mg/kg) once every 21 days, e.g., on day 1 of each 21-day cycle; rituximab at a dose of about 375 mg/m² once every 21 days, e.g., on day 1 of each 21-day cycle; cyclophosphamide once every 21 days, e.g., on day 1 of each 21-day cycle; doxorubicin once every 21 days, e.g., on day 1 of each 21-day cycle; and a corticosteroid (e.g., prednisone, prednisolone, or methylprednisolone) on days 1-5 of each 21-day cycle. In some embodiments, the anti-CD79b immunoconjugate is administered at a dose of about any of 1.0 mg/kg, 1.4 mg/kg, or 1.8 mg/kg.

Another exemplary dosing regimen for the combination therapy of anti-CD79b immunoconjugates (such as huMA79bv28-MC-vc-PAB-MMAE or polatuzumab vedotin) and one or more additional therapeutic agents includes the anti-CD79b immunoconjugate (such as huMA79bv28-MC-vc-PAB-MMAE or polatuzumab vedotin) administered at a dose of about 1.0-1.8 mg/kg (e.g., 1.0 mg/kg, 1.4 mg/kg, or 1.8 mg/kg) once every 21 days, e.g., on day 1 of each 21-day cycle; obinutuzumab at a dose of about 1000 mg once every 21 days, e.g., on day 1 of each 21-day cycle; cyclophosphamide at a dose of between about 375 mg/m² and about 750 mg/m² (e.g., 375 mg/m², 562.5 mg/m², or 750 mg/m²) once every 21 days, e.g., on day 1 of each 21-day cycle; doxorubicin at a dose of between about 25 mg/m² and about 50 mg/m² (e.g., 25 mg/m², 37.5 mg/m², or 50 mg/m²) once every 21 days, e.g., on day 1 of each 21-day cycle; and a corticosteroid (e.g., prednisone at a dose of about 100 mg, prednisolone at a dose of about 100 mg, or methylprednisolone at a dose of about 80 mg) on days 1-5 of each 21-day cycle. In some embodiments, the anti-CD79b immunoconjugate is administered at a dose of about any of 1.0 mg/kg, 1.4 mg/kg, or 1.8 mg/kg. In some embodiments, the anti-CD79b immunoconjugate is administered at a dose of about 1.0 mg/kg. In some embodiments, the anti-CD79b immunoconjugate is administered at a dose of about 1.4 mg/kg. In some embodiments, the anti-CD79b immunoconjugate is administered at a dose of about 1.8 mg/kg. In some embodiments, the cyclophosphamide is administered at a dose of about 750 mg/m². In some embodiments, the doxorubicin is administered at a dose of about 50 mg/m². In some embodiments, the corticosteroid is prednisone administered at a dose of about 100 mg. In some embodiments, the corticosteroid is prednisolone administered at a dose of about 100 mg. In some embodiments, the corticosteroid is methylprednisolone administered at a dose of about 80 mg.

Another exemplary dosing regimen for the combination therapy of anti-CD79b immunoconjugates (such as huMA79bv28-MC-vc-PAB-MMAE or polatuzumab vedotin) and one or more additional therapeutic agents includes the anti-CD79b immunoconjugate (such as huMA79bv28-MC-vc-PAB-MMAE or polatuzumab vedotin) administered at a dose of about 1.0-1.8 mg/kg (e.g., 1.0 mg/kg, 1.4 mg/kg, or 1.8 mg/kg) once every 21 days, e.g., on day 1 of each 21-day cycle; obinutuzumab at a dose of about 1000 mg once every 21 days, e.g., on day 1 of each 21-day cycle; cyclophosphamide once every 21 days, e.g., on day 1 of each 21-day cycle; doxorubicin once every 21 days, e.g., on day 1 of each 21-day cycle; and a corticosteroid (e.g., prednisone, prednisolone, or methylprednisolone) on days 1-5 of each 21-day cycle. In some embodiments, the anti-CD79b immunoconjugate is administered at a dose of about any of 1.0 mg/kg, 1.4 mg/kg, or 1.8 mg/kg.

The terms “co-administration,” “co-administering,” “combination,” or “in combination,” with respect to administration of two or more therapeutic agents, such as the anti-CD79b immunoconjugate and the at least one additional therapeutic agent (e.g., an anti-CD20 antibody, one or more chemotherapeutic agents, and a corticosteroid), refer to the administration of the two or more therapeutic agents as two (or more) separate formulations, or as one single formulation comprising the two or more therapeutic agents. Where separate formulations are used, the co-administration can be simultaneous (i.e., at the same time) or sequential in any order, wherein preferably there is a time period while all active agents simultaneously exert their biological activities. In some embodiments, the two or more therapeutic agents are co-administered either simultaneously or sequentially. In some embodiments, when all therapeutic agents are co-administered sequentially, the dose of each agent is administered either on the same day in two or more separate administrations, or one of the agents is administered on day 1, the other agent(s) are co-administered on subsequent days, e.g., according to any of the treatment regimens described herein.

An immunoconjugate provided herein (and any additional therapeutic agents, e.g., an anti-CD20 antibody, one or more chemotherapeutic agents, and a corticosteroid) for use in any of the therapeutic methods described herein can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including, but not limited to, single or multiple administrations over various time-points, bolus administration, and pulse infusion are contemplated herein. The anti-CD79b immunoconjugate (e.g., huMA79bv28-MC-vc-PAB-MMAE or polatuzumab vedotin), the anti-CD20 antibody (such as obinutuzumab or rituximab), the one or more chemotherapeutic agents (e.g., cyclophosphamide and/or doxorubicin), and the corticosteroid (e.g., prednisone, prednisolone, or methylprednisolone) may be administered by the same route of administration or by different routes of administration. In some embodiments, the anti-CD79b immunoconjugate is administered intravenously, intramuscularly, subcutaneously, topically, orally, transdermally, intraperitoneally, intraorbitally, by implantation, by inhalation, intrathecally, intraventricularly, or intranasally. In some embodiments, the anti-CD20 antibody (such as obinutuzumab or rituximab) is administered intravenously, intramuscularly, subcutaneously, topically, orally, transdermally, intraperitoneally, intraorbitally, by implantation, by inhalation, intrathecally, intraventricularly, or intranasally. In some embodiments, the one or more chemotherapeutic agents (e.g., cyclophosphamide and/or doxorubicin) are administered intravenously, intramuscularly, subcutaneously, topically, orally, transdermally, intraperitoneally, intraorbitally, by implantation, by inhalation, intrathecally, intraventricularly, or intranasally. In some embodiments, the corticosteroid (e.g., prednisone, prednisolone, or methylprednisolone) is administered intravenously, intramuscularly, subcutaneously, topically, orally, transdermally, intraperitoneally, intraorbitally, by implantation, by inhalation, intrathecally, intraventricularly, or intranasally. In some embodiments, the anti-CD79b immunoconjugate, the anti-CD20 antibody (such as obinutuzumab or rituximab), and the one or more chemotherapeutic agents (e.g., cyclophosphamide and/or doxorubicin) are each administered via intravenous infusion, and the corticosteroid (e.g., prednisone or prednisolone) is administered orally. In some embodiments, the anti-CD79b immunoconjugate, the anti-CD20 antibody (such as obinutuzumab or rituximab), the one or more chemotherapeutic agents (e.g., cyclophosphamide and/or doxorubicin), and the corticosteroid (e.g., methylprednisolone) are each administered via intravenous infusion. An effective amount of the anti-CD79b immunoconjugate, the anti-CD20 antibody (such as obinutuzumab or rituximab), the one or more chemotherapeutic agents (e.g., cyclophosphamide and/or doxorubicin), and the corticosteroid (e.g., prednisone, prednisolone, or methylprednisolone) may be administered for prevention or treatment of a disease, e.g., DLBCL.

### H. Administration of an Anti-CD20 Antibody

In some instances, the anti-CD20 antibody (e.g., obinutuzumab or rituximab) or compositions thereof, utilized in the methods, uses, assays, and kits described herein can be formulated for administration or administered by any suitable method, including, for example, intravenously, intramuscularly, subcutaneously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumorally, peritoneally, subconjunctivally, intravesicularly, mucosally, intrapericardially, intraumbilically, intraocularly, intraorbitally, orally, topically, transdermally, intravitreally (e.g., by intravitreal injection), by eye drop, by inhalation, by injection, by implantation, by infusion, by continuous infusion, by localized perfusion bathing target cells directly, by catheter, by lavage, in cremes, or in lipid compositions. The compositions utilized in the methods described herein can also be administered systemically or locally. The method of administration can vary depending on various factors (e.g., the compound or composition being administered and the severity of the condition, disease, or disorder being treated). In some instances, the anti-CD20 antibody (e.g., obinutuzumab or rituximab) is administered intravenously, intramuscularly, subcutaneously, topically, orally, transdermally, intraperitoneally, intraorbitally, by implantation, by inhalation, intrathecally, intraventricularly, or intranasally. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

In some instances, the anti-CD20 antibody (e.g., obinutuzumab or rituximab) and any additional therapeutic agent may be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. In some instances, the anti-CD20 antibody (e.g., obinutuzumab or rituximab) need not be, but is optionally formulated with and/or administered concurrently with, one or more agents currently used to prevent or treat the disorder in question. The effective amount of such other agents depends on the amount of the anti-CD20 antibody (e.g., obinutuzumab or rituximab) present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)), the appropriate dosage of an anti-CD20 antibody (e.g., obinutuzumab or rituximab) described herein (when used alone or in combination with one or more other additional therapeutic agents) will depend on the type of disease to be treated, the severity and course of the disease, whether the anti-CD20 antibody (e.g., obinutuzumab or rituximab) is administered for preventive or therapeutic purposes, previous therapy, the patient's clinical history and response to the anti-CD20 antibody (e.g., obinutuzumab or rituximab), and the discretion of the attending physician. In some instances, the anti-CD20 antibody (e.g., obinutuzumab or rituximab) is suitably administered to the patient at one time or over a series of treatments. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. Such doses may be administered intermittently, e.g., every week, every month, or every two months. An initial higher loading dose followed by one or more lower doses may be administered. However, other dosage regimens may be useful. The progress of this therapy is easily monitored by conventional techniques and assays.

In some instances, an effective amount of the anti-CD20 antibody (e.g., obinutuzumab or rituximab) may be between about 60 mg to about 5000 mg (e.g., between about 60 mg to about 4500 mg, between about 60 mg to about 4000 mg, between about 60 mg to about 3500 mg, between about 60 mg to about 3000 mg, between about 60 mg to about 2500 mg, between about 650 mg to about 2000 mg, between about 60 mg to about 1500 mg, between about 100 mg to about 1500 mg, between about 300 mg to about 1500 mg, between about 500 mg to about 1500 mg, between about 600 mg to about 1400 mg, between about 700 mg to about 1300 mg, between about 800 mg to about 1200 mg, between about 900 mg to about 1100 mg, between about 950 mg to about 1050 mg, between about 975 mg to about 1025 mg, or between about 990 mg to about 1010 mg, e.g., about 1000 mg ±5 mg, about 1000±2.5 mg, about 1000±1.0 mg, about 1000±0.5 mg, about 1000±0.2 mg, or about 1000±0.1 mg). In some instances, the methods include administering to the individual the anti-CD20 antibody (e.g., obinutuzumab or rituximab) at about 1000 mg (e.g., a fixed dose of about 1000 mg).

In some aspects, the effective amount of the anti-CD20 antibody (e.g., rituximab or obinutuzumab) is a dose of between about 250 mg/m² to about 500 mg/m² (e.g., between about 250 mg/m² to about 450 mg/m², e.g., between about 250 mg/m² to about 400 mg/m², e.g., between about 300 mg/m² to about 400 mg/m², e.g., between about 325 mg/m² to about 400 mg/m², e.g., between about 350 mg/m² to about 400 mg/m², e.g., between about 350 mg/m² to about 375 mg/m², e.g., about 375±2 mg/m², about 375±1 mg/m², about 375±0.5 mg/m², about 375±0.2 mg/m², or about 375±0.1 mg/m², e.g., about 375 mg/m²). In some aspects, the effective amount of the anti-CD20 antibody (e.g., rituximab or obinutuzumab) is a dose of about 375 mg/m².

In some instances, the anti-CD20 antibody (e.g., obinutuzumab or rituximab) is administered to the individual (e.g., a human) at 1000 mg intravenously on day 1, 8, and 15 of Cycle 1, 1000 mg on day 1 of Cycles 2-6 or Cycles 2-8, and then 1000 mg every 2 months for up to 2 years. In some instances, the anti-CD20 antibody (e.g., rituximab or obinutuzumab) is administered to the individual (e.g., a human) at 375 mg/m² intravenously on day 1 of Cycles 1-8. The dose may be administered as a single dose or as multiple doses (e.g., 2, 3, 4, 5, 6, 7, or more than 7 doses), such as infusions. In some instances, the anti-CD20 antibody (e.g., obinutuzumab or rituximab) administered to the individual (e.g., a human) may be administered alone or in combination with an additional therapeutic agent described herein (e.g., CHOP), in four to six doses. The dose of the antibody administered in a combination treatment may be reduced as compared to a single treatment. The progress of this therapy is easily monitored by conventional techniques. In one instance, the anti-CD20 antibody (e.g., obinutuzumab or rituximab) is administered as a monotherapy to the individual to treat a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)). In other instances, the anti-CD20 antibody (e.g., obinutuzumab or rituximab) is administered as a combination therapy, as described herein, to the individual to treat a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)).

In some instances, the anti-CD20 antibody (e.g., obinutuzumab or rituximab) is administered as a combination therapy with CHOP. In some instances, the anti-CD20 antibody (e.g., obinutuzumab or rituximab) is administered to the individual (e.g., a human) at 1000 mg intravenously on day 1, 8, and 15 of Cycle 1, 1000 mg on day 1 of Cycles 2-6 or Cycles 2-8, and then 1000 mg every 2 months for up to 2 years and CHOP is administered for 6 or 8 cycles at the following doses: cyclophosphamide 750 mg/m2 IV (Day 1); doxorubicin 50 mg/m² IV (Day 1); vincristine 1.4 mg/m² IV (Day 1, maximum 2.0 mg); and prednisone 100 mg/day orally (Days 1-5). In some instances, the anti-CD20 antibody (e.g., rituximab or obinutuzumab) is administered to the individual (e.g., a human) at 375 mg/m² intravenously on day 1 of Cycles 1-8 and CHOP is administered for 6 or 8 cycles at the following doses: cyclophosphamide 750 mg/m2 IV (Day 1); doxorubicin 50 mg/m² IV (Day 1); vincristine 1.4 mg/m² IV (Day 1, maximum 2.0 mg); and prednisone 100 mg/day orally (Days 1-5).

### J. Indications

The methods and compositions described herein are useful for treating a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) by administering an anti-CD79b immunoconjugate (e.g., polatuzumab vedotin) and an anti-CD20 antibody (e.g., obinutuzumab or rituximab). In some instances, the lymphoma may be indolent lymphoma. In some instances, the lymphoma may be a B-cell lymphoma, e.g., a non-Hodgkin lymphoma, e.g., a diffuse large B-cell lymphoma (e.g., a germinal-center B-cell-like or activated B-cell-like diffuse large B-cell lymphoma). In some instances, the lymphoma may be a CD20-positive lymphoma. In certain instances, the cancer may be a B-cell lymphoma. For example, the B-cell lymphoma may be a non-Hodgkin lymphoma, including but not limited to a diffuse large B-cell lymphoma (e.g., a germinal-center B-cell-like or activated B-cell-like diffuse large B-cell lymphoma)).

In some instances, the individual having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) has not been previously treated for the lymphoma (treatment naïve). For example, in some instances, the individual having a lymphoma has not previously received either an anti-CD79b immunoconjugate (e.g., polatuzumab vedotin) and an anti-CD20 antibody (e.g., obinutuzumab or rituximab) or an anti-CD20 antibody (e.g., obinutuzumab or rituximab).

In some instances, the individual having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) has previously received treatment for the lymphoma. In some instances, the individual having a lymphoma has previously received treatment including either an anti-CD79b immunoconjugate (e.g., polatuzumab vedotin) and an anti-CD20 antibody (e.g., obinutuzumab or rituximab) or an anti-CD20 antibody (e.g., obinutuzumab or rituximab)).

### J. Combination Therapies

In any of the methods or uses herein, an anti-CD79b immunoconjugate (e.g., polatuzumab vedotin) and an anti-CD20 antibody (e.g., obinutuzumab or rituximab) may be administered in combination with an effective amount of one or more additional therapeutic agents. Suitable additional therapeutic agents include, for example, an anti-neoplastic agent, a chemotherapeutic agent, a growth inhibitory agent, a cytotoxic agent, a radiotherapy, or combinations thereof.

In some instances, the methods further involve administering to the patient an effective amount of one or more additional therapeutic agents. In some instances, the additional therapeutic agent is selected from the group consisting of a cytotoxic agent, a chemotherapeutic agent, a growth-inhibitory agent, a radiation therapy agent, an anti-angiogenic agent, and combinations thereof. In some instances, an anti-CD79b immunoconjugate (e.g., polatuzumab vedotin) and an anti-CD20 antibody (e.g., obinutuzumab or rituximab) may be administered in conjunction with a chemotherapy or chemotherapeutic agent. In some instances, anti-CD79b immunoconjugate (e.g., polatuzumab vedotin) and an anti-CD20 antibody (e.g., obinutuzumab or rituximab) may be administered in conjunction with a radiation therapy agent. In some instances, anti-CD79b immunoconjugate (e.g., polatuzumab vedotin) and an anti-CD20 antibody (e.g., obinutuzumab or rituximab) may be administered in conjunction with a targeted therapy or targeted therapeutic agent. In some instances, an anti-CD79b immunoconjugate (e.g., polatuzumab vedotin) and an anti-CD20 antibody (e.g., obinutuzumab or rituximab) may be administered in conjunction with an immunotherapy or immunotherapeutic agent, for example a monoclonal antibody. In some instances, the additional therapeutic agent is an agonist directed against an activating co-stimulatory molecule. In some instances, the additional therapeutic agent is an antagonist directed against an inhibitory co-stimulatory molecule.

Such combination therapies noted above encompass combined administration (where two or more therapeutic agents are included in the same or separate formulations), and separate administration, in which case, administration of an anti-CD79b immunoconjugate (e.g., polatuzumab vedotin) and an anti-CD20 antibody (e.g., obinutuzumab or rituximab) can occur prior to, simultaneously, and/or following, administration of the additional therapeutic agent or agents. In one instance, administration of an anti-CD79b immunoconjugate (e.g., polatuzumab vedotin) and an anti-CD20 antibody (e.g., obinutuzumab or rituximab) and administration of an additional therapeutic agent occur within about one month, or within about one, two or three weeks, or within about one, two, three, four, five, or six days, of each other.

In some instances, an anti-CD79b immunoconjugate (e.g., polatuzumab vedotin) and an anti-CD20 antibody (e.g., obinutuzumab or rituximab) may be administered in conjunction with a CHP chemotherapy or with variants of a CHP chemotherapy.

### V. Pharmaceutical Compositions and Formulations

Pharmaceutical compositions and formulations as described herein can be prepared by mixing the active ingredient(s) (e.g., an anti-CD79b immunoconjugate and an anti-CD20 antibody or an anti-CD20 antibody) having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®; Baxter International, Inc.). Certain exemplary sHASEGPs and methods of use, including rHuPH20, are described in U.S. Patent Publication Nos. 2005/0260186 and 2006/0104968. In one aspect, a sHASEGP is combined with one or more additional glycosaminoglycanases such as chondroitinases. It is understood that any of the above pharmaceutical compositions or formulations may include an immunoconjugate described herein in place of, or in addition to, an anti-CD79b immunoconjugate and an anti-CD20 antibody.

Exemplary lyophilized antibody formulations are described in U.S. Pat. No. 6,267,958. Aqueous antibody formulations include those described in U.S. Pat. No. 6,171,586 and WO 2006/044908, the latter formulations including a histidine-acetate buffer.

The compositions and formulations herein may also contain more than one active ingredients as necessary for the particular indication being treated, preferably those with complementary activities that do not adversely affect each other. For example, it may be desirable to further provide an additional therapeutic agent (e.g., a chemotherapeutic agent, a cytotoxic agent, a growth inhibitory agent, and/or an anti-hormonal agent, such as those recited herein above). Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g., films, or microcapsules. The formulations to be used for in vivo administration are generally sterile. Sterility may be readily accomplished, for example, by filtration through sterile filtration membranes.

### VI. Articles of Manufacture and Kits

In another aspect of the invention, an article of manufacture or kit containing materials useful for the treatment, prevention, and/or diagnosis of patients is provided.

In another embodiment, an article of manufacture or a kit is provided comprising an anti-CD79b immunoconjugate (such as described herein) and at least one additional agent. In some embodiments the at least one additional agent is an anti-CD20 antibody (e.g., rituximab or obinutuzumab), one or more chemotherapeutic agents (e.g., cyclophosphamide and/or doxorubicin), and a corticosteroid (e.g., prednisone, prednisolone, or methylprednisolone). In some embodiments, the article of manufacture or kit further comprises a package insert comprising instructions for using the anti-CD79b immunoconjugate in conjunction at least one additional agent, such as an anti-CD20 antibody (e.g., rituximab or obinutuzumab), one or more chemotherapeutic agents (e.g., cyclophosphamide and/or doxorubicin), and a corticosteroid (e.g., prednisone, prednisolone, or methylprednisolone) to treat or delay progression of a B-cell proliferative disorder (e.g., DLBCL) in an individual. Any of the anti-CD79b immunoconjugates, anti-CD20 antibodies, chemotherapeutic agents, and/or corticosteroids, and optionally one or more additional anti-cancer agents, known in the art or described herein may be included in the article of manufacture or kits. In some embodiments, the kit comprises an immunoconjugate comprising the formula

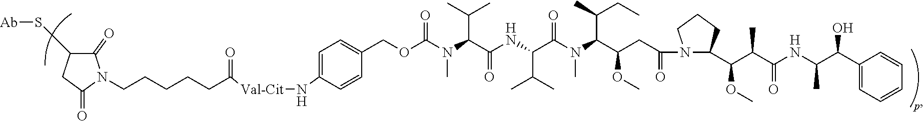

wherein Ab is an anti-CD79b antibody comprising (i) an HVR-H1 that comprises the amino acid sequence of SEQ ID NO: 5; (ii) an HVR-H2 comprising the amino acid sequence of SEQ ID NO: 6; (iii) an HVR-H3 comprising the amino acid sequence of SEQ ID NO: 7; (iv) an HVR-L1 comprising the amino acid sequence of SEQ ID NO: 8; (v) an HVR-L2 comprising the amino acid sequence of SEQ ID NO: 9; and (vi) an HVR-L3 comprising the amino acid sequence of SEQ ID NO:10, and wherein p is between 1 and 8. In some embodiments, the kit comprises an immunoconjugate comprising the formula

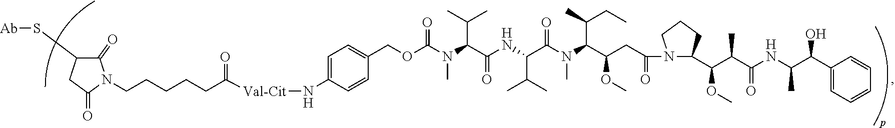

wherein Ab is an anti-CD79b antibody that comprises (i) a heavy chain comprising a VH that comprises the amino acid sequence of SEQ ID NO: 3 and (ii) a light chain comprising a VL that comprises the amino acid sequence of SEQ ID NO: 4, and wherein p is between 2 and 5. In some embodiments, p is between 3 and 4, e.g., 3.4 or 3.5. In some embodiments, the immunoconjugate comprises an anti-CD79b antibody comprising a heavy chain comprising the amino acid sequence of SEQ ID NO: 12, and a light chain comprising the amino acid sequence of SEQ ID NO: 11. In certain embodiments, the anti-CD79b immunoconjugate comprises the structure of Ab-MC-vc-PAB-MMAE. In some embodiments, the anti-CD79b immunoconjugate is polatuzumab vedotin (CAS Number 1313206-42-6). In some embodiments, the at least one additional agent is an anti-CD20 antibody (e.g., rituximab or obinutuzumab), one or more chemotherapeutic agents (e.g., cyclophosphamide and/or doxorubicin), and a corticosteroid (e.g., prednisone, prednisolone, or methylprednisolone). In some embodiments, the kit is for use in the treatment of DLBCL, e.g., previously untreated DLBCL, in an individual, such as a human patient (e.g., a human patient having one or more characteristics described herein) according to a method provided herein.

In some embodiments, the anti-CD79b immunoconjugate, the anti-CD20 antibody (e.g., rituximab or obinutuzumab), one or more chemotherapeutic agents (e.g., cyclophosphamide and/or doxorubicin), and a corticosteroid (e.g., prednisone, prednisolone, or methylprednisolone) are in the same container or in separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation, and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

In some instances, such articles of manufacture or kits can be used to identify a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) who may benefit from a treatment comprising an anti-CD79b immunoconjugate and an anti-CD20 antibody (e.g., obinutuzumab or rituximab). Such articles of manufacture or kits may include (a) reagents for determining a macrophage biomarker in a sample from the individual and (b) instructions for using the reagents to identify a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) who may benefit from a treatment comprising an anti-CD79b immunoconjugate and an anti-CD20 antibody (e.g., obinutuzumab or rituximab).

In some instances, such articles of manufacture or kits include an anti-CD79b immunoconjugate and an anti-CD20 antibody (e.g., obinutuzumab or rituximab) for treating a patient with a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)) who may benefit from a treatment comprising an anti-CD79b immunoconjugate and an anti-CD20 antibody (e.g., obinutuzumab or rituximab). In some instances, the article of manufacture or kit includes (a) an anti-CD79b immunoconjugate and an anti-CD20 antibody (e.g., obinutuzumab or rituximab) and (b) a package insert including instructions for administration of the anti-CD79b immunoconjugate and the anti-CD20 antibody (e.g., obinutuzumab or rituximab) to a patient having a lymphoma (e.g., a B-cell lymphoma, e.g., a non-Hodgkin lymphoma, e.g., a diffuse large B-cell lymphoma (e.g., a germinal-center B-cell-like or activated B-cell-like diffuse large B-cell lymphoma)), wherein, prior to treatment, the macrophage biomarker in a sample from the patient has been determined to be below a reference macrophage biomarker amount or level.

Any of the articles of manufacture or kits described may include a carrier means being compartmentalized to receive in close confinement one or more container means such as vials, tubes, and the like, each of the container means comprising one of the separate elements to be used in the method. Where the article of manufacture or kit utilizes nucleic acid hybridization to detect the target nucleic acid, the kit may also have containers containing nucleotide(s) for amplification of the target nucleic acid sequence and/or a container comprising a reporter-means, such as an enzymatic, florescent, or radioisotope label.

In some instances, the article of manufacture or kit includes the container described above and one or more other containers including materials desirable from a commercial and user standpoint, including buffers, diluents, filters, needles, syringes, and package inserts with instructions for use. A label may be present on the container to indicate that the composition is used for a specific application, and may also indicate directions for either in vivo or in vitro use, such as those described above. For example, the article of manufacture or kit may further include a container including a pharmaceutically-acceptable buffer, such as bacteriostatic water for injection (BWFI), phosphate-buffered saline, Ringer's solution, and dextrose solution.

The articles of manufacture or kits described herein may have a number of embodiments. In one instance, the article of manufacture or kit includes a container, a label on said container, and a composition contained within said container, wherein the composition includes one or more polynucleotides that hybridize to a complement of a gene described herein under stringent conditions, and the label on said container indicates that the composition can be used to evaluate the presence or level of the gene described herein in a sample, and wherein the kit includes instructions for using the polynucleotide(s) for evaluating the presence of the gene RNA or DNA in a particular sample type.

For oligonucleotide-based articles of manufacture or kits, the article of manufacture or kit can include, for example: (1) an oligonucleotide, e.g., a detectably labeled oligonucleotide, which hybridizes to a nucleic acid sequence encoding a protein or (2) a pair of primers useful for amplifying a nucleic acid molecule. The article of manufacture or kit can also include, e.g., a buffering agent, a preservative, or a protein stabilizing agent. The article of manufacture or kit can further include components necessary for detecting the detectable label (e.g., an enzyme or a substrate). The article of manufacture or kit can also contain a control sample or a series of control samples that can be assayed and compared to the test sample. Each component of the article of manufacture or kit can be enclosed within an individual container and all of the various containers can be within a single package, along with instructions for interpreting the results of the assays performed using the kit.

For protein-based articles of manufacture or kits, the article of manufacture or kit can include, for example, an antibody that binds any of the genes described herein. The article of manufacture or kit can further include components necessary for detecting the detectable label (e.g., an enzyme or a substrate). In some instances, the antibody is conjugated to an enzyme (e.g., horseradish peroxidase (HRP)). In some instances, the antibody is conjugated to a fluorophore. The article of manufacture or kit can also contain a control sample or a series of control samples that can be assayed and compared to the test sample. Each component of the article of manufacture or kit can be enclosed within an individual container and all of the various containers can be within a single package, along with instructions for interpreting the results of the assays performed using the kit.

| Sequence Table | SEQ ID NO: |
|---|---|
| RFIARKRGFTVKMHCYMNSASGNVSWLWKQEMDENPQQLKLEKGRMEESQ | 1 |
| NESLATLTIQGIRFEDNGIYFCQQKCNNTSEVYQGCGTELRVMGFSTLAQ | |
| LKQRNTLKDGIIMIQTLLIILFIIVPIFLLLDKDDSKAGMEEDHTYEGLD | |
| IDQTATYEDIVTLRTGEVKWSVGEHPGQE | |
| ARSEDRYRNPKGSACSRIWQSPRFIARKRGFTVKMHCYMNSASGNVSWLW | 2 |
| KQEMDENPQQLKLEKGRMEESQNESLATLTIQGIRFEDNGIYFCQQKCNN | |
| TSEVYQGCGTELRVMGFSTLAQLKQRNTLKDGIIMIQTLLIILFIIVPIF | |
| LLLDKDDSKAGMEEDHTYEGLDIDQTATYEDIVTLRIGEVKWSVGEHPGQ | |
| E | |
| EVQLVESGGGLVQPGGSLRLSCAASGYTFSSYWIEWVRQAPGKGLEWIGE | 3 |
| ILPGGGDTNYNEIFKGRATFSADTSKNTAYLQMNSLRAEDTAVYYCTRRV | |
| PIRLDYWGQGTLVTVSS | |
| DIQLTQSPSSLSASVGDRVTITCKASQSVDYEGDSFLNWYQQKPGKAPKL | 4 |
| LIYAASNLESGVPSRFSGSGSGTDFTLTISSLQPEDFATYYCQQSNEDPL | |
| TFGQGTKVEIKR | |
| GYTFSSYWIE | 5 |
| GEILPGGGDINYNEIFKG | 6 |
| TRRVPIRLDY | 7 |
| KASQSVDYEGDSFLN | 8 |
| AASNLES | 9 |
| QQSNEDPLT | 10 |
| DIQLTQSPSSLSASVGDRVTITCKASQSVDYEGDSFLNWYQQKPGKAPKL | 11 |
| LIYAASNLESGVPSRFSGSGSGTDFTLTISSLQPEDFATYYCQQSNEDPL | |
| TFGQGTKVEIKRTVAAPSVFIFPPSDEQLKSGTASVVCLLNNFYPREAKV | |
| QWKVDNALQSGNSQESVTEQDSKDSTYSLSSTLTLSKADYEKHKVYACEV | |
| THQGLSSPVTKSFNRGEC | |
| EVQLVESGGGLVQPGGSLRLSCAASGYTFSSYWIEWVRQAPGKGLEWIGE | 12 |
| ILPGGGDTNYNEIFKGRATFSADTSKNTAYLQMNSLRAEDTAVYYCTRRV | |
| PIRLDYWGQGTLVTVSSASTKGPSVFPLAPSSKSTSGGTAALGCLVKDYF | |
| PEPVTVSWNSGALISGVHTFPAVLQSSGLYSLSSVVTVPSSSLGTQTYIC | |
| NVNHKPSNTKVDKKVEPKSCDKTHTCPPCPAPELLGGPSVFLFPPKPKDT | |
| LMISRTPEVTCVVVDVSHEDPEVKENWYVDGVEVHNAKTKPREEQYNSTY | |
| RVVSVLTVLHQDWLNGKEYKCKVSNKALPAPIEKTISKAKGQPREPQVYT | |
| LPPSREEMTKNQVSLTCLVKGFYPSDIAVEWESNGQPENNYKTTPPVLDS | |
| DGSFFLYSKLTVDKSRWQQGNVFSCSVMHEALHNHYTQKSLSLSPG | |
| EVQLVESGGGLVQPGGSLRLSCAASGYTFSSYWIEWVRQAPGKGLEWIGE | 13 |
| ILPGGGDTNYNEIFKGRATFSADISKNTAYLQMNSLRAEDTAVYYCTRRV | |
| PIRLDYWGQGTLVTVSSCSTKGPSVFPLAPSSKSTSGGTAALGCLVKDYF | |
| PEPVTVSWNSGALISGVHTFPAVLQSSGLYSLSSVVTVPSSSLGTQTYIC | |
| NVNHKPSNTKVDKKVEPKSCDKTHTCPPCPAPELLGGPSVFLFPPKPKDT | |
| LMISRTPEVTCVVVDVSHEDPEVKFNWYVDGVEVHNAKTKPREEQYNSTY | |
| RVVSVLTVLHQDWLNGKEYKCKVSNKALPAPIEKTISKAKGQPREPQVYT | |
| LPPSREEMTKNQVSLTCLVKGFYPSDIAVEWESNGQPENNYKTTPPVLDS | |
| DGSFFLYSKLTVDKSRWQQGNVFSCSVMHEALHNHYTQKSLSLSPG | |
| DIQLTQSPSSLSASVGDRVTITCKASQSVDYEGDSFLNWYQQKPGKAPKL | 14 |
| LIYAASNLESGVPSRFSGSGSGTDFTLTISSLQPEDFATYYCQQSNEDPL | |
| TFGQGTKVEIKRTVAAPSVFIFPPSDEQLKSGTASVVCLLNNFYPREAKV | |
| QWKVDNALQSGNSQESVTEQDSKDSTYSLSSTLILSKADYEKHKVYACEV | |
| THQGLSSPCTKSENRGEC | |
| EVQLVESGGGLVQPGGSLRLSCAASGYTESSYWIEWVRQAPGKGLEWIGE | 15 |
| ILPGGGDTNYNEIFKGRATFSADTSKNTAYLQMNSLRAEDTAVYYCTRRV | |
| PIRLDYWGQGTLVTVSSASTKGPSVFPLAPSSKSTSGGTAALGCLVKDYF | |
| PEPVTVSWNSGALTSGVHTFPAVLQSSGLYSLSSVVTVPSSSLGTQTYIC | |
| NVNHKPSNTKVDKKVEPKSCDKTHTCPPCPAPELLGGPSVFLFPPKPKDT | |
| LMISRTPEVTCVVVDVSHEDPEVKFNWYVDGVEVHNAKTKPREEQYNSTY | |
| RVVSVLTVLHQDWLNGKEYKCKVSNKALPAPIEKTISKAKGQPREPQVYT | |
| LPPSREEMTKNQVSLTCLVKGFYPSDIAVEWESNGQPENNYKTTPPVLDS | |
| DGSFFLYSKLTVDKSRWQQGNVFSCSVMHEALHNHYTQKSLSLSPGK | |
| GYAFSY | 16 |
| FPGDGDTD | 17 |
| NVFDGYWLVY | 18 |
| RSSKSLLHSNGITYLY | 19 |
| QMSNLVS | 20 |
| AQNLELPYT | 21 |
| QVQLVQSGAEVKKPGSSVKVSCKASGYAFSYSWINWVRQAPGQGLEWMGR | 22 |
| IFPGDGDTDYNGKFKGRVTITADKSTSTAYMELSSLRSEDTAVYYCARNV | |
| FDGYWLVYWGQGTLVTVSS | |
| DIVMTQTPLSLPVTPGEPASISCRSSKSLLHSNGITYLYWYLQKPGQSPQ | 23 |
| LLIYQMSNLVSGVPDRFSGSGSGTDFTLKISRVEAEDVGVYYCAQNLELP | |
| YTFGGGTKVEIKRTV | |
| QVQLVQSGAEVKKPGSSVKVSCKASGYAFSYSWINWVRQAPGQGLEWMGR | 24 |
| IFPGDGDTDYNGKFKGRVTITADKSTSTAYMELSSLRSEDTAVYYCARNV | |
| FDGYWLVYWGQGTLVTVSSASTKGPSVFPLAPSSKSTSGGTAALGCLVKD | |
| YFPEPVTVSWNSGALTSGVHTFPAVLQSSGLYSLSSVVTVPSSSLGTQTY | |
| ICNVNHKPSNTKVDKKVEPKSCDKTHTCPPCPAPELLGGPSVFLFPPKPK | |
| DTLMISRTPEVTCVVVDVSHEDPEVKENWYVDGVEVHNAKIKPREEQYNS | |
| TYRVVSVLTVLHQDWLNGKEYKCKVSNKALPAPIEKTISKAKGQPREPQV | |
| YTLPPSRDELTKNQVSLTCLVKGFYPSDIAVEWESNGQPENNYKTTPPVL | |
| DSDGSFFLYSKLTVDKSRWQQGNVFSCSVMHEALHNHYTQKSLSLSPG | |
| DIVMTQTPLSLPVTPGEPASISCRSSKSLLHSNGITYLYWYLQKPGQSPQ | 25 |
| LLIYQMSNLVSGVPDRFSGSGSGTDFTLKISRVEAEDVGVYYCAQNLELP | |
| YTFGGGTKVEIKRTVAAPSVFIFPPSDEQLKSGTASVVCLLNNFYPREAK | |
| VQWKVDNALQSGNSQESVTEQDSKDSTYSLSSTLTLSKADYEKHKVYACE | |
| VTHQGLSSPVTKSENRGEC | |
| SYNMH | 26 |
| AIYPGNGDTSYNQKFKG | 27 |
| STYYGGDWYFNV | 28 |
| RASSSVSYIH | 29 |
| ATSNLAS | 30 |
| QQWTSNPPT | 31 |
| QIVLSQSPAILSASPGEKVTMTC | 32 |
| WFQQKPGSSPKPWIY | 33 |
| GVPVRFSGSGSGTSYSLTISRVEAEDAATYYC | 34 |
| FGGGTKLEIK | 35 |
| QVQLQQPGAELVKPGASVKMSCKASGYTFT | 36 |
| WVKQTPGRGLEWIG | 37 |
| KATLTADKSSSTAYMQLSSLISEDSAVYYCAR | 38 |
| WGAGTTVTVSA | 39 |
| QVQLQQPGAELVKPGASVKMSCKASGYTFTSYNMHWVKQTPGRGLEWIGA | 40 |
| IYPGNGDTSYNQKFKGKATLTADKSSSTAYMQLSSLISEDSAVYYCARST | |
| YYGGDWYFNVWGAGTTVTVSA | |
| QIVLSQSPAILSASPGEKVTMTCRASSSVSYIHWFQQKPGSSPKPWIYAT | 41 |
| SNLASGVPVRFSGSGSGTSYSLTISRVEAEDAATYYCQQWTSNPPTFGGG | |
| TKLEIK | |
| YSWIN | 42 |
| RIFPGDGDTDYNGKFK | 43 |

### VII. Examples

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1. Immune Contexture Analysis in POLARIX Suggests Response to Pola-R-CHP Treatment Reduces Tumor Microenvironment Dependency

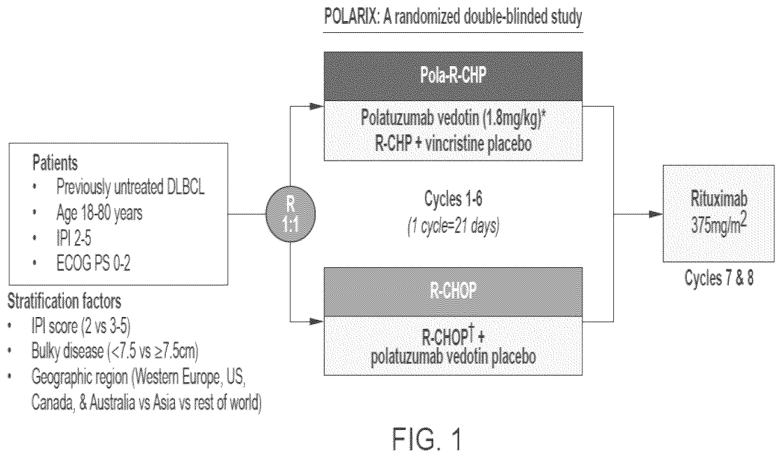

POLARIX trial (Clinical Trial ID No. NCT03274492) is a Phase III, randomized, double-blind, placebo-controlled study that compared the efficacy and safety of drug combination Pola R-CHP (polatuzumab vedotin, rituximab, cyclophosphamide, doxorubicin, and prednisone) with R-CHOP (rituximab, cyclophosphamide, doxorubicin, vincristine, and prednisone) in patients with previously untreated diffuse large B-cell lymphoma (DLBCL) (FIG. 1). The objective of this analysis was to assess clinical outcomes in subgroups defined by a macrophage biomarker that dichotomized patients into a high and low macrophage biomarker subset.

Global gene expression patterns of baseline tumor biopsies from patients treated with Pola-R-CHP or R-CHOP in POLARIX were generated by RNA-seq. Immune and stromal cell tumor content was estimated using the xCell and QuanTIseq algorithms. Association of the infiltration scores with PFS was evaluated. Hazard ratios (HR) were adjusted for International Prognostic Index score (2 vs 3-5), age (<60 vs >60 years), and cell of origin (activated B cell, germinal center B cell, unclassified, unknown).

### Data Set

Biopsy samples, clinical outcomes (overall survival (OS), progression-free survival (PFS)), and high-through transcriptome sequencing data were available for 665 patients from the POLARIX trial. Clinical cut-off date occurred on Jun. 15, 2022 with a median follow-up of 39.7 months.

### RNA-Seq Processing

Applying high-throughput transcriptome sequencing quality control was performed. Adapters were trimmed using ea-utils function fastq-mcf (parameters: --max-ns 4 --qual-mean 25 -H -p 5 -q 7-1 25-1 25). Trimmed reads were aligned to human genome reference GRCh38 using GSNAP version 2013-10⁻¹⁰ (parameters: -M 2 -n 10 -B 2 -i 1 -N 1 -w 200000 -E 1 --pairmax-rna=200000 --clip-overlap). Leveraging RNA-Seq pipeline scripts, exonic gene counts were determined for each sample.

Genes and samples were filtered using an internal RNA-Seq processing package, rnaseqTools (parameters: default). To account for sequencing variability, counts were normalized to transcripts per million (TPM).

### Cell Composition Estimation Via QuanTIseq

Multiple bulk RNA-Seq deconvolution approaches were applied to the final gene by sample count matrix in order to estimate the cellular composition of a sample. In particular, a deconvolution (QuanTIseq) based approach (see, e.g., Finotello et al. Genome Med. 11(1):34 (2019)) was used to estimate the cellular composition of a bulk RNA-Seq patient sample. The deconvolution method identified the relative composition of numerous cell types, including M1 macrophages and M2 macrophages, within/across samples. In particular, the deconvolution based approach articulates the problem as a system of equations solved using constrained least squares for a set of signatures.

The deconvolution based approach leveraged blood and tumor samples using simulated, flow cytometry, and immunohistochemistry data to apply QuanTIseq. The R package immunedeconv (see, e.g., Sturm et al. Bioinformatics. 35(14):i436-i445 (2019)), which serves as a unified interface to multiple immune deconvolution methods, was used to apply QuanTIseq.

Cell Composition Estimation Via xCell

A marker gene (xCell) based approach (see, e.g., Aran et al. Genome Biol. 18(1):220 (2017)) was used to estimate the cellular composition of a bulk RNA-Seq patient sample. The marker gene method identified the relative composition of numerous cell types, including macrophages, M1 macrophages, M2 macrophages, mast cells, and memory B cells, within/across samples. In particular, the expression of genes within a signature, e.g., M1 macrophages are estimated and statistically tested for enrichment within a sample.

The marker gene approach, xCell, was used to build signatures derived from a compendium of six sequencing and microarray sources: FANTOM5 project (see, e.g., Lizio M, et al. Genome Biol. 16:22 (2015)), the ENCODE project (see, e.g., ENCODE Project Consortium. Nature. 489(7414):57-74 (2012)), the Blueprint project (see, e.g., Fernández et al. Cell Syst. 3(5):491-495.e5 (2016)), the IRIS project (see, e.g., Abbas et al. Genes Immun. 6(4):319-31 (2005)), the Novershtern et al. study (Novershtern et al. Cell. 144(2):296-309 (2011)), and the Human Primary Cells Atlas (HPCA) (see, e.g., Uhlén. Science. 347(6220):1260419 (2015)). Details of genes included in the various signatures are listed in Table 2 and Table 3. The R package immunedeconv (see, e.g., Sturm et al. Bioinformatics. 35(14):i436-i445 (2019)), which serves as a unified interface to multiple immune deconvolution methods, was used to apply xCell.

### Statistical Analysis

### Survival Analysis

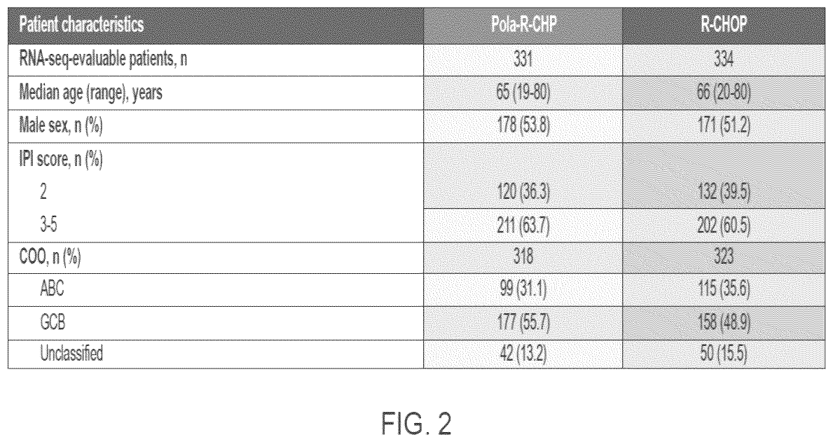

FIG. 2 shows characteristics of RNA-seq-evaluable patients for the Pola-R-CHP and R-CHOP treatment arms. PFS was similar between the RNA-Seq evaluable and non-evaluable populations.

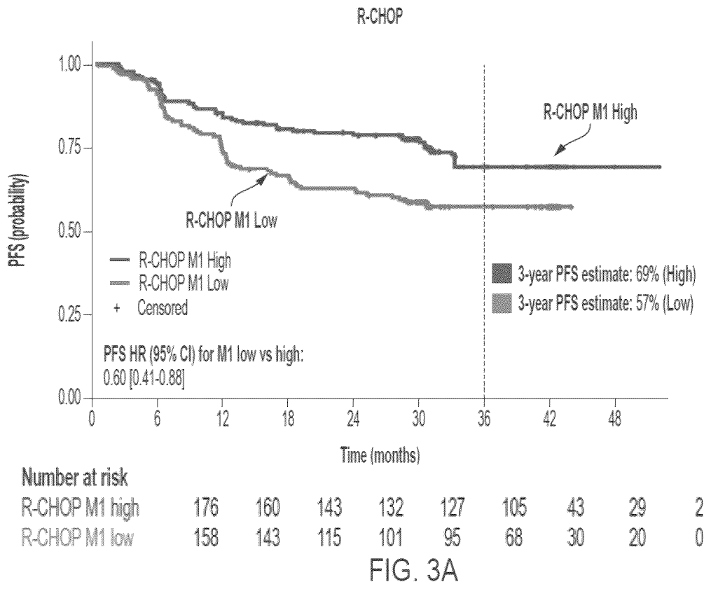

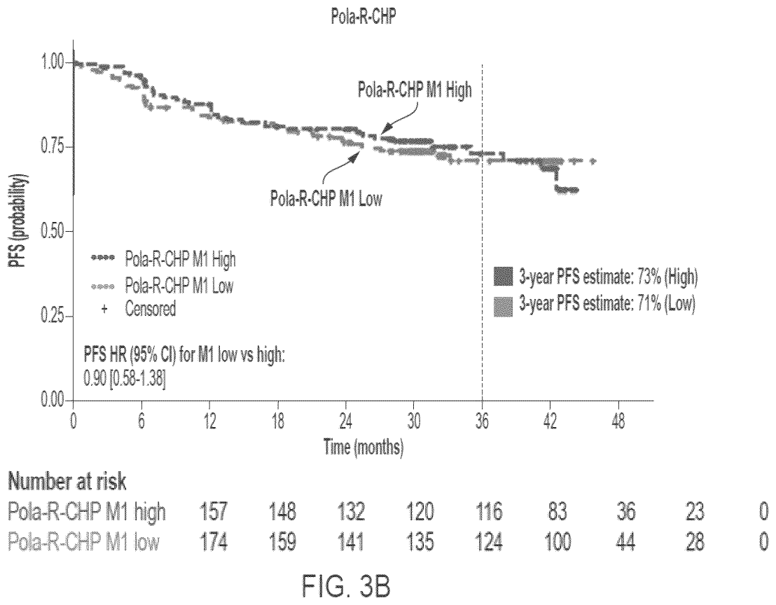

Gene expression data were generated from 665 patients in POLARIX (Pola-R-CHP, n=331; R-CHOP, n=334). M1 macrophage infiltration levels were comparable between treatment arms. Quantity of M1 macrophage infiltration was the primary immune-related positive prognostic factor for PFS in patients treated with R-CHOP (FIG. 3A). High levels (above median) of M1 macrophages were associated with improved PFS when quantified by either QuanTIseq (HR 0.60, 95% confidence interval [CI]: 0.41-0.88) or xCell (HR 0.57, 95% CI: 0.39-0.84) (FIGS. 3A & 6). In contrast, M1 macrophage infiltration did not impact PFS clinical activity of the Pola-R-CHP regimen when quantified by either QuanTIseq (HR 0.90, 95% CI: 0.58-1.38) or xCell (HR 0.95, 95% CI: 0.62-1.46); the treatment benefit in patients with lymphomas with low M1 macrophage levels was similar to that of patients with high M1 macrophage levels (FIGS. 3B & 6).

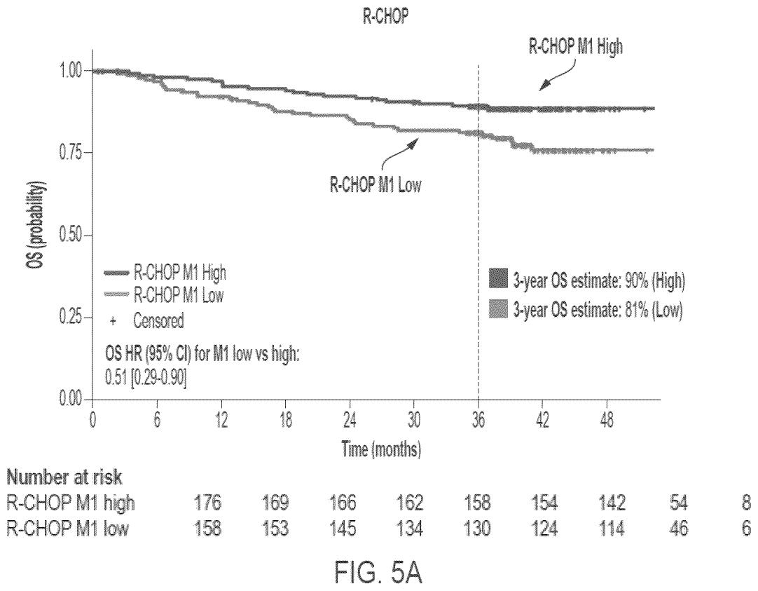

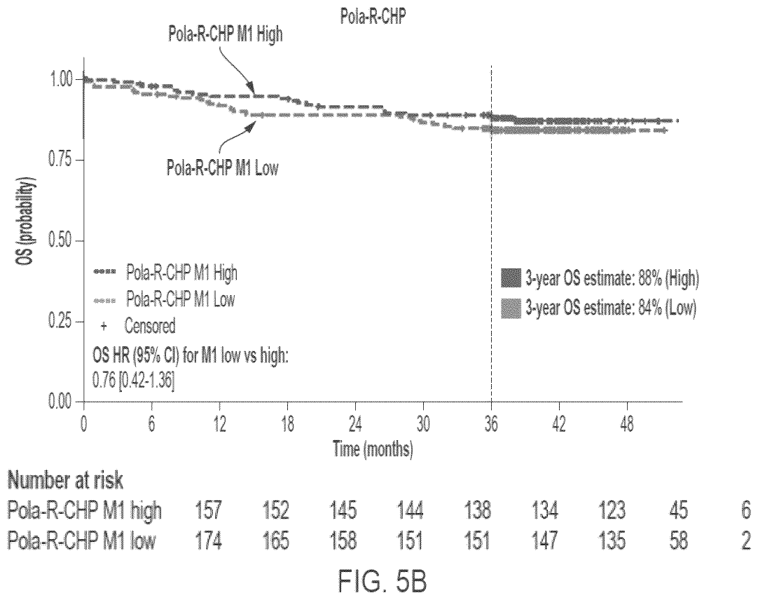

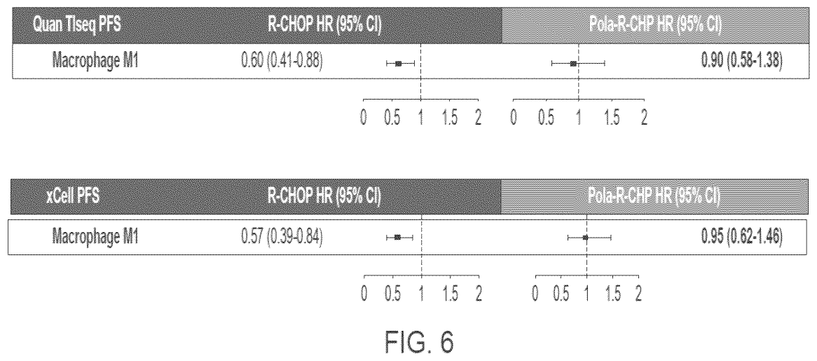

Quantity of M1 macrophage infiltration was the primary immune-related positive prognostic factor for OS in patients treated with R-CHOP (FIG. 5A). High levels (above median) of M1 macrophages were associated with improved OS when quantified by QuanTIseq (HR 0.51, 95% confidence interval [CI]: 0.29-0.90) (FIG. 5A). In contrast, M1 macrophage infiltration did not impact OS clinical activity of the Pola-R-CHP regimen when quantified by QuanTIseq (HR 0.76, 95% CI: 0.42-1.36); the treatment benefit in patients with lymphomas with low M1 macrophage levels was similar to that of patients with high M1 macrophage levels (FIG. 5B).

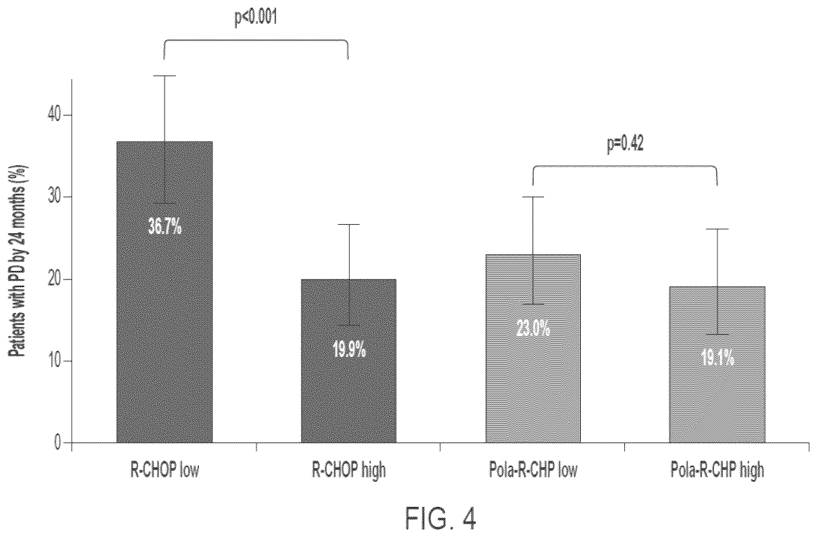

FIG. 4 demonstrates low M1 macrophage levels are associated with lower PFS at 24 months in the R-CHOP treatment arm but not the Pola-R-CHP treatment arm. More patients with low M1 macrophage signature progressed within 24 months compared to those with high M1 signature in the R-CHOP treatment arm. In contrast, patients with high or low M1 macrophage levels were equally as likely to progress within 24 months with the Pola-R-CHP treatment than patients with high M1 levels treated with R-CHOP.

### Conclusions

The results confirm that the efficacy of R-CHOP is associated with a specific lymphoma microenvironment. M1 macrophage levels are the primary positive predictors of treatment outcome. In contrast, Pola-R-CHP may achieve therapeutic responses in patients who lack a favorable immune contexture, suggesting Pola-R-CHP can decouple treatment outcome from the lymphoma microenvironment, likely due to its distinct mechanism of action. Therefore, it may be beneficial to select Pola-R-CHP therapy for a patient that has a low macrophage M1 signature.

### Example 2. Evaluation of a New Gene Signature for Tumor-Associated Macrophages (TAM)

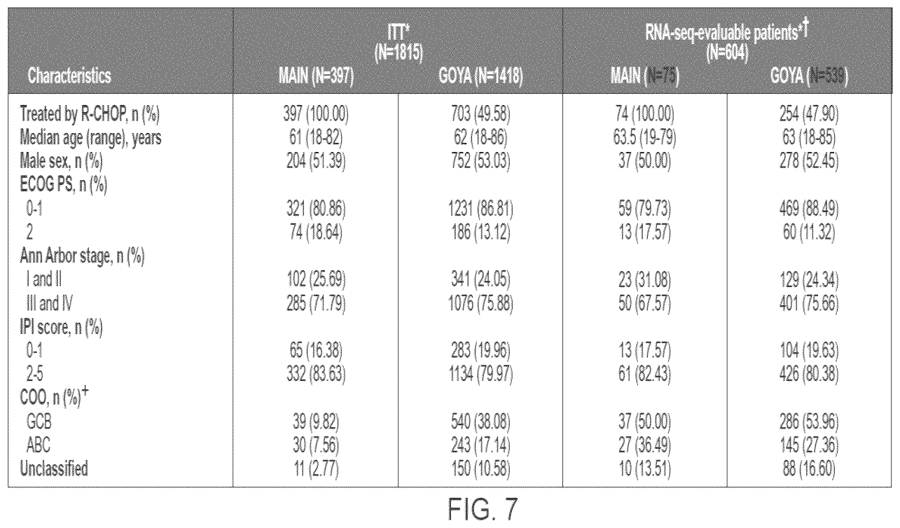

This study leveraged bulk RNA-seq performed on pretreatment formalin-fixed paraffin-embedded (FFPE) lymphoma biopsies collected fronaïveatment naïve DLBCL patients enrolled across three clinical trials. GOYA (NCT01287741) compared the activity of G-CHOP (obinutuzumab plus CHOP) vs R-CHOP (rituximab plus CHOP) showing equivalent clinical efficacy for these CD20-targeted chemo immunotherapy regimens. Among 1,418 patients treated in this study, 539 high quality transcriptomic profiles were examined from biomarker evaluable patients from both treatment arms. POLARIX (NCT03274492) compared Pola-R-CHP to R-CHOP treatment clinical efficacy. Transcriptional profiles from 665 patients (Pola-R-CHP, n=331; R-CHOP, n=334) with baseline characteristics similar to the full cohort (n=879 patients) were generated and analyzed (see FIG. 7). Seventy-five RNA-seq profiles generated from the R-CHOP control arm of the MAIN clinical study (NCT00486759) were leveraged to validate key findings. The resulting global gene expression patterns were analyzed to quantify the various infiltrating cell types for each patient sample using the xCell and quanTIseq algorithms in order to generate maps of lymphoma microenvironment in each patient. Tumor-associated macrophage (TAM) signature scores were calculated based on a macrophage signature (MARCO, ACP5, VSIG4, MRC1, MSR1, MCEMP1, CYP27A1, OLR1, GRN, GLIPR2, ARRDC4, C1QC, APOE, FOLR2, CTSD and SPP1) by first z-score normalization of each gene across patients followed by the calculation of an average z-score across signature genes for each patient (see, e.g., Guan et al, Anti-TIGIT antibody improves PD-L1 blockade through myeloid and T_{reg} cells. Nature (2024); 627:646-654).

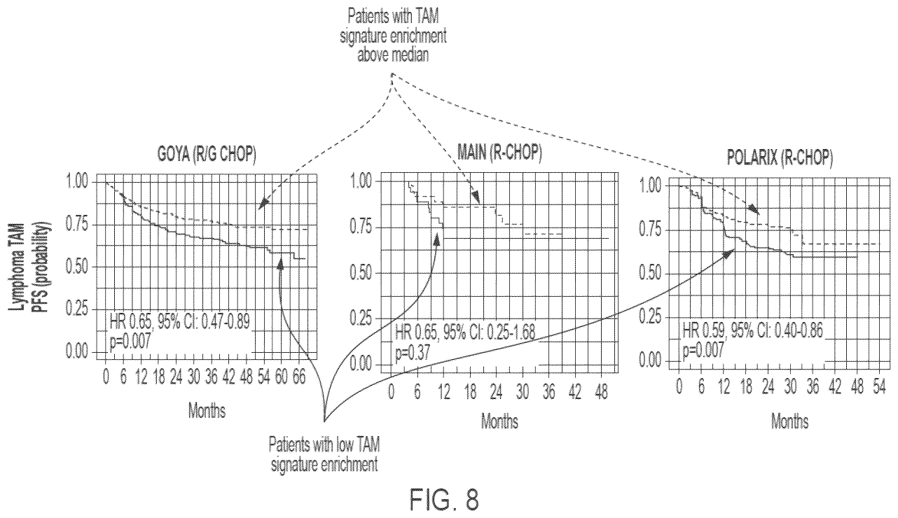

The results described in Example 1 raised the possibility that rather than overall macrophage infiltration of the lymphoma tissue, a particular transcriptional state reflecting macrophage polarization or certain activation or differentiation features may be responsible for the observed effect on CD20-targeted chemoimmunotherapy outcomes. To further validate and extend the result of the lymphoma microenvironment analysis, a recently published macrophage signature derived from single cell transcriptomic data from tumor associated macrophages (TAMs) in solid tumors (NSCLC) was investigated (Guan et al, Anti-TIGIT antibody improves PD-L1 blockade through myeloid and T_{reg} cells. Nature (2024); 627:646-654). In lymphoma, patients with above median TAM signature enrichment showed improved responses to R/G (rituximab/obinutuzumab)−CHOP treated patients compared to TAM low patients in GOYA (HR 0.65, 95% CI: 0.47-0.89, p=0.007) as well as in the R-CHOP control arm of POLARIX (HR 0.59, 95% CI: 0.40-0.86, p=0.007) (FIG. 8).

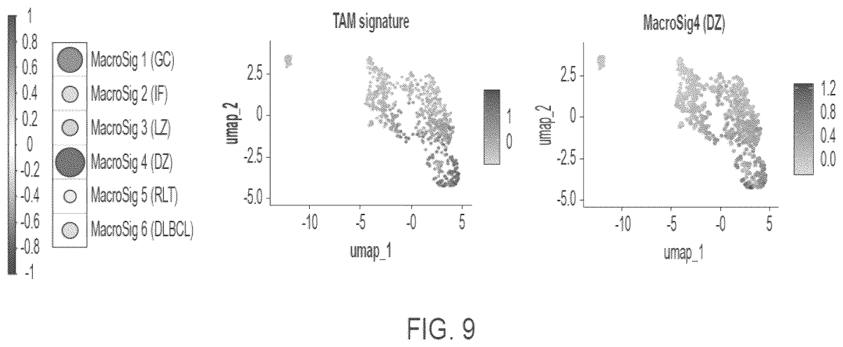

Without being bound by theory, given that this TAM signature encompassed tumor promoting macrophage subsets and markers (i.e. MARCO), CD20-targeted antibodies (e.g., obinutuzumab (G) or rituximab (R)) may leverage FcγR engagement for their clinical activity including engagement of macrophages with tumor promoting functions. To better understand what specific macrophage functional state or feature was associated with improved R-CHOP outcomes a spatial transcriptomics analysis of defined CD68⁺ macrophage subsets from reactive lymph nodes and lymphoma tissues was interrogated in combination with a publicly available single cell pan-lymphoid tissue atlas that included cells from de novo DLBCL and FL biopsies along with cells from lymphoid tumors, healthy tonsils and reactive lymph nodes. The TAM signature previously found to be associated with improved R/G-CHOP outcomes was strongly correlated with the MacroSig4 signature derived from DZ macrophages (R=0.871, Pearson) followed by MacroSig1 derived from GC macrophages (FIG. 9). Projection of the TAM signature onto this scRNA macrophage dataset corroborated preferential enrichment on a subset of lymphoma macrophages that largely overlapped with DZ macrophages (FIG. 9). In addition, mapping of this prognostic TAM signature onto an integrative human scRNA macrophage compendium (MoMac-VERSE) (See Mulder K, Patel A A, Kong W T, et al. Cross-tissue single-cell landscape of human monocytes and macrophages in health and disease. Immunity. 2021; 54(8):1883-1900.e5) suggested that it most closely overlapped with TREM2+ macrophages as well as C1Q macrophages ((FIG. 9). Without being bound by theory, rituximab may rely on eliciting immune effector functions to drive responses in the context of CD20-targeted chemoimmunotherapy via Fc region interactions with specific subsets of lymphoma infiltrating macrophages that resemble DZ macrophages and may contain immunosuppressive or tumor promoting properties.

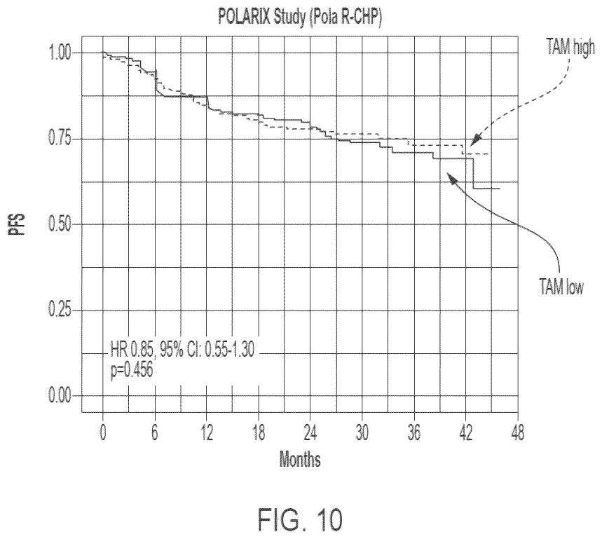

No difference was observed when patients were treated with Pola-R-CHP regardless of TAM enrichment (FIG. 10). In summary, these results show that Pola-R-CHP treatment improved survival outcome (e.g., PFS) in patients with low M1 macrophage infiltration levels. Without being bound by theory, the addition of polatuzumab vedotin to the treatment regimen may alter the effect of preexisting lymphoma microenvironment in a manner that renders preexisting TAM prevalence negligible to treatment outcomes in this cohort with low M1 macrophage infiltration levels.

### Conclusions

The results confirm that the efficacy of R-CHOP is associated with a specific lymphoma microenvironment, and in contrast, Pola-R-CHP may achieve therapeutic responses in patients who lack a favorable immune contexture, suggesting Pola-R-CHP can at least partially decouple treatment outcome from the lymphoma microenvironment. Therefore, it may be beneficial to select Pola-R-CHP therapy for a patient that has a low tumor-associated macrophage signature.

1. A method of identifying, diagnosing, and/or predicting whether a patient having a diffuse large B-cell lymphoma (DLBCL) may benefit from a treatment comprising an immunoconjugate and an anti-CD20 antibody, the method comprising measuring a macrophage biomarker in a sample from the patient, wherein an amount or level of the macrophage biomarker in the sample that is below a reference macrophage biomarker amount or level identifies, diagnoses, and/or predicts the patient as one who may benefit from the treatment comprising the immunoconjugate and the anti-CD20 antibody, wherein the immunoconjugate comprises the formula: 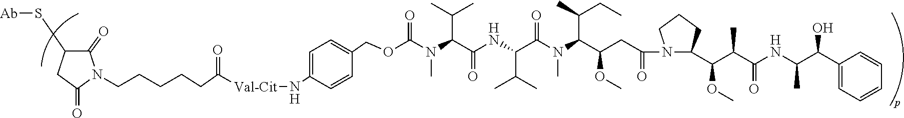
wherein Ab is an anti-CD79b antibody comprising: (i) an HVR-H1 that comprises the amino acid sequence of SEQ ID NO: 5; (ii) an HVR-H2 comprising the amino acid sequence of SEQ ID NO: 6; (iii) an HVR-H3 comprising the amino acid sequence of SEQ ID NO: 7; (iv) an HVR-L1 comprising the amino acid sequence of SEQ ID NO: 8; (v) an HVR-L2 comprising the amino acid sequence of SEQ ID NO: 9; and (vi) an HVR-L3 comprising the amino acid sequence of SEQ ID NO:10, and wherein p is between 1 and 8.

2. A method of selecting a therapy for a patient having a DLBCL, the method comprising measuring a macrophage biomarker in a sample from the patient, wherein an amount or level of the macrophage biomarker in the sample that is below a reference macrophage biomarker amount or level identifies the patient as one who may benefit from a treatment comprising an immunoconjugate and an anti-CD20 antibody, wherein the immunoconjugate comprises the formula: 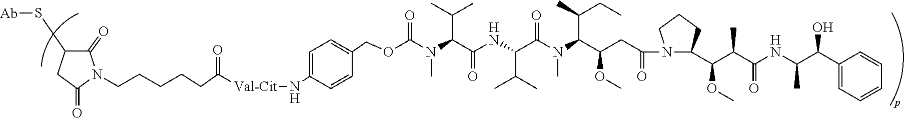
wherein Ab is an anti-CD79b antibody comprising: (i) an HVR-H1 that comprises the amino acid sequence of SEQ ID NO: 5; (ii) an HVR-H2 comprising the amino acid sequence of SEQ ID NO: 6; (iii) an HVR-H3 comprising the amino acid sequence of SEQ ID NO: 7; (iv) an HVR-L1 comprising the amino acid sequence of SEQ ID NO: 8; (v) an HVR-L2 comprising the amino acid sequence of SEQ ID NO: 9; and (vi) an HVR-L3 comprising the amino acid sequence of SEQ ID NO:10, and wherein p is between 1 and 8.

3. The method of claim 1 or 2, wherein the amount or level of the macrophage biomarker from the patient is below the reference macrophage biomarker amount or level, and the method further comprises administering to the patient an effective amount of the immunoconjugate and an effective amount of the anti-CD20 antibody.

4. A method of treating a patient having a DLBCL, the method comprising:
(a) measuring a macrophage biomarker in a sample from the patient, wherein the amount or level of the macrophage biomarker in the sample is below a reference macrophage biomarker amount or level, and
(b) administering an effective amount of an immunoconjugate and an effective amount of an anti-CD20 antibody to the patient based on the macrophage biomarker measured in step (a), and wherein the immunoconjugate comprises the formula: 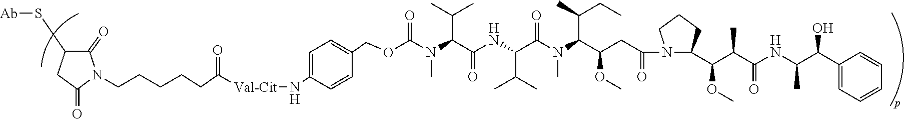
wherein Ab is an anti-CD79b antibody comprising: (i) an HVR-H1 that comprises the amino acid sequence of SEQ ID NO: 5; (ii) an HVR-H2 comprising the amino acid sequence of SEQ ID NO: 6; (iii) an HVR-H3 comprising the amino acid sequence of SEQ ID NO: 7; (iv) an HVR-L1 comprising the amino acid sequence of SEQ ID NO: 8; (v) an HVR-L2 comprising the amino acid sequence of SEQ ID NO: 9; and (vi) an HVR-L3 comprising the amino acid sequence of SEQ ID NO:10, and wherein p is between 1 and 8.

5. A method of treating a patient having a DLBCL, the method comprising administering to the patient an effective amount of an immunoconjugate and an effective amount of an anti-CD20 antibody, wherein prior to treatment the amount or level of a macrophage biomarker in a sample from the patient has been determined to be below a reference macrophage biomarker amount or level, and wherein the immunoconjugate comprises the formula: 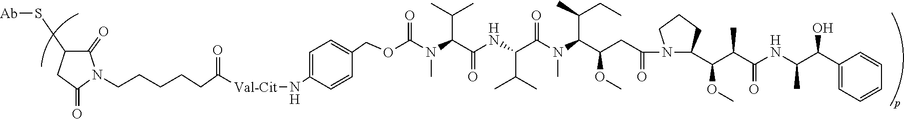
wherein Ab is an anti-CD79b antibody comprising: (i) an HVR-H1 that comprises the amino acid sequence of SEQ ID NO: 5; (ii) an HVR-H2 comprising the amino acid sequence of SEQ ID NO: 6; (iii) an HVR-H3 comprising the amino acid sequence of SEQ ID NO: 7; (iv) an HVR-L1 comprising the amino acid sequence of SEQ ID NO: 8; (v) an HVR-L2 comprising the amino acid sequence of SEQ ID NO: 9; and (vi) an HVR-L3 comprising the amino acid sequence of SEQ ID NO:10, and wherein p is between 1 and 8.

6. A method of treating a patient having a DLBCL and having an amount or level of a macrophage biomarker in a sample from the patient that is below a reference macrophage biomarker amount or level comprising administering to the patient an effective amount of an immunoconjugate and an effective amount of an anti-CD20 antibody, wherein the immunoconjugate comprises the formula: 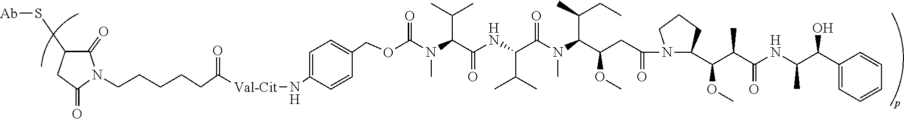
wherein Ab is an anti-CD79b antibody comprising: (i) an HVR-H1 that comprises the amino acid sequence of SEQ ID NO: 5; (ii) an HVR-H2 comprising the amino acid sequence of SEQ ID NO: 6; (iii) an HVR-H3 comprising the amino acid sequence of SEQ ID NO: 7; (iv) an HVR-L1 comprising the amino acid sequence of SEQ ID NO: 8; (v) an HVR-L2 comprising the amino acid sequence of SEQ ID NO: 9; and (vi) an HVR-L3 comprising the amino acid sequence of SEQ ID NO:10, and wherein p is between 1 and 8.

7. The method of any one of claims 1-6, wherein the patient is a human patient.

8. The method of any one of claims 1-7, wherein the reference macrophage biomarker amount or level is a pre-assigned macrophage biomarker amount or level.

9. The method of any one of claims 1-8, wherein the reference macrophage biomarker amount or level is an amount or level of a macrophage biomarker in a reference population.

10. The method of claim 9, wherein the amount or level of the macrophage biomarker in a reference population is a median amount or level of the macrophage biomarker of the reference population.

11. The method of claim 9, wherein the reference macrophage biomarker amount or level is an amount or level of a macrophage biomarker that is at the 25th percentile of the reference population.

12. The method of claim 9, wherein the reference macrophage biomarker amount or level is an amount or level of a macrophage biomarker that is at the 50th percentile of the reference population.

13. The method of claim 9, wherein the reference macrophage biomarker amount or level is an amount or level of a macrophage biomarker that is at the 75th percentile of the reference population.

14. The method of any one of claims 9-13, wherein the reference population is a population of patients having the DLBCL.

15. The method of claim 14, wherein the population of patients having the DLBCL was previously treated with the immunoconjugate and the anti-CD20 antibody.

16. The method of claim 14, wherein the population of patients having the DLBCL was previously treated with the anti-CD20 antibody.

17. The method of claim 14, wherein the reference macrophage biomarker amount or level is the amount or level of macrophage biomarker of the reference population prior to initiating treatment with the immunoconjugate and the anti-CD20 antibody.

18. The method of any one of claims 1-17, wherein the reference macrophage biomarker amount or level is an amount of macrophages as measured by gene expression.

19. The method of claim 18, wherein the amount of macrophages is between about 0% to about 56.5%.

20. The method of any one of claims 1-3, wherein the benefit is an extension of progression-free survival (PFS).

21. The method of any one of claims 1-3, wherein the benefit is an increase in overall survival (OS).

22. The method of any one of claims 4-6, further comprising achieving an improvement of PFS or OS.

23. The method of any one of claims 1-22, wherein the macrophage biomarker is an average of M1 macrophage gene signature set scores of one or more M1 macrophage gene signature sets.

24. The method of claim 23, wherein each M1 macrophage gene signature set score is an average of the expression level of one or more genes of an M1 macrophage gene signature set.

25. The method of claim 24, wherein each M1 macrophage gene signature set score is an average of the normalized expression level of one or more genes of an M1 macrophage gene signature set.

26. The method of claim 24 or 25, wherein the one or more M1 macrophage gene signature sets are:
(a) ACP2, ABCD1, C1QA, FDX1, CCL22, CD163, SCAMP2, ADAMDEC1, ARL8B, and HAMP;
(b) ACP2, ABCD1, FDX1, CCL8, CCL22, CD163, ADAMDEC1, TREM2, and HAMP;
(c) ACP2, ADRA2B, ALCAM, ABCD1, ATOX1, ATP6VOC, ATP6V1E1, BLVRA, C1QA, CD48, CD63, CLCN7, TPP1, CLTC, CCR1, CMKLR1, SLC31A1, COX5B, FCER1G, FDX1, FOLR2, FPR3, FTL, HEXB, HK3, IL10, IL12B, ITGAE, LAIR1, CXCL9, MMP19, NARS, NDUFS2, P2RX7, PDCL, MAPK13, PTGIR, PTPRA, RELA, CCL7, CCL8, CCL19, CCL22, SRC, STX4, TCEB1, TFRC, AGPS, MARCO, SNX3, CD84, USP14, ITGB1BP1, ATP6V1F, TRIP4, CD163, CIAO1, WTAP, ARHGEF11, ABI1, SCAMP2, ACTR2, BCAP31, ZMPSTE24, BCKDK, EXOC5, STIP1, UQCR11, SDS, LILRB4, OGFR, TFEC, FKBP15, DNAJC13, TDRD7, STX12, IL17RA, ABTB2, FAM32A, SIGLEC7, SIGLEC9, ADAMDEC1, CECR5, SLC25A24, NRBP1, MS4A4A, TREM2, OTUD4, PQLC2, HAUS2, ARL8B, NECAP2, WDR11, ZC3H15, CCDC47, UTP3, MRS2, HAMP, MRPL40, VPS33A, CORO7, LIMD2, TMX1, DOT1L, ADO, and ADCK2;
(d) ACP2, ADRA2B, ALCAM, TSPO, C3AR1, DAGLA, CALR, CHIT1, CYBB, CYC1, CYP19A1, DLAT, FCER1G, GP1BA, GPD1, IFNAR1, IL10, KCNJ5, KIFC3, MT2A, MYBPH, MYH11, MYO7A, P2RX7, PRDX1, RAB3IL1, RNH1, MRPL12, CCL1, CCL7, CCL8, CCL24, SRC, VIM, RRP1, MARCO, S1PR2, AP1M2, ACTR3, LILRB1, AFG3L2, SDS, LILRB4, EMILIN1, VSIG4, HSPB7, COQ2, ADAMDEC1, CECR5, WSB2, SLAMF8, DNASE2B, CLPB, MFSD7, and ADCK2;
(e) ACP2, ADCY3, ADRA2B, ALCAM, TSPO, C1QA, C1QB, C3AR1, DAGLA, CD63, CHIT1, CMKLR1, SLC31A1, CSF1, CSF1R, CYBB, CYC1, CYP19A1, FANCE, FCER1G, FDX1, FPR3, FTL, GP1BA, GPD1, HEXB, IL10, KCNJ1, KCNJ5, KIFC3, LAMP1, MMP19, MSR1, MT2A, MYBPH, MYO7A, P2RX7, PRDX1, RAB3IL1, MRPL12, CCL1, CCL7, CCL8, CCL18, CCL19, CCL24, SLC6A12, SPR, SRC, RRP1, MARCO, PKD2L1, S1PR2, CD163, LONP1, AP1M2, IGSF6, LILRB1, SDS, LILRB4, EMILIN1, VSIG4, TFEC, PHLDB1, CYFIP1, FKBP15, NCAPH, MYOF, HSPB7, ADAMDEC1, GLRX2, NDUFAF1, SPG21, MS4A4A, ATP6V1D, ATP6V1H, TREM2, PQLC2, TMEM70, PLEKHB2, TMEM33, SLAMF8, HAMP, DNASE2B, MYOZ1, LONRF3, CLPB, MFSD7, and ADCK2; and/or
(f) ACP2, ADCY3, ADRA2B, ALCAM, ABCD1, ANXA2, ATP6V1A, C1QA, C1QB, C3AR1, DAGLA, CD80, CD63, CHIT1, CMKLR1, SLC31A1, CSF1, CSF1R, CYBB, CYC1, CYP19A1, FANCE, FDX1, FPR2, FPR3, GPD1, HEXB, KCNJ1, KCNJ5, KIFC3, MMP19, MSR1, MT2A, MYBPH, P2RX7, MAPK13, S100A11, CCL1, CCL7, CCL8, CCL18, CCL19, CCL22, CCL24, SLC1A2, SLC6A12, SLC11A1, SIGLEC1, SRC, TIE1, MARCO, HYAL2, CD163, LONP1, IGSF6, LILRB1, CD300C, SDS, LILRB4, EMILIN1, VSIG4, PHLDB1, NCAPH, CLEC4E, MYOF, HSPB7, ADAMDEC1, GLRX2, MS4A4A, ATP6V1H, TREM2, TMEM70, TMEM33, KCNK13, SLAMF8, HAMP, DNASE2B, MYOZ1, MFSD7, ADO, ADCK2, and TBC1D16.

27. The method of any one of claims 1-22, wherein the macrophage biomarker is an average of TAM gene signature set scores of one or more TAM gene signature sets.

28. The method of claim 27, wherein each TAM gene signature set score is an average of the expression level of one or more genes of a TAM gene signature set.

29. The method of claim 28, wherein each TAM gene signature set score is an average of the normalized expression level of one or more genes of a TAM gene signature set.

30. The method of claim 28 or 29, wherein the one or more TAM gene signature sets are:
(a) MARCO, ACP5, VSIG4, MRC1, MSR1, MCEMP1, CYP27A1, OLR1, GRN, GLIPR2, ARRDC4, C1QC, APOE, FOLR2, CTSD and SPP1.

31. The method of any one of claims 1-22, wherein the macrophage biomarker is a gene expression value.

32. The method of claim 31, wherein the gene expression value is a median gene expression value.

33. The method of claim 31 or 32, wherein the gene expression value is measured using a gene signature matrix.

34. The method of claim 33, wherein the gene signature matrix comprises the following genes:
(a) CD200, KLHL14, TCL1A, NRG1, EOMES, PPP2R2B, RNF165, WNT7A, CCR4, PDGFD, EBF1, FCGBP, PCDH9, MLC1, TSHZ2, S1PR5, NCALD, LAYN, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, ABCB4, CD5, HPGD, BLNK, PLCL1, HPSE, SLFN13, HOPX, CD1D, GNG7, TCF4, BANK1, FHIT, FCMR, GNG2, GFRA2, KBTBD11, RALGPS2, TSPOAP1, PLEKHF1, MEF2C, MAOA, TTYH2, HLA-DOB, DGAT2, FXYD6, TMCC3, MGAM, TTC38, LRRC32, ARHGAP24, STAT4, SLC7A8, CD72, FZD1, GK5, DYSF, PLTP, SMARCD3, FAM160B1, PDPN, AKAP2, ACVRL1, KCNJ15, ALDH1A2, ENPP2, COLEC12, PTGS1, TMEM170B, TREM2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, LILRA2, ACE, NUPR1, CISH, EREG, ADAMDEC1, RNASE6, CXCL3, VSIG4, CXCL2, CD86, LILRB4, SERPING1, SQOR, INHBA, and ICAM1; or
(b) CD200, KLHL14, TCL1A, NRG1, CYP4F3, EOMES, PPP2R2B, RNF165, WNT7A, CCR4, PDGFD, EBF1, FCGBP, PCDH9, MLC1, TSHZ2, S1PR5, NCALD, LAYN, CD248, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, NINJ2, ABCB4, CD5, HAL, HPGD, BLNK, PLCL1, CEP19, HPSE, SLFN13, HOPX, CD1D, GNG7, TMEM154, TCF4, BANK1, FHIT, FCMR, GNG2, GFRA2, KBTBD11, TECPR2, RALGPS2, TSPOAP1, PLEKHF1, MEF2C, MAOA, TTYH2, HLA-DOB, NRGN, DGAT2, FXYD6, TMCC3, MGAM, TTC38, LRRC32, ARHGAP24, PPP1R3B, STAT4, SLC7A8, CD72, FZD1, GK5, DYSF, PLTP, SMARCD3, FAM160B1, PDPN, AKAP2, ACVRL1, KCNJ15, CD36, ALDH1A2, ENPP2, COLEC12, PTGS1, TMEM170B, DOCK5, TREM2, C5AR2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, TLR4, LILRA2, ACE, TLR1, LRRK2, LY96, NUPR1, CISH, CSTA, EREG, ADAMDEC1, RNASE6, CXCL3, VSIG4, CXCL2, CD86, LILRB4, SERPING1, SQOR, INHBA, and ICAM1.

35. The method of claim 34, wherein the gene signature matrix consists of the following genes: CD200, KLHL14, TCL1A, NRG1, EOMES, PPP2R2B, RNF165, WNT7A, CCR4, PDGFD, EBF1, FCGBP, PCDH9, MLC1, TSHZ2, S1PR5, NCALD, LAYN, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, ABCB4, CD5, HPGD, BLNK, PLCL1, HPSE, SLFN13, HOPX, CD1D, GNG7, TCF4, BANK1, FHIT, FCMR, GNG2, GFRA2, KBTBD11, RALGPS2, TSPOAP1, PLEKHF1, MEF2C, MAOA, TTYH2, HLA-DOB, DGAT2, FXYD6, TMCC3, MGAM, TTC38, LRRC32, ARHGAP24, STAT4, SLC7A8, CD72, FZD1, GK5, DYSF, PLTP, SMARCD3, FAM160B1, PDPN, AKAP2, ACVRL1, KCNJ15, ALDH1A2, ENPP2, COLEC12, PTGS1, TMEM170B, TREM2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, LILRA2, ACE, NUPR1, CISH, EREG, ADAMDEC1, RNASE6, CXCL3, VSIG4, CXCL2, CD86, LILRB4, SERPING1, SQOR, INHBA, and ICAM1.

36. The method of any one of claims 33-35, wherein the gene signature matrix is used to determine a number of M1 macrophages or tumor-associated macrophages.

37. The method of any one of claims 1-22, wherein the macrophage biomarker is an amount of M1 macrophages or an amount of tumor-associated macrophages.

38. The method of claim 37, wherein the amount of M1 macrophages or tumor-associated macrophages is measured directly or indirectly.

39. The method of claim 38, wherein the amount of M1 macrophages or tumor-associated macrophages is measured directly using flow cytometry, spatial transcriptomics, spatial proteomics, or combination thereof.

40. The method of claim 38, wherein the amount of M1 macrophages or tumor-associated macrophages is measured indirectly using nucleic acid or protein.

41. The method of claim 40, wherein the nucleic acid is measured using RNA-seq, RT-qPCR, qPCR, multiplex qPCR or RT-qPCR, microarray analysis, SAGE, MassARRAY technique, ISH, or a combination thereof.

42. The method of claim 41, wherein the amount of M1 macrophages or tumor-associated macrophages is measured using a marker gene approach or a deconvolution approach.

43. The method of claim 42, wherein the marker gene approach uses xCell.

44. The method of claim 42, wherein the deconvolution approach uses quanTIseq.

45. The method of any one of claims 1-22, wherein the macrophage biomarker in the sample from the patient is measured using nucleic acid or protein.

46. The method of claim 45, wherein the macrophage biomarker in the sample from the patient is determined using a nucleic acid expression level.

47. The method of claim 46, wherein the nucleic acid expression level is determined by RNA-seq, RT-qPCR, qPCR, multiplex qPCR or RT-qPCR, microarray analysis, SAGE, MassARRAY technique, ISH, or a combination thereof.

48. The method of claim 46 or 47, wherein the nucleic acid expression level is an mRNA expression level.

49. The method of claim 48, wherein the mRNA expression level is determined by RNA-seq.

50. The method of any one of claims 1-49, wherein the sample is a tissue sample, tumor sample, whole blood sample, a plasma sample, a serum sample, or a combination thereof.

51. The method of claim 50, wherein the sample is a tissue sample.

52. The method of claim 51, wherein the tissue sample is a tumor tissue sample.

53. The method of claim 52, wherein the tumor tissue sample contains tumor cells, tumor-infiltrating immune cells, stromal cells, normal adjacent tissue (NAT) cells, or a combination thereof.

54. The method of claim 52 or 53, wherein the tumor tissue sample is a biopsy.

55. The method of any one of claims 50-54, wherein the sample is an archival sample, a fresh sample, or a frozen sample.

56. The method of any one of claims 1-55, wherein the DLBCL is a germinal-center B-cell-like (GCB) or activated B-cell-like (ABC) cell-of-origin subgroup of DLBCL.

57. The method of any one of claims 1-56, wherein the DLBCL is a CD79b- and/or CD20-positive DLBCL.

58. The method of any one of claims 1-57, wherein the patient has not been previously treated for the DLBCL.

59. The method of any one of claims 1-58, wherein the patient has not been previously administered the immunoconjugate and the anti-CD20 antibody.

60. The method of any one of claims 1-59, wherein the anti-CD79b antibody comprises a heavy chain variable domain (VH) comprising the amino acid sequence of SEQ ID NO: 3 and a light chain variable domain (VL) comprising the amino acid sequence of SEQ ID NO: 4.

61. The method of any one of claims 1-60, wherein the anti-CD79b antibody comprises:
(a) a heavy chain comprising the amino acid sequence of SEQ ID NO: 13 and a light chain comprising the amino acid sequence of SEQ ID NO: 11;
(b) a heavy chain comprising the amino acid sequence of SEQ ID NO: 12 and a light chain comprising the amino acid sequence of SEQ ID NO: 14; or
(c) a heavy chain comprising the amino acid sequence of SEQ ID NO: 12 and a light chain comprising the amino acid sequence of SEQ ID NO: 11.

62. The method of any one of claims 1-61, wherein p is between 2 and 7, between 2 and 6, between 2 and 5, between 3 and 5, or between 3 and 4.

63. The method of claim 62, wherein p is 3.4.

64. The method of claim 62, wherein p is 3.5.

65. The method of any one of claims 1-64, wherein the immunoconjugate is polatuzumab vedotin.

66. The method of any one of claims 1-65, wherein the anti-CD20 antibody is a type I anti-CD20 antibody or a type II anti-CD20 antibody.

67. The method of claim 66, wherein the anti-CD20 antibody is a type I anti-CD20 antibody.

68. The method of claim 67, wherein the type I anti-CD20 antibody comprises the following CDRs:
(a) a CDR-H1 with an amino acid sequence of SEQ ID NO: 26;
(b) a CDR-H2 with an amino acid sequence of SEQ ID NO: 27;
(c) a CDR-H3 with an amino acid sequence of SEQ ID NO: 28;
(d) a CDR-L1 with an amino acid sequence of SEQ ID NO: 29;
(e) a CDR-L2 with an amino acid sequence of SEQ ID NO: 30; and
(f) a CDR-L3 with an amino acid sequence of SEQ ID NO: 31.

69. The method of claim 68, wherein the type I anti-CD20 antibody comprises a VH domain comprising an amino acid sequence of SEQ ID NO: 40 and a VL domain comprising an amino acid sequence of SEQ ID NO: 41.

70. The method of claim 69, wherein the type I anti-CD20 antibody is rituximab.

71. The method of any one of claims 65-70, wherein polatuzumab vedotin is administered at a dose of about 1.0 mg/kg to about 1.8 mg/kg.

72. The method of claim 71, wherein polatuzumab vedotin is administered at a dose of about 1.8 mg/kg.

73. The method of any one of claims 70-72, wherein rituximab is administered at a dose of about 375 mg/m².

74. The method of any one of claims 65-73, wherein polatuzumab vedotin and/or rituximab is administered intravenously.

75. The method of any one of claims 3-74, further comprising administering to the patient an effective amount of an additional therapeutic agent.

76. The method of claim 75, wherein the additional therapeutic agent is one or more of a chemotherapeutic agent, a corticosteroid, an anti-neoplastic agent, a growth inhibitory agent, an anti-angiogenic agent, a radiation therapy, a cytotoxic agent, or a combination thereof.

77. The method of claim 76, wherein the additional therapeutic agent is a chemotherapeutic agent and a corticosteroid.

78. The method of claim 76 or 77, wherein the chemotherapeutic agent is cyclophosphamide and/or doxorubicin.

79. Th method of any one of claims 76-78, wherein the corticosteroid is prednisone, prednisolone, or methylprednisolone.

80. The method of claim 78 or 79, wherein cyclophosphamide is administered at a dose of about 375 mg/m² to about 750 mg/m².

81. The method of any one of claims 78-80, wherein doxorubicin is administered at a dose of about 25 mg/m² to about 50 mg/m².

82. The method of any one of claims 79-81, wherein
(a) prednisone is administered at a dose of about 100 mg;
(b) prednisolone is administered at a dose of about 100 mg; or
(c) methylprednisolone is administered at a dose of about 80 mg.

83. The method of any one of claims 78-82, wherein cyclophosphamide and/or doxorubicin are administered intravenously.

84. The method of any one of claims 79-83, wherein prednisone, prednisolone, or methylprednisolone is administered orally.

85. The method of any one of claims 79-84, wherein polatuzumab vedotin, rituximab, cyclophosphamide, doxorubicin, and/or prednisone, prednisolone, or methylprednisolone are administered in at least one 21-day cycle.

86. The method of claim 85, wherein:
(a) the polatuzumab vedotin, rituximab, cyclophosphamide, and/or doxorubicin are administered on day 1 of each 21-day cycle; and/or
(b) prednisone, prednisolone, or methylprednisolone is administered on days 1-5 of each 21-day cycle.

87. The method of any one of claims 85-86, wherein polatuzumab vedotin, rituximab, cyclophosphamide, doxorubicin, and/or prednisone, prednisolone, or methylprednisolone are administered for one, two, three, four, five, or six 21-day cycles.

88. Use of an immunoconjugate and an anti-CD20 antibody for treating a patient having an amount or level of a macrophage biomarker in a sample from the patient that is below a reference macrophage biomarker amount or level in the manufacture of a medicament for the treatment of a DLBCL, and wherein the immunoconjugate comprises the formula: 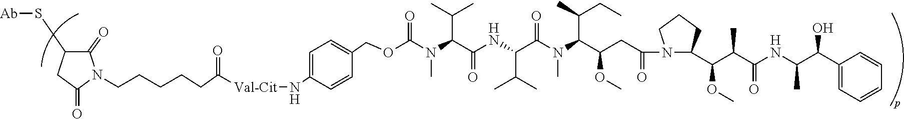
wherein Ab is an anti-CD79b antibody comprising: (i) an HVR-H1 that comprises the amino acid sequence of SEQ ID NO: 5; (ii) an HVR-H2 comprising the amino acid sequence of SEQ ID NO: 6; (iii) an HVR-H3 comprising the amino acid sequence of SEQ ID NO: 7; (iv) an HVR-L1 comprising the amino acid sequence of SEQ ID NO: 8; (v) an HVR-L2 comprising the amino acid sequence of SEQ ID NO: 9; and (vi) an HVR-L3 comprising the amino acid sequence of SEQ ID NO:10, and wherein p is between 1 and 8.

89. The use of claim 88, wherein the patient is a human patient.

90. The use of claim 88 or 89, wherein the reference macrophage biomarker amount or level is a pre-assigned macrophage biomarker amount or level.

91. The use of any one of claims 88-90, wherein the reference macrophage biomarker amount or level is an amount or level of a macrophage biomarker in a reference population.

92. The use of claim 91, wherein the amount or level of the macrophage biomarker in a reference population is a median amount or level of the macrophage biomarker of the reference population.

93. The use of claim 91, wherein the reference macrophage biomarker amount or level is an amount or level of a macrophage biomarker that is at the 25th percentile of the reference population.

94. The use of claim 91, wherein the reference macrophage biomarker amount or level is an amount or level of a macrophage biomarker that is at the 50th percentile of the reference population.

95. The use of claim 91, wherein the reference macrophage biomarker amount or level is an amount or level of a macrophage biomarker that is at the 75th percentile of the reference population.

96. The use of any one of claims 91-95, wherein the reference population is a population of patients having the DLBCL.

97. The use of claim 96, wherein the population of patients having the DLBCL was previously treated with the immunoconjugate and the anti-CD20 antibody.

98. The use of claim 96, wherein the population of patients having the DLBCL was previously treated with the anti-CD20 antibody.

99. The use of claim 96, wherein the reference macrophage biomarker amount or level is the amount or level of macrophage biomarker of the reference population prior to initiating treatment with the immunoconjugate and the anti-CD20 antibody.

100. The use of any one of claims 88-99, wherein the reference macrophage biomarker amount or level is an amount of macrophages as measured by gene expression.

101. The use of claim 100, wherein the amount of macrophages is between about 0% to about 56.5%.

102. The use of any one of claims 88-101, wherein the treatment achieves an improvement of PFS or OS.

103. The use of any one of claims 88-102, wherein the macrophage biomarker is an average of M1 macrophage gene signature set scores of one or more M1 macrophage gene signature sets.

104. The use of claim 103, wherein each M1 macrophage gene signature set score is an average of the expression level of one or more genes of an M1 macrophage gene signature set.

105. The use of claim 104, wherein each M1 macrophage gene signature set score is an average of the normalized expression level of one or more genes of an M1 macrophage gene signature set.

106. The use of claim 104 or 105, wherein the one or more M1 macrophage gene signature sets are:
(a) ACP2, ABCD1, C1QA, FDX1, CCL22, CD163, SCAMP2, ADAMDEC1, ARL8B, and HAMP;
(b) ACP2, ABCD1, FDX1, CCL8, CCL22, CD163, ADAMDEC1, TREM2, and HAMP;
(c) ACP2, ADRA2B, ALCAM, ABCD1, ATOX1, ATP6VOC, ATP6V1E1, BLVRA, C1QA, CD48, CD63, CLCN7, TPP1, CLTC, CCR1, CMKLR1, SLC31A1, COX5B, FCER1G, FDX1, FOLR2, FPR3, FTL, HEXB, HK3, IL10, IL12B, ITGAE, LAIR1, CXCL9, MMP19, NARS, NDUFS2, P2RX7, PDCL, MAPK13, PTGIR, PTPRA, RELA, CCL7, CCL8, CCL19, CCL22, SRC, STX4, TCEB1, TFRC, AGPS, MARCO, SNX3, CD84, USP14, ITGB1BP1, ATP6V1F, TRIP4, CD163, CIAO1, WTAP, ARHGEF11, ABI1, SCAMP2, ACTR2, BCAP31, ZMPSTE24, BCKDK, EXOC5, STIP1, UQCR11, SDS, LILRB4, OGFR, TFEC, FKBP15, DNAJC13, TDRD7, STX12, IL17RA, ABTB2, FAM32A, SIGLEC7, SIGLEC9, ADAMDEC1, CECR5, SLC25A24, NRBP1, MS4A4A, TREM2, OTUD4, PQLC2, HAUS2, ARL8B, NECAP2, WDR11, ZC3H15, CCDC47, UTP3, MRS2, HAMP, MRPL40, VPS33A, CORO7, LIMD2, TMX1, DOT1L, ADO, and ADCK2;
(d) ACP2, ADRA2B, ALCAM, TSPO, C3AR1, DAGLA, CALR, CHIT1, CYBB, CYC1, CYP19A1, DLAT, FCER1G, GP1BA, GPD1, IFNAR1, IL10, KCNJ5, KIFC3, MT2A, MYBPH, MYH11, MYO7A, P2RX7, PRDX1, RAB3IL1, RNH1, MRPL12, CCL1, CCL7, CCL8, CCL24, SRC, VIM, RRP1, MARCO, S1PR2, AP1M2, ACTR3, LILRB1, AFG3L2, SDS, LILRB4, EMILIN1, VSIG4, HSPB7, COQ2, ADAMDEC1, CECR5, WSB2, SLAMF8, DNASE2B, CLPB, MFSD7, and ADCK2;
(e) ACP2, ADCY3, ADRA2B, ALCAM, TSPO, C1QA, C1QB, C3AR1, DAGLA, CD63, CHIT1, CMKLR1, SLC31A1, CSF1, CSF1R, CYBB, CYC1, CYP19A1, FANCE, FCER1G, FDX1, FPR3, FTL, GP1BA, GPD1, HEXB, IL10, KCNJ1, KCNJ5, KIFC3, LAMP1, MMP19, MSR1, MT2A, MYBPH, MYO7A, P2RX7, PRDX1, RAB3IL1, MRPL12, CCL1, CCL7, CCL8, CCL18, CCL19, CCL24, SLC6A12, SPR, SRC, RRP1, MARCO, PKD2L1, S1PR2, CD163, LONP1, AP1M2, IGSF6, LILRB1, SDS, LILRB4, EMILIN1, VSIG4, TFEC, PHLDB1, CYFIP1, FKBP15, NCAPH, MYOF, HSPB7, ADAMDEC1, GLRX2, NDUFAF1, SPG21, MS4A4A, ATP6V1D, ATP6V1H, TREM2, PQLC2, TMEM70, PLEKHB2, TMEM33, SLAMF8, HAMP, DNASE2B, MYOZ1, LONRF3, CLPB, MFSD7, and ADCK2; and/or
(f) ACP2, ADCY3, ADRA2B, ALCAM, ABCD1, ANXA2, ATP6V1A, C1QA, C1QB, C3AR1, DAGLA, CD80, CD63, CHIT1, CMKLR1, SLC31A1, CSF1, CSF1R, CYBB, CYC1, CYP19A1, FANCE, FDX1, FPR2, FPR3, GPD1, HEXB, KCNJ1, KCNJ5, KIFC3, MMP19, MSR1, MT2A, MYBPH, P2RX7, MAPK13, S100A11, CCL1, CCL7, CCL8, CCL18, CCL19, CCL22, CCL24, SLC1A2, SLC6A12, SLC11A1, SIGLEC1, SRC, TIE1, MARCO, HYAL2, CD163, LONP1, IGSF6, LILRB1, CD300C, SDS, LILRB4, EMILIN1, VSIG4, PHLDB1, NCAPH, CLEC4E, MYOF, HSPB7, ADAMDEC1, GLRX2, MS4A4A, ATP6V1H, TREM2, TMEM70, TMEM33, KCNK13, SLAMF8, HAMP, DNASE2B, MYOZ1, MFSD7, ADO, ADCK2, and TBC1D16.

107. The use of any one of claims 88-102, wherein the macrophage biomarker is an average of tumor-associated macrophage gene signature set scores of one or more tumor-associated macrophage gene signature sets.

108. The use of claim 107, wherein each tumor-associated macrophage gene signature set score is an average of the expression level of one or more genes of a tumor-associated macrophage gene signature set.

109. The use of claim 108, wherein each tumor-associated macrophage gene signature set score is an average of the normalized expression level of one or more genes of a tumor-associated macrophage gene signature set.

110. The use of claim 107 or 108, wherein the one or more tumor-associated macrophage gene signature sets are:
(g) MARCO, ACP5, VSIG4, MRC1, MSR1, MCEMP1, CYP27A1, OLR1, GRN, GLIPR2, ARRDC4, C1QC, APOE, FOLR2, CTSD and SPP1.

111. The use of any one of claims 88-102, wherein the macrophage biomarker is a gene expression value.

112. The use of claim 107, wherein the gene expression value is a median gene expression value.

113. The use of claim 107 or 112, wherein the gene expression value is measured using a gene signature matrix.

114. The use of claim 113, wherein the gene signature matrix comprises the following genes:
(a) CD200, KLHL14, TCL1A, NRG1, EOMES, PPP2R2B, RNF165, WNT7A, CCR4, PDGFD, EBF1, FCGBP, PCDH9, MLC1, TSHZ2, S1PR5, NCALD, LAYN, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, ABCB4, CD5, HPGD, BLNK, PLCL1, HPSE, SLFN13, HOPX, CD1D, GNG7, TCF4, BANK1, FHIT, FCMR, GNG2, GFRA2, KBTBD11, RALGPS2, TSPOAP1, PLEKHF1, MEF2C, MAOA, TTYH2, HLA-DOB, DGAT2, FXYD6, TMCC3, MGAM, TTC38, LRRC32, ARHGAP24, STAT4, SLC7A8, CD72, FZD1, GK5, DYSF, PLTP, SMARCD3, FAM160B1, PDPN, AKAP2, ACVRL1, KCNJ15, ALDH1A2, ENPP2, COLEC12, PTGS1, TMEM170B, TREM2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, LILRA2, ACE, NUPR1, CISH, EREG, ADAMDEC1, RNASE6, CXCL3, VSIG4, CXCL2, CD86, LILRB4, SERPING1, SQOR, INHBA, and ICAM1; or
(b) CD200, KLHL14, TCL1A, NRG1, CYP4F3, EOMES, PPP2R2B, RNF165, WNT7A, CCR4, PDGFD, EBF1, FCGBP, PCDH9, MLC1, TSHZ2, S1PR5, NCALD, LAYN, CD248, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, NINJ2, ABCB4, CD5, HAL, HPGD, BLNK, PLCL1, CEP19, HPSE, SLFN13, HOPX, CD1D, GNG7, TMEM154, TCF4, BANK1, FHIT, FCMR, GNG2, GFRA2, KBTBD11, TECPR2, RALGPS2, TSPOAP1, PLEKHF1, MEF2C, MAOA, TTYH2, HLA-DOB, NRGN, DGAT2, FXYD6, TMCC3, MGAM, TTC38, LRRC32, ARHGAP24, PPP1R3B, STAT4, SLC7A8, CD72, FZD1, GK5, DYSF, PLTP, SMARCD3, FAM160B1, PDPN, AKAP2, ACVRL1, KCNJ15, CD36, ALDH1A2, ENPP2, COLEC12, PTGS1, TMEM170B, DOCK5, TREM2, C5AR2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, TLR4, LILRA2, ACE, TLR1, LRRK2, LY96, NUPR1, CISH, CSTA, EREG, ADAMDEC1, RNASE6, CXCL3, VSIG4, CXCL2, CD86, LILRB4, SERPING1, SQOR, INHBA, and ICAM1.

115. The use of claim 114, wherein the gene signature matrix consists of the following genes: CD200, KLHL14, TCL1A, NRG1, EOMES, PPP2R2B, RNF165, WNT7A, CCR4, PDGFD, EBF1, FCGBP, PCDH9, MLC1, TSHZ2, S1PR5, NCALD, LAYN, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, ABCB4, CD5, HPGD, BLNK, PLCL1, HPSE, SLFN13, HOPX, CD1D, GNG7, TCF4, BANK1, FHIT, FCMR, GNG2, GFRA2, KBTBD11, RALGPS2, TSPOAP1, PLEKHF1, MEF2C, MAOA, TTYH2, HLA-DOB, DGAT2, FXYD6, TMCC3, MGAM, TTC38, LRRC32, ARHGAP24, STAT4, SLC7A8, CD72, FZD1, GK5, DYSF, PLTP, SMARCD3, FAM160B1, PDPN, AKAP2, ACVRL1, KCNJ15, ALDH1A2, ENPP2, COLEC12, PTGS1, TMEM170B, TREM2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, LILRA2, ACE, NUPR1, CISH, EREG, ADAMDEC1, RNASE6, CXCL3, VSIG4, CXCL2, CD86, LILRB4, SERPING1, SQOR, INHBA, and ICAM1.

116. The use of any one of claims 113-115, wherein the gene signature matrix is used to determine a number of M1 macrophages or tumor-associated macrophages.

117. The use of any one of claims 88-102, wherein the macrophage biomarker is an amount of M1 macrophages or an amount of tumor-associated macrophages.

118. The use of claim 117, wherein the amount of M1 macrophages or tumor-associated macrophages is measured directly or indirectly.

119. The use of claim 118, wherein the amount of M1 macrophages or tumor-associated macrophages is measured directly using flow cytometry, spatial transcriptomics, spatial proteomics, or combination thereof.

120. The use of claim 118, wherein the amount of M1 macrophages or tumor-associated macrophages is measured indirectly using nucleic acid or protein.

121. The use of claim 120, wherein the nucleic acid is measured using RNA-seq, RT-qPCR, qPCR, multiplex qPCR or RT-qPCR, microarray analysis, SAGE, MassARRAY technique, ISH, or a combination thereof.

122. The use of claim 121, wherein the amount of M1 macrophages or tumor-associated macrophages is measured using a marker gene approach or a deconvolution approach.

123. The use of claim 122, wherein the marker gene approach uses xCell.

124. The use of claim 122, wherein the deconvolution approach uses quanTIseq.

125. The use of any one of claims 88-102, wherein the macrophage biomarker in the sample from the patient is measured using nucleic acid or protein.

126. The use of claim 125, wherein the macrophage biomarker in the sample from the patient is determined using a nucleic acid expression level.

127. The use of claim 126, wherein the nucleic acid expression level is determined by RNA-seq, RT-qPCR, qPCR, multiplex qPCR or RT-qPCR, microarray analysis, SAGE, MassARRAY technique, ISH, or a combination thereof.

128. The use of claim 126 or 127, wherein the nucleic acid expression level is an mRNA expression level.

129. The use of claim 128, wherein the mRNA expression level is determined by RNA-seq.

130. The use of any one of claims 88-129, wherein the sample is a tissue sample, tumor sample, whole blood sample, a plasma sample, a serum sample, or a combination thereof.

131. The use of claim 130, wherein the sample is a tissue sample.

132. The use of claim 131, wherein the tissue sample is a tumor tissue sample.

133. The use of claim 132, wherein the tumor tissue sample contains tumor cells, tumor-infiltrating immune cells, stromal cells, normal adjacent tissue (NAT) cells, or a combination thereof.

134. The use of claim 132 or 133, wherein the tumor tissue sample is a biopsy.

135. The use of any one of claims 130-134, wherein the sample is an archival sample, a fresh sample, or a frozen sample.

136. The use of any one of claims 88-135, wherein the DLBCL is a germinal-center B-cell-like (GCB) or activated B-cell-like (ABC) cell-of-origin subgroup of DLBCL.

137. The use of any one of claims 88-136, wherein the DLBCL is a CD79a- and/or CD20-positive DLBCL.

138. The use of any one of claims 88-137, wherein the patient has not been previously treated for the DLBCL.

139. The use of any one of claims 88-138, wherein the patient has not been previously administered the immunoconjugate and the anti-CD20 antibody.

140. The use of any one of claims 88-139, wherein the anti-CD79b antibody comprises a heavy chain variable domain (VH) comprising the amino acid sequence of SEQ ID NO: 3 and a light chain variable domain (VL) comprising the amino acid sequence of SEQ ID NO: 4.

141. The use of any one of claims 88-140, wherein the anti-CD79b antibody comprises:
(a) a heavy chain comprising the amino acid sequence of SEQ ID NO: 13 and a light chain comprising the amino acid sequence of SEQ ID NO: 11;
(b) a heavy chain comprising the amino acid sequence of SEQ ID NO: 12 and a light chain comprising the amino acid sequence of SEQ ID NO: 14; or
(c) a heavy chain comprising the amino acid sequence of SEQ ID NO: 12 and a light chain comprising the amino acid sequence of SEQ ID NO: 11.

142. The use of any one of claims 88-141, wherein p is between 2 and 7, between 2 and 6, between 2 and 5, between 3 and 5, or between 3 and 4.

143. The use of claim 142, wherein p is 3.4.

144. The use of claim 142, wherein p is 3.5.

145. The use of any one of claims 88-144, wherein the immunoconjugate is polatuzumab vedotin.

146. The use of any one of claims 88-145, wherein the anti-CD20 antibody is a type I anti-CD20 antibody or a type II anti-CD20 antibody.

147. The use of claim 146, wherein the anti-CD20 antibody is a type I anti-CD20 antibody.

148. The use of claim 147, wherein the type I anti-CD20 antibody comprises the following CDRs:
(a) a CDR-H1 with an amino acid sequence of SEQ ID NO: 26;
(b) a CDR-H2 with an amino acid sequence of SEQ ID NO: 27;
(c) a CDR-H3 with an amino acid sequence of SEQ ID NO: 28;
(d) a CDR-L1 with an amino acid sequence of SEQ ID NO: 29;
(e) a CDR-L2 with an amino acid sequence of SEQ ID NO: 30; and
(f) a CDR-L3 with an amino acid sequence of SEQ ID NO: 31.

149. The use of claim 148, wherein the type I anti-CD20 antibody comprises a VH domain comprising an amino acid sequence of SEQ ID NO: 40 and a VL domain comprising an amino acid sequence of SEQ ID NO: 41.

150. The use of claim 149, wherein the type I anti-CD20 antibody is rituximab.

151. The use of any one of claims 145-150, wherein polatuzumab vedotin is administered at a dose of about 1.0 mg/kg to about 1.8 mg/kg.

152. The use of claim 151, wherein polatuzumab vedotin is administered at a dose of about 1.8 mg/kg.

153. The use of any one of claims 150-152, wherein rituximab is administered at a dose of about 375 mg/m².

154. The use of any one of claims 145-153, wherein polatuzumab vedotin and/or rituximab is administered intravenously.

155. The use of any one of claims 88-154, wherein the medicament is to be administered to the patient in combination with an effective amount of an additional therapeutic agent.

156. The use of claim 155, wherein the additional therapeutic agent is one or more of a chemotherapeutic agent, a corticosteroid, an anti-neoplastic agent, a growth inhibitory agent, an anti-angiogenic agent, a radiation therapy, a cytotoxic agent, or a combination thereof.

157. The use of claim 156, wherein the additional therapeutic agent is a chemotherapeutic agent and a corticosteroid.

158. The use of claim 156 or 157, wherein the chemotherapeutic agent is cyclophosphamide and/or doxorubicin.

159. The use of any one of claims 156-158, wherein the corticosteroid is prednisone, prednisolone, or methylprednisolone.

160. The use of claim 158 or 159, wherein cyclophosphamide is administered at a dose of about 375 mg/m² to about 750 mg/m².

161. The use of any one of claims 158-160, wherein doxorubicin is administered at a dose of about 25 mg/m² to about 50 mg/m².

162. The use of any one of claims 159-161, wherein
(a) prednisone is administered at a dose of about 100 mg;
(b) prednisolone is administered at a dose of about 100 mg; or
(c) methylprednisolone is administered at a dose of about 80 mg.

163. The use of any one of claims 158-162, wherein cyclophosphamide and/or doxorubicin are administered intravenously.

164. The use of any one of claims 159-163, wherein prednisone, prednisolone, or methylprednisolone is administered orally.

165. The use of any one of claims 159-164, wherein polatuzumab vedotin, rituximab, cyclophosphamide, doxorubicin, and/or prednisone, prednisolone, or methylprednisolone are administered in at least one 21-day cycle.

166. The use of claim 165, wherein:
(a) the polatuzumab vedotin, rituximab, cyclophosphamide, and/or doxorubicin are administered on day 1 of each 21-day cycle; and/or
(b) prednisone, prednisolone, or methylprednisolone is administered on days 1-5 of each 21-day cycle.

167. The use of any one of claims 165-166, wherein polatuzumab vedotin, rituximab, cyclophosphamide, doxorubicin, and/or prednisone, prednisolone, or methylprednisolone are administered for one, two, three, four, five, or six 21-day cycles.

168. An immunoconjugate and an anti-CD20 antibody for use in the treatment of a patient having a DLBCL and having an amount or level of a macrophage biomarker in a sample from the patient that is below a reference macrophage biomarker amount or level, wherein the immunoconjugate comprises the formula: 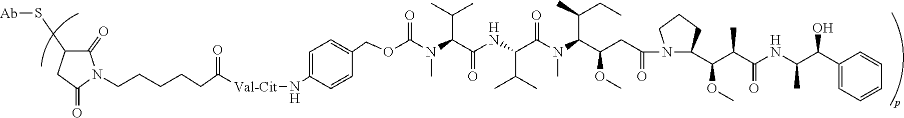
wherein Ab is an anti-CD79b antibody comprising: (i) an HVR-H1 that comprises the amino acid sequence of SEQ ID NO: 5; (ii) an HVR-H2 comprising the amino acid sequence of SEQ ID NO: 6; (iii) an HVR-H3 comprising the amino acid sequence of SEQ ID NO: 7; (iv) an HVR-L1 comprising the amino acid sequence of SEQ ID NO: 8; (v) an HVR-L2 comprising the amino acid sequence of SEQ ID NO: 9; and (vi) an HVR-L3 comprising the amino acid sequence of SEQ ID NO:10, and wherein p is between 1 and 8.

169. The immunoconjugate and the anti-CD20 antibody for use of claim 168, wherein the patient is a human patient.

170. The immunoconjugate and the anti-CD20 antibody for use of claim 168 or 169, wherein the reference macrophage biomarker amount or level is a pre-assigned macrophage biomarker amount or level.

171. The immunoconjugate and the anti-CD20 antibody for use of any one of claims 168-170, wherein the reference macrophage biomarker amount or level is an amount or level of a macrophage biomarker in a reference population.

172. The immunoconjugate and the anti-CD20 antibody for use of claim 171, wherein the amount or level of the macrophage biomarker in a reference population is a median amount or level of the macrophage biomarker of the reference population.

173. The immunoconjugate and the anti-CD20 antibody for use of claim 171, wherein the reference macrophage biomarker amount or level is an amount or level of a macrophage biomarker that is at the 25th percentile of the reference population.

174. The immunoconjugate and the anti-CD20 antibody for use of claim 171, wherein the reference macrophage biomarker amount or level is an amount or level of a macrophage biomarker that is at the 50th percentile of the reference population.

175. The immunoconjugate and the anti-CD20 antibody for use of claim 171, wherein the reference macrophage biomarker amount or level is an amount or level of a macrophage biomarker that is at the 75th percentile of the reference population.

176. The immunoconjugate and the anti-CD20 antibody for use of any one of claims 171-175, wherein the reference population is a population of patients having the DLBCL.

177. The immunoconjugate and the anti-CD20 antibody for use of claim 176, wherein the population of patients having the DLBCL was previously treated with the immunoconjugate and the anti-CD20 antibody.

178. The immunoconjugate and the anti-CD20 antibody for use of claim 176, wherein the population of patients having the DLBCL was previously treated with the anti-CD20 antibody.

179. The immunoconjugate and the anti-CD20 antibody for use of claim 176, wherein the reference macrophage biomarker amount or level is the amount or level of macrophage biomarker of the reference population prior to initiating treatment with the immunoconjugate and the anti-CD20 antibody.

180. The immunoconjugate and the anti-CD20 antibody for use of any one of claims 168-179, wherein the reference macrophage biomarker amount or level is an amount of macrophages as measured by gene expression.

181. The immunoconjugate and the anti-CD20 antibody for use of claim 180, wherein the amount of macrophages is between about 0% to about 56.5%.

182. The immunoconjugate and the anti-CD20 antibody for use of any one of claims 168-181, wherein the treatment achieves an improvement of PFS or OS.

183. The immunoconjugate and the anti-CD20 antibody for use of any one of claims 168-182, wherein the macrophage biomarker is an average of M1 macrophage gene signature set scores of one or more M1 macrophage gene signature sets.

184. The immunoconjugate and the anti-CD20 antibody for use of claim 183, wherein each M1 macrophage gene signature set score is an average of the expression level of one or more genes of an M1 macrophage gene signature set.

185. The immunoconjugate and the anti-CD20 antibody for use of claim 184, wherein each M1 macrophage gene signature set score is an average of the normalized expression level of one or more genes of an M1 macrophage gene signature set.

186. The immunoconjugate and the anti-CD20 antibody for use of claim 184 or 185, wherein the one or more M1 macrophage gene signature sets are:
(a) ACP2, ABCD1, C1QA, FDX1, CCL22, CD163, SCAMP2, ADAMDEC1, ARL8B, and HAMP;
(b) ACP2, ABCD1, FDX1, CCL8, CCL22, CD163, ADAMDEC1, TREM2, and HAMP;
(c) ACP2, ADRA2B, ALCAM, ABCD1, ATOX1, ATP6VOC, ATP6V1E1, BLVRA, C1QA, CD48, CD63, CLCN7, TPP1, CLTC, CCR1, CMKLR1, SLC31A1, COX5B, FCER1G, FDX1, FOLR2, FPR3, FTL, HEXB, HK3, IL10, IL12B, ITGAE, LAIR1, CXCL9, MMP19, NARS, NDUFS2, P2RX7, PDCL, MAPK13, PTGIR, PTPRA, RELA, CCL7, CCL8, CCL19, CCL22, SRC, STX4, TCEB1, TFRC, AGPS, MARCO, SNX3, CD84, USP14, ITGB1BP1, ATP6V1F, TRIP4, CD163, CIAO1, WTAP, ARHGEF11, ABI1, SCAMP2, ACTR2, BCAP31, ZMPSTE24, BCKDK, EXOC5, STIP1, UQCR11, SDS, LILRB4, OGFR, TFEC, FKBP15, DNAJC13, TDRD7, STX12, IL17RA, ABTB2, FAM32A, SIGLEC7, SIGLEC9, ADAMDEC1, CECR5, SLC25A24, NRBP1, MS4A4A, TREM2, OTUD4, PQLC2, HAUS2, ARL8B, NECAP2, WDR11, ZC3H15, CCDC47, UTP3, MRS2, HAMP, MRPL40, VPS33A, CORO7, LIMD2, TMX1, DOT1L, ADO, and ADCK2;
(d) ACP2, ADRA2B, ALCAM, TSPO, C3AR1, DAGLA, CALR, CHIT1, CYBB, CYC1, CYP19A1, DLAT, FCER1G, GP1BA, GPD1, IFNAR1, IL10, KCNJ5, KIFC3, MT2A, MYBPH, MYH11, MYO7A, P2RX7, PRDX1, RAB3IL1, RNH1, MRPL12, CCL1, CCL7, CCL8, CCL24, SRC, VIM, RRP1, MARCO, S1PR2, AP1M2, ACTR3, LILRB1, AFG3L2, SDS, LILRB4, EMILIN1, VSIG4, HSPB7, COQ2, ADAMDEC1, CECR5, WSB2, SLAMF8, DNASE2B, CLPB, MFSD7, and ADCK2;
(e) ACP2, ADCY3, ADRA2B, ALCAM, TSPO, C1QA, C1QB, C3AR1, DAGLA, CD63, CHIT1, CMKLR1, SLC31A1, CSF1, CSF1R, CYBB, CYC1, CYP19A1, FANCE, FCER1G, FDX1, FPR3, FTL, GP1BA, GPD1, HEXB, IL10, KCNJ1, KCNJ5, KIFC3, LAMP1, MMP19, MSR1, MT2A, MYBPH, MYO7A, P2RX7, PRDX1, RAB3IL1, MRPL12, CCL1, CCL7, CCL8, CCL18, CCL19, CCL24, SLC6A12, SPR, SRC, RRP1, MARCO, PKD2L1, S1PR2, CD163, LONP1, AP1M2, IGSF6, LILRB1, SDS, LILRB4, EMILIN1, VSIG4, TFEC, PHLDB1, CYFIP1, FKBP15, NCAPH, MYOF, HSPB7, ADAMDEC1, GLRX2, NDUFAF1, SPG21, MS4A4A, ATP6V1D, ATP6V1H, TREM2, PQLC2, TMEM70, PLEKHB2, TMEM33, SLAMF8, HAMP, DNASE2B, MYOZ1, LONRF3, CLPB, MFSD7, and ADCK2; and/or
(f) ACP2, ADCY3, ADRA2B, ALCAM, ABCD1, ANXA2, ATP6V1A, C1QA, C1QB, C3AR1, DAGLA, CD80, CD63, CHIT1, CMKLR1, SLC31A1, CSF1, CSF1R, CYBB, CYC1, CYP19A1, FANCE, FDX1, FPR2, FPR3, GPD1, HEXB, KCNJ1, KCNJ5, KIFC3, MMP19, MSR1, MT2A, MYBPH, P2RX7, MAPK13, S100A11, CCL1, CCL7, CCL8, CCL18, CCL19, CCL22, CCL24, SLC1A2, SLC6A12, SLC11A1, SIGLEC1, SRC, TIE1, MARCO, HYAL2, CD163, LONP1, IGSF6, LILRB1, CD300C, SDS, LILRB4, EMILIN1, VSIG4, PHLDB1, NCAPH, CLEC4E, MYOF, HSPB7, ADAMDEC1, GLRX2, MS4A4A, ATP6V1H, TREM2, TMEM70, TMEM33, KCNK13, SLAMF8, HAMP, DNASE2B, MYOZ1, MFSD7, ADO, ADCK2, and TBC1D16.

187. The immunoconjugate and the anti-CD20 antibody for use of any one of claims 168-182, wherein the macrophage biomarker is an average of tumor-associated macrophage gene signature set scores of one or more tumor-associated macrophage gene signature sets.

188. The immunoconjugate and the anti-CD20 antibody for use of claim 187, wherein each tumor-associated macrophage gene signature set score is an average of the expression level of one or more genes of a tumor-associated macrophage gene signature set.

189. The immunoconjugate and the anti-CD20 antibody for use of claim 188, wherein each tumor-associated macrophage gene signature set score is an average of the normalized expression level of one or more genes of a tumor-associated macrophage gene signature set.

190. The immunoconjugate and the anti-CD20 antibody for use of claim 188 or 189, wherein the one or more tumor-associated macrophage gene signature sets are:
(a) MARCO, ACP5, VSIG4, MRC1, MSR1, MCEMP1, CYP27A1, OLR1, GRN, GLIPR2, ARRDC4, C1QC, APOE, FOLR2, CTSD and SPP1.

191. The immunoconjugate and the anti-CD20 antibody for use of any one of claims 168-182, wherein the macrophage biomarker is a gene expression value.

192. The immunoconjugate and the anti-CD20 antibody for use of claim 187, wherein the gene expression value is a median gene expression value.

193. The immunoconjugate and the anti-CD20 antibody for use of claim 187 or 192, wherein the gene expression value is measured using a gene signature matrix.

194. The immunoconjugate and the anti-CD20 antibody for use of claim 193, wherein the gene signature matrix comprises the following genes:
(a) CD200, KLHL14, TCL1A, NRG1, EOMES, PPP2R2B, RNF165, WNT7A, CCR4, PDGFD, EBF1, FCGBP, PCDH9, MLC1, TSHZ2, S1PR5, NCALD, LAYN, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, ABCB4, CD5, HPGD, BLNK, PLCL1, HPSE, SLFN13, HOPX, CD1D, GNG7, TCF4, BANK1, FHIT, FCMR, GNG2, GFRA2, KBTBD11, RALGPS2, TSPOAP1, PLEKHF1, MEF2C, MAOA, TTYH2, HLA-DOB, DGAT2, FXYD6, TMCC3, MGAM, TTC38, LRRC32, ARHGAP24, STAT4, SLC7A8, CD72, FZD1, GK5, DYSF, PLTP, SMARCD3, FAM160B1, PDPN, AKAP2, ACVRL1, KCNJ15, ALDH1A2, ENPP2, COLEC12, PTGS1, TMEM170B, TREM2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, LILRA2, ACE, NUPR1, CISH, EREG, ADAMDEC1, RNASE6, CXCL3, VSIG4, CXCL2, CD86, LILRB4, SERPING1, SQOR, INHBA, and ICAM1; or
(b) CD200, KLHL14, TCL1A, NRG1, CYP4F3, EOMES, PPP2R2B, RNF165, WNT7A, CCR4, PDGFD, EBF1, FCGBP, PCDH9, MLC1, TSHZ2, S1PR5, NCALD, LAYN, CD248, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, NINJ2, ABCB4, CD5, HAL, HPGD, BLNK, PLCL1, CEP19, HPSE, SLFN13, HOPX, CD1D, GNG7, TMEM154, TCF4, BANK1, FHIT, FCMR, GNG2, GFRA2, KBTBD11, TECPR2, RALGPS2, TSPOAP1, PLEKHF1, MEF2C, MAOA, TTYH2, HLA-DOB, NRGN, DGAT2, FXYD6, TMCC3, MGAM, TTC38, LRRC32, ARHGAP24, PPP1R3B, STAT4, SLC7A8, CD72, FZD1, GK5, DYSF, PLTP, SMARCD3, FAM160B1, PDPN, AKAP2, ACVRL1, KCNJ15, CD36, ALDH1A2, ENPP2, COLEC12, PTGS1, TMEM170B, DOCK5, TREM2, C5AR2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, TLR4, LILRA2, ACE, TLR1, LRRK2, LY96, NUPR1, CISH, CSTA, EREG, ADAMDEC1, RNASE6, CXCL3, VSIG4, CXCL2, CD86, LILRB4, SERPING1, SQOR, INHBA, and ICAM1.

195. The immunoconjugate and the anti-CD20 antibody for use of claim 194, wherein the gene signature matrix consists of the following genes: CD200, KLHL14, TCL1A, NRG1, EOMES, PPP2R2B, RNF165, WNT7A, CCR4, PDGFD, EBF1, FCGBP, PCDH9, MLC1, TSHZ2, S1PR5, NCALD, LAYN, GCNT4, FASLG, TRAT1, ADAM6, GUCY1A3, LRRC4, TSPAN18, SBK1, ICOS, BTNL8, WNT5B, AUTS2, SH2D2A, ADGRG3, PNOC, SPIB, VPREB3, DPEP3, MME, ZBTB16, FOXP3, SEMA3G, CD8A, TOGARAM2, COLGALT2, ABCB1, STAP1, SAMD3, FAM46C, BLK, CTLA4, CD19, REPS2, RTKN2, POU2AF1, DAPK2, PYHIN1, NLRC3, GATM, KLRD1, AFF3, FCRLA, AATBC, REM2, YPEL1, TXK, CD8B, P2RX5, CEACAM1, BCL11A, ABCB4, CD5, HPGD, BLNK, PLCL1, HPSE, SLFN13, HOPX, CD1D, GNG7, TCF4, BANK1, FHIT, FCMR, GNG2, GFRA2, KBTBD11, RALGPS2, TSPOAP1, PLEKHF1, MEF2C, MAOA, TTYH2, HLA-DOB, DGAT2, FXYD6, TMCC3, MGAM, TTC38, LRRC32, ARHGAP24, STAT4, SLC7A8, CD72, FZD1, GK5, DYSF, PLTP, SMARCD3, FAM160B1, PDPN, AKAP2, ACVRL1, KCNJ15, ALDH1A2, ENPP2, COLEC12, PTGS1, TMEM170B, TREM2, ECM1, SLC1A3, ABHD5, MS4A4A, CLIC2, IL1R1, SLC2A6, GAS7, RNF144B, SLC6A12, FPR2, ADAM28, GRK3, KDM1B, MATK, LMO2, CFB, CCRL2, CLEC4A, LILRA2, ACE, NUPR1, CISH, EREG, ADAMDEC1, RNASE6, CXCL3, VSIG4, CXCL2, CD86, LILRB4, SERPING1, SQOR, INHBA, and ICAM1.

196. The immunoconjugate and the anti-CD20 antibody for use of any one of claims 193-195, wherein the gene signature matrix is used to determine a number of M1 macrophages or tumor-associated macrophages.

197. The immunoconjugate and the anti-CD20 antibody for use of any one of claims 168-182, wherein the macrophage biomarker is an amount of M1 macrophages or tumor-associated macrophages.

198. The immunoconjugate and the anti-CD20 antibody for use of claim 197, wherein the amount of M1 macrophages or tumor-associated macrophages is measured directly or indirectly.

199. The immunoconjugate and the anti-CD20 antibody for use of claim 198, wherein the amount of M1 macrophages or tumor-associated macrophages is measured directly using flow cytometry, spatial transcriptomics, spatial proteomics, or combination thereof.

200. The immunoconjugate and the anti-CD20 antibody for use of claim 198, wherein the amount of M1 macrophages or tumor-associated macrophages is measured indirectly using nucleic acid or protein.

201. The immunoconjugate and the anti-CD20 antibody for use of claim 200, wherein the nucleic acid is measured using RNA-seq, RT-qPCR, qPCR, multiplex qPCR or RT-qPCR, microarray analysis, SAGE, MassARRAY technique, ISH, or a combination thereof.

202. The immunoconjugate and the anti-CD20 antibody for use of claim 201, wherein the amount of M1 macrophages or tumor-associated macrophages is measured using a marker gene approach or a deconvolution approach.

203. The immunoconjugate and the anti-CD20 antibody for use of claim 202, wherein the marker gene approach uses xCell.

204. The immunoconjugate and the anti-CD20 antibody for use of claim 202, wherein the deconvolution approach uses quanTIseq.

205. The immunoconjugate and the anti-CD20 antibody for use of any one of claims 168-182, wherein the macrophage biomarker in the sample from the patient is measured using nucleic acid or protein.

206. The immunoconjugate and the anti-CD20 antibody for use of claim 205, wherein the macrophage biomarker in the sample from the patient is determined using a nucleic acid expression level.

207. The immunoconjugate and the anti-CD20 antibody for use of claim 206, wherein the nucleic acid expression level is determined by RNA-seq, RT-qPCR, qPCR, multiplex qPCR or RT-qPCR, microarray analysis, SAGE, MassARRAY technique, ISH, or a combination thereof.

208. The immunoconjugate and the anti-CD20 antibody for use of claim 206 or 207, wherein the nucleic acid expression level is an mRNA expression level.

209. The immunoconjugate and the anti-CD20 antibody for use of claim 208, wherein the mRNA expression level is determined by RNA-seq.

210. The immunoconjugate and the anti-CD20 antibody for use of any one of claims 168-209, wherein the sample is a tissue sample, tumor sample, whole blood sample, a plasma sample, a serum sample, or a combination thereof.

211. The immunoconjugate and the anti-CD20 antibody for use of claim 210, wherein the sample is a tissue sample.

212. The immunoconjugate and the anti-CD20 antibody for use of claim 211, wherein the tissue sample is a tumor tissue sample.

213. The immunoconjugate and the anti-CD20 antibody for use of claim 212, wherein the tumor tissue sample contains tumor cells, tumor-infiltrating immune cells, stromal cells, normal adjacent tissue (NAT) cells, or a combination thereof.

214. The immunoconjugate and the anti-CD20 antibody for use of claim 212 or 213, wherein the tumor tissue sample is a biopsy.

215. The immunoconjugate and the anti-CD20 antibody for use of any one of claims 210-214, wherein the sample is an archival sample, a fresh sample, or a frozen sample.

216. The immunoconjugate and the anti-CD20 antibody for use of any one of claims 168-215, wherein the DLBCL is a germinal-center B-cell-like (GCB) or activated B-cell-like (ABC) cell-of-origin subgroup of DLBCL.

217. The immunoconjugate and the anti-CD20 antibody for use of any one of claims 168-216, wherein the DLBCL is a CD79a- and/or CD20-positive DLBCL.

218. The immunoconjugate and the anti-CD20 antibody for use of any one of claims 168-217, wherein the patient has not been previously treated for the DLBCL.

219. The immunoconjugate and the anti-CD20 antibody for use of any one of claims 168-218, wherein the patient has not been previously administered the immunoconjugate and the anti-CD20 antibody.

220. The immunoconjugate and the anti-CD20 antibody for use of any one of claims 168-219, wherein the anti-CD79b antibody comprises a heavy chain variable domain (VH) comprising the amino acid sequence of SEQ ID NO: 3 and a light chain variable domain (VL) comprising the amino acid sequence of SEQ ID NO: 4.

221. The immunoconjugate and the anti-CD20 antibody for use of any one of claims 168-220, wherein the anti-CD79b antibody comprises:
(a) a heavy chain comprising the amino acid sequence of SEQ ID NO: 13 and a light chain comprising the amino acid sequence of SEQ ID NO: 11;
(b) a heavy chain comprising the amino acid sequence of SEQ ID NO: 12 and a light chain comprising the amino acid sequence of SEQ ID NO: 14; or
(c) a heavy chain comprising the amino acid sequence of SEQ ID NO: 12 and a light chain comprising the amino acid sequence of SEQ ID NO: 11.

222. The immunoconjugate and the anti-CD20 antibody for use of any one of claims 168-221, wherein p is between 2 and 7, between 2 and 6, between 2 and 5, between 3 and 5, or between 3 and 4.

223. The immunoconjugate and the anti-CD20 antibody for use of claim 222, wherein p is 3.4.

224. The immunoconjugate and the anti-CD20 antibody for use of claim 222, wherein p is 3.5.

225. The immunoconjugate and the anti-CD20 antibody for use of any one of claims 168-224, wherein the immunoconjugate is polatuzumab vedotin.

226. The immunoconjugate and the anti-CD20 antibody for use of any one of claims 168-225, wherein the anti-CD20 antibody is a type I anti-CD20 antibody or a type II anti-CD20 antibody.

227. The immunoconjugate and the anti-CD20 antibody for use of claim 226, wherein the anti-CD20 antibody is a type I anti-CD20 antibody.

228. The immunoconjugate and the anti-CD20 antibody for use of claim 227, wherein the type I anti-CD20 antibody comprises the following CDRs:
(a) a CDR-H1 with an amino acid sequence of SEQ ID NO: 26;
(b) a CDR-H2 with an amino acid sequence of SEQ ID NO: 27;
(c) a CDR-H3 with an amino acid sequence of SEQ ID NO: 28;
(d) a CDR-L1 with an amino acid sequence of SEQ ID NO: 29;
(e) a CDR-L2 with an amino acid sequence of SEQ ID NO: 30; and
(f) a CDR-L3 with an amino acid sequence of SEQ ID NO: 31.

229. The immunoconjugate and the anti-CD20 antibody for use of claim 228, wherein the type I anti-CD20 antibody comprises a VH domain comprising an amino acid sequence of SEQ ID NO: 40 and a VL domain comprising an amino acid sequence of SEQ ID NO: 41.

230. The immunoconjugate and the anti-CD20 antibody for use of claim 229, wherein the type I anti-CD20 antibody is rituximab.

231. The immunoconjugate and the anti-CD20 antibody for use of any one of claims 225-230, wherein polatuzumab vedotin is for use at a dose of about 1.0 mg/kg to about 1.8 mg/kg.

232. The immunoconjugate and the anti-CD20 antibody for use of claim 231, wherein polatuzumab vedotin is for use at a dose of about 1.8 mg/kg.

233. The immunoconjugate and the anti-CD20 antibody for use of any one of claims 230-232, wherein rituximab is for use at a dose of about 375 mg/m².

234. The immunoconjugate and the anti-CD20 antibody for use of any one of claims 225-233, wherein polatuzumab vedotin and/or rituximab is for intravenous use.

235. The immunoconjugate and the anti-CD20 antibody for use of any one of claims 168-234, wherein the treatment further comprises use of an effective amount of an additional therapeutic agent.

236. The immunoconjugate and the anti-CD20 antibody for use of claim 235, wherein the additional therapeutic agent is one or more of a chemotherapeutic agent, a corticosteroid, an anti-neoplastic agent, a growth inhibitory agent, an anti-angiogenic agent, a radiation therapy, a cytotoxic agent, or a combination thereof.

237. The immunoconjugate and the anti-CD20 antibody for use of claim 236, wherein the additional therapeutic agent is a chemotherapeutic agent and a corticosteroid.

238. The immunoconjugate and the anti-CD20 antibody for use of claim 236 or 237, wherein the chemotherapeutic agent is cyclophosphamide and/or doxorubicin.

239. Th immunoconjugate and the anti-CD20 antibody for use of any one of claims 236-238, wherein the corticosteroid is prednisone, prednisolone, or methylprednisolone.

240. The immunoconjugate and the anti-CD20 antibody for use of claim 238 or 239, wherein cyclophosphamide is for use at a dose of about 375 mg/m² to about 750 mg/m².

241. The immunoconjugate and the anti-CD20 antibody for use of any one of claims 238-240, wherein doxorubicin is for use at a dose of about 25 mg/m² to about 50 mg/m².

242. The immunoconjugate and the anti-CD20 antibody for use of any one of claims 239-241, wherein
(a) prednisone is for use at a dose of about 100 mg;
(b) prednisolone is for use at a dose of about 100 mg; or
(c) methylprednisolone for use at a dose of about 80 mg.

243. The immunoconjugate and the anti-CD20 antibody for use of any one of claims 238-242, wherein cyclophosphamide and/or doxorubicin is for intravenous use.

244. The immunoconjugate and the anti-CD20 antibody for use of any one of claims 239-243, wherein prednisone, prednisolone, or methylprednisolone is for oral use.

245. The immunoconjugate and the anti-CD20 antibody for use of any one of claims 225-244, wherein polatuzumab vedotin, rituximab, cyclophosphamide, doxorubicin and/or prednisone, prednisolone, or methylprednisolone are for use in at least one 21-day cycle.

246. The immunoconjugate and the anti-CD20 antibody for use of claim 245, wherein:
(a) the polatuzumab vedotin, rituximab, cyclophosphamide, and/or doxorubicin are for use on day 1 of each 21-day cycle; and/or
(b) prednisone, prednisolone, or methylprednisolone is for use on days 1-5 of each 21-day cycle.

247. The immunoconjugate and the anti-CD20 antibody for use of claim 245 or 246, wherein polatuzumab vedotin, rituximab, cyclophosphamide, doxorubicin, and/or prednisone, prednisolone, or methylprednisolone are for use for one, two, three, four, five, or six 21-day cycles.

248. A method of identifying, diagnosing, and/or predicting whether a patient having a diffuse large B-cell lymphoma (DLBCL) may benefit from a treatment comprising an immunoconjugate, an anti-CD20 antibody, a chemotherapeutic agent, and a corticosteroid, the method comprising measuring a macrophage biomarker in a sample from the patient, wherein an amount or level of the macrophage biomarker in the sample that is below a reference macrophage biomarker amount or level identifies, diagnoses, and/or predicts the patient as one who may benefit from the treatment comprising the immunoconjugate, the anti-CD20 antibody, the chemotherapeutic agent, and the corticosteroid, wherein the immunoconjugate comprises the formula: 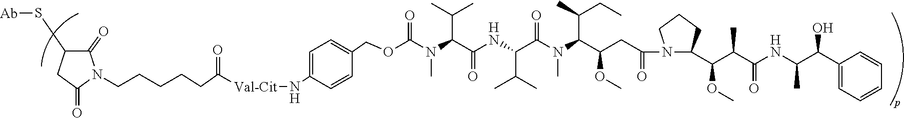
wherein Ab is an anti-CD79b antibody comprising a heavy chain comprising the amino acid sequence of SEQ ID NO: 12 and a light chain comprising the amino acid sequence of SEQ ID NO: 11 and wherein p is 3.5,
wherein the anti-CD20 antibody is rituximab,
wherein the chemotherapeutic agent comprises cyclophosphamide and doxorubicin, and
wherein the corticosteroid comprises prednisone.

249. A method of selecting a therapy for a patient having a DLBCL, the method comprising measuring a macrophage biomarker in a sample from the patient, wherein an amount or level of the macrophage biomarker in the sample that is below a reference macrophage biomarker amount or level identifies the patient as one who may benefit from a treatment comprising an immunoconjugate, an anti-CD20 antibody, a chemotherapeutic agent, and a corticosteroid, wherein the immunoconjugate comprises the formula: 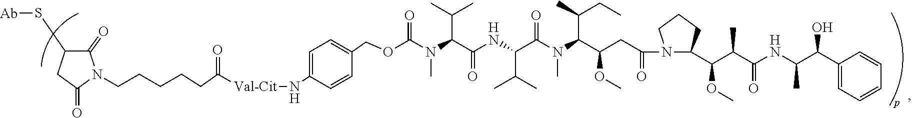
wherein Ab is an anti-CD79b antibody comprising a heavy chain comprising the amino acid sequence of SEQ ID NO: 12 and a light chain comprising the amino acid sequence of SEQ ID NO: 11 and wherein p is 3.5,
wherein the anti-CD20 antibody is rituximab,
wherein the chemotherapeutic agent comprises cyclophosphamide and doxorubicin, and
wherein the corticosteroid comprises prednisone.

250. The method of claim 248 or 249, wherein the amount or level of the macrophage biomarker from the patient is below the reference macrophage biomarker amount or level, and the method further comprises administering to the patient an effective amount of the immunoconjugate, an effective amount of the anti-CD20 antibody, an effective amount of the chemotherapeutic agent, and an effective amount of the corticosteroid.

251. A method of treating a patient having a DLBCL, the method comprising:
(a) measuring a macrophage biomarker in a sample from the patient, wherein the amount or level of the macrophage biomarker in the sample is below a reference macrophage biomarker amount or level, and
(b) administering an effective amount of an immunoconjugate, an effective amount of an anti-CD20 antibody, an effective amount of a chemotherapeutic agent, and an effective amount of a corticosteroid, to the patient based on the macrophage biomarker measured in step (a), and wherein the immunoconjugate comprises the formula: 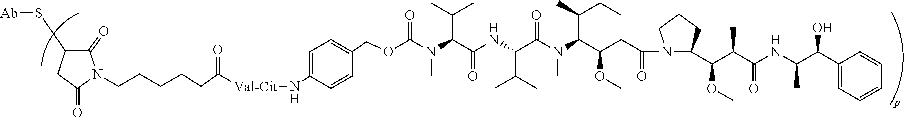
wherein Ab is an anti-CD79b antibody comprising a heavy chain comprising the amino acid sequence of SEQ ID NO: 12 and a light chain comprising the amino acid sequence of SEQ ID NO: 11 and wherein p is 3.5,
wherein the anti-CD20 antibody is rituximab,
wherein the chemotherapeutic agent comprises cyclophosphamide and doxorubicin, and
wherein the corticosteroid comprises prednisone.

252. A method of treating a patient having a DLBCL, the method comprising administering to the patient an effective amount of an immunoconjugate, an effective amount of an anti-CD20 antibody, an effective amount of a chemotherapeutic agent, and an effective amount of a corticosteroid, wherein prior to treatment the amount or level of a macrophage biomarker in a sample from the patient has been determined to be below a reference macrophage biomarker amount or level, and wherein the immunoconjugate comprises the formula: 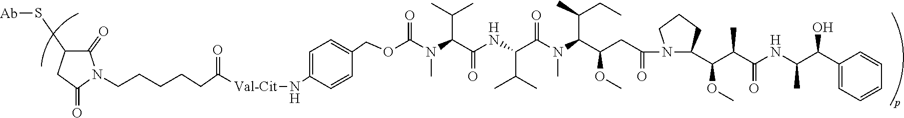
wherein Ab is an anti-CD79b antibody comprising a heavy chain comprising the amino acid sequence of SEQ ID NO: 12 and a light chain comprising the amino acid sequence of SEQ ID NO: 11 and wherein p is 3.5,
wherein the anti-CD20 antibody is rituximab,
wherein the chemotherapeutic agent comprises cyclophosphamide and doxorubicin, and
wherein the corticosteroid comprises prednisone.

253. A method of treating a patient having a DLBCL and having an amount or level of a macrophage biomarker in a sample from the patient that is below a reference macrophage biomarker amount or level comprising administering to the patient an effective amount of an immunoconjugate, an effective amount of an anti-CD20 antibody, an effective amount of a chemotherapeutic agent, and an effective amount of a corticosteroid, wherein the immunoconjugate comprises the formula: 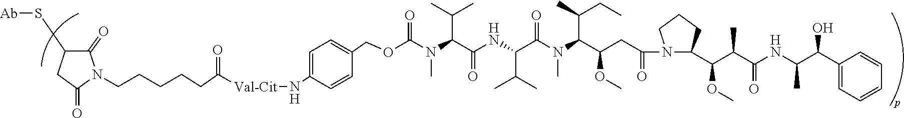
wherein Ab is an anti-CD79b antibody comprising a heavy chain comprising the amino acid sequence of SEQ ID NO: 12 and a light chain comprising the amino acid sequence of SEQ ID NO: 11 and wherein p is 3.5,
wherein the anti-CD20 antibody is rituximab,
wherein the chemotherapeutic agent comprises cyclophosphamide and doxorubicin, and
wherein the corticosteroid comprises prednisone.

254. The method of any one of claims 248-253, wherein the immunoconjugate is polatuzumab vedotin.

255. The method of any one of claims 248-254, wherein the immunoconjugate is administered at a dose of about 1.8 mg/kg, the rituximab is administered at a dose of about 375 mg/m², the cyclophosphamide is administered at a dose of about 750 mg/m², the doxorubicin is administered at a dose of about 50 mg/m², and the prednisone is administered at a dose of about 100 mg.
